(19) United States
(12) Patent Application Publication
(10) Pub. No.: US 2026/0297044 A1
(43) Pub. Date: Oct. 1, 2026

(54) INHIBITORS OF SPINSTER HOMOLOG 2 (SPNS2) FOR USE IN THERAPY

(71) Applicants: University of Virginia Patent Foundation, Charlottesville, VA (US); Virginia Tech Intellectual Properties, Inc., Blacksburg, VA (US)

(72) Inventors: Kevin R. Lynch, Charlottesville, VA (US); Yugesh Kharel, Charlottesville, VA (US); Tao Huang, Charlottesville, VA (US); Webster L. Santos, Blacksburg, VA (US); Christopher Shrader, Blacksburg, VA (US); Kyle Dunnavant, Christiansburg, VA (US); Ariel Louise Burgio, Christiansburg, VA (US); Andrew D. Bage, Blacksburg, VA (US)

(21) Appl. No.: 19/168,683

(22) PCT Filed: Mar. 22, 2024
(86) PCT No.: PCT/US2024/021158

§ 371 Date: Sep. 24, 2025

Related U.S. Application Data
(60) Provisional application No. 63/492,062, filed on Mar. 24, 2023.
(60) Provisional application No. 63/512,698, filed on Jul. 10, 2023.

(51) Int. Cl. C07D 295/215; A61K 31/397; A61K 31/423; A61K 31/495; A61K 31/496; A61K 31/498; A61K 31/499; A61K 31/506; A61P 37/06; C07D 213/65; C07D 213/75; C07D 235/24; C07D 239/42; C07D 241/38; C07D 295/195; C07D 413/12; C07D 487/10

(52) U.S. Cl. CPC C07D 295/215; A61K 31/397; A61K 31/423; A61K 31/495; A61K 31/496; A61K 31/498; A61K 31/499; A61K 31/506; A61P 37/06; C07D 213/65; C07D 213/75; C07D 235/24; C07D 239/42; C07D 241/38; C07D 295/195; C07D 413/12; C07D 487/10

(57) ABSTRACT

The present disclosure provides SPNS2 inhibitor compounds according to Formula I: where T, U, V, W, X, R¹, R², and m are defined herein. The present disclosure also provides pharmaceutically acceptable salts, and/or tautomers of Formula I compounds as described in the disclosure. Also provided are their methods of use in therapy, such as in diseases for which immunomodulation and/or anti-fibrotic activity is indicated.

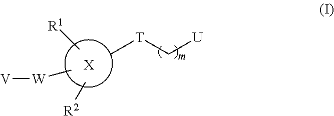

This application claims the benefit of priority to U.S. Provisional Patent Application No. 63/492,062, filed Mar. 24, 2023, and U.S. Provisional Patent Application No. 63/512,698, filed Jul. 10, 2023, which applications are incorporated in their entirety as if fully set forth herein.

### GOVERNMENT LICENSE RIGHTS

This invention was made with government support under grant #RO1 AI144026 awarded by the National Institutes of Health. The government has certain rights in the invention.

### BACKGROUND

Sphingosine 1-phosphate (S1P) is a chemotactic lipid. This role requires compartmentalization, which is provided in part by cell surface S1P transporters. There are two well characterized S1P transporters, SPNS2 (endothelium, microglia) and MFSD2B (erythrocytes, platelets). When S1P release is coupled with S1P degradation in tissue parenchyma, a differential is generated between the extracellular (high) and intracellular (low) S1P concentrations. The S1P gradient in blood functions to maintain endothelial barrier integrity, while other S1P concentration gradients control immune cell positioning.

S1P is particularly important for egress of lymphocytes from secondary lymphoid tissue into efferent lymph cells. S1P receptor modulator (SRM) drugs mask lymphocytes from S1P by desensitizing S1P1 receptors. However, SRM agonist activity at endothelial and cardiac S1P1 receptors drives adverse events such as first dose bradycardia and macular edema. Therefore, other methods to modulate immune cell trafficking by interdicting S1P signaling are needed.

### SUMMARY

The present disclosure satisfies this need and others by providing, in various embodiments, compounds and their pharmaceutically acceptable salts conforming to Formula I as SPNS2 inhibitors that avoid on-target adverse activity:

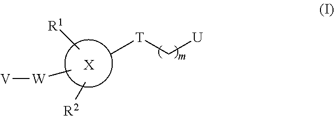

X is a C₆-C₁₀-aryl or 5- to 10-membered heteroaryl (wherein 1 to 4 heteroaryl ring members are independently selected from N, O, and S).

R¹ and R² are independently selected from the group consisting of H, C₁-C₆-alkyl, C₁-C₆-alkoxy, C₁-C₆-haloalkoxy, C₃-C₈-cycloalkyl, C₁-C₆-haloalkyl, CN, and halo.

W is a bond, O, NH, —NHC(O)—, or —O—(N═)C(R)— (wherein R is H or C₁-C₆-alkyl).

In some embodiments, U is (A) an optionally fused, bridged, or spiro-fused 6- to 8-membered heterocycloalkyl wherein 2 ring atoms are N. In these embodiments, V is selected from the group consisting of H, C₁-C₁₄-alkyl, C₂-C₁₂-alkenyl, (C₆-C₁₀)aryl, (C₆-C₁₀)heteroaryl, —C₁-C₁₀-alkyl-(C₆-C₁₀)aryl, —C₂-C₁₂-alkenyl-(C₆-C₁₀)aryl, —C₁-C₁₀-alkyl-(C₃-C₈)cycloalkyl, -(3- to 14-membered heterocycloalkyl) (wherein 1-4 heterocycloalkyl members are independently selected from N, O, and S), —(C₁-C₁₀)alkyl-(3- to 14-membered heterocycloalkyl) (wherein 1-4 heterocycloalkyl members are independently selected from N, O, and S).

In other embodiments, U is (B) a monocyclic 4- to 7-membered heterocycloalkyl (wherein 1-4 heterocycloalkyl members are independently selected from N, O, and S). In these embodiments, V is selected from the group consisting of H, —C₁-C₁₄-alkyl, —C₁-C₁₀-alkyl-O—(C₃-C₁₄)cycloalkyl, —C₁-C₁₀-alkyl-O—(C₆-C₁₀)aryl, —C₁-C₁₀-alkyl-NR^{x}—(C₆-C₁₀)aryl, —C₁-C₁₀-alkyl-NR^{x}C(O)(C₆-C₁₀)aryl, —C₁-C₁₀-alkyl-C(O)NR^{x}(C₆-C₁₀)aryl, —C₂-C₁₂-alkenyl-O—(C₆-C₁₀)aryl, —C₁-C₁₀-alkyl-O—(C₆-C₁₀)heteroaryl (wherein 1 to 4 heteroaryl ring members are independently selected from N, O, and S), and —C₂-C₁₂-alkenyl-O—(C₆-C₁₀)heteroaryl (wherein 1 to 4 heteroaryl ring members are independently selected from N, O, and S).

T is —C(O)— or —NR^{x}C(O)—.

R^{x} is H or C₁-C₆-alkyl.

m is an integer selected from 0, 1, 2, 3, 4, 5, and 6.

In Formula I, each alkyl, alkoxy, alkenyl, aryl, cycloalkyl, heterocycloalkyl, and heteroaryl is optionally substituted with 1-5 substituents independently selected from the group consisting of hydroxy, halo, C₁-C₆-alkyl, C₁-C₆-haloalkoxy, C₁-C₆-haloalkyl, —NR′₂, —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkoxy, C₃-C₁₄-cycloalkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl).

Each R′ is independently selected from the group consisting of C₁-C₆-alkyl, C₂-C₆-alkenyl, C₂-C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S.

Another embodiment of the disclosure is a pharmaceutical composition comprising a compound or a pharmaceutically acceptable salt thereof as described herein.

The disclosure also provides, in an embodiment, a method of inhibiting spinster homolog 2 (SPNS2), comprising contacting SPNS2 with an effective amount of a compound or a pharmaceutically acceptable salt thereof as described herein.

In still another embodiment, the present disclosure provides a method of treating a patient afflicted by a neoplastic disease, comprising administering to the patient a therapeutically effective amount of a compound or a pharmaceutically acceptable salt thereof as described herein.

In still another embodiment, the present disclosure provides a method of treating a patient afflicted by an allergic disease, comprising administering to the patient a therapeutically effective amount of a compound or a pharmaceutically acceptable salt thereof as described herein.

In another embodiment, the disclosure provides a method of treating a patient afflicted with an autoimmune disease, comprising administering to the patient a therapeutically effective amount of a compound or a pharmaceutically acceptable salt thereof as described herein.

In another embodiment, the disclosure provides a method of treating a patient afflicted with a fibrotic disease, comprising administering to the patient a therapeutically effective amount of a compound or a pharmaceutically acceptable salt thereof as described herein.

### BRIEF DESCRIPTION OF THE DRAWINGS

FIG. 1. shows a representative data set for Spns2-dependent S1P transport blockers (STBs) in the EAE (Experimental Autoimmune Encephalomyelitis) model of multiple sclerosis. Mice (female, C57BL/6 strain, 11 weeks old) were vaccinated with MOG35-55 peptide and treated with pertussis toxin on day 0. As animals became symptomatic, they were assigned randomly to treatment groups (N=12) and the clinical scores determined until day 35. Treatments were administered once daily by oral gavage. Positive control (FTY720, which is fingolimod) dose was 3 mg/kg/d. STBs Compd A and Compd B, two representative species of the present disclosure, were administered at 30 and 10 mg/kg/d respectively.

### DETAILED DESCRIPTION

Correct temporal positioning of immune cells is necessary for a properly functioning immune system. The chemotactic lipid, sphingosine 1-phosphate (S1P), is required for the correct positioning of immune cells. Lymphocyte egress from secondary lymphoid tissues such as lymph nodes is, for example, dependent on S1P signaling. S1P's role in lymphocyte trafficking was discovered when the mechanism of action of the immunosuppressive drug fingolimod (FTY720) was investigated. Fingolimod's active metabolite, phospho-FTY720, desensitizes lymphocyte S1P1 receptors; thereby, rendering these cells unable to detect S1P¹. The resulting lymphopenia is a pharmacodynamic marker of such S1P1 receptor agonists. Although fingolimod and other S1P receptor modulators are medicines currently used for treating autoimmune diseases such as relapsing remitting multiple sclerosis², this drug class has several on target liabilities including initial dose bradycardia and compromised endothelial barrier function². Therefore, alternative strategies to modulate the immune system at the level of S1P signaling are needed. The present disclosure meets this need.

While S1P is synthesized ubiquitously, its intracellular accumulation is limited by degradation and export. In lymph nodes (LN), brisk catabolic activity by, for example, S1P lyase keeps S1P concentrations low¹. The S1P in lymph is nearly undetectable in mouse strains deficient in the S1P transporter SPNS2, suggesting that lymph endothelial cells extrude S1P into lymph via SPNS2³, resulting in a lymph—LN S1P concentration gradient. Plasma S1P gradients are likewise maintained by prominent S1P catabolic activity in tissue parenchyma (excepting blood) coupled with the extrusion of S1P into plasma by red blood cells (RBCs). Mice strains rendered deficient in the S1P transporter MFSd2b, have extraordinarily high RBC S1P and diminished plasma S1P, indicating that MFSd2b, which is distantly related to SPNS2, provides S1P to plasma^{4,5}. Unlike SPNS2 null mice, Mfsd2b null mice do not have diminished numbers for circulating lymphocytes⁴. The plasma-tissue S1P concentration gradient is important for maintaining endothelial barrier integrity^{6,7}.

The role of the catabolic enzyme S1P lyase in maintaining low LN S1P predicts that S1P lyase inhibitors will eliminate the lymph-LN S1P gradient, which will modulate the immune system by disrupting lymphocyte trafficking analogous to S1P1 agonists. Indeed, S1P lyase deficiency, whether accomplished through genetic manipulation of mice or S1P lyase inhibitor administration, raises S1P levels in tissues, including lymph nodes, with a resulting lymphopenia^{1,8}. However, administering a selective S1P lyase inhibitor to rats and inducing global deletion of the S1P lyase gene (Sgpl1) in mice were both found to be nephrotoxic⁸.

Mice rendered deficient in SPNS2 either through germ line or endothelium-specific deletion of Spns2, have nearly undetectable (i.e. >10-fold decrease) S1P levels in thoracic duct lymph, but the vascular S1P gradient is less affected (SPNS2 null mouse strains are variously reported as having a 0-45% reduction in plasma S1P⁹⁻¹²). These results, when considered in the context of the accepted mechanism of action of S1P receptor modulators (vide supra), leads to the prediction that SPNS2 inhibitors, like S1P lyase inhibitors, will diminish the lymph S1P gradient and thereby inhibit lymphocyte trafficking and recapitulate the therapeutic efficacy of S1P1 receptor agonists. Indeed, studies with the present inventors' previously disclosed inhibitors of SPNS2-dependent S1P transport document that numbers of circulating lymphocytes in mice and rats are diminished as a function of administration of said inhibitors^{13,14}.

Use of an SPNS2 inhibitor in immuno-oncology can arise from another mouse genetics study¹⁵. In a screen of 810 mouse strains with different germ line gene deletions, Spns2^{-/-} mice were found to have remarkably low metastatic colonization of the lungs when injected intravenously with B16-F10 melanoma cells. This effect was observed with other lung metastatic colonization models and in similar models in liver. As expected, the total number of immune cells in the lung was reduced in the lymphopenic Spns2^{-/-} mice, but the lung resident population was proportionally enriched in natural killer (NK) and CD8⁺ effector cells¹⁵.

Use of an Spns2 inhibitor in anti-fibrotic therapy can arise from another study employing both mutant mouse strains and one of the present inventors' Spns2 inhibitors¹⁶. Specifically, in two models of kidney fibrosis, mice rendered deficient in Spns2 in kidney pericytes as well as mice treated with an Spns2 inhibitor (SLF1081851)¹³, had significantly less fibrosis and improved function of the injured kidney¹⁶.

Thus, results from the study of mice rendered deficient in Spns2 indicate that SPNS2 inhibitors will be immunomodulatory. The SPNS2 inhibitors of the disclosure recapitulate the SPNS2 null phenotype regarding immunomodulation, and therefore they enable S1P transport inhibition as a viable therapeutic strategy as well as providing heretofore unavailable chemical biology tools to explore S1P physiology in vivo.

### Definitions

“Alkyl” refers to straight or branched chain hydrocarbyl including from 1 to about 20 carbon atoms. For instance, an alkyl can have from 1 to 14 carbon atoms, 1 to 10 carbon atoms, or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂C H(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, and the like. Thus, alkyl groups include primary alkyl groups, secondary alkyl groups, and tertiary alkyl groups. An alkyl group can be unsubstituted or optionally substituted with one or more substituents as described herein below.

The phrase “substituted alkyl” refers to alkyl substituted at one or more positions, for example, 1, 2, 3, 4, 5, or even 6 positions, which substituents are attached at any available atom to produce a stable compound, with substitution as described herein. “Optionally substituted alkyl” refers to alkyl or substituted alkyl.

A “haloalkyl” is an alkyl, as defined herein, that is substituted with at least one, such as 1-8, halo substituents.

Each of the terms “halogen,” “halide,” and “halo” refers to —F, —Cl, —Br, or —I.

The term “alkenyl” refers to straight or branched chain hydrocarbyl groups including from 2 to about 20 carbon atoms having 1-3, 1-2, or at least one carbon to carbon double bond. An alkenyl group can be unsubstituted or optionally substituted with one or more substituents as described herein below.

“Substituted alkenyl” refers to alkenyl substituted at 1 or more, e.g., 1, 2, 3, 4, 5, or even 6 positions, which substituents are attached at any available atom to produce a stable compound, with substitution as described herein. “Optionally substituted alkenyl” refers to alkenyl or substituted alkenyl.

“Alkyne or “alkynyl” refers to a straight or branched chain unsaturated hydrocarbon having the indicated number of carbon atoms and at least one triple bond. Examples of a (C₂-C₈)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein below.

“Substituted alkynyl” refers to an alkynyl substituted at 1 or more, e.g., 1, 2, 3, 4, 5, or even 6 positions, which substituents are attached at any available atom to produce a stable compound, with substitution as described herein. “Optionally substituted alkynyl” refers to alkynyl or substituted alkynyl.

The term “alkoxy” refers to an —O-alkyl group having the indicated number of carbon atoms. For example, a (C₁-C₆)alkoxy group includes —O-methyl, —O-ethyl, —O-propyl, —O-isopropyl, —O-butyl, —O-sec-butyl, —O-tert-butyl, —O-pentyl, —O-isopentyl, —O-neopentyl, —O-hexyl, —O-isohexyl, and —O-neohexyl.

A “haloalkoxy” is an alkoxy, as defined herein, that is substituted with at least one, such as 1-8, halo substituents.

The term “cycloalkyl” refers to a monocyclic, bicyclic, tricyclic, or polycyclic, 3- to 14-membered ring system, which is either saturated, such as “cycloalkyl,” or unsaturated, such as “cycloalkenyl.” The term “cycloalkenyl” refers specifically to cyclic alkenyl, such as C₃-C₆-cycloalkenyl. The cycloalkyl may be attached via any atom. Cycloalkyl, for instance, also contemplates fused rings wherein, for instance, a cycloalkyl is fused to an aryl or heteroaryl ring as defined herein. Representative examples of cycloalkyl include, but are not limited to, cyclopropyl, cyclobutyl, cyclopentyl, cyclohexyl, cyclopropenyl, cyclobutenyl, cyclopentenyl, and cyclohexenyl. A cycloalkyl group can be unsubstituted or optionally substituted with one or more substituents as described herein.

“Substituted cycloalkyl” refers to cycloalkyl substituted at 1 or more, e.g., 1, 2, 3, 4, 5, or even 6 positions, which substituents are attached at any available atom to produce a stable compound, with substitution as described herein. “Optionally substituted cycloalkyl” refers to cycloalkyl or substituted cycloalkyl.

“Aryl” when used alone or as part of another term means a carbocyclic aromatic group whether or not fused having the number of carbon atoms designated or if no number is designated, up to 14 carbon atoms, such as a C₆-C₁₄-aryl. Particular aryl groups are phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). A particular aryl is phenyl. “Aryl” also includes aromatic ring systems that are optionally fused with a cycloalkyl ring, as herein defined. An aryl group can be unsubstituted or optionally substituted with one or more substituents as described herein below.

A “substituted aryl” is an aryl that is independently substituted with one or more substituents attached at any available atom to produce a stable compound, wherein the substituents are as described herein. “Optionally substituted aryl” refers to aryl or substituted aryl.

The term “heteroatom” refers to N, O, and S. Disclosed compounds that contain N or S atoms can be optionally oxidized to the corresponding N-oxide, sulfoxide, or sulfone compounds.

“Heteroaryl,” alone or in combination with any other moiety described herein, refers to a monocyclic aromatic ring structure containing 5 to 10, such as 5 or 6 ring atoms, or a bicyclic aromatic group having 8 to 10 atoms, containing one or more, such as 1-4, 1-3, or 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A heteroaryl group can be unsubstituted or optionally substituted with one or more substituents as described herein below.

A “substituted heteroaryl” is a heteroaryl that is independently substituted, unless indicated otherwise, with one or more, e.g., 1, 2, 3, 4 or 5, also 1, 2, or 3 substituents, also 1 substituent, attached at any available atom to produce a stable compound, wherein the substituents are as described herein. “Optionally substituted heteroaryl” refers to heteroaryl or substituted heteroaryl.

“Heterocycloalkyl” means a saturated or unsaturated non-aromatic monocyclic, bicyclic, tricyclic or polycyclic ring system that has from 3 to 14, such as 3 to 6, atoms in which from 1 to 3 carbon atoms in the ring are replaced by heteroatoms of O, S or N. A heterocycloalkyl is optionally fused with aryl or heteroaryl of 5-6 ring members, and includes oxidized S or N, such as sulfinyl, sulfonyl and N-oxide of a tertiary ring nitrogen. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl. A hetercycloalkyl group can be unsubstituted or optionally substituted with one or more substituents as described herein below.

“Optionally substituted heterocycloalkyl” denotes a heterocycloalkyl that is substituted with 1 to 3 substituents, e.g., 1, 2 or 3 substituents, attached at any available atom to produce a stable compound, wherein the substituents are as described herein.

The term “nitrile” or “cyano” can be used interchangeably and refer to a —CN group which is bound to a carbon atom of a heteroaryl ring, aryl ring and a heterocycloalkyl ring.

The term “oxo” refers to a ═O atom attached to a saturated or unsaturated moiety. The ═O atom can be attached to a carbon, sulfur, or nitrogen atom that is part of a cyclic or acyclic moiety.

A “hydroxyl” or “hydroxy” refers to an —OH group.

The substituent —CO₂H may be replaced with bioisosteric replacements such as:

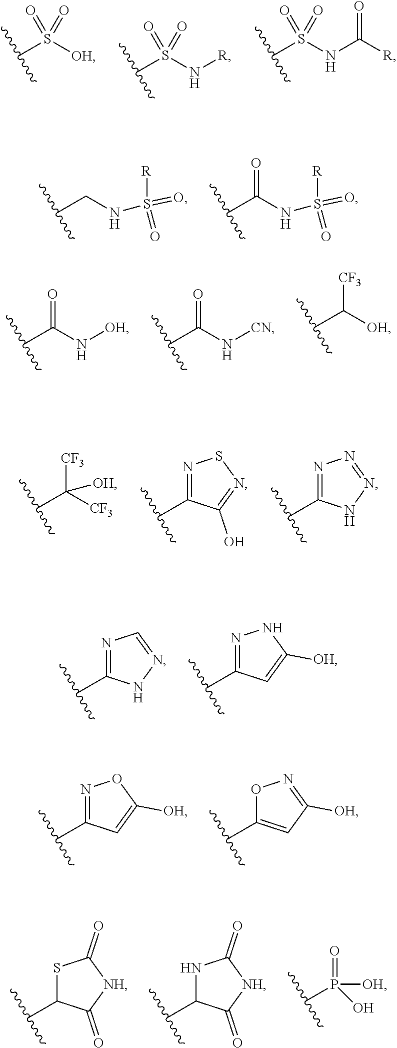

and the like, wherein R has the same definition as R^{A} as defined herein. See, e.g., THE PRACTICE OF MEDICINAL CHEMISTRY (Academic Press: New York, 1996), at page 203.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified to the contrary, the term “compound” is inclusive in that it encompasses a compound or a pharmaceutically acceptable salt, stereoisomer, and/or tautomer thereof. Thus, for instance, a compound of Formula I includes a pharmaceutically acceptable salt of the compound.

In this disclosure, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2, 2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In certain embodiments, such terms refer to minimizing the spread or worsening of the disease resulting from the administration of one or more prophylactic or therapeutic agents to a patient with such a disease.

The terms “prevent,” “preventing,” and “prevention” refer to the prevention of the onset, recurrence, or spread of the disease in a patient resulting from the administration of a prophylactic or therapeutic agent.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or synergies with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult.

“Inhibitor” means a compound that induces dose dependent lymphopenia and a modest decrease in plasma S1P. In an embodiment, an inhibitor binds to SPNS2.

### Compounds

As described generally above, the present disclosure provides compounds, pharmaceutically acceptable salts, and/or tautomers thereof, wherein the compounds conform to Formula I.

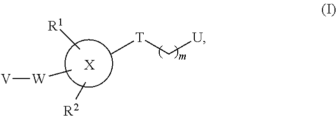

X is a C₆-C₁₀-aryl or 5- to 10-membered heteroaryl (wherein 1 to 4 heteroaryl ring members are independently selected from N, O, and S).

R¹ and R² are independently selected from the group consisting of H, C₁-C₆-alkyl, C₁-C₆-alkoxy, C₁-C₆-haloalkoxy, C₃-C₈-cycloalkyl, C₁-C₆-haloalkyl, CN, and halo.

W is a bond, O, NH, —NHC(O)—, or —O—(N═)C(R)— (wherein R is H or C₁-C₆-alkyl).

In some embodiments, U is (A) an optionally fused, bridged, or spiro-fused 6- to 8-membered heterocycloalkyl wherein 2 ring atoms are N. In these embodiments, V is selected from the group consisting of H, C₁-C₁₄-alkyl, C₂-C₁₂-alkenyl, (C₆-C₁₀)aryl, (C₆-C₁₀)heteroaryl, —C₁-C₁₀-alkyl-(C₆-C₁₀)aryl, —C₂-C₁₂-alkenyl-(C₆-C₁₀)aryl, —C₁-C₁₀-alkyl-(C₃-C₈)cycloalkyl, -(3- to 14-membered heterocycloalkyl) (wherein 1-4 heterocycloalkyl members are independently selected from N, O, and S), —(C₁-C₁₀)alkyl-(3- to 14-membered heterocycloalkyl) (wherein 1-4 heterocycloalkyl members are independently selected from N, O, and S);

In other embodiments, U is (B) a monocyclic 4- to 7-membered heterocycloalkyl (wherein 1-4 heterocycloalkyl members are independently selected from N, O, and S). In these embodiments, V is selected from the group consisting of H, —C₁-C₁₄-alkyl, —C₁-C₁₀-alkyl-O—(C₆-C₁₀)aryl, —C₁-C₁₀-alkyl-NR^{x}—(C₆-C₁₀)aryl, —C₁-C₁₀-alkyl-NR^{x}C(O)(C₆-C₁₀)aryl, —C₁-C₁₀-alkyl-C(O)NR^{x}(C₆-C₁₀)aryl, —C₂-C₁₂-alkenyl-O—(C₆-C₁₀)aryl, —C₁-C₁₀-alkyl-O—(C₆-C₁₀)heteroaryl (wherein 1 to 4 heteroaryl ring members are independently selected from N, O, and S), and —C₂-C₁₂-alkenyl-O—(C₆-C₁₀)heteroaryl (wherein 1 to 4 heteroaryl ring members are independently selected from N, O, and S).

T is —C(O)— or —NR^{x}C(O)—.

R^{x} is H or C₁-C₆-alkyl.

m is an integer selected from 0, 1, 2, 3, 4, 5, and 6.

In Formula I, each alkyl, alkoxy, alkenyl, aryl, cycloalkyl, heterocycloalkyl, and heteroaryl is optionally substituted with 1-5 substituents independently selected from the group consisting of hydroxy, halo, C₁-C₆-alkyl, C₁-C₆-haloalkoxy, C₁-C₆-haloalkyl, —NR′₂, —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkoxy, C₁-C₆-alkyl, C₃-C₁₄-cycloalkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl).

Each R′ is independently selected from the group consisting of C₁-C₆-alkyl, C₂-C₆-alkenyl, C₂-C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S.

In various embodiments, U is (A) an optionally substituted and optionally fused, bridged, or spiro-fused 6- to 8-membered heterocycloalkyl wherein 2 ring atoms are N, and V is selected from the group consisting of H, C₁-C₁₄-alkyl, C₂-C₁₂-alkenyl, (C₆-C₁₀)aryl, (C₆-C₁₀)heteroaryl, —C₁-C₁₀-alkyl-(C₆-C₁₀)aryl, —C₂-C₁₂-alkenyl-(C₆-C₁₀)aryl, —C₁-C₁₀-alkyl-(C₃-C₈)cycloalkyl, -(3- to 14-membered heterocycloalkyl) (wherein 1-4 heterocycloalkyl members are independently selected from N, O, and S), —(C₁-C₁₀)alkyl-(3- to 14-membered heterocycloalkyl) (wherein 1-4 heterocycloalkyl members are independently selected from N, O, and S).

In some embodiments wherein U conforms to (A), V is C₁-C₁₄-alkyl. In an illustrative embodiment, V is C₈-C₁₂-alkyl.

In additional embodiments wherein U conforms to (A), U is selected from the group consisting of optionally substituted:

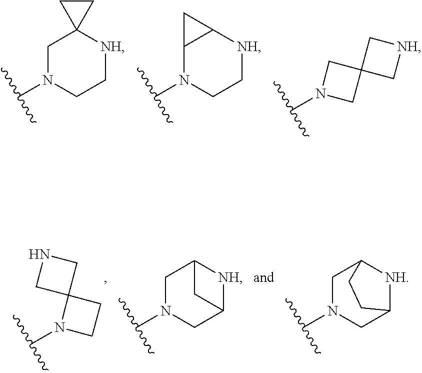

In various embodiments, U is (B) an optionally substituted monocyclic 4- to 7-membered heterocycloalkyl (wherein 1-4 heterocycloalkyl members are independently selected from N, O, and S), and V is selected from the group consisting of H, —C₁-C₁₄-alkyl, —C₁-C₁₀-alkyl-O—(C₃-C₁₄)cycloalkyl, —C₁-C₁₀-alkyl-O—(C₆-C₁₀)aryl, —C₁-C₁₀-alkyl-NR^{x}—(C₆-C₁₀)aryl, —C₁-C₁₀-alkyl-NR^{x}C(O)(C₆-C₁₀)aryl, —C₁-C₁₀-alkyl-C(O)NR^{x}—(C₆-C₁₀)aryl, —C₂-C₁₂-alkenyl-O—(C₆-C₁₀)aryl, —C₁-C₁₀-alkyl-O—(C₆-C₁₀)heteroaryl (wherein 1 to 4 heteroaryl ring members are independently selected from N, O, and S), and —C₂-C₁₂-alkenyl-O—(C₆-C₁₀)heteroaryl (wherein 1 to 4 heteroaryl ring members are independently selected from N, O, and S).

In various embodiments wherein U conforms to (B), U is a monocyclic 5- to 7-membered heterocycloalkyl (wherein 1 or 2 heterocycloalkyl members are independently selected from N and O). Examples of the heterocycloalkyl include optionally substituted pyrrolidinyl, oxazolidinyl, isoxazolidinyl, imidazolinyl, pyrazolidinyl, piperidinyl, piperazinyl, and morpholinyl. In an illustrative embodiment, U is piperazinyl.

In some embodiments wherein U conforms to (B), V is —C₁-C₁₀-alkyl-O—(C₆-C₁₀)aryl. In an illustrative embodiment, V is —C₃-C₈-alkyl-O-phenyl. In an illustrative embodiment, V is —C₃-C₈-alkyl-O-phenyl and the phenyl is substituted with a C₃-C₁₄-cycloalkyl. In some embodiments, the cycloalkyl is a cyclopropyl.

In various embodiments, optionally in combination with any other embodiment described herein, X is C₆-C₁₀-aryl. In an exemplary embodiment, X is phenyl.

In additional embodiments, optionally in combination with any other embodiment described herein, W is a bond or O. For example, in one embodiment W is a bond.

In still additional embodiments, optionally in combination with any other embodiment described herein T is —NR^{x}C(O)—. In some embodiments, R^{x} is H.

In other embodiments, optionally in combination with any other embodiment described herein T is —C(O)—.

In still further embodiments, optionally in combination with any other embodiment described herein m is 0.

In various embodiments, the present disclosure provides a compound or pharmaceutically acceptable salt thereof wherein:
U is (A) an optionally substituted and optionally fused, bridged, or spiro-fused 6- to 8-membered heterocycloalkyl wherein 2 ring atoms are N;
X is C₆-C₁₀-aryl;
T is —NR^{x}C(O)— wherein R^{x} is H;
V is C₈-C₁₂-alkyl; and
W is a bond; and
m is 0.

In various embodiments, the present disclosure also provides a compound or pharmaceutically acceptable salt thereof wherein:
U is (B) an optionally substituted 5- to 7-membered heterocycloalkyl (wherein 1 or 2 heterocycloalkyl members are independently selected from N and O);
X is C₆-C₁₀-aryl;
T is —NR^{x}C(O)— wherein R^{x} is H;
V is —C₁-C₁₀-alkyl-O—(C₆-C₁₀)aryl; and
W is a bond; and
m is 0.

The present disclosure provides specific examples of Formula I compounds, and their pharmaceutically acceptable salts, and/or tautomers thereof as set forth in Table 1 and Table 2 below. Inhibition of sphingosine 1-phosphate transporter spinster homolog 2 (Spns2), reported in the tables as IC₅₀ for each compound, was determined in accordance with literature protocol. Y. K. Kharel et al. SLAS Discovery 28 (2023) 284-287. See also R. G. Fritzemeier et al. J. Med Chem. 65 (2022) 7656-7681; S. Tanaka et al. Sci. Transl. Med. 14 (2022) 658; A. L. Burgio et al. J. Med. Chem. 66 (8) (2023) 5873-5891.

TABLE 1
Examples of Formula I Compounds (A: IC₅₀ < 1 μM; B: IC₅₀ > 1 μM)

| | | |
|---|---|---|
| 3a | 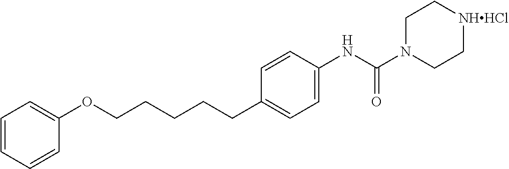 | A |
| 3b | 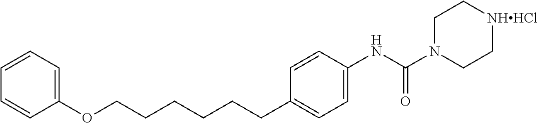 | A |
| 3c | 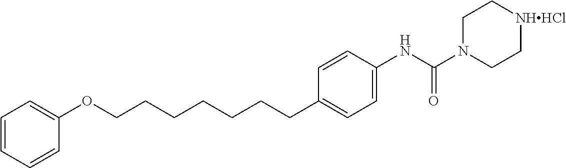 | A |
| 3d | 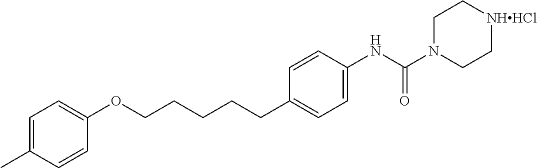 | A |
| 3e | 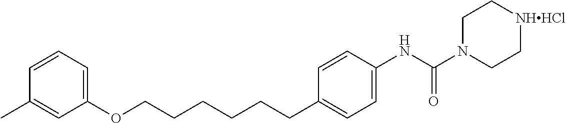 | A |
| 3f | 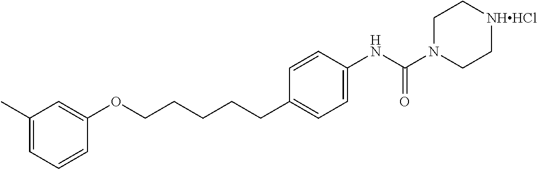 | A |
| 3g | 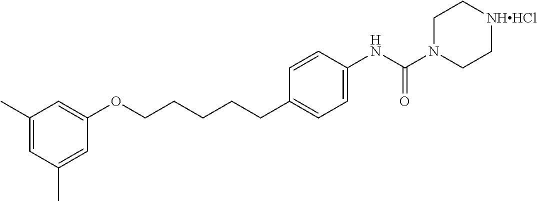 | B |
| 3h | 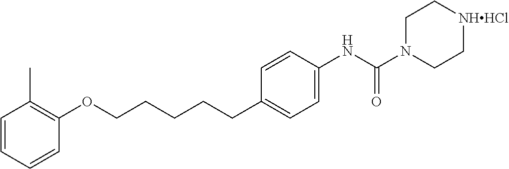 | A |
| 3i | 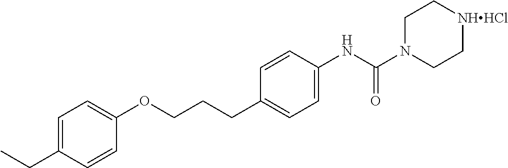 | B |
| 3j | 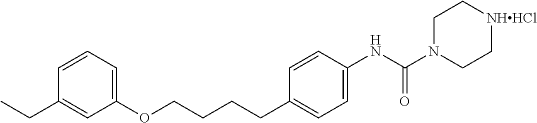 | A |
| 3k | 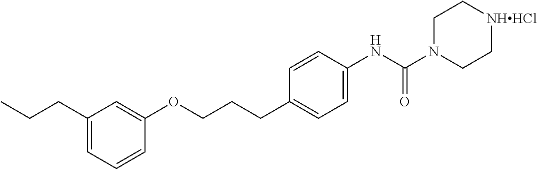 | A |
| 3l | 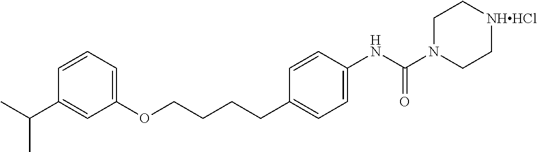 | A |
| 3m | 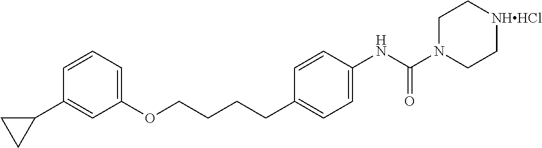 | A |
| 3n | 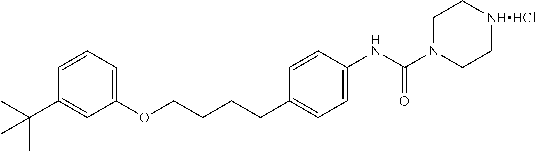 | A |
| 3o | 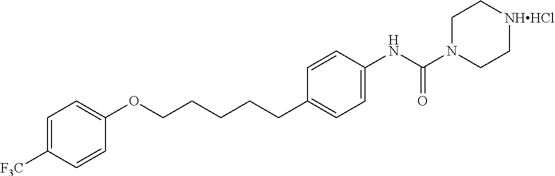 | B |
| 3p | 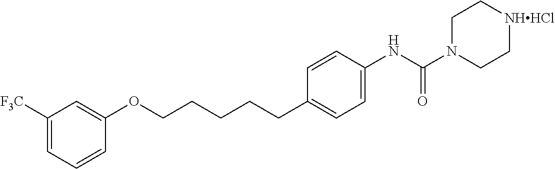 | B |
| 3q | 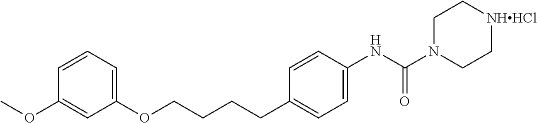 | A |
| 3r | 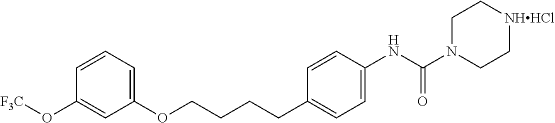 | B |
| 3s | 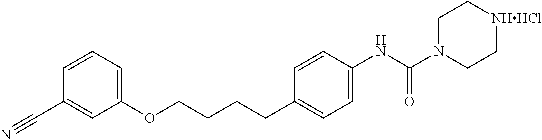 | B |
| 3t | 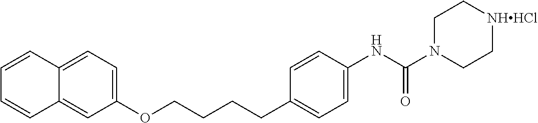 | B |
| 3u | 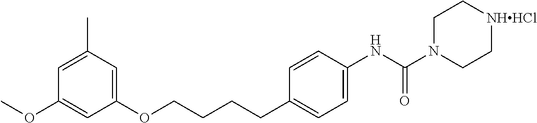 | B |
| 3v | 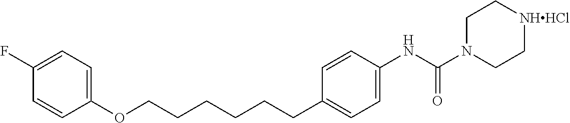 | A |
| 3w | 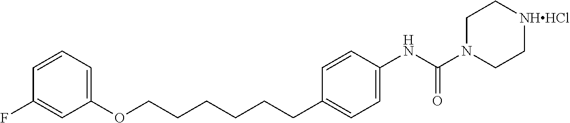 | A |
| 3x | 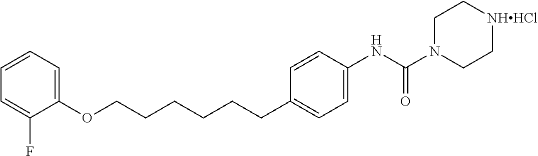 | A |
| 3y | 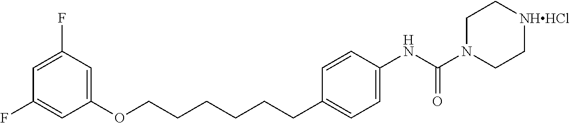 | A |
| 3z | 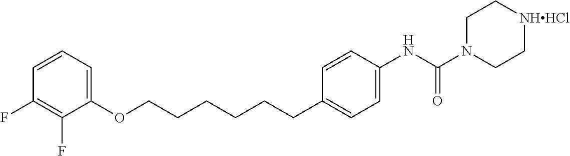 | A |
| 3aa | 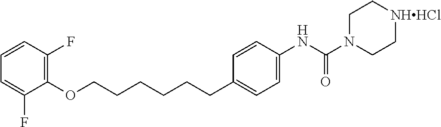 | A |
| 3ab | 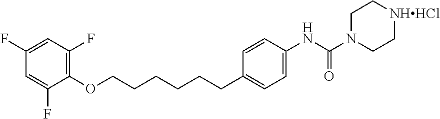 | A |
| 3ac | 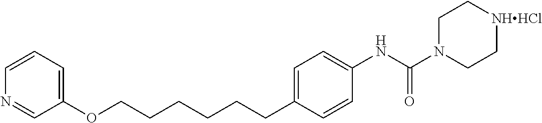 | B |
| 3ad | 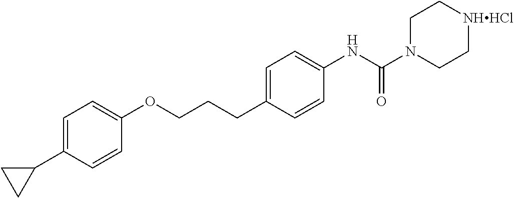 | B |
| 3ae | 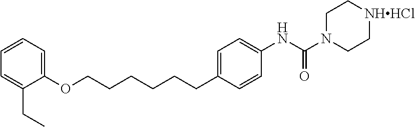 | B |
| 3af | 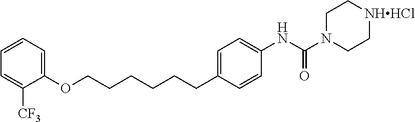 | B |
| 3ag | 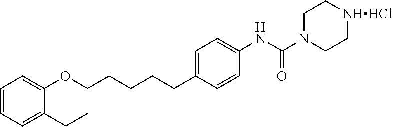 | B |
| 3ah | 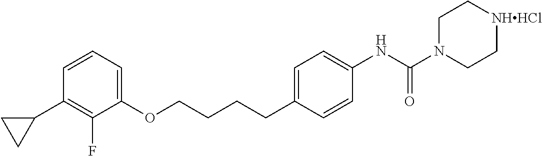 | A |
| 3ai | 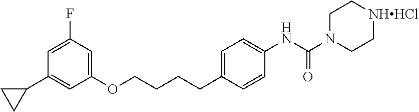 | A |
| 3aj | 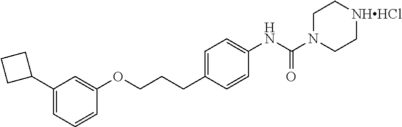 | A |
| 3ak | 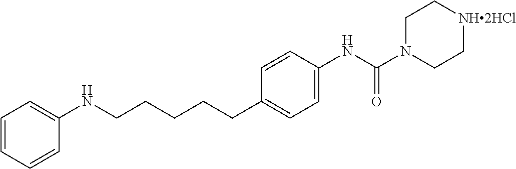 | A |
| 3al | 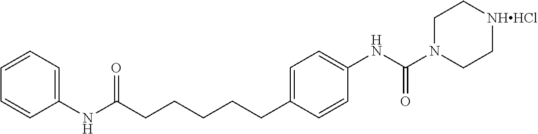 | B |
| 3am | 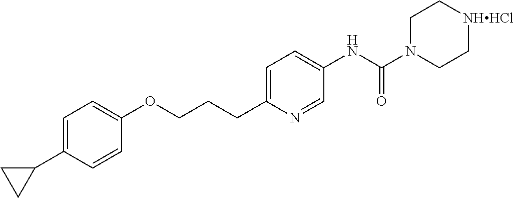 | B |
| 3an | 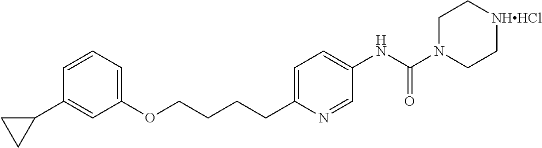 | B |
| 3ao | 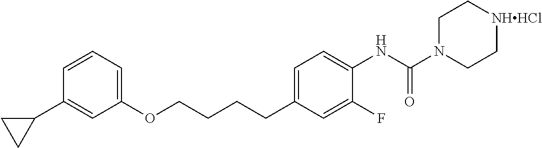 | A |
| 3ap | 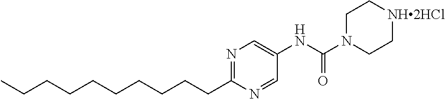 | B |
| 3aq | 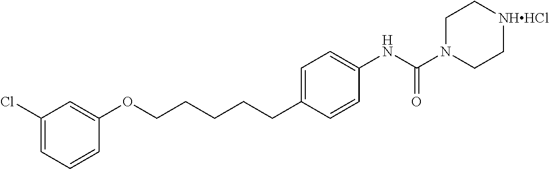 | A |
| 3ar | 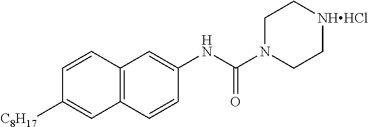 | B |
| 3as | 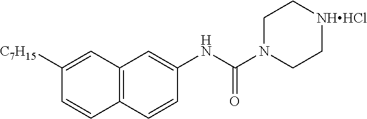 | B |
| 3at | 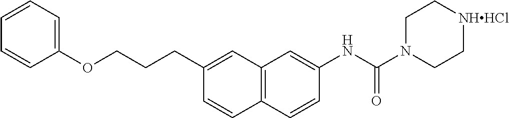 | B |
| 3au | 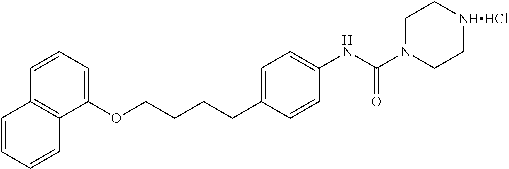 | A |
| 3av | 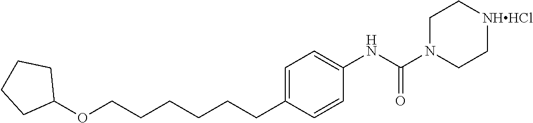 | A |
| 3aw | 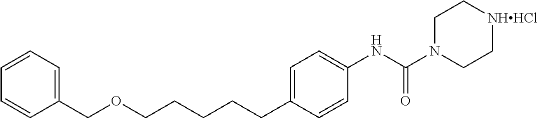 | A |
| 3ax | 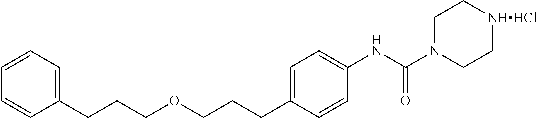 | A |
| 3ay | 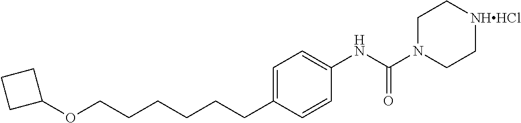 | A |
| 3az | 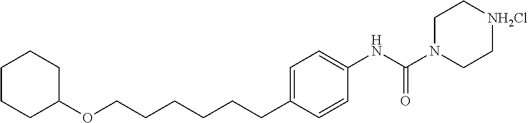 | A |
| 3ba | 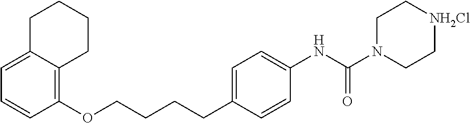 | A |
| 3bb | 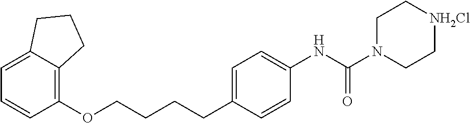 | A |
| 3bc | 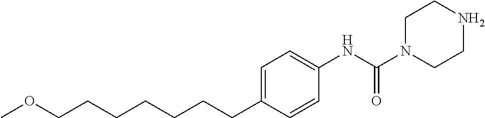 [SMILES: COCCCCCCCc1ccc(NC(=O)N2CCNCC2)cc1] | A |
| 3bd | 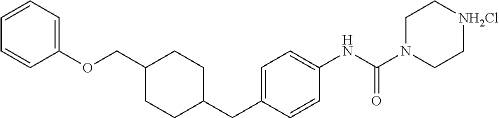 | A |
| 3be | 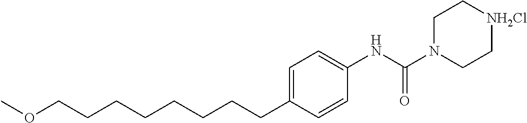 | A |
| 3bf | 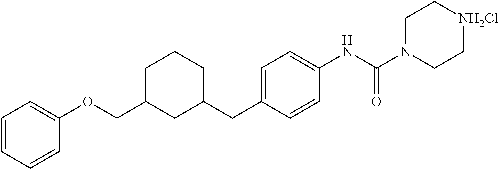 | A |
| 3bg | 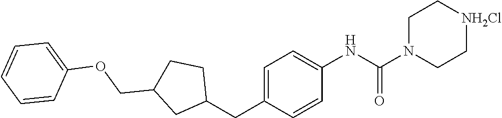 | A |
| 3bh | 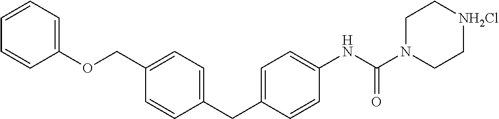 | A |
| 3bi | 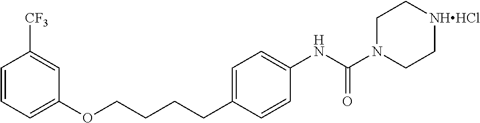 | A |
| 5a | 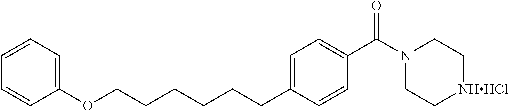 | A |
| 5b | 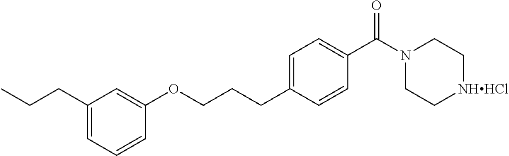 | A |
| 5c | 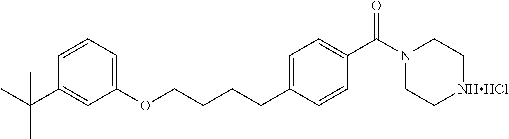 | A |
| 18a | 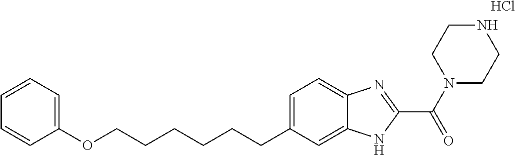 [SMILES: Cl ; O=C(c1nc2ccc(CCCCCCOc3ccccc3)cc2[nH]1)N1CCNCC1] | A |
| 18b | 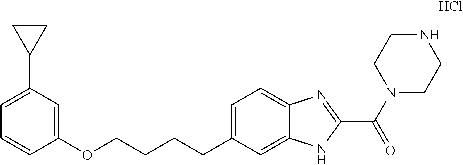 [SMILES: Cl ; O=C(c1nc2ccc(CCCCOc3cccc(C4CC4)c3)cc2[nH]1)N1CCNCC1] | A |
| 18e | 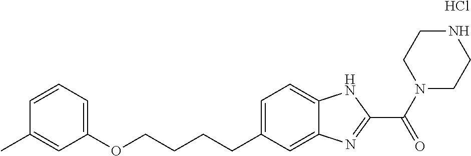 [SMILES: Cl ; Cc1cccc(OCCCCc2ccc3[nH]c(C(=O)N4CCNCC4)nc3c2)c1] | A |
| 18f | 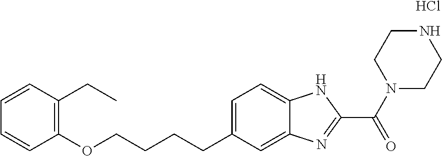 [SMILES: Cl ; CCc1ccccc1OCCCCc1ccc2[nH]c(C(=O)N3CCNCC3)nc2c1] | A |
| 18g | 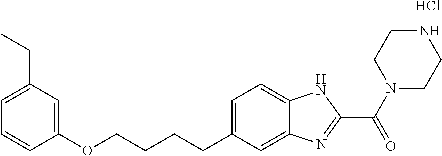 [SMILES: Cl ; CCc1cccc(OCCCCc2ccc3[nH]c(C(=O)N4CCNCC4)nc3c2)c1] | A |
| 18h | 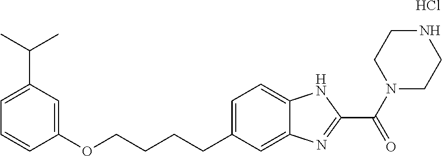 [SMILES: Cl ; CC(C)c1cccc(OCCCCc2ccc3[nH]c(C(=O)N4CCNCC4)nc3c2)c1] | A |
| 18i | 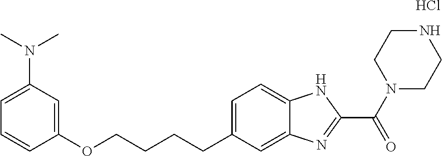 [SMILES: Cl ; CN(C)c1cccc(OCCCCc2ccc3[nH]c(C(=O)N4CCNCC4)nc3c2)c1] | A |
| 18j | 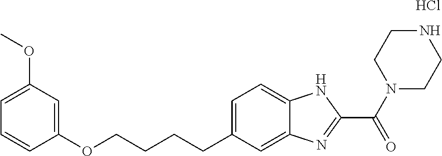 [SMILES: Cl ; COc1cccc(OCCCCc2ccc3[nH]c(C(=O)N4CCNCC4)nc3c2)c1] | A |
| 18k | 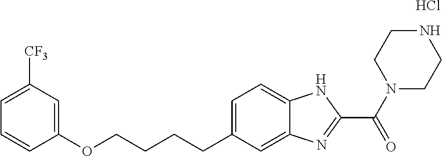 [SMILES: Cl ; O=C(c1nc2cc(CCCCOc3cccc(C(F)(F)F)c3)ccc2[nH]1)N1CCNCC1] | A |
| 18l | 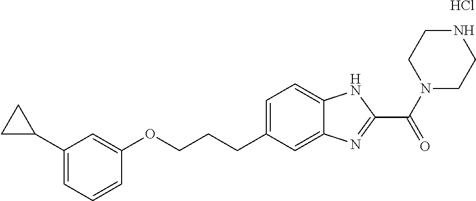 [SMILES: Cl ; O=C(c1nc2cc(CCCOc3cccc(C4CC4)c3)ccc2[nH]1)N1CCNCC1] | A |
| 21a | 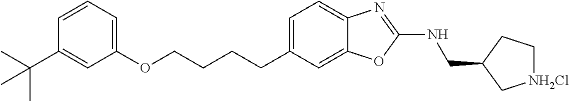 | A |
| 21b | 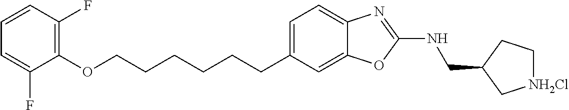 | A |
| 21c | 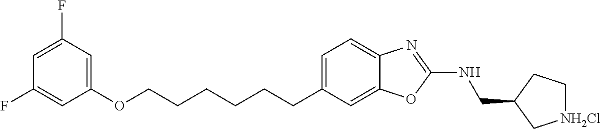 | A |
| 21d | 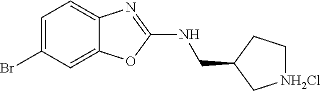 | A |
| 21e | 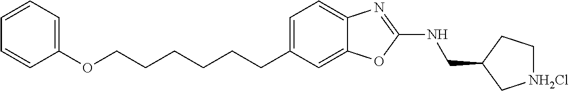 | B |
| 21f | 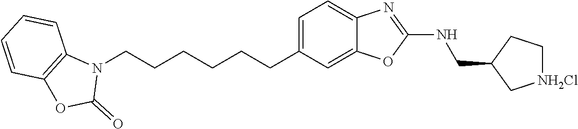 | A |
| 21g | 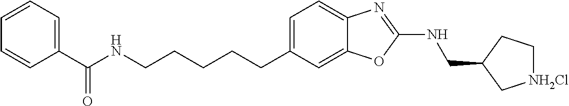 | A |
| 21h | 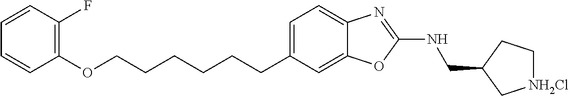 | B |
| 21i | 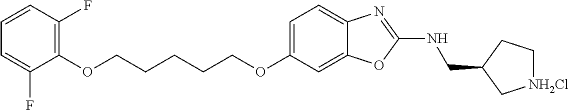 | B |
| 21j | 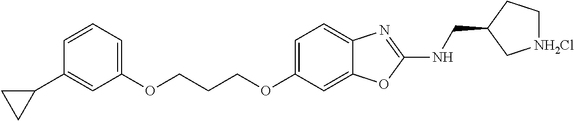 | A |
| 21k | 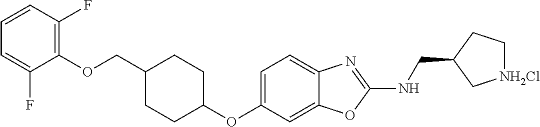 | B |
| 21l | 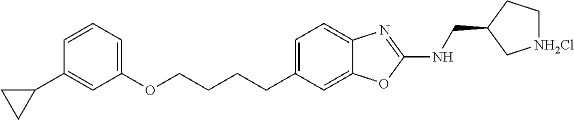 | B |

TABLE 2
Examples of Formula I Compounds (A: IC₅₀ < 1 μM; B: IC₅₀ > 1 μM)

| Compound | Structure | Activity |
|---|---|---|
| 7a | 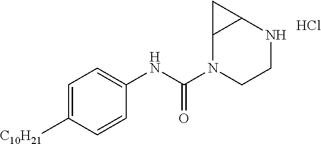 | B |
| 7b | 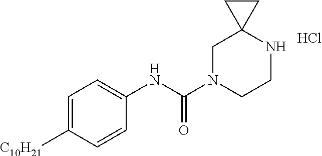 | B |
| 7c | 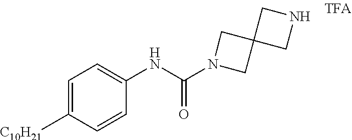 | B |
| 7d | 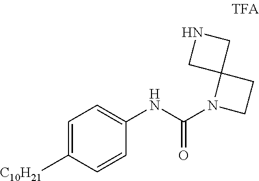 | B |
| 7e | 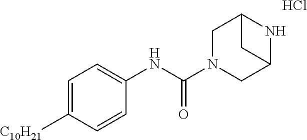 | B |
| 9a | 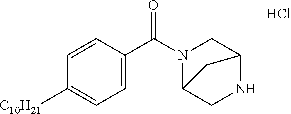 | A |
| 9b | 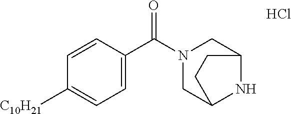 | B |
| 7f | 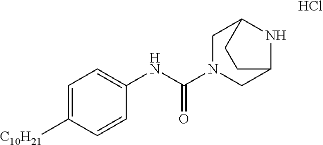 | A |

### Pharmaceutical Composition

The disclosure also provides a pharmaceutical composition comprising a therapeutically effective amount of one or more compounds according to Formula I or a pharmaceutically acceptable salt, stereoisomer, and/or tautomer thereof in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises a compound selected from those illustrated in Table 1 or Table 2 or a pharmaceutically acceptable salt, stereoisomer, and/or tautomer thereof, and a pharmaceutically acceptable carrier.

The pharmaceutical composition of the present disclosure is formulated, dosed, and administered in a manner consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of a compound (or a pharmaceutically acceptable salt, stereoisomer, and/or tautomer thereof that is administered is governed by such considerations, and is the minimum amount necessary to induce dose dependent lymphopenia and a modest decrease in plasma S1P, or to inhibit SPNS2 activity, or both. Such amount may be below the amount that is toxic to normal cells, or the subject as a whole. Generally, the initial therapeutically effective amount of a compound (or a pharmaceutically acceptable salt, stereoisomer, or tautomer thereof) of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 1 mg to about 1000 mg of a compound (or a pharmaceutically acceptable salt, stereoisomer, or tautomer thereof) of the present disclosure. In another embodiment, such dosage forms contain from about 50 mg to about 500 mg of a compound (or a pharmaceutically acceptable salt, stereoisomer, or tautomer thereof) of the present disclosure. In yet another embodiment, such dosage forms contain from about 25 mg to about 200 mg of a compound (or a pharmaceutically acceptable salt, stereoisomer, or tautomer thereof) of the present disclosure. In still another embodiment, such dosage forms contain from about 10 mg to about 100 mg of a compound (or a pharmaceutically acceptable salt, stereoisomer, or tautomer thereof) of the present disclosure. In a further embodiment such dosage forms contain from about 5 mg to about 50 mg of a compound (or a pharmaceutically acceptable salt, stereoisomer, or tautomer thereof) of the present disclosure.

The disclosed compositions can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

Also encompassed by the present disclosure are pharmaceutical compositions suitable for single unit dosages that comprise a compound of the disclosure or its pharmaceutically acceptable stereoisomer, salt, or tautomer and a pharmaceutically acceptable carrier.

Compositions suitable for oral use may be prepared according to any method known to the art for the manufacture of pharmaceutical compositions. For instance, liquid formulations of the inventive compounds contain one or more agents selected from the group consisting of sweetening agents, flavoring agents, coloring agents and preserving agents in order to provide pharmaceutically palatable preparations of the SPNS2 inhibitor.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### Methods of Use

S1P gradients are chemotactic, a property that enables correct positioning of immune cells, and they help to maintain endothelial barrier integrity. Accordingly, S1P gradients are manipulated for therapeutic benefit using Formula I compounds because they target the endothelial S1P exporter, SPNS2.

Thus, in one embodiment, the disclosure provides a method of inhibiting spinster homolog 2 (SPNS2). The method comprises contacting SPNS2 with an effective amount of a compound as described herein. In some embodiments, the contacting occurs in vitro. In other embodiments, the contacting occurs ex vivo or in vivo.

Another embodiment is a method of treating a patient afflicted by a neoplastic disease, comprising administering to the patient a therapeutically effective amount a compound as described herein. In some embodiments, the neoplastic disease is metastatic neoplasms.

An additional embodiment is a method of treating a patient afflicted with an allergic disease, comprising administering to the patient a therapeutically effective amount of a compound as described herein. An illustrative allergic disease is asthma.

Formula I compounds also are useful in a method of treating a patient afflicted with an autoimmune disease, comprising administering to the patient a therapeutically effective amount of the compound. In various embodiments, the autoimmune disease is chosen from multiple sclerosis, type I diabetes, inflammatory bowel diseases including Crohn's disease and ulcerative colitis, Grave's disease, Addison's disease, dermatomyositis, myasthenia gravis, systemic lupus erythematosus, scleroderma, psoriasis, and atopic dermatitis. An exemplary autoimmune disease is multiple sclerosis. In accordance with some embodiments, multiple sclerosis comprises one or more progressive forms of multiple sclerosis as well as the remitting relapsing form of the disease.

Additional embodiments include a method of treating a patient afflicted with atherosclerosis or pulmonary arterial hypertension. The method comprises administering to the patient a therapeutically effective amount of a compound as described herein.

In an additional embodiment, the present disclosure provides a method for treating a patient afflicted with a fibrotic disease. The method comprises administering to the patient a therapeutically effective amount of a compound as described herein. In some embodiments, the fibrotic disease is a renal fibrosis, such as chronic kidney disease.^{10,11}

### LITERATURE CITED IN THE DISCLOSURE

Numbered citations in the present disclosure are as follows, and are incorporated by reference as if fully set forth herein:
1. Schwab S R, Pereira J P, Matloubian M, Xu Y, Huan, Y, Cyster J G. Lymphocyte sequestration through S1P lyase inhibition and disruption of S1P gradients. Science 309, 1735-1739 (2005)
2. Cusak K P, Stoffel R H. S1P(1) receptor agonists: Assessment of selectivity and current clinical activity. Current Opinion Drug Discovery & Development 13, 481-488 (2010)
3. Mendoza A, Breat B, Ramoz-Perez W D, Pitt L A, Gobert M, Sunkara M, Lafaille J J, Morris A J, Schwab S R. The transporter SPNS2 is required for secretion of lymph by not plasma sphingosine-1-phosphate. Cell Reports 2, 1104-1110 (2012)
4. Vu T M, Ishizu A-N, Foo J C, Toh X R, Zhang F, Whee D M, Torta F, Cazenave-Gassiot A, Matsumura T, Kim S, To S-AES, Suda T, Silver D L, Wenk M R, Nguyen L N. MFSD2B is essential for the sphingosine-1-phosphate export in erythrocytes and platelets Nature 550, 524-528 (2017)
5. Kobayashi N, Kawasaki-Nishi S, Otsuka M, Hisano Y, Yamaguchi A, Nishi T MFSD2B is a sphingosine 1-phosphate transporter in erythroid cells. Scientific Reports 8, 4969-4974 (2018)
6. Camerer E, Regard J B, Cornelissen I, Srinivasan T, Duang D N, Palmer D, Pham T H, Wong J S, Pappu R, Coughlin S R. Sphingosine-1-phosphate in the plasma compartment regulates basal and inflammation-induced vascular leak in mice. J Clinical Investigation 119, 1871-1879 (2009)
7. Xiong Y, Hla T. S1P control of endothelial integrity. Current Topics in Microbiology & Immunology 378, 85-105 (2014)
8. Schumann J, Grevot A, Ledieu D, Wolf A, Schubart A, Piaia A, Sutter E, Côte S, Beerli C, Pognan F, Billich A, Moulin P, Walker U J. Reduced activity of sphingosine-1-phosphate lyase induces podocyte-related glomerular proteinuria, skin irritation and platelet activation. Toxicologic Pathology 43, 694-703 (2015)
9. Nijnik A, Clare S, Hale C, Chen J, Raisen C, Mottram L, Lucas M, Estabel J, Ryder E, Adissu H, Adama N C, Ramirez-Solis R, White J K, Steel K P, Dougan, Hancock R E W. Role of sphingosine-1-phosphate transporter Spns2 in immune system function. J Immunology 89, 102-111 (2012)
10. Fukuhara S, Simmons S, Kawamura S, Inoue A, Orba Y, Tokudome T, Sunden Y, Atai Y, Moriwaki K, Ishida J, Uemura A, Abe T, Fukamizu A, Hirashima, Sawa H, Aoki J, Ishii M, Mochizuki N. The sphingosine-1-phosphate transporter Spns2 expressed on endothelial cells regulates lymphocyte trafficking in mice. J Clinical Investigation 122, 1416-1426 (2012)
11. Mendoza A, Breart B, Ramos-Perez W D, Pitt L A, Gobert M, Sunkara M, Lafaille J J, Morris A J, Schwab S R. The transporter Spns2 is required for secretion of lymph but not plasma sphingosine-1-phosphate. Cell Reports 2, 1104-1110 (2012)
12. Le T N U, Nguyen T Q, Kalailingam P, Nguyen Y T K, Sukumar V K, Tan C K H, Tukijan F, Couty L, Hasan Z, Gaudio I D, Wenk M R, Cazenave-Gassiot A, Camerer E, Nguyen L N. Mfsd2b and Spns2 are essential for maintenance of blood vessels during development and in anaphylactic shock. Cell Reports 40, 111208 (2022)
13. Fritzemeier R G, Foster D, Peralta A, Payette M, Kharel Y, Huang T, Lynch K R, Santos W L. Discovery of In Vivo Active Sphingosine-1-phosphate Transporter (Spns2) Inhibitors. J. Medicinal Chemistry 65, 7656-7681 (2022)
13. Burgio A, Shrader C W, Kharel Y, Huang T, Salamoun J M, Lynch K R, Santos W L. 2-Aminobenzoxazole derivatives as potent inhibitors of the sphingosine 1-phosphate transporter spinster homolog 2 (Spns2). J Medicinal Chemistry 66, 5873-5891 (2023)
14. Van der Weyden L, Arends M J, Campbell A D, Bald T, Wardle-Jones H, Griggs N, Velasco-Herrera M D, Tuting T, Sansom O J, Karp N A, Clare S, Gleeson D, Rider E, Galli A, Tuck E, Cambridge E L, Voet T, Macaulay I C, Wong K, Sanger Mouse Genetics Project, Spiegel S, Speak A O, Adams D J. Genome-wide in vio screen identifies novel new regulators for metastatic colonization. Nature 531, 233-236 (2017)
15. Tanaka S, Zheng S, Kharel Y, Fritzemeie R G, Huang T, Foster D, Poudel N, Goggins E, Yamaoka Y, Rudnicka K P, Lipsey J E, Radel H V, Ryuh S M, Inoue T, Yao J, Rosin D L, Schwab S R, Santos W L, Lynch K R, Okusa M D. Sphingosine 1-phosphate signaling in perivascular cells enhances inflammation and fibrosis in the kidney. Science Translational Medicine 14, eabj2681 (2022)

### EXAMPLES

The present disclosure will be more fully understood by reference to the following examples. The examples should not, however, be construed as limiting the scope of the present disclosure.

### General Procedure 1: Nucleophilic Substitution A

Nucleophile (1.0 equiv.), K₂CO₃ (2.0 equiv.), and alkyl halide (1.2 equiv.) were added to a 6-dram vial containing MeCN at room temperature. The resulting solution was allowed to stir at 80° C. for 16 hours or until complete consumption of the starting material was observed on TLC. The reaction was diluted with water and extracted with ethyl acetate (3×). The organic extracts were combined, washed with brine (2×), dried with MgSO₄, filtered, and concentrated in vacuo. The residue was purified by flash column chromatography to give the desired compound.

### General Procedure 2: Urea Synthesis A

1-iodo-4-isocyanatobenzene (1.0 equiv.) was added to an oven dried 6-dram vial containing a stir bar and purged with nitrogen. Anhydrous DCM was added to the vial and the solution was cooled to 0° C. with an ice bath. Mono-N-Boc-piperazine (1.05 equiv.) was dissolved in DCM and added dropwise to the 1-iodo-4-isocyanatobenzene solution. The reaction mixture was then allowed to warm to room temperature slowly over the course of 16 hours. After complete consumption of starting material was observed by TLC, the crude reaction mixture was concentrated under reduced pressure and subjected to flash chromatography with an appropriate ethyl acetate and hexanes mobile phase to yield purified product.

### General Procedure 3: Suzuki Cross-Coupling

To a pressure-sealed tube purged with nitrogen was added the necessary alkene (1.0 equiv.) and 0.5 M 9-BBN in THE (1.5 equiv.). The resulting mixture was allowed to stir at 70° C. for 2 hours. Upon completion of the hydroboration step, sequential addition of an aryl iodide (1.0 equiv.), Pd(dppf)Cl₂*CH₂Cl₂ (0.05 equiv.), and 3 M KOH (3.0 equiv.) was performed. The resulting mixture was allowed to stir at 70° C. for 16 hours or until complete consumption of the starting material was observed on TLC. The resulting mixture was concentrated in vacuo to afford a brown oil which was the subjected to flash chromatography with an appropriate ethyl acetate and hexane solvent system to afford the purified product.

### General Procedure 4: HCl Boc Deprotection

To a 6-dram vial containing a Boc-protected amine (1.0 equiv.) was added hydrogen chloride (30-100 equiv., 4 M in dioxane). The resulting mixture was allowed to stir until consumption of starting material as monitored by TLC (0.5-6 hours). A thick white precipitate forms during the course of the reaction. This precipitate was either vacuum filtered over a filter frit and washed with diethyl ether or triturated with diethyl ether and/or ethyl acetate to afford the pure product as an HCl salt.

### General Procedure 5: TFA Boc Deprotection

To a 6-dram vial containing a Boc-protected amine (1.0 equiv.) dissolved in DCM was added TFA (30 equiv.). The resulting solution was allowed to stir until consumption of starting material as monitored by TLC (1-6 hours). Concentration in vacuo and filtration of the resulting off-white solid, followed by washing with diethyl ether afforded the corresponding TFA salt.

### General Procedure 6: HCTU Coupling

To a 6-dram vial containing 4-iodobenzoic acid (1.1 equiv.) was added DCM (0.2 M), DIEA (1.8 equiv.) and HCTU (1.1 equiv.). The resulting mixture was allowed to stir at room temperature for 5 minutes, followed by addition of mono-N-Boc-piperazine. The resulting mixture was allowed to stir at room temperature for 18 hours or until complete consumption of starting material was observed on TLC. The resulting mixture was concentrated in vacuo to afford a yellow oil which was then subjected to flash chromatography with an appropriate ethyl acetate in hexanes solvent system to afford the pure product.

### General Procedure 7: Nucleophilic Substitution B

Nucleophile (1.0 equiv.), TEA (2.0 equiv), and alkyl halide (1.2 equiv.) were added to a 6-dram vial containing MeCN (0.2 M) at room temperature. The resulting solution was allowed to stir at 80° C. for 16 hours or until complete consumption of the starting material was observed on TLC. The resulting mixture was vacuum filtered before concentration of the filtrate in vacuo to afford a crude yellow oil, which was then subjected to flash chromatography with an appropriate ethyl acetate and hexane solvent system to afford the purified product.

### General Procedure 8: DCC Coupling

To a 6-dram vial containing the appropriate carboxylic acid (1.0 equiv.) was added DCM (0.2 M), appropriate aniline (1.0 equiv.), and DMAP (10 mol %). The resulting mixture was cooled to 0° C., followed by the addition of DCC (1.1 equiv.). The resulting mixture was allowed warm to room temperature and stir for 18 hours or until complete consumption of starting material was observed on TLC. The resulting mixture was concentrated in vacuo to afford a yellow solid which was then subjected to flash chromatography with an appropriate ethyl acetate in hexanes solvent system to afford the pure product.

### General Procedure 9: Benzimidazole Amide Synthesis

To a solution of 6-bromo-2-(trichloromethyl)-1H-benzo[d]imidazole (1.0 equiv.) in a 1:2 mixture of water:THF (0.2 M) was added N-Boc-piperazine (1.1 equiv.), followed by sodium bicarbonate (10.0 equiv.). The reaction was allowed to stir at 50° C. for 16 hours. Once complete, the reaction was allowed to cool, and the reaction was concentrated down. DCM was added and the organic layer was washed 3× with water. After, the organic layer was dried over anhydrous sodium sulfate, filtered, and purified via silica chromatography to afford the desired compound.

### General Procedure 10: Benzimidazole Boc Protection

To a solution of tert-butyl 4-(6-bromo-1H-benzo[d]imidazole-2-carbonyl)piperazine-1-carboxylate (1.0 equiv.) in DCM (0.2 M) was added 4-dimethylaminopyridine (0.1 equiv.). Boc anhydride (1.1 equiv.) was added dropwise, and the reaction was allowed to stir at room temperature for 16 hours. Once complete, the reaction was concentrated down and purified via silica chromatography to afford the desired compound.

### General Procedure 11: Urea Synthesis B

Triphosgene (0.5 equiv.) was dissolved in anhydrous DCM (0.1 M) under argon atmosphere, and the appropriate aniline (1.0 equiv.) in DCM (0.3 M) was added dropwise. This mixture was stirred for 30 min at room temperature and then treated with triethylamine (2.3 equiv.) in DCM (0.6 M). After 30 min, N-Boc-protected amine (1.5 equiv.) was added. The reaction mixture was stirred for 16 hours at room temperature, evaporated to dryness, treated with water (10 mL) and extracted with DCM (3×20 mL). The organic phase was dried over anhydrous sodium sulphate, filtered, and purified via silica chromatography.

### General Procedure 12: Suzuki Cross-Coupling B

To a pressure-sealed tube purged with nitrogen was added the necessary alkene (2.0 equiv.) and 0.5 M 9-BBN in THE (2.2 equiv.). The resulting mixture was allowed to stir at 70° C. for 2 hours. Upon completion of the hydroboration step, sequential addition of an aryl bromide (1.0 equiv.), Pd(dppf)Cl₂*CH₂Cl₂ (0.075 equiv.), and 3 M KOH (3.0 equiv.) was performed. The resulting mixture was allowed to stir at 70° C. for 16 hours or until complete consumption of the starting material was observed on TLC. The resulting mixture was concentrated in vacuo to afford a brown oil which was the subjected to flash chromatography with an appropriate ethyl acetate and hexane solvent system to afford the purified product.

### General Procedure 13: Methoxy Ether Synthesis

To a stirring solution of sodium methoxide in methanol (25% wt, 3.5 equiv.) was added bromo-1-alkene (1.0 equiv.). The reaction was stirred at 25° C., monitoring by TLC, then concentrated in vacuo, and redissolved in diethyl ether and water. The aqueous phase was washed with diethyl ether (3×) and the organic extracts were combined, washed with brine (2×), dried with MgSO₄, filtered, and concentrated in vacuo to give the desired compound.

### General Procedure 14: Miyaura Borylation

Bis(pinacolato)diboron (1.1 equiv.), potassium acetate (3.0 equiv.), aryl halide (1.0 equiv.) and 1,1′-bis(diphenylphosphino)ferrocenedichloro palladium(II) dichloromethane complex (3 mol %) were dissolved in DMSO (0.33 M) under a nitrogen atmosphere with stirring. The reaction was heated to 80° C. monitoring by TLC. The reaction was allowed to cool to room temperature, diluted with water, extracted with ethyl acetate (3×), washed with brine (2×), dried with MgSO₄, filtered, and concentrated in vacuo. The brown residue was purified by flash column chromatography to give the desired compound.

### General Procedure 15: Wittig Olefination

To a stirring suspension of methyltriphenylphosphonium bromide (2.0 equiv.) in tetrahydrofuran (0.25 M) was added potassium tert-butoxide (2.0 eq.) at 0° C. under a nitrogen atmosphere, immediately forming a yellow solution. The reaction was stirred for 30 minutes, then ketone (1.0 equiv.) was added, and the reaction was allowed to warm to 25° C. and stirred, monitoring by TLC. The reaction was quenched with water, extracted with ethyl acetate (3×), and the organic extracts were combined, washed with brine (2×) dried with MgSO₄, filtered, and concentrated in vacuo. The residue was purified by flash column chromatography to give the desired compound.

### General Procedure 16: Tosylation

To a stirring solution of alcohol (1.0 equiv.) in dichloromethane (0.77 M) was added triethylamine (1.5 equiv.), DMAP (0.10 equiv.) and 4-methylbenzenesulfonyl chloride (0.97 equiv.) sequentially at 0° C. under a nitrogen atmosphere. The reaction was allowed to warm to room temperature and stirred, monitoring by TLC. The reaction was diluted with diethyl ether and washed with water and aqueous HCl (1 M). The combined aqueous extracts were washed with diethyl ether (3×), and the organic extracts were combined, washed with brine (2×), dried with MgSO₄, filtered, and concentrated in vacuo to give the desired compound.

### General Procedure 17: Nucleophilic Substitution C

To a stirring solution of alcohol (1.0 equiv.) and potassium carbonate (2.0 equiv.) in acetonitrile (0.20 M) was added tosylated alcohol (1.2 equiv.). The reaction was heated to 80° C., monitoring by TLC. The reaction was diluted with water and extracted with ethyl acetate (3×). The organic extracts were combined, washed with brine (2×), dried with MgSO₄, filtered, and concentrated in vacuo. The residue was purified by flash column chromatography to give the desired compound.

### General Procedure 18: Carbonyl Reduction

To a stirring solution of carbonyl (1.0 eq.) in THE (0.25 M) was added lithium aluminum hydride in THE (1.0 M, 0.60 eq.) under a nitrogen atmosphere at 0° C. The reaction was allowed to warm to 25° C. and stirred, monitoring by TLC. The reaction was cooled to 0° C. and quenched with water, then extracted with ethyl acetate (3×). The organic extracts were combined, washed with brine (2×), dried with MgSO₄, filtered, and concentrated in vacuo to give the desired compound.

### General Procedure 19: Benzimidazole Ring Closure

To a flame-dried vial with stir bar was added phenyldiamine (1.0 equiv.) followed by acetic acid (0.2 M). This was allowed to cool to 0° C. for 15 minutes. Next, methyl 2,2,2-trichloromethylacetimidate (1.1 equiv.) was added dropwise. The reaction was allowed to warm to room temperature and react for 3 hours or until complete. Once complete, the reaction mixture was diluted with cold water and allowed to stir for 10 minutes. The resulting precipitate was then filtered and washed with cold water to afford the desired pure product.

### General Procedure 20: SEM Protection

To a flame-dried vial purged with argon was added benzimidazole (1.0 equiv.) followed by dry THE (0.2 M). This was cooled to 0° C. and allowed to stir for 10 minutes. Sodium hydride (1.1 equiv.) was added and the reaction mixture was allowed to stir for an additional 10 minutes. After, SEM-Cl (1.1 equiv.) was added dropwise and the reaction was allowed to warm to room temperature for 2 h. Once complete, the reaction mixture was concentrated and water was added. This was allowed to stir for 10 minutes to quench any remaining SEM-Cl. Ethyl acetate was then added and the organic layer was collected, dried over anhydrous sodium sulfate, and concentrated down to afford pure final product.

### General Procedure 21: TBAF SEM Deprotection

To a vial containing SEM-protected benzimidazole (1.0 equiv.) was added THE (0.2 M) followed by TBAF (3.0 equiv.). This was allowed to reflux for 16 hours until complete. Once complete, saturated ammonium chloride and ethyl acetate were added to the reaction mixture. The organic layer was washed with water additionally twice, dried over anhydrous sodium sulfate, and concentrated down. The resulting mixture was then purified by silica gel chromatography with an appropriate ethyl acetate/hexanes solvent system to afford the pure product.

### General Procedure 22: Nucleophilic Substitution D

Nucleophile (1.0 equiv.) and alkyl halide (1.2 equiv.) were added to a flame dried 6-dram vial containing dry THE at 0° C. before the addition of sodium hydride (60% wt., dispersion in Paraffin Liquid, 1.0 equiv.). The resulting solution was allowed to stir and raise to room temperature for 16 hours or until complete consumption of the starting material was observed on TLC. The reaction was diluted with water and extracted with ethyl acetate (3×). The organic extracts were combined, washed with brine (2×), dried with MgSO₄, filtered, and concentrated in vacuo. The residue was purified by flash column chromatography to give the desired compound.

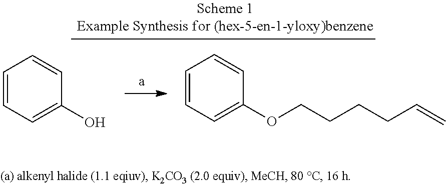

### (pent-4-en-1-yloxy)benzene (1a)

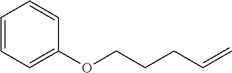

Synthesized according to procedure 1, clear oil, 122 mg, 71% yield. ¹H NMR (500 MHz, CDCl₃) δ 7.33-7.27 (m, 2H), 6.99-6.94 (m, 1H), 6.94-6.91 (m, 2H), 5.88 (ddt, J=16.9, 10.2, 6.6 Hz, 1H), 5.09 (dq, J=17.1, 1.7 Hz, 1H), 5.04-5.01 (m, 1H), 3.99 (t, J=6.4 Hz, 2H), 2.30-2.24 (m, 2H), 1.94-1.88 (m, 2H). ¹³C NMR (126 MHz, CDCl₃) δ 159.2, 138.0, 129.5, 120.7, 115.3, 114.6, 67.2, 30.3, 28.6. HRMS: (ESI) [M+H]⁺ calc. for C₁₁H₁₅O, 163.1117, observed, 163.1120.

### (hex-5-en-1-yloxy)benzene (1b)

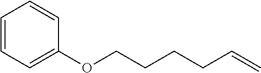

Synthesized according to procedure 1, clear oil, 283 mg, 76% yield. ¹H NMR (500 MHz, CDCl₃) δ 7.32-7.26 (m, 2H), 6.97-6.92 (m, 1H), 6.92-6.89 (m, 2H), 5.85 (ddt, J=16.9, 10.2, 6.6 Hz, 1H), 5.05 (dq, J=17.1, 1.7 Hz, 1H), 5.01-4.97 (m, 1H), 3.98 (t, J=6.5 Hz, 2H), 2.18-2.11 (m, 2H), 1.86-1.78 (m, 2H), 1.63-1.55 (m, 2H). ¹³C NMR (126 MHz, CDCl₃) δ 159.2, 138.7, 129.5, 120.6, 114.9, 114.6, 67.7, 33.6, 28.9, 25.5. HRMS: (ESI) [M+H]⁺ calc. for C₁₂H₁₇O, 177.1274, observed, 177.1282.

### (hept-6-en-1-yloxy)benzene (1c)

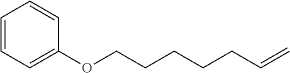

Synthesized according to procedure 1, clear oil, 190 mg, 94% yield. ¹H NMR (600 MHz, CDCl₃) δ 7.32-7.27 (m, 2H), 6.97-6.93 (m, 1H), 6.93-6.90 (m, 2H), 5.91-5.76 (m, 1H), 5.06-5.01 (m, 1H), 4.99-4.96 (m, 1H), 3.97 (t, J=6.5 Hz, 2H), 2.13-2.07 (m, 2H), 1.84-1.78 (m, 2H), 1.53-1.43 (m, 4H). ¹³C NMR (151 MHz, CDCl₃) δ 159.2, 139.0, 129.5, 120.6, 114.6, 67.8, 33.8, 29.3, 28.8, 25.7. HRMS: (ESI) [M+H]⁺ calc. for C₁₃H₁₉O, 191.1430, observed, 191.1441.

### 1-methyl-4-(pent-4-en-1-yloxy)benzene (1d)

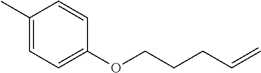

Synthesized according to procedure 1, clear oil, 110 mg, 68% yield. ¹H NMR (600 MHz, CDCl₃) δ 7.10-7.06 (m, 2H), 6.83-6.79 (m, 2H), 5.86 (ddt, J=16.9, 10.2, 6.7 Hz, 1H), 5.07 (dq, J=17.1, 1.7 Hz, 1H), 5.02-4.99 (m, 1H), 3.95 (t, J=6.5 Hz, 2H), 2.29 (s, 3H), 2.27-2.22 (m, 2H), 1.91-1.85 (m, 2H). ¹³C NMR (151 MHz, CDCl₃) δ 157.0, 138.0, 130.0, 129.8, 115.3, 114.4, 67.3, 30.3, 28.6, 20.6. HRMS: (ESI) [M+CH₃₀H₂]⁺ calc. for C₁₃H₂₁O₂, 209.1536, observed, 209.1558.

### 1-(hex-5-en-1-yloxy)-3-methylbenzene (1e)

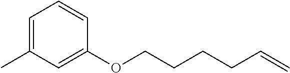

Synthesized according to procedure 1, clear oil, 170 mg, 97% yield. ¹H NMR (500 MHz, CDCl₃) δ 7.16 (t, J=7.8 Hz, 1H), 6.77-6.74 (m, 1H), 6.74-6.72 (m, 1H), 6.72-6.69 (m, 1H), 5.84 (ddt, J=16.9, 10.2, 6.7 Hz, 1H), 5.04 (dq, J=17.1, 1.7 Hz, 1H), 5.00-4.96 (m, 1H), 3.95 (t, J=6.5 Hz, 2H), 2.33 (s, 3H), 2.16-2.10 (m, 2H), 1.84-1.76 (m, 2H), 1.61-1.54 (m, 2H). ¹³C NMR (126 MHz, CDCl₃) δ 159.2, 139.6, 138.7, 129.3, 121.5, 115.5, 114.8, 111.5, 67.7, 33.6, 28.9, 25.5, 21.7. HRMS: (ESI) [M−H]⁻ calc. for C₁₃H₁₇O, 189.1285, observed, 189.1284.

### 1-methyl-3-(pent-4-en-1-yloxy)benzene (1f)

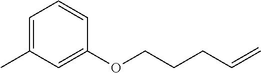

Synthesized according to procedure 1, clear oil, 127 mg, 78% yield. ¹H NMR (500 MHz, CDCl₃) δ 7.17 (t, J=7.8 Hz, 1H), 6.78-6.75 (m, 1H), 6.74-6.73 (m, 1H), 6.72-6.70 (m, 1H), 5.87 (ddt, J=16.9, 10.2, 6.6 Hz, 1H), 5.07 (dq, J=17.1, 1.7 Hz, 1H), 5.02-4.99 (m, 1H), 3.96 (t, J=6.4 Hz, 2H), 2.34 (s, 3H), 2.28-2.22 (m, 2H), 1.92-1.85 (m, 2H). ¹³C NMR (126 MHz, CDCl₃) δ 159.2, 139.6, 138.0, 129.3, 121.5, 115.5, 115.3, 111.5, 67.1, 30.3, 28.6, 21.7. HRMS: (ESI) [M+H]⁺ calc. for C₁₂H₁₇O, 177.1274, observed, 177.1270.

### 1,3-dimethyl-3-(pent-4-en-1-yloxy)benzene (1g)

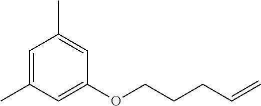

Synthesized according to procedure 1, clear oil, 230 mg, 74% yield. ¹H NMR (500 MHz, CDCl₃) δ 6.62-6.60 (m, 1H), 6.57-6.55 (m, 2H), 5.93-5.84 (m, 1H), 5.09 (dq, J=17.1, 1.8 Hz, 1H), 5.03 (dq, J=10.2, 1.4 Hz, 1H), 3.96 (t, J=6.4 Hz, 2H), 2.31 (s, 6H), 2.29-2.23 (m, 2H), 1.92-1.86 (m, 2H). ¹³C NMR (126 MHz, CDCl₃) δ 159.2, 139.3, 138.1, 122.5, 115.2, 112.4, 67.1, 30.3, 28.7, 21.6. HRMS: (ESI) [M+H]⁺ calc. for C₁₃H₁₉O, 191.1430, observed, 191.1437.

### 1-methyl-2-(pent-4-en-1-yloxy)benzene (1h)

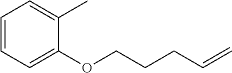

Synthesized according to procedure 1, clear oil, 213 mg, 65% yield. ¹H NMR (500 MHz, CDCl₃) δ 7.18-7.14 (m, 2H), 6.89-6.85 (m, 1H), 6.84-6.81 (m, 1H), 5.90 (ddt, J=16.9, 10.2, 6.7 Hz, 1H), 5.09 (dq, J=17.1, 1.7 Hz, 1H), 5.02 (dq, J=10.2, 1.4 Hz, 1H), 4.00 (t, J=6.3 Hz, 2H), 2.32-2.27 (m, 2H), 2.26 (s, 3H), 1.96-1.90 (m, 2H). ¹³C NMR (126 MHz, CDCl₃) δ 157.3, 138.1, 130.7, 127.0, 126.8, 120.3, 115.2, 111.0, 67.1, 30.4, 28.7, 16.4. HRMS: (ESI) [M+H]⁺ calc. for C₁₂H₁₇O, 177.1274, observed, 177.1288.

### 1-(allyloxy)-4-ethylbenzene (1i)

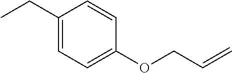

Synthesized according to procedure 1, clear oil, 54 mg, 25% yield. ¹H NMR (500 MHz, CDCl₃) δ 7.16-7.11 (m, 2H), 6.89-6.84 (m, 2H), 5.94 (ddt, J=17.0, 10.2, 6.7 Hz, 1H), 5.20 (dq, J=17.1, 1.7 Hz, 1H), 5.13 (dq, J=10.2, 1.3 Hz, 1H), 4.02 (t, J=6.7 Hz, 2H), 2.62 (q, J=7.6 Hz, 2H), 2.59-2.54 (m, 2H), 1.24 (t, J=7.6 Hz, 3H). ¹³C NMR (126 MHz, CDCl₃) δ 157.1, 136.5, 134.7, 128.8, 117.0, 114.6, 67.4, 33.9, 28.1, 16.0. HRMS: (ESI) [M+K]⁺ calc. for C₁₂H₁₆KO, 215.0833, observed, 215.0851.

### 1-(but-3-en-1-yloxy)-3-ethylbenzene (1j)

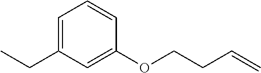

Synthesized according to procedure 1, clear oil, 60 mg, 42% yield. ¹H NMR (500 MHz, CDCl₃) δ 7.20 (t, J=7.8 Hz, 1H), 6.81-6.79 (m, 1H), 6.78-6.77 (m, 1H), 6.75-6.72 (m, 1H), 5.93 (ddt, J=17.1, 10.3, 6.7 Hz, 1H), 5.19 (dq, J=17.2, 1.7 Hz, 1H), 5.12 (dq, J=10.3, 1.3 Hz, 1H), 4.02 (t, J=6.7 Hz, 2H), 2.63 (q, J=7.6 Hz, 2H), 2.58-2.53 (m, 2H), 1.24 (t, J=7.6 Hz, 3H). ¹³C NMR (126 MHz, CDCl₃) δ 159.1, 146.0, 134.7, 129.4, 120.5, 117.1, 114.5, 111.6, 67.2, 33.9, 29.1, 15.6. HRMS: (ESI) [M+H]⁺ calc. for C₁₂H₁₇O, 177.1274, observed, 177.1304.

### 1-(allyloxy)-3-propylbenzene (1k)

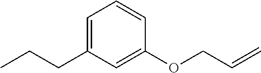

Synthesized according to procedure 1, clear oil, 250 mg, 97% yield. ¹H NMR (500 MHz, CDCl₃) δ 7.19 (t, J=7.8 Hz, 1H), 6.81-6.73 (m, 3H), 6.08 (ddt, J=17.2, 10.5, 5.3 Hz, 1H), 5.43 (dq, J=17.3, 1.6 Hz, 1H), 5.29 (dq, J=10.5, 1.4 Hz, 1H), 4.55-4.53 (m, 2H), 2.59-2.55 (m, 2H), 1.70-1.61 (m, 2H), 0.95 (t, J=7.3 Hz, 3H). ¹³C NMR (126 MHz, CDCl₃) δ 158.7, 144.5, 133.6, 129.2, 121.2, 117.7, 115.2, 111.8, 68.8, 38.2, 24.6, 14.0. HRMS: (ESI) [M+H]⁺ calc. for C₁₂H₁₇O, 177.1274, observed, 177.1279.

### 1-(but-3-en-1-yloxy)-3-isopropylbenzene (1l)

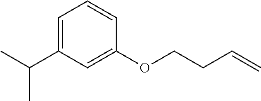

Synthesized according to procedure 1, clear oil, 103 mg, 37% yield. ¹H NMR (500 MHz, CDCl₃) δ 7.21 (t, J=7.9 Hz, 1H), 6.84-6.82 (m, 1H), 6.80-6.79 (m, 1H), 6.74-6.71 (m, 1H), 5.93 (ddt, J=17.0, 10.3, 6.7 Hz, 1H), 5.19 (dq, J=17.2, 1.7 Hz, 1H), 5.12 (dq, J=10.3, 1.4 Hz, 1H), 4.02 (t, J=6.7 Hz, 2H), 2.88 (p, J=6.9 Hz, 1H), 2.58-2.53 (m, 2H), 1.25 (d, J=6.9 Hz, 6H). ¹³C NMR (126 MHz, CDCl₃) δ 159.1, 150.8, 134.7, 129.4, 119.1, 117.1, 113.3, 111.4, 67.2, 34.3, 33.9, 24.1. HRMS: (ESI) [M+H]⁺ calc. for C₃H₁₉O, 191.1430, observed, 191.1429.

### 1-(but-3-en-1-yloxy)-3-cyclopropylbenzene (1m)

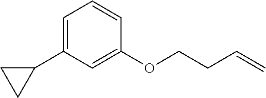

Synthesized according to procedure 1, clear oil, 140 mg, 40% yield. ¹H NMR (600 MHz, CDCl₃) δ 7.16 (t, J=7.9 Hz, 1H), 6.70-6.66 (m, 2H), 6.63-6.61 (m, 1H), 5.97-5.85 (m, 1H), 5.17 (dq, J=17.1, 1.6 Hz, 1H), 5.11 (dq, J=10.2, 1.4 Hz, 1H), 4.00 (t, J=6.7 Hz, 2H), 2.58-2.49 (m, 2H), 1.89-1.83 (m, 1H), 0.96-0.92 (m, 2H), 0.71-0.67 (m, 2H). ¹³C NMR (151 MHz, CDCl₃) δ 159.2, 145.9, 134.7, 129.3, 118.3, 117.1, 112.3, 111.3, 67.2, 33.8, 15.6, 9.4. HRMS: (ESI) [M+H]⁺ calc. for C₁₃H₁₇O, 189.1274, observed, 189.1279.

### 1-(but-3-en-1-yloxy)-3-(tert-butyl)benzene (1n)

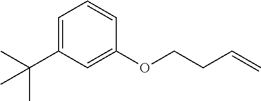

Synthesized according to procedure 1, clear oil, 127 mg, 47% yield. ¹H NMR (500 MHz, CDCl₃) δ 7.22 (t, J=8.0 Hz, 1H), 7.00-6.97 (m, 1H), 6.96-6.94 (m, 1H), 6.73-6.70 (m, 1H), 5.93 (ddt, J=17.0, 10.2, 6.7 Hz, 1H), 5.18 (dq, J=17.2, 1.7 Hz, 1H), 5.12 (dq, J=10.3, 1.4 Hz, 1H), 4.02 (t, J=6.7 Hz, 2H), 2.56 (qt, J=6.8, 1.4 Hz, 2H), 1.31 (s, 9H). ¹³C NMR (126 MHz, CDCl₃) δ 158.9, 153.1, 134.7, 129.1, 118.0, 117.1, 112.8, 110.7, 67.2, 34.9, 33.9, 31.5. HRMS: (ESI) [M+H]⁺ calc. for C₁₄H₂₁O, 205.1587, observed, 205.1596.

### 1-(pent-4-en-1-yloxy)-4-(trifluoromethyl)benzene (1o)

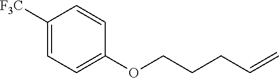 [SMILES: C=CCCCOc1ccc(C(F)(F)F)cc1]

Synthesized according to procedure 1, clear oil, 83 mg, 58% yield. ¹H NMR (600 MHz, CDCl₃) δ 7.55-7.51 (m, 2H), 6.97-6.93 (m, 2H), 5.89-5.81 (m, 1H), 5.07 (dq, J=17.1, 1.7 Hz, 1H), 5.02 (dq, J=10.1, 1.4 Hz, 1H), 4.01 (t, J=6.4 Hz, 2H), 2.28-2.22 (m, 2H), 1.91 (p, J=6.6 Hz, 2H). ¹⁹F NMR (565 MHz, CDCl₃) δ −61.40. ¹³C NMR (151 MHz, CDCl₃) δ 161.6 (q, J=1.1 Hz), 137.7, 127.0 (q, J=3.7 Hz), 124.6 (q, J=271.3 Hz), 122.8 (q, J=32.7 Hz), 115.6, 114.5, 67.4, 30.1, 28.3. HRMS: (ESI) [M+CH₃₀H₂]⁺ calc. for C₁₃H₁₈F₃O₂, 263.1253, observed, 263.1256.

### 1-(pent-4-en-1-yloxy)-3-(trifluoromethyl)benzene (1p)

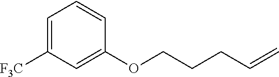 [SMILES: C=CCCCOc1cccc(C(F)(F)F)c1]

Synthesized according to procedure 1, clear oil, 104 mg, 73% yield. ¹H NMR (500 MHz, CDCl₃) δ 7.40-7.35 (m, 1H), 7.21-7.17 (m, 1H), 7.13 (t, J=2.1 Hz, 1H), 7.06 (dd, J=8.3, 2.6 Hz, 1H), 5.86 (ddt, J=16.9, 10.2, 6.7 Hz, 1H), 5.08 (dq, J=17.1, 1.7 Hz, 1H), 5.02 (dq, J=10.2, 1.4 Hz, 1H), 4.00 (t, J=6.4 Hz, 2H), 2.29-2.23 (m, 2H), 1.95-1.87 (m, 2H). ¹⁹F NMR (565 MHz, CDCl₃) δ −62.66. ¹³C NMR (126 MHz, CDCl₃) δ 159.3, 137.7, 131.9 (q, J=32.2 Hz), 130.0, 124.2 (q, J=272.3 Hz), 118.1 (q, J=1.1 Hz), 117.4 (q, J=4.0 Hz), 115.5, 111.4 (q, J=3.9 Hz), 67.5, 30.2, 28.4. HRMS: (ESI) [M+H]⁺ calc. for C₁₂H₁₄F₃O, 231.0991, observed, 231.0983.

### 1-(but-3-en-1-yloxy)-3-methoxybenzene (1q)

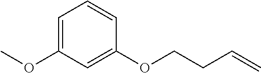

Synthesized according to procedure 1, clear oil, 94 mg, 33% yield. ¹H NMR (600 MHz, CDCl₃) δ 7.17 (t, J=8.2 Hz, 1H), 6.51 (dd, J=8.2, 2.4 Hz, 2H), 6.47 (t, J=2.4 Hz, 1H), 5.91 (ddt, J=17.0, 10.3, 6.7 Hz, 1H), 5.17 (dq, J=17.2, 1.7 Hz, 1H), 5.11 (dq, J=10.3, 1.4 Hz, 1H), 4.00 (t, J=6.7 Hz, 2H), 3.79 (s, 3H), 2.56-2.50 (m, 2H). ¹³C NMR (151 MHz, CDCl₃) δ 161.0, 160.3, 134.6, 130.0, 117.2, 106.8, 106.5, 101.1, 67.3, 55.4, 33.8. HRMS: (ESI) [M+H]⁺ calc. for C₁₁H₁₃O₂, 179.1067, observed, 179.1065.

### 1-(but-3-en-1-yloxy)-3-(trifluoromethoxy)benzene (1r)

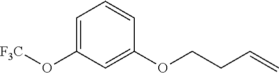 [SMILES: C=CCCOc1cccc(OC(F)(F)F)c1]

Synthesized according to procedure 1, clear oil, 70 mg, 27% yield. ¹H NMR (500 MHz, CDCl₃) δ 7.27 (t, J=8.3 Hz, 1H), 6.85-6.79 (m, 2H), 6.77-6.75 (m, 1H), 5.90 (ddt, J=17.0, 10.3, 6.7 Hz, 1H), 5.18 (dq, J=17.2, 1.7 Hz, 1H), 5.13 (dq, J=10.3, 1.4 Hz, 1H), 4.01 (t, J=6.7 Hz, 2H), 2.55 (qt, J=6.7, 1.4 Hz, 2H). ¹⁹F NMR (376 MHz, CDCl₃) δ −57.77. ¹³C NMR (126 MHz, CDCl₃) δ 160.1, 150.3 (q, J=2.0 Hz), 134.2, 130.3, 120.6 (q, J=257.0 Hz), 117.4, 113.1, 113.0, 107.8, 67.7, 33.6. HRMS: (ESI) [M+H]⁺ calc. for C₁₁H₁₂F₃O₂, 233.0784, observed, 233.0784.

### 3-(but-3-en-1-yloxy)benzonitrile (1s)

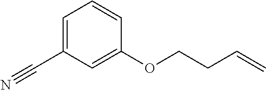

Synthesized according to procedure 1, clear oil, 128 mg, 44% yield. ¹H NMR (500 MHz, CDCl₃) δ 7.38-7.33 (m, 1H), 7.25-7.22 (m, 1H), 7.15-7.11 (m, 2H), 5.88 (ddt, J=17.0, 10.2, 6.7 Hz, 1H), 5.18 (dq, J=17.1, 1.6 Hz, 1H), 5.13 (dq, J=10.3, 1.4 Hz, 1H), 4.02 (t, J=6.7 Hz, 2H), 2.56 (qt, J=6.5, 1.3 Hz, 2H). ¹³C NMR (126 MHz, CDCl₃) δ 159.1, 134.0, 130.4, 124.6, 120.0, 118.9, 117.6, 117.6, 113.3, 67.7, 33.5. HRMS: (ESI) [M+H]⁺ calc. for C₁₁H₁₂NO, 174.0913, observed, 174.091.

### 2-(but-3-en-1-yloxy)naphthalene (1t)

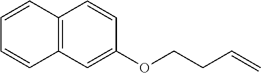

Synthesized according to procedure 1, clear oil, 150 mg, 55% yield. ¹H NMR (500 MHz, CDCl₃) δ 7.80-7.77 (m, 1H), 7.77-7.73 (m, 2H), 7.47-7.43 (m, 1H), 7.37-7.33 (m, 1H), 7.19-7.17 (m, 1H), 7.16-7.15 (m, 1H), 5.98 (ddt, J=17.0, 10.3, 6.7 Hz, 1H), 5.26-5.21 (m, 1H), 5.18-5.15 (m, 1H), 4.15 (t, J=6.7 Hz, 2H), 2.66-2.61 (m, 2H). ¹³C NMR (126 MHz, CDCl₃) δ 157.0, 134.7, 134.6, 129.5, 129.1, 127.8, 126.8, 126.5, 123.7, 119.1, 117.2, 106.8, 67.3, 33.8. HRMS: (ESI) [M+H]⁺ calc. for C₁₄H₁₅O, 199.1117, observed, 199.1120.

### 1-(but-3-en-1-yloxy)-3-methoxy-5-methylbenzene (1u)

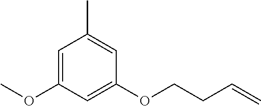

Synthesized according to procedure 1, clear oil, 94 mg, 34% yield. ¹H NMR (500 MHz, CDCl₃) δ 6.35-6.32 (m, 2H), 6.30-6.28 (m, 1H), 5.95-5.86 (m, 1H), 5.17 (dq, J=17.1, 1.8 Hz, 1H), 5.11 (dq, J=10.4, 1.4 Hz, 1H), 3.99 (t, J=6.7 Hz, 2H), 3.77 (s, 3H), 2.56-2.50 (m, 2H), 2.30 (s, 3H). ¹³C NMR (126 MHz, CDCl₃) δ 160.8, 160.1, 140.3, 134.6, 117.1, 107.8, 107.3, 98.2, 67.3, 55.4, 33.8, 21.9. HRMS: (ESI) [M+H]⁺ calc. for C₁₂H₁₇O₂, 193.1223, observed, 193.1223.

### 1-fluoro-4-(hex-5-en-1-yloxy)benzene (1v)

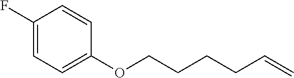

Synthesized according to procedure 1, clear oil, 303 mg, 87% yield. ¹H NMR (500 MHz, CDCl₃) δ 7.00-6.93 (m, 2H), 6.85-6.80 (m, 2H), 5.83 (ddt, J=16.9, 10.2, 6.6 Hz, 1H), 5.04 (dq, J=17.2, 1.7 Hz, 1H), 5.00-4.96 (m, 1H), 3.92 (t, J=6.4 Hz, 2H), 2.16-2.10 (m, 2H), 1.82-1.75 (m, 2H), 1.60-1.53 (m, 2H). ¹⁹F NMR (376 MHz, CDCl₃) δ −124.41-−124.48 (m). ¹³C NMR (126 MHz, CDCl₃) δ 157.3 (d, J=237.9 Hz), 155.3 (d, J=2.2 Hz), 138.6, 115.9 (d, J=22.9 Hz), 115.5 (d, J=7.9 Hz), 114.9, 68.5, 33.6, 28.9, 25.4. HRMS: (ESI) [M+H]⁺ calc. for C₁₂H₁₆FO, 195.1180, observed, 195.1174.

### 1-fluoro-3-(hex-5-en-1-yloxy)benzene (1w)

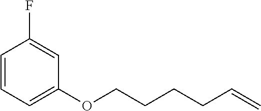

Synthesized according to procedure 1, clear oil, 291 mg, 84% yield. ¹H NMR (500 MHz, CDCl₃) δ 7.23-7.18 (m, 1H), 6.69-6.66 (m, 1H), 6.66-6.59 (m, 2H), 5.83 (ddt, J=16.9, 10.1, 6.6 Hz, 1H), 5.04 (dq, J=17.1, 1.7 Hz, 1H), 5.00-4.97 (m, 1H), 3.95 (t, J=6.5 Hz, 2H), 2.16-2.11 (m, 2H), 1.83-1.77 (m, 2H), 1.61-1.54 (m, 2H). ¹⁹F NMR (376 MHz, CDCl₃) δ −111.87-−111.96 (m). ¹³C NMR (126 MHz, CDCl₃) δ 163.8 (d, J=244.8 Hz), 160.6 (d, J=10.9 Hz), 138.6, 130.3 (d, J=10.2 Hz), 115.0, 110.5 (d, J=2.8 Hz), 107.4 (d, J=21.3 Hz), 102.2 (d, J=24.6 Hz), 68.2, 33.5, 28.7, 25.4. HRMS: (ESI) [M+NH₄]⁺ calc. for C₁₂H₁₉FNO, 212.1445, observed, 212.1428.

### 1-fluoro-2-(hex-5-en-1-yloxy)benzene (1x)

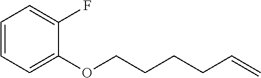

Synthesized according to procedure 1, clear oil, 340 mg, 98% yield. ¹H NMR (500 MHz, CDCl₃) δ 7.10-7.02 (m, 2H), 6.98-6.94 (m, 1H), 6.90-6.86 (m, 1H), 5.83 (ddt, J=17.0, 10.2, 6.6 Hz, 1H), 5.04 (dq, J=17.1, 1.7 Hz, 1H), 5.00-4.96 (m, 1H), 4.04 (t, J=6.5 Hz, 2H), 2.17-2.11 (m, 2H), 1.88-1.81 (m, 2H), 1.63-1.56 (m, 2H). ¹⁹F NMR (376 MHz, CDCl₃) δ −134.77-−134.85 (m). ¹³C NMR (126 MHz, CDCl₃) δ 152.9 (d, J=245.3 Hz), 147.3 (d, J=10.6 Hz), 138.6, 124.4 (d, J=3.9 Hz), 121.0 (d, J=6.8 Hz), 116.3 (d, J=18.4 Hz), 115.0 (d, J=1.9 Hz), 114.9, 69.3, 33.5, 28.8, 25.3. HRMS: (ESI) [M+H]⁺ calc. for C₁₂H₁₆FO, 195.1180, observed, 195.1196.

### 1,3-difluoro-5-(hex-5-en-1-yloxy)benzene (1y)

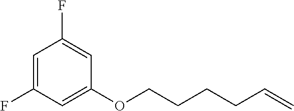

Synthesized according to procedure 1, clear oil, 317 mg, 97% yield. ¹H NMR (500 MHz, CDCl₃) δ 6.44-6.37 (m, 3H), 5.83 (ddt, J=16.9, 10.2, 6.6 Hz, 1H), 5.05 (dq, J=17.1, 1.8 Hz, 1H), 5.00 (dq, J=10.2, 1.5 Hz, 1H), 3.92 (t, J=6.4 Hz, 2H), 2.13 (q, J=7.1 Hz, 2H), 1.80 (dt, J=15.1, 6.5 Hz, 2H), 1.61-1.52 (m, 2H). ¹⁹F NMR (376 MHz, CDCl₃) δ −109.61-−109.69 (m). ¹³C NMR (126 MHz, CDCl₃) δ 163.8 (dd, J=245.8, 15.8 Hz), 161.2 (t, J=13.6 Hz), 138.5, 115.0, 98.5-98.2 (m), 96.2 (t, J=26.0 Hz), 68.5, 33.5, 28.5, 25.3. HRMS: (ESI) [M+H]⁺ calc. for C₁₂H₁₅F₂O, 213.1085, observed, 213.1086.

### 1,2-difluoro-3-(hex-5-en-1-yloxy)benzene (1z)

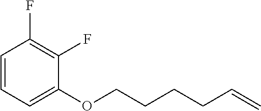

Synthesized according to procedure 1, clear oil, 340 mg, 98% yield. ¹H NMR (500 MHz, CDCl₃) δ 6.99-6.93 (m, 1H), 6.78-6.70 (m, 2H), 5.83 (ddt, J=17.0, 10.2, 6.7 Hz, 1H), 5.04 (dq, J=17.2, 1.8 Hz, 1H), 5.00-4.97 (m, 1H), 4.04 (t, J=6.5 Hz, 2H), 2.17-2.11 (m, 2H), 1.88-1.81 (m, 2H), 1.63-1.55 (m, 2H). ¹⁹F NMR (376 MHz, CDCl₃) δ −137.40-−137.51 (m), −159.51-−159.63 (m). ¹³C NMR (126 MHz, CDCl₃) δ 151.6 (dd, J=246.4, 10.4 Hz), 148.9 (dd, J=7.8, 3.2 Hz), 141.58 (dd, J=247.1, 14.1 Hz), 138.5, 123.2 (dd, J=8.7, 5.3 Hz), 115.0, 109.9 (d, J=2.9 Hz), 109.1 (d, J=17.7 Hz), 69.7, 33.5, 28.7, 25.3. HRMS: (ESI) [M+H]⁺ calc. for C₁₂H₁₅F₂O, 213.1085, observed, 213.1091.

### 1,3-difluoro-2-(hex-5-en-1-yloxy)benzene (1aa)

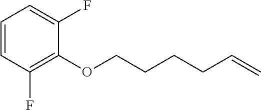

Synthesized according to procedure 1, clear oil, 293 mg, 90% yield. ¹H NMR (600 MHz, CDCl₃) δ 6.96-6.90 (m, 1H), 6.90-6.85 (m, 2H), 5.83 (ddt, J=16.9, 10.2, 6.7 Hz, 1H), 5.03 (dq, J=17.1, 1.6 Hz, 1H), 4.98-4.96 (m, 1H), 4.13 (t, J=6.5 Hz, 2H), 2.15-2.10 (m, 2H), 1.81-1.76 (m, 2H), 1.62-1.57 (m, 2H). ¹⁹F NMR (565 MHz, CDCl₃) δ −128.32-−128.39 (m). ¹³C NMR (126 MHz, CDCl₃) δ 156.5 (dd, J=248.1, 5.5 Hz), 138.7, 136.0 (t, J=14.2 Hz), 122.7 (t, J=9.3 Hz), 114.8, 112.2 (dd, J=17.3, 5.5 Hz), 74.7, 33.5, 29.5, 25.1. HRMS: (ESI) [M+H]⁺ calc. for C₁₂H₁₅F₂O, 213.1085, observed, 213.1082.

### 1,3,5-trifluoro-2-(hex-5-en-1-yloxy)benzene (1ab)

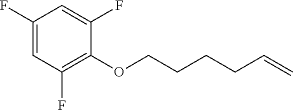 [SMILES: C=CCCCCOc1c(F)cc(F)cc1F]

Synthesized according to procedure 1, clear oil, 123 mg, 40% yield. ¹H NMR (600 MHz, CDCl₃) δ 6.70-6.63 (m, 2H), 5.82 (ddt, J=17.0, 10.2, 6.7 Hz, 1H), 5.03 (dq, J=17.1, 1.7 Hz, 1H), 4.97 (ddt, J=10.2, 2.3, 1.3 Hz, 1H), 4.06 (t, J=6.6 Hz, 2H), 2.14-2.07 (m, 2H), 1.79-1.73 (m, 2H), 1.61-1.53 (m, 2H). ¹⁹F NMR (565 MHz, CDCl₃) δ −115.20-−115.24 (m), −125.17-−125.22 (m). ¹³C NMR (126 MHz, CDCl₃) δ 157.1 (dt, J=245.1, 14.4 Hz), 156.4 (ddd, J=249.4, 14.9, 7.8 Hz), 138.6, 132.8 (td, J=14.6, 5.1 Hz), 114.9, 101.0-100.6 (m), 75.1 (t, J=2.9 Hz), 33.5, 29.4, 25.1. HRMS: (ESI) [M+H]⁺ calc. for C₁₂H₁₄F₃O, 231.0991, observed, 231.1022.

### 3-(hex-5-en-1-yloxy)pyridine (1ac)

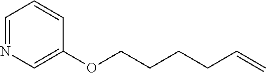

Synthesized according to procedure 1, clear oil, 106 mg, 28% yield. ¹H NMR (500 MHz, CDCl₃) δ 8.32-8.28 (m, 1H), 8.21-8.19 (m, 1H), 7.22-7.15 (m, 2H), 5.82 (ddt, J=16.9, 10.2, 6.7 Hz, 1H), 5.04 (dq, J=17.1, 1.7 Hz, 1H), 4.99-4.96 (m, 1H), 4.00 (t, J=6.4 Hz, 2H), 2.16-2.09 (m, 2H), 1.85-1.77 (m, 2H), 1.61-1.53 (m, 2H). ¹³C NMR (126 MHz, CDCl₃) δ 155.4, 142.1, 138.5, 138.1, 123.9, 121.2, 115.0, 68.2, 33.5, 28.7, 25.3.

### 1-(allyloxy)-4-cyclopropylbenzene (1ad)

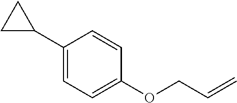

Synthesized according to procedure 1, clear oil, 320 mg, 99% yield. ¹H NMR (500 MHz, CDCl₃) δ 7.04-6.99 (m, 2H), 6.85-6.81 (m, 2H), 6.06 (ddt, J=17.2, 10.6, 5.3 Hz, 1H), 5.41 (dq, J=17.3, 1.6 Hz, 1H), 5.28 (dq, J=10.5, 1.4 Hz, 1H), 4.52 (dt, J=5.3, 1.5 Hz, 2H), 1.89-1.83 (m, 1H), 0.93-0.88 (m, 2H), 0.65-0.61 (m, 2H). ¹³C NMR (126 MHz, CDCl₃) δ 156.7, 136.2, 133.6, 126.9, 117.6, 114.8, 69.0, 14.7, 8.7. HRMS: (ESI) [M+H]⁺ calc. for C₁₂H₁₅O, 175.1117, observed, 175.1116.

### 1-ethyl-2-(hex-5-en-1-yloxy)benzene (1ae)

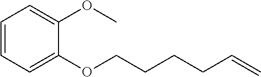

Synthesized according to procedure 1, clear oil, 232 mg, 69% yield. ¹H NMR (600 MHz, CDCl₃) δ 7.18-7.14 (m, 2H), 6.91-6.88 (m, 1H), 6.83 (d, J=8.2 Hz, 1H), 5.86 (ddt, J=16.9, 10.2, 6.6 Hz, 1H), 5.06 (dq, J=17.1, 1.6 Hz, 1H), 5.01-4.98 (m, 1H), 3.98 (t, J=6.3 Hz, 2H), 2.67 (q, J=7.5 Hz, 2H), 2.16 (q, J=7.2 Hz, 2H), 1.87-1.81 (m, 3H), 1.65-1.59 (m, 2H), 1.21 (t, J=7.5 Hz, 3H). ¹³C NMR (151 MHz, CDCl₃) δ 156.9, 138.8, 132.9, 129.0, 126.8, 120.3, 114.8, 111.0, 67.6, 33.6, 29.0, 25.6, 23.5, 14.3.

### 1-(hex-5-en-1-yloxy)-2-(trifluoromethyl)benzene (1af)

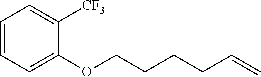 [SMILES: C=CCCCCOc1ccccc1C(F)(F)F]

Synthesized according to procedure 1, clear oil, 281 mg, 93% yield. ¹H NMR (600 MHz, CDCl₃) δ 7.56 (d, J=7.5 Hz, 1H), 7.49-7.45 (m, 1H), 7.00-6.96 (m, 2H), 5.83 (ddt, J=16.9, 10.2, 6.6 Hz, 1H), 5.04 (dt, J=17.1, 1.8 Hz, 1H), 5.00-4.97 (m, 1H), 4.05 (t, J=6.3 Hz, 2H), 2.13 (q, J=7.2 Hz, 2H), 1.87-1.81 (m, 2H), 1.63-1.58 (m, 2H). ¹⁹F NMR (565 MHz, CDCl₃) δ −62.42. ¹³C NMR (151 MHz, CDCl₃) δ 157.2, 157.2, 138.7, 133.3, 127.2 (q, J=5.2 Hz), 123.9 (q, J=272.3 Hz), 119.9, 119.0 (q, J=30.7 Hz), 114.9, 112.8, 68.5, 33.4, 28.6, 25.2. HRMS: (ESI) [M+H]⁺ calc. for C₁₃H₁₆F₃O, 245.1148, observed, 245.1131.

### 1-ethyl-2-(pent-4-en-1-yloxy)benzene (1ag)

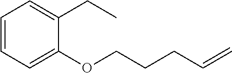

Synthesized according to procedure 1, clear oil, 158 mg, 51% yield. ¹H NMR (600 MHz, CDCl₃) δ 7.18-7.15 (m, 2H), 6.90 (t, J=7.4 Hz, 1H), 6.84 (d, J=7.9 Hz, 1H), 5.89 (ddt, J=16.9, 10.2, 6.7 Hz, 1H), 5.09 (dq, J=17.1, 1.6 Hz, 1H), 5.03 (dq, J=10.2, 1.2 Hz, 1H), 3.99 (t, J=6.3 Hz, 2H), 2.68 (q, J=7.5 Hz, 2H), 2.29 (q, J=6.9 Hz, 2H), 1.96-1.89 (m, 2H), 1.23 (t, J=7.5 Hz, 3H). ¹³C NMR (151 MHz, CDCl₃) δ 156.9, 138.1, 132.9, 129.1, 126.8, 120.4, 115.2, 111.1, 67.0, 30.4, 28.8, 23.5, 14.4. HRMS: (ESI) [M+H]⁺ calc. for C₁₃H₁₉O, 191.1430, observed, 191.1434.

### 1-(but-3-en-1-yloxy)-3-cyclopropyl-2-fluorobenzene (1ah)

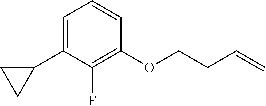

Synthesized according to procedure 1, clear oil, 182 mg, 54% yield. ¹H NMR (600 MHz, CDCl₃) δ 6.92 (td, J=8.0, 1.5 Hz, 1H), 6.76 (td, J=8.0, 1.5 Hz, 1H), 6.48-6.44 (m, 1H), 5.92 (ddt, J=17.1, 10.3, 6.7 Hz, 1H), 5.19 (dq, J=17.2, 1.6 Hz, 1H), 5.14-5.10 (m, 1H), 4.07 (t, J=6.8 Hz, 2H), 2.61-2.55 (m, 2H), 2.13-2.07 (m, 1H), 1.00-0.95 (m, 2H), 0.73-0.69 (m, 2H). ¹⁹F NMR (565 MHz, CDCl₃) δ −142.22 (t, J=7.1 Hz). ¹³C NMR (151 MHz, CDCl₃) δ 152.1 (d, J=244.1 Hz), 146.9 (d, J=11.2 Hz), 134.3, 131.9 (d, J=11.9 Hz), 123.6 (d, J=4.7 Hz), 117.5 (d, J=3.3 Hz), 117.3, 112.0 (d, J=1.4 Hz), 68.8, 33.8, 8.8, 8.7, 8.0. HRMS: (ESI) [M+H]⁺ calc. for C₁₃H₁₆FO, 207.1180, observed, 207.1189.

### 1-(but-3-en-1-yloxy)-3-cyclopropyl-5-fluorobenzene (1ai)

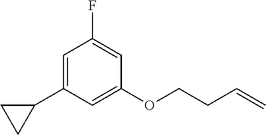

Synthesized according to procedure 1, clear oil, 150 mg, 44% yield. ¹H NMR (600 MHz, CDCl₃) δ 6.42 (s, 1H), 6.39 (dt, J=10.6, 2.3 Hz, 1H), 6.36-6.33 (m, 1H), 5.89 (ddt, J=17.0, 10.3, 6.7 Hz, 1H), 5.19-5.15 (m, 1H), 5.13-5.10 (m, 1H), 3.97 (t, J=6.7 Hz, 2H), 2.53 (q, J=6.7 Hz, 2H), 1.87-1.80 (m, 1H), 0.98-0.92 (m, 2H), 0.70-0.65 (m, 2H). ¹⁹F NMR (565 MHz, CDCl₃) δ −112.71 (t, J=10.2 Hz). ¹³C NMR (151 MHz, CDCl₃) δ 171.3, 163.8 (d, J=243.6 Hz), 160.2 (d, J=11.7 Hz), 147.6 (d, J=9.8 Hz), 134.4, 117.3, 108.2 (d, J=2.5 Hz), 104.8 (d, J=22.1 Hz), 99.1 (d, J=25.2 Hz), 67.5, 60.5, 33.6, 21.2, 15.7, 15.7, 14.3, 9.6. HRMS: (ESI) [M+H]⁺ calc. for C₁₃H₁₆FO, 207.1180, observed, 207.1188.

### 1-(allyloxy)-3-cyclobutylbenzene (1aj)

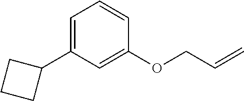

Synthesized according to procedure 1, clear oil, 120 mg, 38% yield. ¹H NMR (500 MHz, CDCl₃) δ 7.21 (t, J=7.8 Hz, 1H), 6.83-6.79 (m, 2H), 6.74 (dd, J=8.1, 2.3 Hz, 1H), 6.08 (ddt, J=17.2, 10.6, 5.3 Hz, 1H), 5.43 (dq, J=17.2, 1.6 Hz, 1H), 5.29 (dq, J=10.5, 1.3 Hz, 1H), 4.54 (dt, J=5.3, 1.4 Hz, 2H), 3.53 (p, J=8.8 Hz, 1H), 2.37-2.30 (m, 2H), 2.20-2.10 (m, 2H), 2.06-1.95 (m, 1H), 1.89-1.81 (m, 1H). ¹³C NMR (126 MHz, CDCl₃) δ 158.8, 148.2, 133.6, 129.3, 119.0, 117.7, 113.1, 111.7, 68.9, 40.4, 29.8, 18.4. HRMS: (ESI) [M+H]⁺ calc. for C₁₃H₁₇O, 189.1274, observed, 189.1263.

### 1-chloro-3-(pent-4-en-1-yloxy)benzene (1aq)

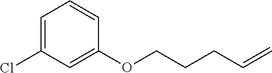

Synthesized according to procedure 1, clear oil, 160 mg, 52% yield. ¹H NMR (600 MHz, CDCl₃) δ 7.19 (t, J=8.1 Hz, 1H), 6.93-6.91 (m, 1H), 6.89 (t, J=2.2 Hz, 1H), 6.78 (dd, J=8.3, 2.4 Hz, 1H), 5.90-5.80 (m, 1H), 5.09-5.05 (m, 1H), 5.03-5.00 (m, 2H), 3.95 (t, J=6.4 Hz, 2H), 2.24 (q, J=7.2 Hz, 2H), 1.88 (dt, J=13.6, 6.6 Hz, 2H). ¹³C NMR (151 MHz, CDCl₃) δ 159.9, 137.8, 134.9, 130.3, 120.8, 115.5, 114.9, 113.2, 67.4, 30.2, 28.4. HRMS: (ESI) [M+H]⁺ calc. for C₁₁H₁₄ClO, 197.0728, observed, 197.0717.

### (allyloxy)benzene (1at)

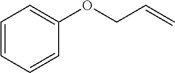

Synthesized according to procedure 1, clear oil, 204 mg, 95% yield. ¹H NMR (500 MHz, CDCl₃) δ 7.31-7.27 (m, 2H), 6.98-6.91 (m, 2H), 6.12-6.02 (m, 1H), 5.43 (dq, J=17.3, 1.5 Hz, 1H), 5.31-5.28 (m, 1H), 4.55 (dt, J=5.3, 1.4 Hz, 2H). ¹³C NMR (126 MHz, CDCl₃) δ 158.7, 133.5, 129.6, 121.0, 117.8, 114.8, 68.8.

### 1-(but-3-en-1-yloxy)naphthalene (1au)

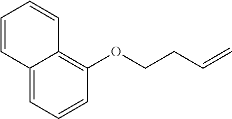

Synthesized according to procedure 1, clear oil, 176 mg, 51% yield. ¹H NMR (600 MHz, CDCl₃) δ 8.32-8.29 (m, 1H), 7.83-7.80 (m, 1H), 7.52-7.47 (m, 2H), 7.45-7.43 (m, 1H), 7.40-7.36 (m, 1H), 6.82 (d, J=7.5 Hz, 1H), 6.07-5.99 (m, 1H), 5.29-5.24 (m, 1H), 5.19-5.15 (m, 1H), 4.21 (t, J=6.5 Hz, 2H), 2.71 (q, J=6.6 Hz, 2H). ¹³C NMR (151 MHz, CDCl₃) δ 154.8, 134.8, 134.6, 127.5, 126.5, 126.0, 125.8, 125.2, 122.2, 120.3, 117.2, 104.7, 67.5, 33.9. HRMS: (ESI) [M+H]⁺ calc. for C₁₄H₁₅O, 199.1117, observed, 199.1104.

### (hex-5-en-1-yloxy)cyclopentane (1av)

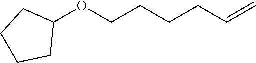

Synthesized according to procedure 21, clear oil, 42 mg, 11% yield. ¹H NMR (500 MHz, CDCl₃) δ 5.86-5.76 (m, 1H), 5.02-4.97 (m, 1H), 4.95-4.92 (m, 1H), 3.89-3.83 (m, 1H), 3.36 (t, J=6.7 Hz, 2H), 2.06 (q, J=7.1 Hz, 2H), 1.75-1.64 (m, 4H), 1.63-1.53 (m, 4H), 1.52-1.48 (m, 2H), 1.47-1.39 (m, 2H). ¹³C NMR (126 MHz, CDCl₃) δ 139.0, 114.6, 81.4, 68.8, 33.8, 32.4, 29.7, 25.8, 23.7. HRMS: (ESI) [M+Na]⁺ calc. for C₁₁H₂₀ONa, 191.1406, observed, 191.1427.

### ((pent-4-en-1-yloxy)methyl)benzene (1aw)

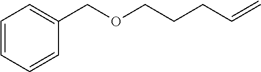

Synthesized according to procedure 21, white solid, 61 mg, 86% yield. ¹H NMR (600 MHz, CDCl₃) δ 7.36-7.34 (m, 4H), 7.31-7.27 (m, 1H), 5.87-5.78 (m, 1H), 5.05-5.01 (m, 2H), 4.98-4.95 (m, 1H), 4.51 (s, 2H), 3.49 (t, J=6.5 Hz, 2H), 2.15 (q, J=7.1 Hz, 2H), 1.72 (p, J=6.8 Hz, 2H). ¹³C NMR (151 MHz, CDCl₃) δ 138.7, 138.4, 128.5, 127.8, 127.6, 114.9, 73.0, 69.9, 30.5, 29.1. HRMS: (ESI) [M+H]⁺ calc. for C₁₂H₁₇O, 177.1274, observed, 177.1277.

### (3-(allyloxy)propyl)benzene (1ax)

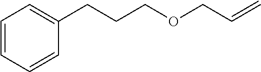

Synthesized according to procedure 21, clear oil, 34 mg, 13% yield. ¹H NMR (600 MHz, CDCl₃) δ 7.29 (t, J=7.6 Hz, 2H), 7.22-7.17 (m, 3H), 5.94 (ddt, J=16.3, 10.8, 5.6 Hz, 1H), 5.31-5.26 (m, 1H), 5.20-5.17 (m, 1H), 3.98 (d, J=5.6 Hz, 2H), 3.45 (t, J=6.4 Hz, 2H), 2.73-2.69 (m, 2H), 1.95-1.89 (m, 2H). ¹³C NMR (151 MHz, CDCl₃) δ 142.1, 135.1, 128.6, 128.4, 125.9, 116.9, 72.0, 69.6, 32.5, 31.5. HRMS: (ESI) [M+H]⁺ calc. for C₁₂H₁₇O, 177.1274, observed, 177.1289.

### (hex-5-en-1-yloxy)cyclobutene (1ay)

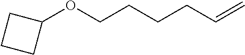

Synthesized according to procedure 21, clear oil, 188 mg, 35% yield. ¹H NMR (600 MHz, CDCl₃) δ 5.80 (ddt, J=16.9, 10.2, 6.7 Hz, 1H), 5.00 (dq, J=17.1, 1.6 Hz, 1H), 4.95-4.92 (m, 1H), 3.93-3.84 (m, 1H), 3.31 (t, J=6.7 Hz, 1H), 2.22-2.15 (m, 2H), 2.06 (q, J=7.1 Hz, 2H), 1.93-1.86 (m, 2H), 1.71-1.64 (m, 1H), 1.59-1.52 (m, 2H), 1.53-1.40 (m, 3H). ¹³C NMR (151 MHz, CDCl₃) δ 138.9, 114.6, 73.2, 67.7, 33.7, 30.6, 29.4, 25.7, 12.7. HRMS: (ESI) [M+H]⁺ calc. for C₁₀H₁₉O, 155.1430, observed, 155.1442.

### N-(pent-4-en-1-yl)benzamide, 1az

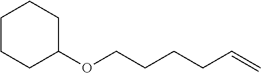

To a stirring solution of hex-5-en-1-ol (0.20 g, 0.24 mL, 1.0 eq., 2.0 mmol), triethylsilane (0.47 g, 0.64 mL, 2.0 eq., 4.0 mmol) and ytterbium(III) trifluoromethanesulfonate hydrate (51 mg, 0.04 eq., 80 mol) in 1,2-Dichloroethane (8.0 mL) was added cyclohexanone (0.39 g, 0.41 mL, 2.0 eq., 4.0 mmol) under a nitrogen atmosphere. The reaction was heated under reflux for 24 hours, allowed to cool to room temperature, diluted with water (15 mL), then extracted with diethyl ether (3×5 mL). The organic extracts were combined, washed with brine (2×15 mL), dried with MgSO₄, filtered, and concentrated in vacuo. The residue was purified by flash column chromatography (Teledyne ISCO, silica 10 g, Ø=2.5 cm, hexane/ethyl acetate 0→10%) to give (hex-5-en-1-yloxy)cyclohexane (161 mg, 44%) as a colorless oil. ¹H NMR (600 MHz, CDCl₃) δ 5.86-5.77 (m, 1H), 5.04-4.97 (m, 1H), 4.96-4.91 (m, 1H), 3.44 (t, J=6.7 Hz, 2H), 3.22-3.16 (m, 1H), 2.10-2.04 (m, 2H), 1.89 (tt, J=13.0, 5.7 Hz, 2H), 1.76-1.68 (m, 2H), 1.61-1.51 (m, 4H), 1.48-1.42 (m, 2H), 1.27-1.20 (m, 4H). ¹³C NMR (101 MHz, cdcl₃) δ 138.9, 114.4, 77.4, 67.7, 33.6, 32.4, 29.7, 25.9, 25.6, 24.2.

### 5-(But-3-en-1-yloxy)-1,2,3,4-tetrahydronaphthalene, 1ba

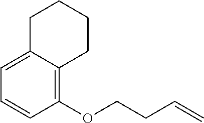

Synthesized according to procedure 1. 57 mg, 28%, colorless oil. ¹H NMR (600 MHz, CDCl₃) δ 7.04 (t, J=7.9 Hz, 1H), 6.69 (d, J=7.6 Hz, 1H), 6.63 (d, J=8.0 Hz, 1H), 5.93 (ddt, J=17.0, 10.2, 6.7 Hz, 1H), 5.17 (dq, J=17.1, 1.6 Hz, 1H), 5.12-5.06 (m, 1H), 4.00 (t, J=6.5 Hz, 2H), 2.75 (t, J=6.0 Hz, 2H), 2.66 (t, J=6.2 Hz, 2H), 2.55 (qt, J=6.6, 1.4 Hz, 2H), 1.82-1.72 (m, 4H). ¹³C NMR (151 MHz, CDCl₃) δ 156.7, 138.7, 135.0, 126.3, 125.7, 121.5, 116.9, 107.8, 67.2, 34.0, 29.8, 23.2, 23.0, 23.0.

### 4-(But-3-en-1-yloxy)-2,3-dihydro-1H-indene, 1bb

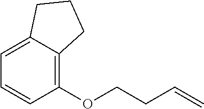

Synthesized according to procedure 1. 166 mg, 44%, colorless oil. ¹H NMR (600 MHz, CDCl₃) δ 7.10 (ddd, J=8.2, 7.4, 0.9 Hz, 1H), 6.85 (d, J=7.4 Hz, 1H), 6.65 (d, J=8.1 Hz, 1H), 5.92 (ddt, J=17.0, 6.7, 0.8 Hz, 1H), 5.19-5.14 (m, 1H), 5.12-5.07 (m, 1H), 4.04 (t, J=6.7 Hz, 2H), 2.92 (t, J=7.4 Hz, 2H), 2.87 (t, J=7.4 Hz, 2H), 2.55 (qq, J=6.7, 1.2 Hz, 2H), 2.10-2.02 (m, 2H). ¹³C NMR (151 MHz, CDCl₃) δ 155.5, 146.4, 134.8, 132.3, 127.5, 117.0, 108.9, 67.3, 34.0, 33.4, 29.5, 25.1.

### 7-Methoxyhept-1-ene, 1bc

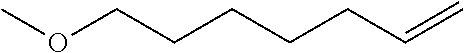

Synthesized according to procedure 13. 491 mg, 44%, 57% in Et₂O, colorless solution. ¹H NMR (500 MHz, CDCl₃) δ 5.81 (ddt, J=16.9, 10.1, 6.6 Hz, 1H), 4.99 (dq, J=17.0, 1.8 Hz, 1H), 4.96-4.91 (m, 1H), 3.36 (t, J=6.6 Hz, 2H), 3.33 (d, J=0.6 Hz, 3H), 2.10-2.02 (m, 2H), 1.63-1.53 (m, 2H), 1.45-1.32 (m, 4H). ¹³C NMR (151 MHz, CDCl₃) δ 137.8, 112.9, 71.8, 56.8, 34.3, 32.7, 28.3, 27.7, 24.5.

### (4-Oxocyclohexyl)methyl 4-methylbenzenesulfonate, XXbd

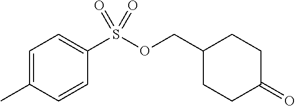 [SMILES: Cc1ccc(S(=O)(=O)OCC2CCC(=O)CC2)cc1]

To a solution of 4-(hydroxymethyl)cyclohexan-1-one (0.26 g, 1 eq., 2.0 mmol) in pyridine (2.0 mL) was added slowly 4-methylbenzenesulfonyl chloride (0.43 g, 1.12 eq., 2.2 mmol) at 0° C. The reaction was left for 3 days. The reaction was diluted with water (40 mL) and extracted with diethyl ether (3×40 mL). The organic extracts were combined, washed with saturated aqueous CuSO₄ until the washings remained light blue, then washed with water (80 mL), brine (3×80 mL), dried with MgSO₄, filtered, and concentrated in vacuo to give (4-oxocyclohexyl)methyl 4-methylbenzenesulfonate 4m (0.30 g, 1.1 mmol, 53%) as an orange oil that was used without further purification. ¹H NMR (400 MHz, CDCl₃) δ 7.82-7.77 (m, 2H), 7.39-7.34 (m, 2H), 3.94 (d, J=6.5 Hz, 2H), 2.46 (d, J=1.6 Hz, 3H), 2.43-2.25 (m, 3H), 2.21-2.10 (m, 1H), 2.10-2.01 (m, 3H), 1.42 (qd, J=12.5, 4.9 Hz, 2H).

### 4-(Phenoxymethyl)cyclohexan-1-one, XXbd

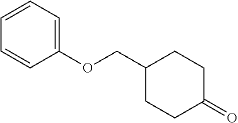 [SMILES: O=C1CCC(COc2ccccc2)CC1]

Synthesized according to procedure 17, carried forward without purification. 96 mg, 47%, colorless oil.

### ((4-Methylenecyclohexyl)methoxy)benzene, 1bd

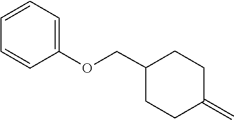

Synthesized according to procedure 15. 23 mg, 52%, colorless oil. ¹H NMR (500 MHz, CDCl₃) δ 7.31-7.26 (m, 2H), 6.93 (tt, J=7.4, 1.1 Hz, 1H), 6.91-6.87 (m, 2H), 4.65 (t, J=1.6 Hz, 2H), 3.78 (d, J=6.1 Hz, 2H), 2.39-2.32 (m, 2H), 2.14-2.05 (m, 2H), 2.03-1.91 (m, 3H), 1.23-1.12 (m, 2H). ¹³C NMR (126 MHz, CDCl₃) δ 159.1, 149.2, 129.4, 120.5, 114.4, 107.2, 72.6, 37.3, 34.1, 31.1.

### 8-Methoxyoct-1-ene, 1be

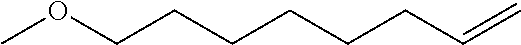

Synthesized according to procedure 13. 260 mg, 91%, colorless oil. ¹H NMR (600 MHz, CDCl₃) δ 5.80 (ddt, J=16.9, 10.2, 6.7 Hz, 1H), 5.01-4.97 (m, 1H), 4.93 (ddt, J=10.2, 2.3, 1.2 Hz, 1H), 3.36 (t, J=6.6 Hz, 2H), 3.33 (s, 3H), 2.07-2.01 (m, 2H), 1.60-1.53 (m, 2H), 1.42-1.28 (m, 6H). ¹³C NMR (151 MHz, CDCl₃) δ 139.1, 114.2, 72.9, 65.9, 58.6, 33.7, 29.6, 29.0, 28.8, 26.0, 15.3.

### Methyl 3-methylenecyclohexane-1-carboxylate, XXbf

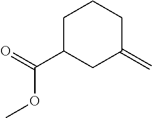

Synthesized according to procedure 15, carried forward without purification. 59 mg, 38%, colorless oil.

### (3-Methylenecyclohexyl)methanol, XXbf

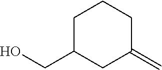

Synthesized according to procedure 18. 48 mg, 99%, colorless oil. ¹H NMR (600 MHz, CDCl₃) δ 4.64 (d, J=1.5 Hz, 2H), 3.50 (d, J=6.3 Hz, 2H), 2.39-2.32 (m, 1H), 2.30-2.23 (m, 1H), 2.01-1.93 (m, 1H), 1.84-1.77 (m, 2H), 1.77-1.73 (m, 1H), 1.65-1.58 (m, 1H), 1.50 (s, 1H), 1.39-1.31 (m, 1H), 1.17-1.10 (m, 1H). ¹³C NMR (151 MHz, CDCl₃) δ 148.4, 107.6, 67.9, 41.7, 37.9, 35.0, 28.8, 26.6.

### (3-Methylenecyclohexyl)methyl 4-methylbenzenesulfonate, XXbf

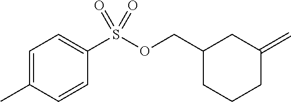

Synthesized according to procedure 16. 103 mg, 97%, colorless oil. ¹H NMR (600 MHz, CDCl₃) δ 7.78 (d, J=8.2 Hz, 2H), 7.34 (d, J=8.0 Hz, 2H), 4.62 (s, 1H), 4.58 (s, 1H), 3.89-3.81 (m, 2H), 2.44 (s, 3H), 2.29-2.24 (m, 1H), 2.21 (dt, J=13.4, 4.1 Hz, 1H), 1.91 (td, J=12.3, 4.3 Hz, 1H), 1.82-1.67 (m, 4H), 1.35-1.24 (m, 1H), 1.16-1.07 (m, 1H). ¹³C NMR (151 MHz, CDCl₃) δ 146.8, 144.7, 132.9, 129.8, 127.8, 108.5, 74.3, 38.1, 37.3, 34.5, 28.2, 26.0, 21.6.

### ((3-Methylenecyclohexyl)methoxy)benzene, 1bf

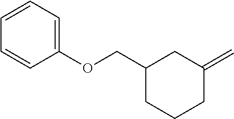

Synthesized according to procedure 17. 32 mg, 47%, colorless oil. ¹H NMR (500 MHz, CDCl₃) δ 7.36-7.28 (m, 2H), 7.00-6.92 (m, 3H), 4.74-4.70 (m, 2H), 3.85 (d, J=5.9 Hz, 2H), 2.54-2.49 (m, 1H), 2.34 (dt, J=13.3, 3.9 Hz, 1H), 2.06 (td, J=12.6, 4.5 Hz, 1H), 2.02-1.85 (m, 4H), 1.50-1.39 (m, 1H), 1.37-1.27 (m, 1H). ¹³C NMR (126 MHz, CDCl₃) δ 159.2, 148.1, 129.4, 120.5, 114.5, 107.9, 72.4, 38.9, 38.2, 35.0, 29.1, 26.5.

### Ethyl 3-methylenecyclopentane-1-carboxylate, XXbg

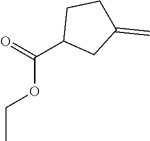

Synthesized according to procedure 15, carried forward without purification. 0.46 g, 99%, colorless oil.

### (3-Methylenecyclopentyl)methanol, XXbg

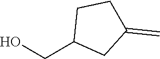

Synthesized according to procedure 18. 0.31 g, 93%, colorless oil. ¹H NMR (500 MHz, CDCl₃) δ 4.84 (dq, J=3.9, 2.0 Hz, 2H), 3.55 (d, J=6.8 Hz, 2H), 2.46 (dd, J=16.2, 7.7 Hz, 1H), 2.41-2.25 (m, 2H), 2.25-2.16 (m, 1H), 2.07-2.00 (m, 1H), 1.90-1.82 (m, 1H), 1.47 (s, 1H), 1.47-1.36 (m, 1H). ¹³C NMR (126 MHz, CDCl₃) δ 151.9, 105.5, 68.0, 42.4, 36.2, 32.0, 29.0.

### (3-Methylenecyclopentyl)methyl 4-methylbenzenesulfonate, XXbg

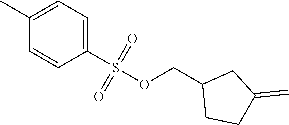

Synthesized according to procedure 16. 0.397 g, 54%, colorless oil. ¹H NMR (600 MHz, CDCl₃) δ 7.79 (d, J=8.3 Hz, 2H), 7.35 (d, J=8.0 Hz, 2H), 4.85-4.79 (m, 2H), 3.96-3.88 (m, 2H), 2.45 (s, 3H), 2.42 (dd, J=15.9, 7.4 Hz, 1H), 2.35-2.21 (m, 3H), 1.99-1.92 (m, 1H), 1.87-1.79 (m, 1H), 1.38-1.31 (m, 1H). ¹³C NMR (151 MHz, CDCl₃) δ 150.4, 144.7, 133.0, 129.8, 127.9, 106.2, 73.3, 38.9, 35.9, 31.6, 28.8, 21.7.

### ((3-Methylenecyclopentyl)methoxy)benzene, 1bg

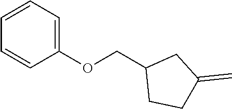

Synthesized according to procedure 17. 85 mg, 31%, colorless oil. ¹H NMR (600 MHz, CDCl₃) δ 7.28 (t, J=8.0 Hz, 2H), 6.94 (t, J=7.3 Hz, 1H), 6.90 (d, J=8.8 Hz, 2H), 4.90-4.85 (m, 2H), 3.90-3.84 (m, 2H), 2.56 (dd, J=16.2, 7.7 Hz, 1H), 2.49 (dq, J=14.7, 7.2 Hz, 1H), 2.44-2.38 (m, 1H), 2.37-2.30 (m, 1H), 2.20-2.13 (m, 1H), 2.00-1.93 (m, 1H), 1.59-1.51 (m, 1H). ¹³C NMR (151 MHz, CDCl₃) δ 159.1, 151.7, 129.4, 120.5, 114.4, 105.7, 71.2, 39.5, 36.6, 31.9, 29.4.

### Methyl 4-(phenoxymethyl)benzoate, XXbh

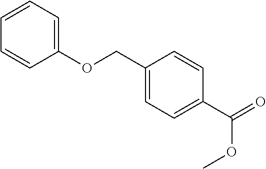 [SMILES: COC(=O)c1ccc(COc2ccccc2)cc1]

To a stirring solution of phenol (0.23 g, 1 eq., 2.4 mmol) and potassium carbonate (1.0 g, 3.0 eq., 7.2 mmol) in acetone (12 mL) was added methyl 4-(bromomethyl)benzoate (0.55 g, 1.0 eq., 2.4 mmol). The reaction was heated to 56° C., monitoring by TLC. The reaction was diluted with water (10 mL), extracted with ethyl acetate (3×10 mL). The organic extracts were combined, washed with brine (2×30 mL), dried with MgSO₄, filtered, and concentrated in vacuo. The residue was purified by flash column chromatography (Teledyne ISCO, silica 40 g, Ø=5 cm, hexane/ethyl acetate 0→30%) to give methyl 4-(phenoxymethyl)benzoate 8t (0.542 g, 2.24 mmol, 93%) as a white amorphous solid. ¹H NMR (600 MHz, CDCl₃) δ 8.06 (d, J=8.3 Hz, 2H), 7.51 (d, J=8.0 Hz, 2H), 7.33-7.28 (m, 2H), 7.01-6.95 (m, 3H), 5.13 (s, 2H), 3.92 (s, 3H). ¹³C NMR (151 MHz, CDCl₃) δ 166.8, 158.4, 142.3, 129.9, 129.6, 129.5, 126.9, 121.2, 114.8, 69.2, 52.1.

### (4-(Phenoxymethyl)phenyl)methanol, XXbh

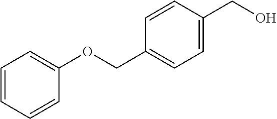 [SMILES: OCc1ccc(COc2ccccc2)cc1]

Synthesized according to procedure 18. 0.436 g, 93%, colorless oil. ¹H NMR (600 MHz, CDCl₃) δ 7.44 (d, J=7.9 Hz, 2H), 7.39 (d, J=7.9 Hz, 2H), 7.33-7.27 (m, 2H), 7.01-6.95 (m, 3H), 5.07 (s, 2H), 4.70 (s, 2H), 1.74 (s, 1H). ¹³C NMR (151 MHz, CDCl₃) δ 158.6, 140.6, 136.5, 129.5, 127.7, 127.2, 120.9, 114.8, 69.6, 65.1.

### 1-(Bromomethyl)-4-(phenoxymethyl)benzene, XXbh

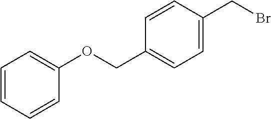 [SMILES: BrCc1ccc(COc2ccccc2)cc1]

To a stirring solution of carbon tetrabromide (0.33 g, 1.1 eq., 0.99 mmol) and (4-(phenoxymethyl)phenyl)methanol (0.19 g, 1.0 eq., 0.90 mmol) in dichloromethane (0.90 mL) was added slowly triphenylphosphine (0.26 g, 1.1 eq., 0.99 mmol) at 0° C. The reaction was allowed to warm to room temperature and stirred for 16 hours. The reaction was diluted with hexane (5 mL), filtered through celite, and concentrated in vacuo. The residue was purified by flash column chromatography (Teledyne ISCO, silica 25 g, Ø=2.5 cm, hexane/ethyl acetate 0→3%) to give 1-(bromomethyl)-4-(phenoxymethyl)benzene 2t (0.155 g, 0.559 mmol, 62%) as a white amorphous solid. ¹H NMR (500 MHz, CDCl₃) δ 7.42 (s, 4H), 7.33-7.27 (m, 2H), 7.00-6.95 (m, 3H), 5.06 (s, 2H), 4.51 (s, 2H). ¹³C NMR (126 MHz, CDCl₃) δ 158.6, 137.4, 129.5, 129.3, 127.8, 121.0, 114.8, 69.4, 33.2.

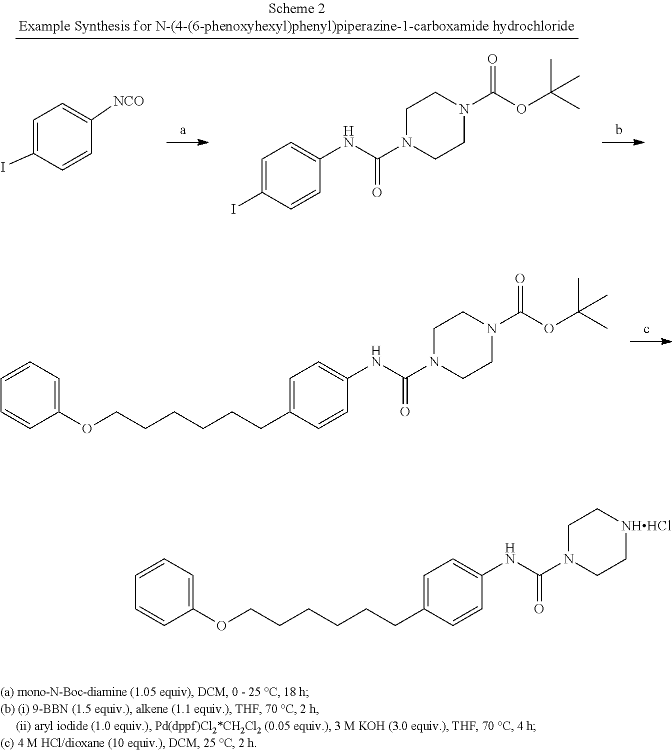

### tert-butyl 4-((4-iodophenyl)carbamoyl)piperazine-1-carboxylate

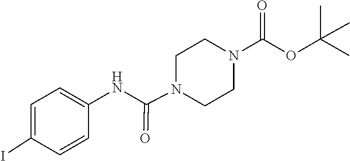 [SMILES: CC(C)(C)OC(=O)N1CCN(C(=O)Nc2ccc(I)cc2)CC1]

Synthesized according to procedure 2, white solid, 865 mg, 98% yield. ¹H NMR (500 MHz, MeOD) δ 7.63-7.51 (m, 2H), 7.26-7.11 (m, 2H), 3.61-3.39 (m, 8H), 1.48 (s, 9H). ¹³C NMR (126 MHz, MeOD) δ 157.4, 156.3, 141.0, 138.6, 123.8, 86.4, 81.6, 45.0, 28.6. HRMS: (ESI) [M+H]⁺ calc. for C₁₆H₂₃IN₃O₃, 432.0779, observed, 432.0791.

### tert-butyl 4-((4-(5-phenoxypentyl)phenyl)carbamoyl)piperazine-1-carboxylate (2a)

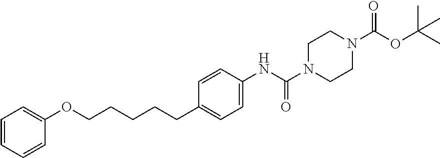 [SMILES: CC(C)(C)OC(=O)N1CCN(C(=O)Nc2ccc(CCCCCOc3ccccc3)cc2)CC1]

Synthesized according to procedure 3, white solid, 82 mg, 76% yield. ¹H NMR (600 MHz, CDCl₃) δ 7.29-7.22 (m, 4H), 7.12-7.09 (m, 2H), 6.94-6.91 (m, 1H), 6.90-6.87 (m, 2H), 6.34 (s, 1H), 3.94 (t, J=6.5 Hz, 2H), 3.51-3.44 (m, 8H), 2.59 (t, J=7.7 Hz, 2H), 1.83-1.77 (m, 2H), 1.68-1.63 (m, 2H), 1.52-1.46 (m, 11H). ¹³C NMR (151 MHz, CDCl₃) δ 159.2, 155.3, 154.8, 137.8, 136.5, 129.5, 129.0, 120.6, 120.4, 114.6, 80.4, 67.8, 44.0, 35.3, 31.4, 29.3, 28.5, 25.8. HRMS: (ESI) [M+H]⁺ calc. for C₂₇H₃₈N₃O₄, 468.2857, observed, 468.2863.

### tert-butyl 4-((4-(6-phenoxyhexyl)phenyl)carbamoyl)piperazine-1-carboxylate (2b)

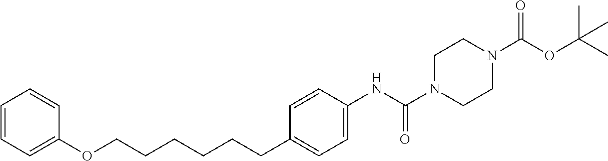 [SMILES: CC(C)(C)OC(=O)N1CCN(C(=O)Nc2ccc(CCCCCCOc3ccccc3)cc2)CC1]

Synthesized according to procedure 3, white solid, 466 mg, 64% yield. ¹H NMR (600 MHz, CDCl₃) δ 7.29-7.26 (m, 2H), 7.25-7.22 (m, 2H), 7.11-7.08 (m, 2H), 6.94-6.91 (m, 1H), 6.90-6.87 (m, 2H), 6.27 (s, 1H), 3.94 (t, J=6.5 Hz, 2H), 3.52-3.45 (m, 8H), 2.57 (t, J=7.7 Hz, 2H), 1.80-1.73 (m, 2H), 1.65-1.58 (m, 2H), 1.51-1.45 (m, 11H), 1.41-1.36 (m, 2H). ¹³C NMR (151 MHz, CDCl₃) δ 159.2, 155.2, 154.8, 138.1, 136.4, 129.5, 129.0, 120.6, 120.4, 114.6, 80.5, 67.9, 35.3, 31.6, 29.3, 29.0, 28.5, 26.1. HRMS: (ESI) [M+H]⁺ calc. for C₂₈H₄₀N₃O₄, 482.3013, observed, 482.3009.

### tert-butyl 4-((4-(7-phenoxyheptyl)phenyl)carbamoyl)piperazine-1-carboxylate (2c)

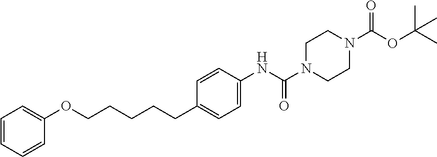 [SMILES: CC(C)(C)OC(=O)N1CCN(C(=O)Nc2ccc(CCCCCOc3ccccc3)cc2)CC1]

Synthesized according to procedure 3, white solid, 186 mg, 81% yield. ¹H NMR (600 MHz, CDCl₃) δ 7.29-7.25 (m, 2H), 7.25-7.22 (m, 2H), 7.11-7.07 (m, 2H), 6.94-6.90 (m, 1H), 6.90-6.87 (m, 2H), 6.41 (d, J=2.8 Hz, 1H), 3.93 (t, J=6.6 Hz, 2H), 3.50-3.44 (m, 8H), 2.55 (t, J=7.7 Hz, 2H), 1.76 (p, J=6.7 Hz, 2H), 1.63-1.55 (m, 2H), 1.48 (s, 9H), 1.46-1.41 (m, 2H), 1.40-1.30 (m, 4H). ¹³C NMR (151 MHz, CDCl₃) δ 159.2, 155.3, 154.7, 138.1, 136.4, 129.5, 128.9, 120.5, 120.4, 114.6, 80.4, 67.9, 43.9, 35.4, 31.6, 29.4, 29.4, 29.2, 28.5, 26.1. HRMS: (ESI) [M+H]⁺ calc. for C₂₉H₄₂N₃O₄, 496.3170, observed, 496.3181.

### tert-butyl 4-((4-(5-(p-tolyloxy)pentyl)phenyl)carbamoyl)piperazine-1-carboxylate (2d)

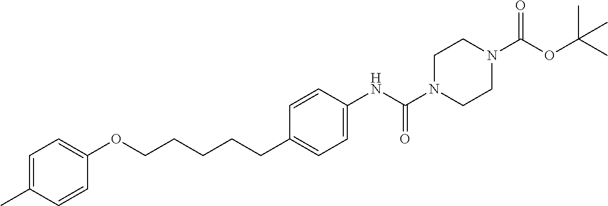 [SMILES: Cc1ccc(OCCCCCc2ccc(NC(=O)N3CCN(C(=O)OC(C)(C)C)CC3)cc2)cc1]

Synthesized according to procedure 3, white solid, 112 mg, 67% yield. ¹H NMR (400 MHz, CDCl₃) δ 7.27-7.21 (m, 2H), 7.11-7.03 (m, 4H), 6.81-6.75 (m, 2H), 6.69 (s, 1H), 3.91 (t, J=6.5 Hz, 2H), 3.47-3.40 (m, 8H), 2.58 (t, J=7.6 Hz, 2H), 2.28 (s, 3H), 1.82-1.74 (m, 2H), 1.69-1.60 (m, 2H), 1.55-1.43 (m, 11H). ¹³C NMR (101 MHz, CDCl₃) δ 157.0, 155.4, 154.7, 137.7, 136.5, 129.9, 129.7, 128.8, 120.6, 114.4, 80.4, 68.0, 43.9, 35.2, 31.3, 29.3, 28.5, 25.7, 20.5. HRMS: (ESI) [M+H]⁺ calc. for C₂₈H₄₀N₃O₄, 482.3013, observed, 482.3021.

### tert-butyl 4-((4-(6-(m-tolyloxy)hexyl)phenyl)carbamoyl)piperazine-1-carboxylate (2e)

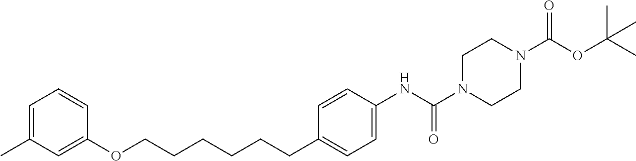 [SMILES: Cc1cccc(OCCCCCCc2ccc(NC(=O)N3CCN(C(=O)OC(C)(C)C)CC3)cc2)c1]

Synthesized according to procedure 3, white solid, 205 mg, 89% yield. ¹H NMR (500 MHz, CDCl₃) δ 7.25-7.22 (m, 2H), 7.15 (t, J=7.8 Hz, 1H), 7.11-7.07 (m, 2H), 6.76-6.72 (m, 1H), 6.72-6.70 (m, 1H), 6.70-6.67 (m, 1H), 6.41 (s, 1H), 3.92 (t, J=6.5 Hz, 2H), 3.49-3.44 (m, 8H), 2.56 (t, J=7.7 Hz, 2H), 2.32 (s, 3H), 1.79-1.72 (m, 2H), 1.65-1.57 (m, 2H), 1.51-1.44 (m, 11H), 1.42-1.33 (m, 2H). ¹³C NMR (126 MHz, CDCl₃) δ 159.2, 155.3, 154.8, 139.5, 138.0, 136.4, 129.2, 128.9, 121.4, 120.4, 115.5, 111.4, 80.4, 67.8, 44.0, 35.3, 31.5, 29.4, 29.0, 28.5, 26.0, 21.7. HRMS: (ESI) [M+H]⁺ calc. for C₂₉H₄₂N₃O₄, 496.3170, observed, 496.3171.

### tert-butyl 4-((4-(5-(m-tolyloxy)pentyl)phenyl)carbamoyl)piperazine-1-carboxylate (2f)

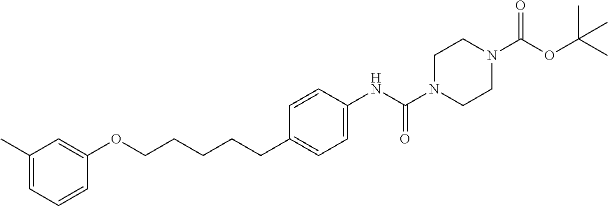 [SMILES: Cc1cccc(OCCCCCc2ccc(NC(=O)N3CCN(C(=O)OC(C)(C)C)CC3)cc2)c1]

Synthesized according to procedure 3, white solid, 191 mg, 86% yield. ¹H NMR (400 MHz, cdcl₃) δ 7.26-7.22 (m, 2H), 7.15 (t, J=7.8 Hz, 1H), 7.13-7.08 (m, 2H), 6.77-6.72 (m, 1H), 6.72-6.67 (m, 2H), 6.29 (s, 1H), 3.92 (t, J=6.5 Hz, 2H), 3.53-3.44 (m, 8H), 2.59 (t, J=7.6 Hz, 2H), 2.32 (s, 3H), 1.83-1.75 (m, 2H), 1.70-1.61 (m, 2H), 1.53-1.44 (m, 11H). ¹³C NMR (126 MHz, CDCl₃) δ 159.2, 155.3, 154.8, 139.6, 137.9, 136.4, 129.3, 129.0, 121.4, 120.4, 115.5, 111.5, 80.4, 67.8, 44.0, 35.3, 31.4, 29.3, 28.5, 25.8, 21.7. HRMS: (ESI) [M+H]⁺ calc. for C₂₈H₄₀N₃O₄, 482.3013, observed, 482.3013.

### tert-butyl 4-((4-(5-(m-tolyloxy)pentyl)phenyl)carbamoyl)piperazine-1-carboxylate (2g)

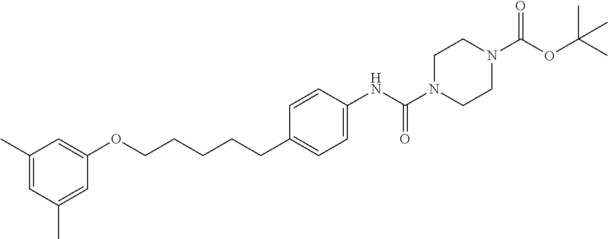 [SMILES: Cc1cc(C)cc(OCCCCCc2ccc(NC(=O)N3CCN(C(=O)OC(C)(C)C)CC3)cc2)c1]

Synthesized according to procedure 3, white solid, 144 mg, 63% yield. ¹H NMR (500 MHz, CDCl₃) δ 7.26-7.22 (m, 2H), 7.12-7.08 (m, 2H), 6.59-6.57 (m, 1H), 6.52-6.51 (m, 2H), 6.40-6.38 (m, 1H), 3.91 (t, J=6.6 Hz, 2H), 3.51-3.44 (m, 8H), 2.58 (t, J=7.6 Hz, 2H), 2.28 (s, 6H), 1.81-1.74 (m, 2H), 1.69-1.60 (m, 2H), 1.53-1.42 (m, 9H). ¹³C NMR (126 MHz, CDCl₃) δ 159.2, 155.3, 154.8, 139.2, 137.8, 136.5, 129.0, 122.4, 120.4, 112.4, 80.4, 67.7, 44.1, 35.3, 31.4, 29.3, 28.5, 25.8, 21.6. HRMS: (ESI) [M+H]⁺ calc. for C₂₉H₄₂N₃O₄, 496.3170, observed, 496.3163.

### tert-butyl 4-((4-(5-(o-tolyloxy)pentyl)phenyl)carbamoyl)piperazine-1-carboxylate (2h)

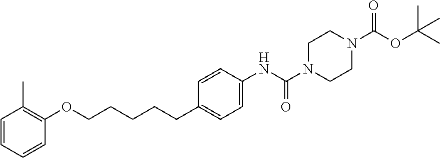 [SMILES: Cc1ccccc1OCCCCCc1ccc(NC(=O)N2CCN(C(=O)OC(C)(C)C)CC2)cc1]

Synthesized according to procedure 3, white solid, 73 mg, 65% yield. ¹H NMR (500 MHz, CDCl₃) δ 7.26-7.22 (m, 2H), 7.15-7.08 (m, 4H), 6.85-6.82 (m, 1H), 6.81-6.77 (m, 1H), 6.28 (s, 1H), 3.94 (t, J=6.4 Hz, 2H), 3.52-3.46 (m, 8H), 2.60 (t, J=7.7 Hz, 2H), 2.22 (s, 3H), 1.85-1.78 (m, 2H), 1.70-1.63 (m, 2H), 1.55-1.45 (m, 11H). ¹³C NMR (126 MHz, CDCl₃) δ 157.3, 155.3, 154.8, 137.9, 136.4, 130.7, 129.0, 127.0, 126.8, 120.4, 120.2, 111.0, 80.5, 67.9, 43.9, 35.3, 31.4, 29.4, 28.5, 25.9, 16.4. HRMS: (ESI) [M+H]⁺ calc. for C₂₈H₃₄₀N₃O₄, 482.3013, observed, 482.3020.

### tert-butyl 4-((4-(3-(4-ethylphenoxy)propyl)phenyl)carbamoyl)piperazine-1-carboxylate (2i)

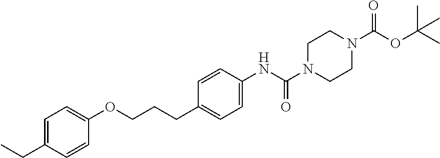 [SMILES: CCc1ccc(OCCCc2ccc(NC(=O)N3CCN(C(=O)OC(C)(C)C)CC3)cc2)cc1]

Synthesized according to procedure 3, white solid, 73 mg, 65% yield. ¹H NMR (500 MHz, CDCl₃) δ 7.25 (d, J=8.5 Hz, 2H), 7.15-7.12 (m, 2H), 7.11-7.08 (m, 2H), 6.83-6.80 (m, 2H), 6.28 (s, 1H), 3.92 (t, J=6.2 Hz, 2H), 3.52-3.45 (m, 8H), 2.78-2.73 (m, 2H), 2.58 (q, J=7.7 Hz, 2H), 2.09-2.02 (m, 2H), 1.48 (s, 9H), 1.21 (t, J=7.6 Hz, 3H). ¹³C NMR (126 MHz, CDCl₃) δ 157.2, 155.2, 154.8, 136.9, 136.7, 136.5, 129.1, 128.8, 120.4, 114.5, 80.5, 66.9, 44.0, 31.6, 31.1, 28.5, 28.1, 16.0. HRMS: (ESI) [M+H]⁺ calc. for C₂₇H₃₈N₃O₄, 468.2857, observed, 468.2862.

### tert-butyl 4-((4-(4-(3-ethylphenoxy)butyl)phenyl)carbamoyl)piperazine-1-carboxylate (2j)

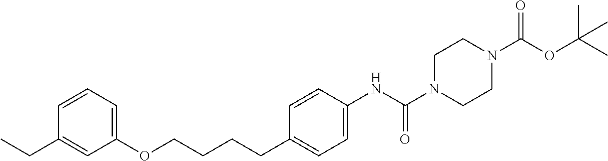 [SMILES: CCc1cccc(OCCCCc2ccc(NC(=O)N3CCN(C(=O)OC(C)(C)C)CC3)cc2)c1]

Synthesized according to procedure 3, white solid, 71 mg, 64% yield. ¹H NMR (400 MHz, CDCl₃) δ 7.27-7.23 (m, 2H), 7.20-7.15 (m, 1H), 7.14-7.10 (m, 2H), 6.79-6.76 (m, 1H), 6.75-6.73 (m, 1H), 6.72-6.68 (m, 1H), 6.31 (s, 1H), 3.97-3.93 (m, 2H), 3.57-3.41 (m, 8H), 2.68-2.56 (m, 2H), 1.82-1.75 (m, 4H), 1.48 (d, J=0.9 Hz, 11H), 1.24-1.21 (m, 3H). ¹³C NMR (126 MHz, CDCl₃) δ 159.3, 155.3, 154.8, 146.0, 137.6, 136.6, 129.3, 129.0, 120.4, 120.2, 114.4, 111.5, 80.4, 67.7, 44.0, 35.0, 29.0, 28.5, 28.1, 15.6. HRMS: (ESI) [M+H]⁺ calc. for C₂₈H₄₀N₃O₄, 482.3013, observed, 482.3007.

### tert-butyl 4-((4-(3-(3-propylphenoxy)propyl)phenyl)carbamoyl)piperazine-1-carboxylate (2k)

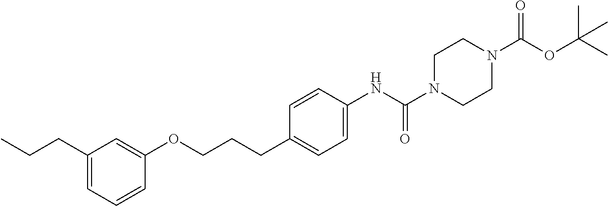 [SMILES: CCCc1cccc(OCCCc2ccc(NC(=O)N3CCN(C(=O)OC(C)(C)C)CC3)cc2)c1]

Synthesized according to procedure 3, white solid, 121 mg, 36% yield. ¹H NMR (500 MHz, CDCl₃) δ 7.29-7.26 (m, 2H), 7.19 (t, J=7.8 Hz, 1H), 7.17-7.14 (m, 2H), 6.80-6.77 (m, 1H), 6.76-6.74 (m, 1H), 6.74-6.71 (m, 1H), 6.45 (s, 1H), 3.96 (t, J=6.2 Hz, 2H), 3.52-3.47 (m, 8H), 2.78 (t, J=7.6 Hz, 2H), 2.59-2.55 (m, 2H), 2.12-2.05 (m, 2H), 1.70-1.61 (m, 2H), 1.50 (s, 9H), 0.96 (t, J=7.3 Hz, 3H). ¹³C NMR (126 MHz, CDCl₃) δ 159.1, 155.3, 154.8, 144.4, 136.8, 136.7, 129.2, 129.1, 121.0, 120.5, 115.0, 111.6, 80.4, 66.7, 43.9, 38.2, 31.6, 31.1, 28.5, 24.6, 14.0. HRMS: (ESI) [M+H]⁺ calc. for C₂₈H₄₀N₃O₄, 482.3013, observed, 482.3013.

### tert-butyl 4-((4-(4-(3-isopropylphenoxy)butyl)phenyl)carbamoyl)piperazine-1-carboxylate (2l)

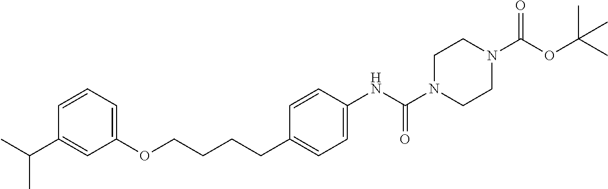 [SMILES: CC(C)c1cccc(OCCCCc2ccc(NC(=O)N3CCN(C(=O)OC(C)(C)C)CC3)cc2)c1]

Synthesized according to procedure 3, white solid, 158 mg, 92% yield. ¹H NMR (500 MHz, CDCl₃) δ 7.26-7.23 (m, 2H), 7.19 (t, J=7.9 Hz, 1H), 7.14-7.10 (m, 2H), 6.82-6.79 (m, 1H), 6.78-6.76 (m, 1H), 6.72-6.68 (m, 1H), 6.37 (s, 1H), 3.98-3.93 (m, 2H), 3.52-3.44 (m, 8H), 2.86 (p, J=6.9 Hz, 1H), 2.64 (t, J=7.0 Hz, 2H), 1.83-1.74 (m, 4H), 1.48 (s, 9H), 1.24 (d, J=7.0 Hz, 6H). ¹³C NMR (126 MHz, CDCl₃) δ 159.2, 155.3, 154.8, 150.7, 137.5, 136.6, 129.3, 129.0, 120.4, 118.9, 113.2, 111.3, 80.4, 67.7, 44.0, 35.0, 34.3, 29.0, 28.5, 28.1, 24.1. HRMS: (ESI) [M+H]⁺ calc. for C₂₉H₄₂N₃O₄, 496.3170, observed, 496.3171.

### tert-butyl 4-((4-(4-(3-cyclopropylphenoxy)butyl)phenyl)carbamoyl)piperazine-1-carboxylate (2m)

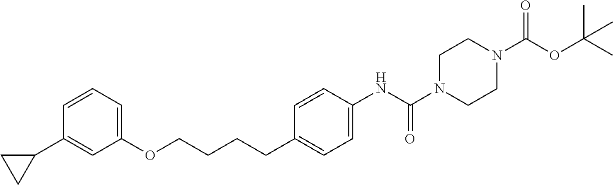 [SMILES: CC(C)(C)OC(=O)N1CCN(C(=O)Nc2ccc(CCCCOc3cccc(C4CC4)c3)cc2)CC1]

Synthesized according to procedure 3, white solid, 124 mg, 54% yield. ¹H NMR (500 MHz, CDCl₃) δ 7.26-7.23 (m, 2H), 7.16-7.10 (m, 3H), 6.68-6.64 (m, 2H), 6.60-6.58 (m, 1H), 6.34 (s, 1H), 3.94 (t, J=5.9 Hz, 2H), 3.52-3.44 (m, 8H), 2.63 (t, J=7.1 Hz, 2H), 1.88-1.83 (m, 1H), 1.81-1.73 (m, 4H), 1.48 (s, 11H), 0.95-0.91 (m, 2H), 0.70-0.66 (m, 2H). ¹³C NMR (126 MHz, CDCl₃) δ 159.3, 155.3, 154.8, 145.8, 137.5, 136.6, 129.3, 129.0, 120.4, 118.1, 112.2, 111.2, 80.4, 67.7, 44.0, 35.0, 29.0, 28.5, 28.0, 15.6, 9.4. HRMS: (ESI) [M+H]⁺ calc. for C₂₉H₄₀N₃O₄, 494.3013, observed, 494.3025.

### tert-butyl 4-((4-(4-(3-(tert-butyl)phenoxy)butyl)phenyl)carbamoyl)piperazine-1-carboxylate (2n)

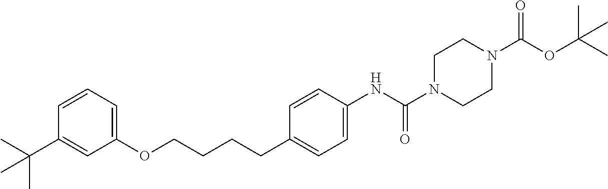 [SMILES: CC(C)(C)OC(=O)N1CCN(C(=O)Nc2ccc(CCCCOc3cccc(C(C)(C)C)c3)cc2)CC1]

Synthesized according to procedure 3, white solid, 188 mg, 80% yield. ¹H NMR (500 MHz, CDCl₃) δ 7.26-7.23 (m, 2H), 7.20 (t, J=8.0 Hz, 1H), 7.14-7.10 (m, 2H), 6.98-6.95 (m, 1H), 6.94-6.91 (m, 1H), 6.71-6.67 (m, 1H), 6.38 (s, 1H), 3.96 (t, J=6.1 Hz, 2H), 3.50-3.44 (m, 8H), 2.64 (t, J=7.1 Hz, 2H), 1.83-1.75 (m, 4H), 1.48 (s, 9H), 1.30 (s, 9H). ¹³C NMR (126 MHz, CDCl₃) δ 159.0, 155.3, 154.8, 153.0, 137.5, 136.6, 129.0, 120.4, 117.8, 112.7, 110.6, 80.4, 67.7, 44.0, 35.1, 34.9, 31.4, 29.0, 28.5, 28.1.

### tert-butyl 4-((4-(5-(4-(trifluoromethyl)phenoxy)pentyl)phenyl)carbamoyl)piperazine-1-carboxylate (2o)

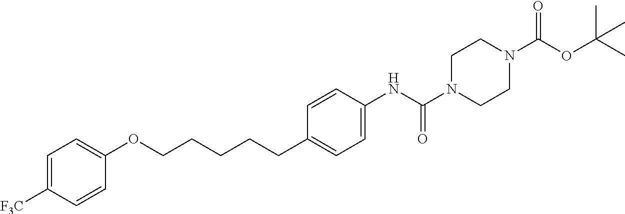 [SMILES: CC(C)(C)OC(=O)N1CCN(C(=O)Nc2ccc(CCCCCOc3ccc(C(F)(F)F)cc3)cc2)CC1]

Synthesized according to procedure 3, white solid, 98 mg, 53% yield. ¹H NMR (400 MHz, CDCl₃) δ 7.54-7.48 (m, 2H), 7.26-7.21 (m, 2H), 7.09-7.05 (m, 2H), 6.94-6.89 (m, 2H), 6.70 (s, 1H), 3.96 (t, J=6.5 Hz, 2H), 3.45-3.41 (m, 9H), 2.58 (t, J=7.6 Hz, 2H), 1.84-1.75 (m, 2H), 1.65 (p, J=7.6 Hz, 2H), 1.52-1.42 (m, 11H). ¹⁹F NMR (376 MHz, CDCl₃) δ −61.39. ¹³C NMR (101 MHz, CDCl₃) δ 161.6 (q, J=1.1 Hz), 155.4, 154.7, 137.5, 136.7, 128.8, 126.9 (q, J=3.8 Hz), 124.6 (q, J=262.7 Hz), 123.2, 122.6 (q, J=32.7 Hz), 120.6, 120.4, 114.5, 80.3, 68.1, 43.9, 35.2, 31.2, 29.0, 28.5, 25.6. HRMS: (ESI) [M+H]⁺ calc. for C₂₈H₃₇F₃N₃O₄, 536.2731, observed, 536.2736.

### tert-butyl 4-((4-(5-(3-(trifluoromethyl)phenoxy)pentyl)phenyl)carbamoyl)piperazine-1-carboxylate (2p)

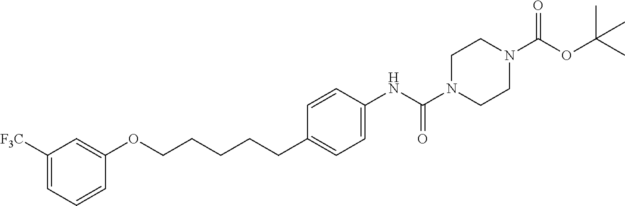 [SMILES: CC(C)(C)OC(=O)N1CCN(C(=O)Nc2ccc(CCCCCOc3cccc(C(F)(F)F)c3)cc2)CC1]

Synthesized according to procedure 3, white solid, 85 mg, 46% yield. ¹H NMR (400 MHz, CDCl₃) δ 7.35-7.29 (m, 1H), 7.22-7.19 (m, 2H), 7.16-7.13 (m, 1H), 7.08-7.05 (m, 3H), 7.02-6.98 (m, 1H), 6.45 (s, 1H), 3.93 (t, J=6.5 Hz, 2H), 3.44-3.40 (m, 8H), 2.56 (t, J=7.6 Hz, 2H), 1.81-1.74 (m, 2H), 1.67-1.58 (m, 2H), 1.50-1.41 (m, 11H). ¹⁹F NMR (376 MHz, CDCl₃) δ −62.69. ¹³C NMR (101 MHz, CDCl₃) δ 159.3, 155.4, 154.8, 137.7, 136.6, 131.9 (124.1 (q, J=272.6 Hz), 120.5, 118.1 (q, J=1.3 Hz), 117.3 (q, J=3.8 Hz), 111.4 (d, J=3.8 Hz), 80.4, 68.2, 43.9, 35.2, 31.3, 29.1, 28.5, 25.7. HRMS: (ESI) [M+H]⁺ calc. for C₂₈H₃₇F₃N₃O₄, 536.2731, observed, 536.2733.

### tert-butyl 4-((4-(4-(3-methoxyphenoxy)butyl)phenyl)carbamoyl)piperazine-1-carboxylate (2q)

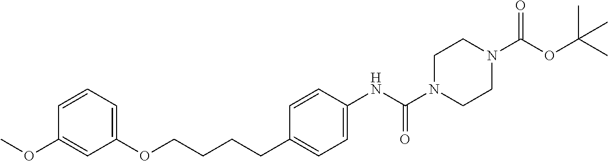 [SMILES: COc1cccc(OCCCCc2ccc(NC(=O)N3CCN(C(=O)OC(C)(C)C)CC3)cc2)c1]

Synthesized according to procedure 3, white solid, 116 mg, 69% yield. ¹H NMR (500 MHz, CDCl₃) δ 7.26-7.23 (m, 2H), 7.19-7.14 (m, 1H), 7.13-7.10 (m, 2H), 6.51-6.47 (m, 3H), 6.47-6.44 (m, 1H), 6.32 (s, 1H), 3.94 (t, J=6.1 Hz, 2H), 3.78 (s, 3H), 3.52-3.44 (m, 8H), 2.63 (t, J=7.1 Hz, 2H), 1.83-1.74 (m, 4H), 1.48 (s, 9H). ¹³C NMR (126 MHz, CDCl₃) δ 160.9, 160.5, 155.3, 154.8, 137.5, 136.6, 130.0, 129.0, 120.4, 106.8, 106.8, 106.3, 101.0, 80.4, 67.9, 55.4, 35.0, 28.9, 28.5, 28.0. HRMS: (ESI) [M+H]⁺ calc. for C₂₇H₃₈N₃O₅, 484.2806, observed, 484.2805.

### tert-butyl 4-((4-(4-(3-(trifluoromethoxy)phenoxy)butyl)phenyl)carbamoyl)piperazine-1-carboxylate (2r)

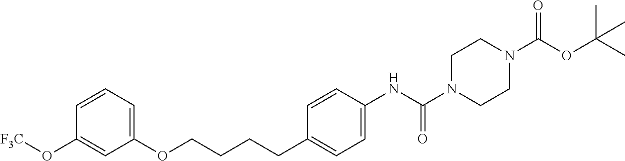 [SMILES: CC(C)(C)OC(=O)N1CCN(C(=O)Nc2ccc(CCCCOc3cccc(OC(F)(F)F)c3)cc2)CC1]

Synthesized according to procedure 3, white solid, 75 mg, 40% yield. ¹H NMR (600 MHz, CDCl₃) δ 7.35-7.28 (m, 1H), 7.26-7.24 (m, 2H), 7.14-7.11 (m, 2H), 6.83-6.78 (m, 2H), 6.75-6.72 (m, 1H), 6.28 (s, 1H), 3.94 (t, J=6.0 Hz, 2H), 3.51-3.46 (m, 8H), 2.64 (t, J=7.1 Hz, 2H), 1.84-1.75 (m, 4H), 1.48 (s, 9H). ¹³C NMR (126 MHz, CDCl₃) δ 160.2, 155.2, 154.8, 150.2 (d, J=1.8 Hz), 138.8, 137.3, 136.6, 130.2, 129.0, 120.6 (d, J=257.1 Hz), 120.4, 113.0, 107.7, 80.5, 68.2, 43.9, 35.0, 28.7, 28.5, 27.9. HRMS: (ESI) [M+H]⁺ calc. for C₂₇H₃₅F₃N₃O₄, 538.2523, observed, 538.2525.

### tert-butyl 4-((4-(4-(3-cyanophenoxy)butyl)phenyl)carbamoyl)piperazine-1-carboxylate (2s)

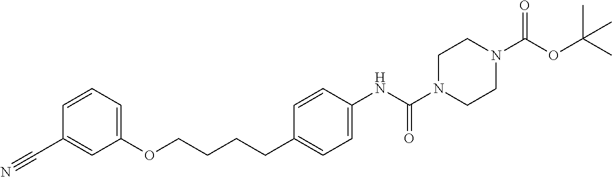 [SMILES: CC(C)(C)OC(=O)N1CCN(C(=O)Nc2ccc(CCCCOc3cccc(C#N)c3)cc2)CC1]

Synthesized according to procedure 3, white solid, 160 mg, 72% yield. ¹H NMR (500 MHz, CDCl₃) δ 7.34 (t, J=7.9 Hz, 1H), 7.28-7.24 (m, 2H), 7.22-7.20 (m, 1H), 7.13-7.10 (m, 3H), 7.09-7.06 (m, 1H), 6.39 (s, 1H), 3.95 (t, J=6.0 Hz, 2H), 3.52-3.44 (m, 8H), 2.64 (t, J=7.0 Hz, 2H), 1.84-1.72 (m, 4H), 1.48 (s, 9H). ¹³C NMR (126 MHz, CDCl₃) δ 159.2, 155.3, 154.8, 137.2, 136.7, 130.4, 129.0, 124.5, 120.6, 120.0, 119.0, 117.4, 113.2, 80.4, 68.3, 43.9, 34.9, 28.5, 27.8. HRMS: (ESI) [M+H]⁺ calc. for C₂₇H₃₅N₄O₄, 479.2653, observed, 479.2654.

### tert-butyl 4-((4-(4-(naphthalen-2-yloxy)butyl)phenyl)carbamoyl)piperazine-1-carboxylate (2t)

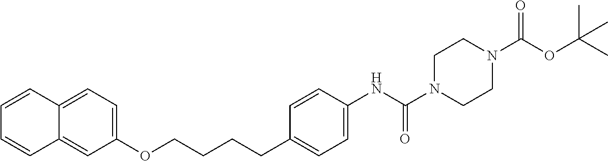 [SMILES: CC(C)(C)OC(=O)N1CCN(C(=O)Nc2ccc(CCCCOc3ccc4ccccc4c3)cc2)CC1]

Synthesized according to procedure 3, white solid, 193 mg, 83% yield. ¹H NMR (500 MHz, CDCl₃) δ 7.78-7.68 (m, 3H), 7.44-7.40 (m, 1H), 7.34-7.30 (m, 1H), 7.27-7.24 (m, 2H), 7.16-7.09 (m, 4H), 6.30 (s, 1H), 4.08 (t, J=6.2 Hz, 2H), 3.53-3.44 (m, 8H), 2.67 (t, J=7.3 Hz, 2H), 1.93-1.78 (m, 4H), 1.48 (s, 9H). ¹³C NMR (126 MHz, CDCl₃) δ 157.2, 155.2, 154.8, 137.5, 136.6, 134.7, 129.4, 129.0, 129.0, 127.8, 126.8, 126.4, 123.6, 120.4, 119.1, 106.7, 80.5, 67.9, 44.0, 35.1, 28.9, 28.5, 28.1. HRMS: (ESI) [M+H]⁺ calc. for C₃₀H₃₈N₃O₄, 504.2857, observed, 504.2866.

### tert-butyl 4-((4-(4-(3-methoxy-5-methylphenoxy)butyl)phenyl)carbamoyl)piperazine-1-carboxylate (2u)

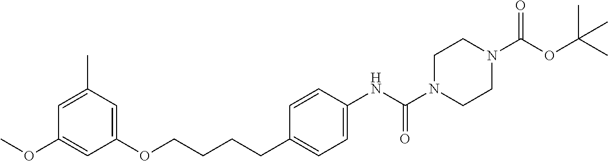 [SMILES: COc1cc(C)cc(OCCCCc2ccc(NC(=O)N3CCN(C(=O)OC(C)(C)C)CC3)cc2)c1]

Synthesized according to procedure 3, white solid, 104 mg, 60% yield. ¹H NMR (500 MHz, CDCl₃) δ 7.26-7.23 (m, 2H), 7.13-7.10 (m, 2H), 6.32-6.31 (m, 2H), 6.30 (s, 1H), 6.27-6.26 (m, 1H), 3.94-3.90 (m, 2H), 3.76 (s, 3H), 3.53-3.45 (m, 8H), 2.63 (t, J=7.2 Hz, 2H), 2.29 (s, 3H), 1.82-1.73 (m, 4H), 1.48 (s, 9H). ¹³C NMR (126 MHz, CDCl₃) δ 160.8, 160.3, 155.2, 154.8, 140.3, 137.5, 136.6, 129.0, 120.4, 107.8, 107.2, 98.1, 80.4, 67.8, 55.4, 44.0, 35.0, 28.9, 28.5, 28.0, 21.9. HRMS: (ESI) [M+H]⁺ calc. for C₂₈H₄₀N₃O₅, 498.2962, observed, 498.2977.

### tert-butyl 4-((4-(6-(4-fluorophenoxy)hexyl)phenyl)carbamoyl)piperazine-1-carboxylate (2v)

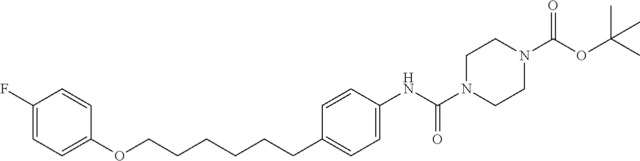 [SMILES: CC(C)(C)OC(=O)N1CCN(C(=O)Nc2ccc(CCCCCCOc3ccc(F)cc3)cc2)CC1]

Synthesized according to procedure 3, white solid, 183 mg, 53% yield. ¹H NMR (500 MHz, CDCl₃) δ 7.25-7.21 (m, 2H), 7.11-7.08 (m, 2H), 6.97-6.93 (m, 2H), 6.83-6.79 (m, 2H), 6.32 (s, 1H), 3.89 (t, J=6.5 Hz, 2H), 3.50-3.45 (m, 8H), 2.56 (t, J=7.7 Hz, 2H), 1.79-1.71 (m, 2H), 1.64-1.58 (m, 2H), 1.51-1.42 (m, 11H), 1.42-1.33 (m, 2H). ¹⁹F NMR (376 MHz, CDCl₃) δ −124.44-−124.54 (m). ¹³C NMR (126 MHz, CDCl₃) δ 157.2 (d, J=237.6 Hz), 155.3 (d, J=2.1 Hz), 155.3, 154.8, 138.0, 136.4, 129.0, 120.4, 115.8 (d, J=23.0 Hz), 115.5 (d, J=8.1 Hz), 80.5, 68.6, 44.0, 35.3, 31.5, 29.3, 29.0, 28.5, 26.0. HRMS: (ESI) [M+H]⁺ calc. for C₂₈H₃₉FN₃O₄, 500.2919, observed, 500.2914.

### tert-butyl 4-((4-(6-(3-fluorophenoxy)hexyl)phenyl)carbamoyl)piperazine-1-carboxylate (2w)

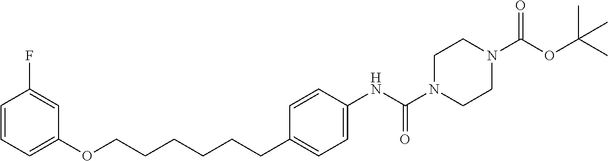 [SMILES: CC(C)(C)OC(=O)N1CCN(C(=O)Nc2ccc(CCCCCCOc3cccc(F)c3)cc2)CC1]

Synthesized according to procedure 3, white solid, 256 mg, 74% yield. ¹H NMR (500 MHz, CDCl₃) δ 7.25-7.22 (m, 2H), 7.22-7.17 (m, 1H), 7.10-7.07 (m, 2H), 6.67-6.62 (m, 2H), 6.61-6.57 (m, 1H), 6.46 (s, 1H), 3.90 (t, J=6.5 Hz, 2H), 3.52-3.42 (m, 8H), 2.56 (t, J=7.7 Hz, 2H), 1.80-1.72 (m, 2H), 1.64-1.57 (m, 2H), 1.50-1.42 (m, 11H), 1.41-1.34 (m, 2H). ¹⁹F NMR (376 MHz, CDCl₃) δ −111.87-−111.98 (m). ¹³C NMR (126 MHz, CDCl₃) δ 163.7 (d, J=244.7 Hz), 160.6 (d, J=10.9 Hz), 155.4, 154.8, 137.9, 136.4, 130.2 (d, J=10.1 Hz), 128.9, 120.5, 110.4 (d, J=2.8 Hz), 107.3 (d, J=21.3 Hz), 102.2 (d, J=24.7 Hz), 80.4, 68.3, 43.9, 35.3, 31.5, 29.1, 29.0, 28.5, 26.0. HRMS: (ESI) [M+H]⁺ calc. for C₂₈H₃₉FN₃O₄, 500.2919, observed, 500.2925.

### tert-butyl 4-((4-(6-(2-fluorophenoxy)hexyl)phenyl)carbamoyl)piperazine-1-carboxylate (2x)

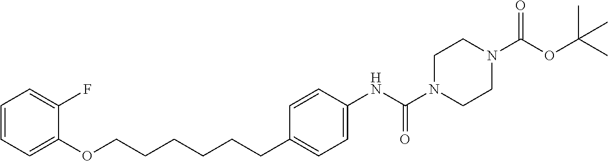 [SMILES: CC(C)(C)OC(=O)N1CCN(C(=O)Nc2ccc(CCCCCCOc3ccccc3F)cc2)CC1]

Synthesized according to procedure 3, white solid, 205 mg, 59% yield. ¹H NMR (500 MHz, CDCl₃) δ 7.25-7.21 (m, 2H), 7.11-7.08 (m, 2H), 7.08-7.00 (m, 1H), 6.97-6.92 (m, 1H), 6.90-6.85 (m, 1H), 6.31 (s, 1H), 4.03-3.98 (m, 2H), 3.52-3.45 (m, 8H), 2.59-2.54 (m, 2H), 1.84-1.77 (m, 2H), 1.65-1.58 (m, 2H), 1.53-1.46 (m, 11H), 1.42-1.35 (m, 2H). ¹⁹F NMR (376 MHz, CDCl₃) δ −134.81-−134.91 (m). ¹³C NMR (126 MHz, CDCl₃) δ 155.3, 154.8, 152.9 (d, J=245.5 Hz), 147.3 (d, J=10.4 Hz), 138.0, 136.4, 129.0, 124.4 (d, J=3.8 Hz), 120.9 (d, J=6.8 Hz), 120.4, 116.3 (d, J=18.2 Hz), 115.0 (d, J=1.6 Hz), 80.4, 69.4, 44.0, 35.3, 31.5, 29.3, 29.0, 28.5, 25.9. HRMS: (ESI) [M+H]⁺ calc. for C₂₈H₃₉FN₃O₄, 500.2919, observed, 500.2914.

### tert-butyl 4-((4-(6-(3,5-difluorophenoxy)hexyl)phenyl)carbamoyl)piperazine-1-carboxylate (2y)

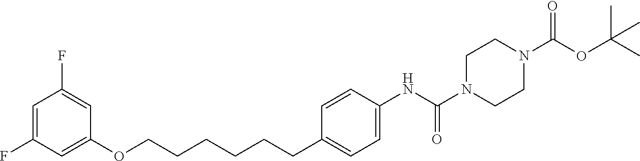 [SMILES: CC(C)(C)OC(=O)N1CCN(C(=O)Nc2ccc(CCCCCCOc3cc(F)cc(F)c3)cc2)CC1]

Synthesized according to procedure 3, white solid, 250 mg, 69% yield. ¹H NMR (500 MHz, CDCl₃) δ 7.25-7.21 (m, 2H), 7.10-7.06 (m, 2H), 6.47 (s, 1H), 6.42-6.35 (m, 3H), 3.88 (t, J=6.5 Hz, 2H), 3.46 (d, J=2.4 Hz, 8H), 2.56 (t, J=7.6 Hz, 2H), 1.78-1.71 (m, 2H), 1.64-1.57 (m, 2H), 1.47 (s, 11H), 1.40-1.32 (m, 2H). ¹⁹F NMR (376 MHz, CDCl₃) δ −109.64-−109.73 (m). ¹³C NMR (126 MHz, CDCl₃) δ 163.8 (dd, J=245.7, 15.9 Hz), 161.2 (t, J=13.8 Hz), 155.3, 154.7, 137.9, 136.5, 128.9, 120.5, 98.5-98.2 (m), 96.1 (t, J=26.0 Hz), 80.4, 68.6, 43.9, 35.2, 31.5, 29.0, 28.9, 28.5, 25.9. HRMS: (ESI) [M+H]⁺ calc. for C₂₈H₃₈F₂N₃O₄, 518.2825, observed, 518.2808.

### tert-butyl 4-((4-(6-(2,3-difluorophenoxy)hexyl)phenyl)carbamoyl)piperazine-1-carboxylate (2z)

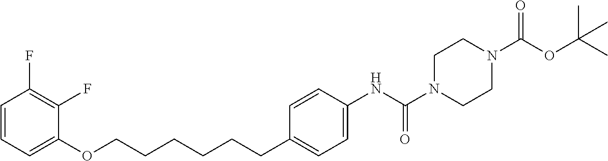 [SMILES: CC(C)(C)OC(=O)N1CCN(C(=O)Nc2ccc(CCCCCCOc3cccc(F)c3F)cc2)CC1]

Synthesized according to procedure 3, white solid, 287 mg, 80% yield. ¹H NMR (500 MHz, CDCl₃) δ 7.25-7.20 (m, 2H), 7.09-7.05 (m, 2H), 6.97-6.91 (m, 1H), 6.76-6.67 (m, 2H), 6.58 (s, 1H), 4.00 (t, J=6.6 Hz, 2H), 3.49-3.41 (m, 8H), 2.55 (t, J=7.6 Hz, 2H), 1.84-1.74 (m, 2H), 1.64-1.56 (m, 2H), 1.52-1.42 (m, 11H), 1.42-1.32 (m, 2H). ¹⁹F NMR (376 MHz, CDCl₃) δ −137.71-−137.83 (m), −159.77-−160.00 (m). ¹³C NMR (126 MHz, CDCl₃) δ 155.4, 154.7, 151.5 (dd, J=246.3, 10.4 Hz), 148.9 (dd, J=7.8, 3.2 Hz), 141.5 (dd, J=247.0, 14.1 Hz), 137.8, 136.5, 128.9, 123.2 (dd, J=8.8, 5.1 Hz), 120.5, 109.9 (d, J=2.8 Hz), 109.0 (d, J=17.7 Hz), 80.4, 69.8, 43.9, 35.2, 31.4, 29.1, 28.9, 28.5, 25.8. HRMS: (ESI) [M+H]⁺ calc. for C₂₈H₃₈F₂N₃O₄, 518.2825, observed, 518.2834.

### tert-butyl 4-((4-(6-(2,6-difluorophenoxy)hexyl)phenyl)carbamoyl)piperazine-1-carboxylate (2aa)

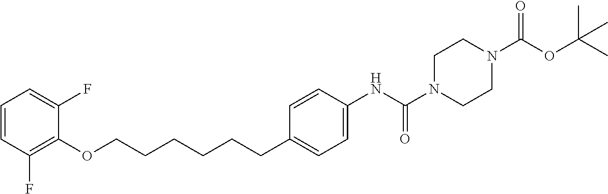 [SMILES: CC(C)(C)OC(=O)N1CCN(C(=O)Nc2ccc(CCCCCCOc3c(F)cccc3F)cc2)CC1]

Synthesized according to procedure 3, white solid, 202 mg, 56% yield. ¹H NMR (600 MHz, CDCl₃) δ 7.25-7.22 (m, 2H), 7.10-7.08 (m, 2H), 6.95-6.89 (m, 1H), 6.89-6.84 (m, 2H), 6.38 (s, 1H), 4.10 (t, J=6.6 Hz, 2H), 3.50-3.44 (m, 8H), 2.58-2.54 (m, 2H), 1.77-1.71 (m, 2H), 1.60 (p, J=7.7 Hz, 2H), 1.52-1.44 (m, 11H), 1.37 (p, J=7.6 Hz, 2H). ¹⁹F NMR (565 MHz, CDCl₃) δ −128.28-−128.33 (m). ¹³C NMR (126 MHz, CDCl₃) δ 156.4 (dd, J=248.0, 5.7 Hz), 155.3, 154.8, 138.0, 136.4, 135.9 (t, J=14.2 Hz), 128.9, 122.7 (t, J=9.3 Hz), 120.4, 112.2 (dd, J=17.2, 5.6 Hz), 80.4, 74.8 (t, J=2.8 Hz), 44.0, 35.3, 31.5, 30.0, 29.0, 28.5, 25.6. HRMS: (ESI) [M+H]⁺ calc. for C₂₈H₃₈F₂N₃O₄, 518.2825, observed, 518.2836.

### tert-butyl 4-((4-(6-(2,4,6-trifluorophenoxy)hexyl)phenyl)carbamoyl)piperazine-1-carboxylate (2ab)

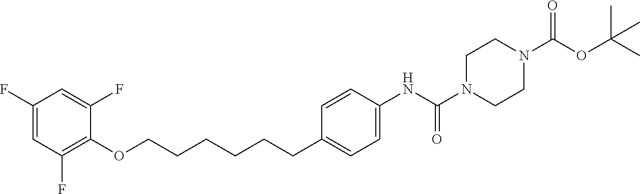 [SMILES: CC(C)(C)OC(=O)N1CCN(C(=O)Nc2ccc(CCCCCCOc3c(F)cc(F)cc3F)cc2)CC1]

Synthesized according to procedure 3, white solid, 239 mg, 64% yield. ¹H NMR (600 MHz, CDCl₃) δ 7.25-7.21 (m, 2H), 7.13-7.05 (m, 2H), 6.66 (t, J=8.5 Hz, 2H), 6.35 (s, 1H), 4.03 (t, J=6.5 Hz, 2H), 3.55-3.42 (m, 8H), 2.56 (t, J=7.7 Hz, 2H), 1.77-1.68 (m, 2H), 1.64-1.56 (m, 2H), 1.50-1.44 (m, 11H), 1.39-1.32 (m, 2H). ¹⁹F NMR (565 MHz, CDCl₃) δ −115.23-−115.31 (m), −125.10-−125.20 (m). ¹³C NMR (126 MHz, CDCl₃) δ 157.1 (dt, J=244.8, 14.5 Hz), 156.3 (ddd, J=249.7, 14.9, 7.8 Hz), 155.3, 155.2, 154.8, 138.0, 136.4, 132.8 (td, J=14.6, 5.4 Hz), 129.0, 120.4, 101.1-100.5 (m), 80.4, 75.3, 75.2, 75.2, 44.0, 35.3, 31.5, 29.9, 29.0, 28.5, 25.6. HRMS: (ESI) [M+H]⁺ calc. for C₂₈H₃₇F₃N₃O₄, 536.2731, observed, 536.2731.

### tert-butyl 4-((4-(6-(pyridin-3-yloxy)hexyl)phenyl)carbamoyl)piperazine-1-carboxylate (2ac)

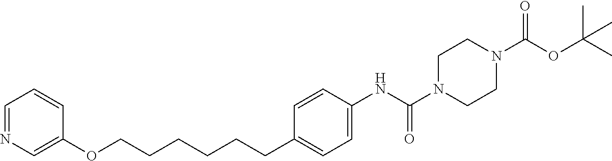 [SMILES: CC(C)(C)OC(=O)N1CCN(C(=O)Nc2ccc(CCCCCCOc3cccnc3)cc2)CC1]

Synthesized according to procedure 3, white solid, 176 mg, 63% yield. ¹H NMR (600 MHz, CDCl₃) δ 8.28-8.25 (m, 1H), 8.19 (d, J=4.0 Hz, 1H), 7.24-7.21 (m, 2H), 7.21-7.15 (m, 2H), 7.08 (d, J=8.4 Hz, 2H), 6.50 (s, 1H), 3.96 (t, J=6.5 Hz, 2H), 3.50-3.43 (m, 8H), 2.56 (t, J=7.6 Hz, 2H), 1.81-1.74 (m, 2H), 1.61 (p, J=7.6 Hz, 2H), 1.50-1.44 (m, 11H), 1.40-1.34 (m, 2H). ¹³C NMR (151 MHz, CDCl₃) δ 155.3, 154.7, 142.0, 138.1, 137.8, 136.5, 128.9, 124.0, 121.2, 120.5, 80.4, 68.3, 44.1, 35.2, 31.4, 29.1, 28.9, 28.5, 25.9. HRMS: (ESI) [M+H]⁺ calc. for C₂₇H₃₉N₄O₄, 483.2966, observed, 483.2963.

### tert-butyl 4-((4-(3-(4-cyclopropylphenoxy)propyl)phenyl)carbamoyl)piperazine-1-carboxylate (2ad)

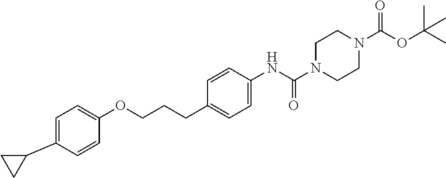 [SMILES: CC(C)(C)OC(=O)N1CCN(C(=O)Nc2ccc(CCCOc3ccc(C4CC4)cc3)cc2)CC1]

Synthesized according to procedure 3, white solid, 250 mg, 75% yield. ¹H NMR (600 MHz, CDCl₃) δ 7.24 (d, J=8.4 Hz, 2H), 7.11 (d, J=8.3 Hz, 2H), 6.99 (d, J=8.6 Hz, 2H), 6.80-6.77 (m, 2H), 6.62 (d, J=4.6 Hz, 1H), 3.90 (t, J=6.2 Hz, 2H), 3.44 (s, 8H), 2.74 (t, J=7.6 Hz, 2H), 2.07-2.00 (m, 2H), 1.87-1.81 (m, 1H), 1.48 (s, 9H), 0.91-0.86 (m, 2H), 0.63-0.58 (m, 2H). ¹³C NMR (151 MHz, CDCl₃) δ 157.0, 155.3, 154.7, 136.7, 136.7, 135.8, 129.0, 126.8, 120.6, 114.5, 80.4, 66.9, 44.0, 31.5, 31.0, 28.5, 14.7, 8.6. HRMS: (ESI) [M+H]⁺ calc. for C₂₈H₃₈N₃O₄, 480.2857, observed, 480.2870.

### tert-butyl 4-((4-(6-(2-ethylphenoxy)hexyl)phenyl)carbamoyl)piperazine-1-carboxylate (2ae)

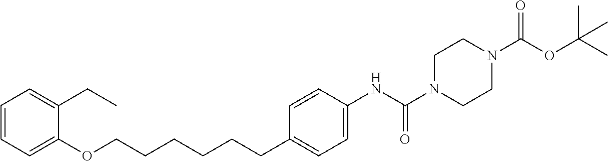 [SMILES: CCc1ccccc1OCCCCCCc1ccc(NC(=O)N2CCN(C(=O)OC(C)(C)C)CC2)cc1]

Synthesized according to procedure 3, white solid, 158 mg, 67% yield. ¹H NMR (600 MHz, CDCl₃) δ 7.25-7.22 (m, 2H), 7.15-7.12 (m, 2H), 7.11-7.09 (m, 2H), 6.87 (td, J=7.4, 0.9 Hz, 1H), 6.83-6.79 (m, 1H), 6.32 (s, 1H), 3.94 (t, J=6.3 Hz, 2H), 3.52-3.45 (m, 8H), 2.64 (q, J=7.5 Hz, 2H), 2.60-2.53 (m, 2H), 1.83-1.73 (m, 2H), 1.62 (p, J=7.7 Hz, 2H), 1.54-1.46 (m, 11H), 1.39 (p, J=7.4 Hz, 2H), 1.19 (t, J=7.5 Hz, 3H). ¹³C NMR (151 MHz, CDCl₃) δ 156.9, 155.3, 154.8, 138.1, 136.4, 132.8, 129.0, 129.0, 126.8, 120.4, 120.3, 111.0, 80.4, 67.8, 35.3, 31.6, 29.4, 29.0, 28.5, 26.2, 23.5. HRMS: (ESI) [M+H]⁺ calc. for C₃₀H₄₄N₃O₄, 510.3326, observed, 510.3329.

### tert-butyl 4-((4-(6-(2-(trifluoromethyl)phenoxy)hexyl)phenyl)carbamoyl)piperazine-1-carboxylate (2af)

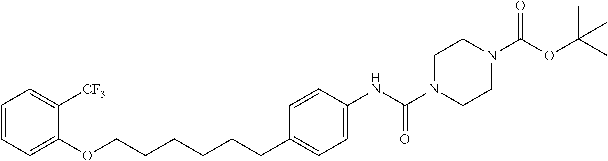 [SMILES: CC(C)(C)OC(=O)N1CCN(C(=O)Nc2ccc(CCCCCCOc3ccccc3C(F)(F)F)cc2)CC1]

Synthesized according to procedure 3, white solid, 126 mg, 49% yield. ¹H NMR (600 MHz, CDCl₃) δ 7.54 (d, J=7.6 Hz, 1H), 7.47-7.43 (m, 1H), 7.23 (d, J=8.4 Hz, 2H), 7.09 (d, J=8.4 Hz, 2H), 6.96 (t, J=8.0 Hz, 2H), 6.44 (s, 1H), 4.01 (t, J=6.3 Hz, 2H), 3.46 (s, 8H), 2.58-2.53 (m, 2H), 1.79 (dt, J=14.2, 6.3 Hz, 2H), 1.61 (p, J=7.7 Hz, 2H), 1.53-1.45 (m, 11H), 1.37 (p, J=7.6, 7.2 Hz, 2H). ¹⁹F NMR (565 MHz, CDCl₃) δ −62.39. ¹³C NMR (151 MHz, CDCl₃) δ 157.1, 155.3, 154.7, 138.0, 136.4, 133.3, 128.9, 127.1 (q, J=5.2 Hz), 123.9 (q, J=272.3 Hz), 120.4, 119.8, 118.9 (q, J=30.6 Hz), 112.8, 80.4, 68.6, 44.0, 35.2, 31.5, 29.1, 28.8, 28.5, 25.8. HRMS: (ESI) [M+H]⁺ calc. for C₂₉H₃₉F₃N₃O₄, 550.2887, observed, 550.2904.

### tert-butyl 4-((4-(5-(2-ethylphenoxy)pentyl)phenyl)carbamoyl)piperazine-1-carboxylate (2ag)

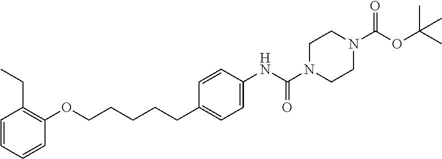 [SMILES: CCc1ccccc1OCCCCCc1ccc(NC(=O)N2CCN(C(=O)OC(C)(C)C)CC2)cc1]

Synthesized according to procedure 3, white solid, 158 mg, 69% yield. ¹H NMR (600 MHz, CDCl₃) δ 7.24 (d, J=8.4 Hz, 2H), 7.16-7.09 (m, 4H), 6.87 (t, J=7.4 Hz, 1H), 6.80 (d, J=8.3 Hz, 1H), 6.30 (s, 1H), 3.94 (t, J=6.4 Hz, 2H), 3.52-3.46 (m, 8H), 2.63 (q, J=7.6 Hz, 2H), 2.61-2.58 (m, 2H), 1.84-1.79 (m, 2H), 1.67 (p, J=7.7 Hz, 2H), 1.54-1.47 (m, 11H), 1.18 (t, J=7.5 Hz, 3H). ¹³C NMR (151 MHz, CDCl₃) δ 156.9, 155.2, 154.8, 137.9, 136.4, 132.9, 129.0, 129.0, 126.8, 120.4, 120.3, 111.1, 80.5, 67.7, 35.3, 31.4, 29.4, 28.5, 25.9, 23.5, 14.4. HRMS: (ESI) [M+H]⁺ calc. for C₂₉H₄₂N₃O₄, 496.3170, observed, 496.3168.

### tert-butyl 4-((4-(4-(3-cyclopropyl-2-fluorophenoxy)butyl)phenyl)carbamoyl)piperazine-1-carboxylate (2ah)

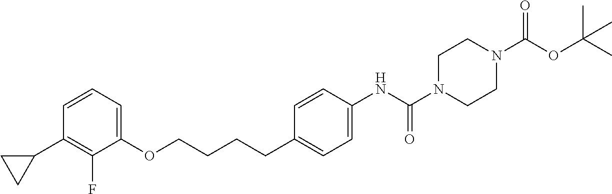 [SMILES: CC(C)(C)OC(=O)N1CCN(C(=O)Nc2ccc(CCCCOc3cccc(C4CC4)c3F)cc2)CC1]

Synthesized according to procedure 3, white solid, 124 mg, 54% yield. ¹H NMR (600 MHz, CDCl₃) δ 7.24 (d, J=8.4 Hz, 2H), 7.12 (d, J=8.4 Hz, 2H), 6.90 (td, J=8.0, 1.2 Hz, 1H), 6.73 (td, J=8.0, 1.2 Hz, 1H), 6.46-6.41 (m, 1H), 6.39 (s, 1H), 4.00 (t, J=6.2 Hz, 2H), 3.47 (s, 8H), 2.64 (t, J=7.4 Hz, 2H), 2.13-2.06 (m, 1H), 1.85-1.75 (m, 4H), 1.48 (s, 9H), 0.98-0.94 (m, 2H), 0.72-0.67 (m, 2H). ¹⁹F NMR (565 MHz, CDCl₃) δ −142.41 (t, J=7.0 Hz). ¹³C NMR (151 MHz, CDCl₃) δ 155.3, 154.8, 152.0 (d, J=243.8 Hz), 147.0 (d, J=11.1 Hz), 137.0 (d, J=130.3 Hz), 131.8 (d, J=11.9 Hz), 129.0, 123.5 (d, J=4.6 Hz), 120.4, 117.2 (d, J=3.2 Hz), 111.7, 80.4, 69.3, 44.0, 35.0, 28.8, 28.5, 27.9, 8.8, 8.7, 8.0. HRMS: (ESI) [M+H]⁺ calc. for C₂₉H₃₉FN₃O₄, 512.2919, observed, 512.2930.

### tert-butyl 4-((4-(4-(3-cyclopropyl-5-fluorophenoxy)butyl)phenyl)carbamoyl)piperazine-1-carboxylate (2ai)

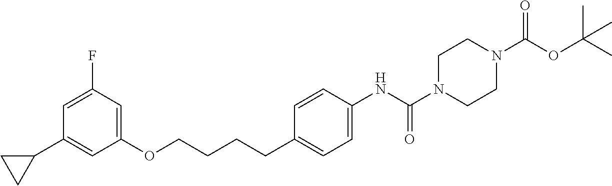 [SMILES: CC(C)(C)OC(=O)N1CCN(C(=O)Nc2ccc(CCCCOc3cc(F)cc(C4CC4)c3)cc2)CC1]

Synthesized according to procedure 3, white solid, 185 mg, 62% yield. ¹H NMR (600 MHz, CDCl₃) δ 7.25 (d, J=8.5 Hz, 2H), 7.11 (d, J=8.5 Hz, 2H), 6.44 (s, 1H), 6.41-6.38 (m, 1H), 6.36 (dt, J=10.7, 2.3 Hz, 1H), 6.32 (dt, J=9.8, 1.8 Hz, 1H), 3.90 (t, J=6.0 Hz, 2H), 3.50-3.44 (m, 8H), 2.62 (t, J=7.1 Hz, 2H), 1.85-1.80 (m, 1H), 1.79-1.72 (m, 4H), 1.47 (s, 9H), 0.96-0.93 (m, 2H), 0.69-0.65 (m, 2H). ¹⁹F NMR (565 MHz, CDCl₃) δ −112.76 (t, J=10.2 Hz). ¹³C NMR (151 MHz, CDCl₃) δ 163.8 (d, J=243.4 Hz), 160.3 (d, J=11.8 Hz), 155.3, 154.7, 147.5 (d, J=9.8 Hz), 137.4, 136.6, 129.0, 120.5, 108.1 (d, J=2.3 Hz), 104.6 (d, J=22.1 Hz), 98.9 (d, J=25.2 Hz), 80.4, 68.0, 44.0, 35.0, 28.8, 28.5, 27.9, 15.7, 15.7, 9.6. HRMS: (ESI) [M+H]⁺ calc. for C₂₉H₃₉FN₃O₄, 512.2919, observed, 512.2923.

### tert-butyl 4-((4-(3-(3-cyclobutylphenoxy)propyl)phenyl)carbamoyl)piperazine-1-carboxylate (2aj)

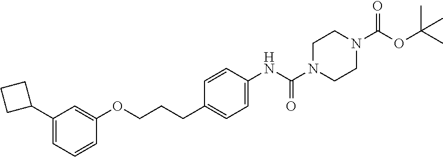 [SMILES: CC(C)(C)OC(=O)N1CCN(C(=O)Nc2ccc(CCCOc3cccc(C4CCC4)c3)cc2)CC1]

Synthesized according to procedure 3, white solid, 70 mg, 41% yield. ¹H NMR (600 MHz, CDCl₃) δ 7.28-7.26 (m, 2H), 7.21 (t, J=7.9 Hz, 1H), 7.16 (d, J=8.4 Hz, 2H), 6.82 (d, J=7.6 Hz, 1H), 6.78 (s, 1H), 6.72 (dd, J=8.1, 2.3 Hz, 1H), 6.39 (s, 1H), 3.96 (t, J=6.2 Hz, 2H), 3.57-3.47 (m, 10H), 2.79 (t, J=7.6 Hz, 2H), 2.38-2.30 (m, 2H), 2.20-2.12 (m, 2H), 2.11-2.06 (m, 2H), 2.05-1.98 (m, 1H), 1.86 (q, J=8.8 Hz, 2H), 1.50 (s, 9H). ¹³C NMR (151 MHz, CDCl₃) δ 159.1, 155.2, 154.7, 148.1, 136.8, 136.7, 129.2, 129.1, 120.5, 118.8, 112.9, 111.5, 80.4, 66.7, 44.1, 40.4, 31.6, 31.1, 29.8, 28.5, 18.3. HRMS: (ESI) [M+H]⁺ calc. for C₂₉H₄₀N₃O₄, 494.3013, observed, 494.3015.

### tert-butyl 4-((4-(5-(3-chlorophenoxy)pentyl)phenyl)carbamoyl)piperazine-1-carboxylate (2aq)

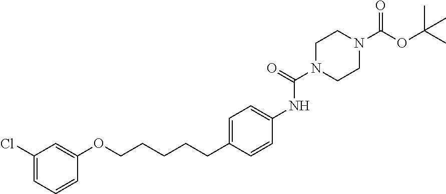 [SMILES: CC(C)(C)OC(=O)N1CCN(C(=O)Nc2ccc(CCCCCOc3cccc(Cl)c3)cc2)CC1]

Synthesized according to procedure 3, white solid, 152 mg, 65% yield. ¹H NMR (500 MHz, CDCl₃) δ 7.25-7.23 (m, 2H), 7.17 (t, J=8.1 Hz, 1H), 7.10 (d, J=8.5 Hz, 2H), 6.92-6.89 (m, 1H), 6.87 (t, J=2.2 Hz, 1H), 6.78-6.74 (m, 1H), 6.35 (brs, 1H), 3.91 (t, J=6.5 Hz, 2H), 3.47 (dd, J=8.2, 3.2 Hz, 8H), 2.59 (t, J=7.6 Hz, 2H), 1.82-1.74 (m, 2H), 1.65 (p, J=7.6 Hz, 2H), 1.52-1.42 (m, 11H). ¹³C NMR (126 MHz, CDCl₃) δ 160.0, 155.3, 154.8, 137.7, 136.5, 134.9, 130.3, 129.0, 120.8, 120.4, 115.0, 113.2, 68.2, 43.9, 35.3, 31.3, 29.1, 28.5, 25.7. FIRMS: (ESI) [M+H]⁺ calc. for C₂₇H₃₇ClN₃O₄, 502.2467, observed, 502.2482.

### tert-butyl 4-((4-(4-(naphthalen-1-yloxy)butyl)phenyl)carbamoyl)piperazine-1-carboxylate (2au)

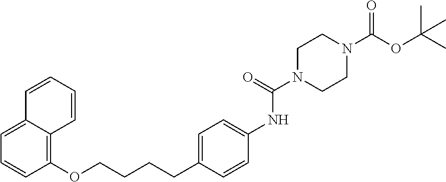 [SMILES: CC(C)(C)OC(=O)N1CCN(C(=O)Nc2ccc(CCCCOc3cccc4ccccc34)cc2)CC1]

Synthesized according to procedure 3, white solid, 111 mg, 38% yield. ¹H NMR (500 MHz, CDCl₃) δ 8.31-8.28 (m, 1H), 7.83-7.80 (m, 1H), 7.52-7.46 (m, 2H), 7.44-7.42 (m, 1H), 7.40-7.36 (m, 1H), 7.30-7.28 (m, 2H), 7.17 (d, J=8.3 Hz, 2H), 6.81 (d, J=7.5 Hz, 1H), 6.44 (brs, 1H), 4.17 (t, J=6.0 Hz, 2H), 3.53-3.48 (m, 8H), 2.72 (t, J=7.4 Hz, 2H), 2.01-1.87 (m, 4H), 1.51 (s, 9H). ¹³C NMR (126 MHz, CDCl₃) δ 155.2, 154.9, 154.7, 137.5, 136.6, 134.6, 129.0, 127.5, 126.4, 126.0, 125.8, 125.2, 122.2, 120.5, 120.1, 104.7, 80.4, 68.0, 44.0, 35.0, 28.9, 28.5, 28.2. HRMS: (ESI) [M+H]⁺ calc. for C₃₀H₃₈N₃O₄, 504.2857, observed, 504.2859.

### tert-butyl 4-((4-(6-(cyclopentyloxy)hexyl)phenyl)carbamoyl)piperazine-1-carboxylate (2av)

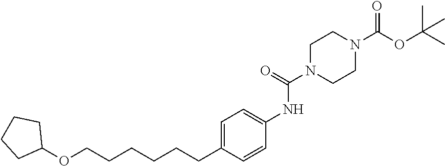 [SMILES: CC(C)(C)OC(=O)N1CCN(C(=O)Nc2ccc(CCCCCCOC3CCCC3)cc2)CC1]

Synthesized according to procedure 3, white solid, 69 mg, 79% yield. ¹H NMR (600 MHz, CDCl₃) δ 7.22 (d, J=8.2 Hz, 2H), 7.08 (d, J=8.2 Hz, 2H), 6.42 (brs, 1H), 3.87-3.82 (m, 1H), 3.46 (s, 8H), 3.34 (t, J=6.8 Hz, 2H), 2.53 (t, J=7.6 Hz, 2H), 1.74-1.64 (m, 4H), 1.62-1.45 (m, 17H), 1.37-1.29 (m, 4H). ¹³C NMR (151 MHz, CDCl₃) δ 155.3, 154.7, 138.1, 136.3, 128.9, 120.4, 81.3, 80.4, 69.0, 44.0, 35.3, 32.4, 31.6, 30.1, 29.2, 28.5, 26.2, 23.6. HRMS: (ESI) [M+H]⁺ calc. for C₂₇H₄₄N₃O₄, 474.3326, observed, 474.3330.

### tert-butyl 4-((4-(5-(benzyloxy)pentyl)phenyl)carbamoyl)piperazine-1-carboxylate (2aw)

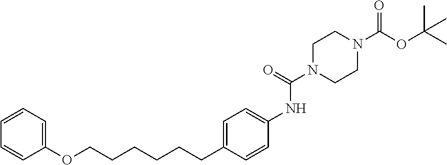 [SMILES: CC(C)(C)OC(=O)N1CCN(C(=O)Nc2ccc(CCCCCCOc3ccccc3)cc2)CC1]

Synthesized according to procedure 3, white solid, 82 mg, 73% yield. ¹H NMR (600 MHz, CDCl₃) δ 7.36-7.31 (m, 4H), 7.29-7.27 (m, 1H), 7.23 (d, J=8.2 Hz, 2H), 7.09 (d, J=8.2 Hz, 2H), 6.31 (s, 1H), 4.49 (s, 2H), 3.52-3.43 (m, 10H), 2.55 (t, J=7.7 Hz, 2H), 1.67-1.57 (m, 6H), 1.48 (s, 9H), 1.42-1.35 (m, 4H). ¹³C NMR (151 MHz, CDCl₃) δ 155.2, 154.7, 138.7, 138.0, 136.3, 129.0, 128.5, 127.8, 127.6, 120.3, 80.5, 73.0, 70.4, 43.9, 35.3, 31.5, 29.7, 28.5, 25.9. HRMS: (ESI) [M+H]⁺ calc. for C₂₈H₄₀N₃O₄, 482.3013, observed, 482.2997.

### tert-butyl 4-((4-(3-(3-phenylpropoxy)propyl)phenyl)carbamoyl)piperazine-1-carboxylate (2ax)

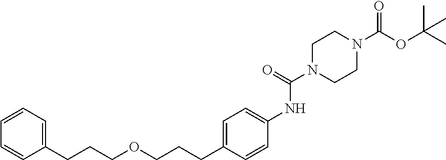 [SMILES: CC(C)(C)OC(=O)N1CCN(C(=O)Nc2ccc(CCCOCCCc3ccccc3)cc2)CC1]

Synthesized according to procedure 3, white solid, 87 mg, 78% yield. ¹H NMR (600 MHz, CDCl₃) δ 7.28 (t, J=7.6 Hz, 2H), 7.24 (d, J=8.4 Hz, 2H), 7.21-7.17 (m, 3H), 7.12 (d, J=8.4 Hz, 2H), 6.35 (s, 1H), 3.48 (s, 8H), 3.40 (q, J=6.3 Hz, 4H), 2.72-2.67 (m, 2H), 2.66-2.64 (m, 2H), 1.92-1.84 (m, 4H), 1.47 (s, 9H). ¹³C NMR (151 MHz, CDCl₃) δ 155.2, 154.7, 142.1, 137.2, 136.5, 129.0, 128.6, 128.4, 125.9, 120.4, 80.5, 70.1, 70.0, 44.2, 32.5, 31.8, 31.5, 31.4, 28.5. HRMS: (ESI) [M+H]⁺ calc. for C₂₈H₄₀N₃O₄, 482.3013, observed, 482.3015.

### tert-butyl 4-((4-(6-cyclobutoxyhexyl)phenyl)carbamoyl)piperazine-1-carboxylate (2ay)

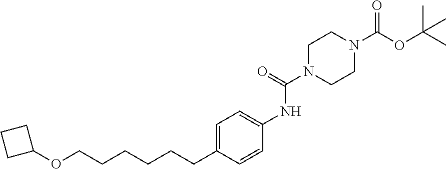 [SMILES: CC(C)(C)OC(=O)N1CCN(C(=O)Nc2ccc(CCCCCCOC3CCC3)cc2)CC1]

Synthesized according to procedure 3, white solid, 89 mg, 42% yield. ¹H NMR (400 MHz, CDCl₃) δ 7.23 (d, J=8.2 Hz, 2H), 7.08 (d, J=8.2 Hz, 2H), 6.35 (brs, 1H), 3.94-3.82 (m, 1H), 3.53-3.42 (m, 8H), 3.29 (t, J=6.7 Hz, 2H), 2.54 (t, J=7.7 Hz, 2H), 2.24-2.12 (m, 2H), 1.96-1.82 (m, 2H), 1.73-1.61 (m, 2H), 1.60-1.45 (m, 13H), 1.40-1.29 (m, 4H). ¹³C NMR (151 MHz, CDCl₃) δ 155.3, 154.8, 138.1, 136.3, 129.0, 120.4, 80.4, 73.2, 67.8, 44.0, 43.8, 35.3, 31.6, 30.6, 29.9, 29.2, 28.5, 26.2, 12.7. HRMS: (ESI) [M+H]⁺ calc. for C₂₆H₄₂N₃O₄, 460.3170, observed, 460.3170.

### tert-Butyl 4-((4-(6-(cyclohexyloxy)hexyl)phenyl)carbamoyl)piperazine-1-carboxylate, 2az

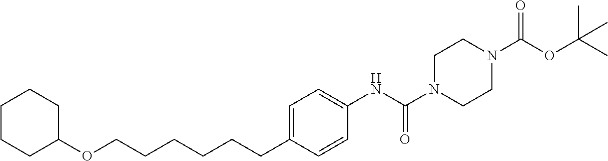 [SMILES: CC(C)(C)OC(=O)N1CCN(C(=O)Nc2ccc(CCCCCCOC3CCCCC3)cc2)CC1]

Synthesized according to procedure 3. 0.133 g, 84%, amorphous white solid. ¹H NMR (600 MHz, CDCl₃) δ 7.23 (d, J=8.4 Hz, 1H), 7.09 (d, J=8.4 Hz, 2H), 6.32 (s, 1H), 3.51-3.44 (m, 8H), 3.41 (t, J=6.8 Hz, 2H), 3.18 (dt, J=9.6, 5.0 Hz, 1H), 2.57-2.51 (m, 2H), 1.93-1.86 (m, 2H), 1.76-1.69 (m, 2H), 1.67-1.62 (m, 2H), 1.61-1.50 (m, 5H), 1.48 (s, 9H), 1.38-1.29 (m, 4H), 1.26-1.19 (m, 4H). ¹³C NMR (151 MHz, CDCl₃) δ 155.1, 154.6, 138.0, 136.2, 128.8, 120.2, 80.3, 67.8, 43.9, 35.2, 32.4, 31.4, 30.1, 29.0, 28.4, 26.1, 25.8, 24.3, 15.3. HRMS (ESI⁺) found 488.3476 (C₂₈H₄₆N₃O₄), required 488.3483 [M+H]⁺.

### tert-Butyl 4-((4-(4-((5,6,7,8-tetrahydronaphthalen-1-yl)oxy)butyl)phenyl)carbamoyl)piperazine-1-carboxylate, 2ba

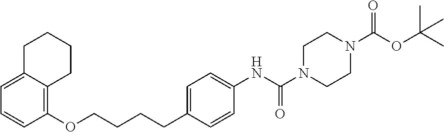 [SMILES: CC(C)(C)OC(=O)N1CCN(C(=O)Nc2ccc(CCCCOc3cccc4c3CCCC4)cc2)CC1]

Synthesized according to procedure 3. 0.093 g, 66%, amorphous white solid. ¹H NMR (600 MHz, CDCl₃) δ 7.25 (d, J=8.3 Hz, 2H), 7.12 (d, J=8.3 Hz, 2H), 7.03 (t, J=7.9 Hz, 1H), 6.67 (d, J=7.6 Hz, 1H), 6.61 (d, J=8.1 Hz, 1H), 6.30 (s, 1H), 3.94 (t, J=5.6 Hz, 2H), 3.52-3.45 (m, 8H), 2.74 (t, J=6.0 Hz, 2H), 2.64 (q, J=6.2 Hz, 4H), 1.84-1.71 (m, 8H), 1.48 (s, 9H). ¹³C NMR (151 MHz, CDCl₃) δ 156.7, 155.1, 154.6, 138.5, 137.5, 136.3, 128.9, 126.1, 125.5, 121.1, 120.3, 107.5, 80.3, 67.4, 43.9, 34.9, 29.6, 28.9, 28.4, 28.0, 23.1, 22.8, 22.8.

### tert-Butyl 4-((4-(4-((2,3-dihydro-1H-inden-4-yl)oxy)butyl)phenyl)carbamoyl)piperazine-1-carboxylate, 2bb

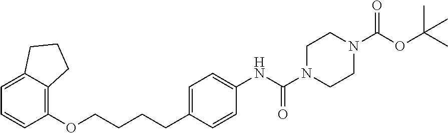 [SMILES: CC(C)(C)OC(=O)N1CCN(C(=O)Nc2ccc(CCCCOc3cccc4c3CCC4)cc2)CC1]

Synthesized according to procedure 3. 0.123 g, 83%, amorphous white solid. ¹H NMR (500 MHz, CDCl₃) δ 7.27-7.23 (m, 2H), 7.12 (d, J=8.2 Hz, 2H), 7.09 (t, J=7.8 Hz, 1H), 6.83 (d, J=7.4 Hz, 1H), 6.63 (d, J=8.1 Hz, 1H), 6.34 (s, 1H), 3.98 (t, J=5.8 Hz, 2H), 3.53-3.44 (m, 8H), 2.89 (dt, J=23.1, 7.4 Hz, 4H), 2.64 (t, J=7.0 Hz, 2H), 2.05 (p, J=7.5 Hz, 2H), 1.85-1.73 (m, 4H), 1.48 (s, 9H). ¹³C NMR (126 MHz, CDCl₃) δ 155.5, 155.1, 154.6, 146.2, 137.5, 136.4, 132.0, 128.8, 127.3, 120.3, 116.7, 108.7, 80.3, 67.6, 43.9, 34.8, 33.2, 29.4, 28.8, 28.4, 27.9, 24.9. HRMS (ESI⁺) found 516.2843 (C₂₉H₃₉N₃O₄Na), required 516.2833 [M+Na]⁺.

### tert-Butyl 4-((4-(7-methoxyheptyl)phenyl)carbamoyl)piperazine-1-carboxylate, 2bc

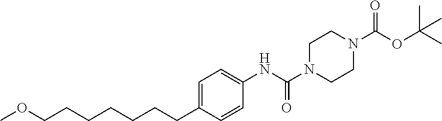 [SMILES: COCCCCCCCc1ccc(NC(=O)N2CCN(C(=O)OC(C)(C)C)CC2)cc1]

Synthesized according to procedure 3. 93 mg, 56%, amorphous white solid. ¹H NMR (500 MHz, CDCl₃) δ 7.23 (d, J=8.3 Hz, 2H), 7.09 (d, J=8.2 Hz, 2H), 6.32 (s, 1H), 3.52-3.44 (m, 8H), 3.35 (t, J=6.7 Hz, 2H), 3.32 (s, 3H), 2.60-2.50 (m, 2H), 1.61-1.51 (m, 4H), 1.48 (d, J=0.6 Hz, 9H), 1.35-1.28 (m, 6H). ¹³C NMR (126 MHz, CDCl₃) δ 155.1, 154.6, 138.0, 136.2, 128.8, 120.2, 80.3, 72.9, 58.5, 43.9, 35.2, 31.4, 29.6, 29.3, 29.1, 28.4, 26.0.

### tert-Butyl 4-((4-((4-(phenoxymethyl)cyclohexyl)methyl)phenyl)carbamoyl)piperazine-1-carboxylate, 2bd

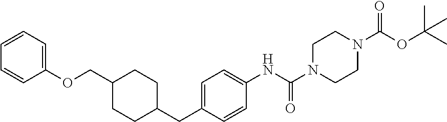 [SMILES: CC(C)(C)OC(=O)N1CCN(C(=O)Nc2ccc(CC3CCC(COc4ccccc4)CC3)cc2)CC1]

Synthesized according to procedure 3. 21 mg, 25%, d.r. 80:20, amorphous white solid. ¹H NMR (500 MHz, CDCl₃) δ 7.26-7.23 (m, 2H), 7.07 (d, J=8.2 Hz, 2H), 6.95-6.89 (m, 1H), 6.89-6.85 (m, 2H), 6.32 (s, 1H), 3.88 (d, J=7.0 Hz, 0.4H), 3.73 (d, J=6.4 Hz, 1.6H), 3.54-3.44 (m, 8H), 2.54 (d, J=7.6 Hz, 0.4H), 2.46 (d, J=7.1 Hz, 1.6H), 1.94-1.84 (m, 2H), 1.80-1.70 (m, 3H), 1.69-1.56 (m, 4H), 1.48 (s, 9H), 1.08-0.95 (m, 3H).

### tert-Butyl 4-((4-(8-methoxyoctyl)phenyl)carbamoyl)piperazine-1-carboxylate, 2be

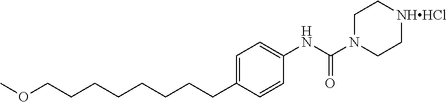

Synthesized according to procedure 3. 0.11 g, 71%, amorphous white solid. ¹H NMR (500 MHz, CDCl₃) δ 7.22 (d, J=8.5 Hz, 2H), 7.08 (d, J=8.5 Hz, 2H), 6.39 (s, 1H), 3.50-3.43 (m, 8H), 3.35 (t, J=6.6 Hz, 2H), 3.32 (s, 3H), 2.57-2.51 (m, 2H), 1.61-1.50 (m, 4H), 1.47 (s, 9H), 1.32-1.28 (m, 8H). ¹³C NMR (126 MHz, CDCl₃) δ 155.2, 154.6, 138.0, 136.2, 128.8, 120.2, 80.2, 72.9, 58.5, 43.8, 35.2, 31.5, 29.6, 29.4, 29.4, 29.1, 28.4, 26.1.

### tert-Butyl 4-((4-((3-(phenoxymethyl)cyclohexyl)methyl)phenyl)carbamoyl)piperazine-1-carboxylate, 2bf

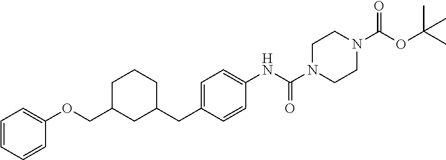 [SMILES: CC(C)(C)OC(=O)N1CCN(C(=O)Nc2ccc(CC3CCCC(COc4ccccc4)C3)cc2)CC1]

Synthesized according to procedure 3. 16 mg, 24%, d.r. 68:32, white amorphous solid. ¹H NMR (500 MHz, CDCl₃) δ 7.29-7.25 (m, 2H), 7.25-7.22 (m, 2H), 7.07 (d, J=8.1 Hz, 2H), 6.94-6.85 (m, 3H), 6.34 (s, 1H), 3.85-3.78 (m, 0.64H), 3.76-3.69 (m, 1.36H), 3.53-3.43 (m, 8H), 2.63-2.43 (m, 2H), 1.93-1.83 (m, 2H), 1.83-1.37 (m, 14H), 1.32-1.20 (m, 1H), 0.93 (dqd, J=37.9, 12.8, 3.6 Hz, 1.36H), 0.76 (q, J=12.1 Hz, 0.64H). ¹³C NMR (126 MHz, CDCl₃) δ 159.2, 159.2, 155.1, 154.6, 136.5, 136.3, 136.1, 129.6, 129.5, 129.4, 129.3, 126.2, 120.4, 120.3, 120.1, 120.1, 119.9, 114.5, 114.4, 80.3, 73.3, 71.5, 43.8, 43.4, 40.2, 39.3, 37.7, 36.3, 34.8, 33.5, 32.8, 32.5, 30.9, 30.0, 29.8, 29.3, 28.7, 28.4, 28.2, 27.4, 26.9, 26.1, 25.6, 21.0. HRMS (ESI⁺) found 530.2974 (C₃₀H₄₁N₃O₄Na), required 530.2989 [M+Na]f.

### tert-Butyl 4-((4-((3-(phenoxymethyl)cyclopentyl)methyl)phenyl)carbamoyl)piperazine-1-carboxylate, 2bg

Synthesized according to procedure 3. 0.086 g, 54%, d.r. 60:40, white amorphous solid. ¹H NMR (600 MHz, CDCl₃) δ 7.29-7.25 (m, 2H), 7.25-7.23 (m, 2H), 7.09 (d, J=8.0 Hz, 2H), 6.94-6.90 (m, 1H), 6.90-6.86 (m, 2H), 6.37 (s, 1H), 3.82 (d, J=7.1 Hz, 1H), 3.77 (d, J=7.0 Hz, 1H), 3.51-3.44 (m, 8H), 2.60 (ddd, J=18.2, 12.1, 7.3 Hz, 2H), 2.52-2.44 (m, OH), 2.37 (ddd, J=16.1, 9.0, 6.8 Hz, 1H), 2.21-2.10 (m, 1H), 2.01-1.91 (m, 1H), 1.85-1.71 (m, 2H), 1.54-1.43 (m, 10H), 1.38-1.22 (m, 1H), 1.03-0.93 (m, 1H). ¹³C NMR (151 MHz, CDCl₃) δ 159.1, 159.1, 155.1, 154.6, 137.1, 137.1, 136.3, 129.3, 129.2, 129.1, 120.4, 120.2, 120.1, 114.4, 80.3, 72.3, 72.2, 43.9, 43.8, 42.3, 41.4, 41.3, 41.0, 38.8, 37.8, 36.7, 35.2, 32.4, 31.5, 29.1, 28.4, 28.3. HRMS (ESI⁺) found 516.2829 (C₂₉H₃₉N₃O₄Na), required 516.2833 [M+Na]⁺.

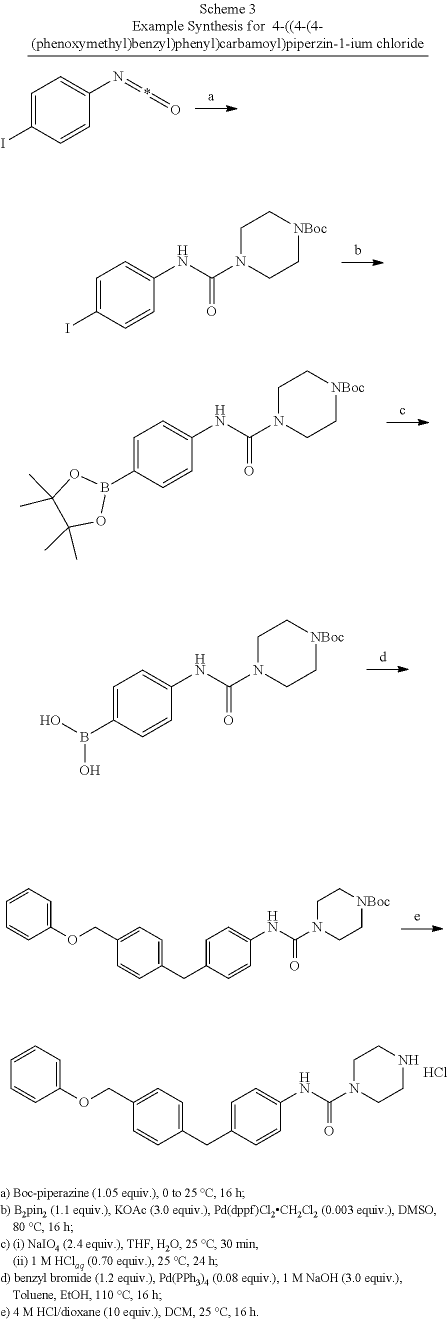

### tert-Butyl 4-((4-(4,4,5,5-tetramethyl-1,3,2-dioxaborolan-2-yl)phenyl)carbamoyl)piperazine-1-carboxylate

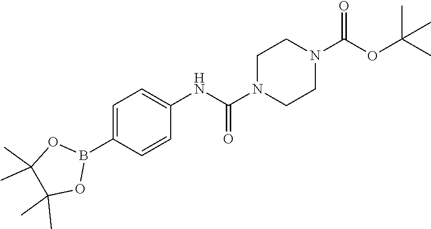 [SMILES: CC(C)(C)OC(=O)N1CCN(C(=O)Nc2ccc(B3OC(C)(C)C(C)(C)O3)cc2)CC1]

Synthesized according to procedure 14. 0.276 g, 71%, off-white amorphous solid. ¹H NMR (600 MHz, CDCl₃) δ 7.74 (d, J=8.2 Hz, 2H), 7.37 (d, J=8.3 Hz, 2H), 6.43 (s, 1H), 3.55-3.45 (m, 8H), 1.48 (s, 9H), 1.33 (s, 12H). ¹³C NMR (151 MHz, CDCl₃) δ 154.6, 154.4, 141.5, 135.8, 118.4, 83.6, 80.3, 44.0, 28.4, 24.8. ¹¹B NMR (128 MHz, CDCl₃) δ 29.8. HRMS (ESI⁺) found 454.2474 (C₂₂H₃₄BN₃O₅Na), required 454.2484 [M+Na]⁺.

### (4-(4-(tert-Butoxycarbonyl)piperazine-1-carboxamido)phenyl)boronic acid

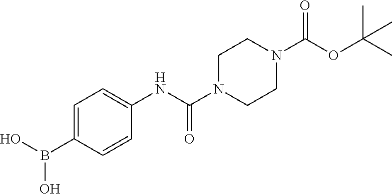 [SMILES: CC(C)(C)OC(=O)N1CCN(C(=O)Nc2ccc(B(O)O)cc2)CC1]

To a stirring biphasic solution of tert-butyl 4-((4-(4,4,5,5-tetramethyl-1,3,2-dioxaborolan-2-yl)phenyl)carbamoyl)piperazine-1-carboxylate (0.35 g, 1.0 eq., 0.81 mmol) in THE (2.6 mL) and water (0.65 mL) was added sodium periodate (0.52 g, 3.0 eq., 2.4 mmol). The reaction was stirred at 25° C. for 30 minutes, then aqueous hydrogen chloride (21 mg, 0.57 mL, 1.0 molar, 0.70 eq., 0.57 mmol) was added and the reaction was stirred at 25° C. for 24 hours. The reaction was diluted with water (25 mL) and extracted with ethyl acetate (3×25 mL). The organic extracts were combined, washed with brine (2×50 mL), dried with sodium sulfate, filtered, and concentrated in vacuo. The residue was purified by flash column chromatography (Teledyne ISCO, silica 25 g, Ø=2.5 cm, dichloromethane/methanol 0→5%) to give (4-(4-(tert-butoxycarbonyl)piperazine-1-carboxamido)phenyl)boronic acid (0.129 g, 369 mol, 46%). ¹H NMR (600 MHz, MeOD) δ 7.58-7.55 (m, 2H), 7.39-7.36 (m, 2H), 3.55-3.46 (m, 8H), 1.48 (s, 9H). ¹³C NMR (151 MHz, MeOD) δ 157.7, 156.3, 142.4, 135.5, 120.7, 81.6, 45.0, 28.6. ¹¹B NMR (128 MHz, MeOD) δ 27.5. HRMS (ESI⁺) found 372.1689 (C₁₆H₂₄BN₃O₅Na), required 372.1701 [M+Na]⁺.

### tert-Butyl 4-((4-(4-(phenoxymethyl)benzyl)phenyl)carbamoyl)piperazine-1-carboxylate, 2bh

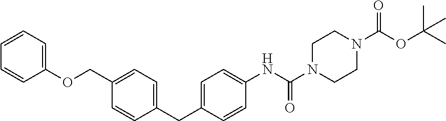 [SMILES: CC(C)(C)OC(=O)N1CCN(C(=O)Nc2ccc(Cc3ccc(COc4ccccc4)cc3)cc2)CC1]

To a stirring solution of 1-(bromomethyl)-4-(phenoxymethyl)benzene (83 mg, 1.2 eq., 0.30 mmol), (4-(4-(tert-butoxycarbonyl)piperazine-1-carboxamido)phenyl)boronic acid (87 mg, 1.0 eq., 0.25 mmol), and tetrakis(triphenylphosphine)palladium(0) (23 mg, 0.08 eq., 20 mol) in toluene (4.5 mL) and ethanol (0.39 mL) was added sodium hydroxide in water (10 mg, 0.25 mL, 1 molar, 1 eq., 0.25 mmol) under a nitrogen atmosphere. The reaction mixture was stirred at 110° C. for 16 hours. The reaction mixture was allowed to cool to room temperature, then concentrated in vacuo. The residue was purified by flash column chromatography (Teledyne ISCO, silica 25 g, Ø=2.5 cm, hexane/ethyl acetate 0→35%) then triturated with ethyl acetate to give tert-butyl 4-((4-(4-(phenoxymethyl)benzyl)phenyl)carbamoyl)piperazine-1-carboxylate 16t (0.013 g, 10%) as a white amorphous solid. ¹H NMR (500 MHz, CDCl₃) δ 7.34 (d, J=7.9 Hz, 2H), 7.31-7.27 (m, 2H), 7.26 (d, J=8.5 Hz, 2H), 7.18 (d, J=7.9 Hz, 2H), 7.11 (d, J=8.5 Hz, 2H), 6.99-6.93 (m, 3H), 6.39 (s, 1H), 5.02 (s, 2H), 3.93 (s, 2H), 3.51-3.43 (m, 8H), 1.48 (s, 9H). ¹³C NMR (126 MHz, CDCl₃) δ 158.8, 155.0, 154.6, 141.1, 136.8, 136.0, 134.7, 129.4, 129.4, 129.0, 127.8, 120.8, 120.3, 114.8, 80.3, 69.7, 43.8, 40.9, 28.4. HRMS (ESI⁺) found 524.2545 (C₃₀H₃₅N₃O₄Na), required 524.2520 [M+Na]⁺.

### N-(4-(5-phenoxypentyl)phenyl)piperazine-1-carboxamide hydrochloride (3a)

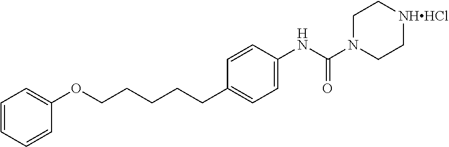

Synthesized according to procedure 4, purified via trituration with diethyl ether, white solid, 66 mg, 93% yield. ¹H NMR (600 MHz, MeOD) δ 7.28-7.21 (m, 4H), 7.14-7.11 (m, 2H), 6.91-6.85 (m, 3H), 3.94 (t, J=6.4 Hz, 2H), 3.80-3.75 (m, 4H), 3.28-3.25 (m, 4H), 2.61 (t, J=7.6 Hz, 2H), 1.81-1.76 (m, 2H), 1.70-1.64 (m, 2H), 1.53-1.47 (m, 2H). ¹³C NMR (126 MHz, MeOD) δ 160.5, 157.6, 139.2, 138.0, 130.4, 129.7, 122.4, 121.5, 115.5, 68.8, 44.5, 42.4, 36.2, 32.5, 30.3, 26.7. HRMS: (ESI) [M+H]⁺ calc. for C₂₂H₃₀N₃O₂, 368.2333, observed, 368.2333.

### N-(4-(6-phenoxyhexyl)phenyl)piperazine-1-carboxamide hydrochloride (3b)

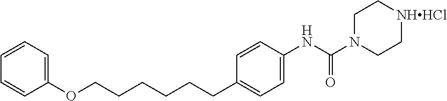

Synthesized according to procedure 4, purified via trituration with diethyl ether, white solid, 367 mg, 75% yield. ¹H NMR (500 MHz, MeOD) δ 7.28-7.21 (m, 4H), 7.14-7.09 (m, 2H), 6.92-6.84 (m, 3H), 3.94 (t, J=6.4 Hz, 2H), 3.79-3.75 (m, 4H), 3.30-3.25 (m, 4H), 2.59 (t, J=7.6 Hz, 2H), 1.81-1.70 (m, 2H), 1.69-1.58 (m, 2H), 1.55-1.46 (m, 2H), 1.45-1.35 (m, 2H). ¹³C NMR (126 MHz, MeOD) δ 160.6, 157.6, 139.4, 137.9, 130.4, 129.7, 122.4, 121.5, 115.5, 68.8, 44.5, 42.4, 36.2, 32.7, 30.4, 30.0, 27.0. HRMS: (ESI) [M+H]⁺ calc. for C₂₃H₃₂N₃O₂, 382.2489, observed, 382.2494.

### N-(4-(7-phenoxyheptyl)phenyl)piperazine-1-carboxamide hydrochloride (3c)

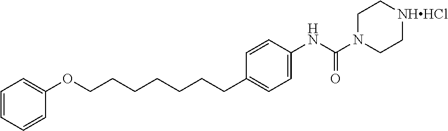

Synthesized according to procedure 4, purified via trituration with diethyl ether, white solid, 122 mg, 88% yield. ¹H NMR (600 MHz, MeOD) δ 7.27-7.21 (m, 4H), 7.13-7.09 (m, 2H), 6.91-6.86 (m, 3H), 3.94 (t, J=6.4 Hz, 2H), 3.77 (dd, J=6.1, 4.5 Hz, 4H), 3.29-3.25 (m, 4H), 2.58 (t, J=7.6 Hz, 2H), 1.78-1.71 (m, 2H), 1.64-1.57 (m, 2H), 1.51-1.44 (m, 2H), 1.43-1.33 (m, 2H). ¹³C NMR (151 MHz, MeOD) δ 160.6, 157.6, 139.4, 137.9, 130.4, 129.6, 122.4, 121.5, 115.5, 68.8, 44.5, 42.4, 36.2, 32.7, 30.4, 30.3, 30.2, 27.1. HRMS: (ESI) [M+H]⁺ calc. for C₂₄H₃₄N₃O₂, 396.2646, observed, 396.2645.

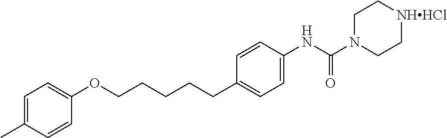

N-(4-(5-(p-tolyloxy)pentyl)phenyl)piperazine-1-carboxamide hydrochloride (3d): Synthesized according to procedure 4, purified via trituration with diethyl ether, white solid, 69 mg, 84% yield. ¹H NMR (500 MHz, MeOD) δ 7.27-7.23 (m, 2H), 7.13-7.09 (m, 2H), 7.05-7.01 (m, 2H), 6.77-6.73 (m, 2H), 3.90 (t, J=6.4 Hz, 2H), 3.79-3.75 (m, 4H), 3.28-3.25 (m, 4H), 2.60 (t, J=7.6 Hz, 2H), 2.24 (s, 3H), 1.80-1.72 (m, 2H), 1.70-1.62 (m, 2H), 1.52-1.44 (m, 2H). ¹³C NMR (126 MHz, MeOD) δ 158.4, 157.6, 139.2, 138.0, 130.8, 130.7, 129.7, 122.4, 115.3, 68.9, 44.5, 42.4, 36.2, 32.5, 30.3, 26.7, 20.5. HRMS: (ESI) [M+H]⁺ calc. for C₂₃H₃₂N₃O₂, 382.2489, observed, 382.2486.

### N-(4-(6-(m-tolyloxy)hexyl)phenyl)piperazine-1-carboxamide hydrochloride (3e)

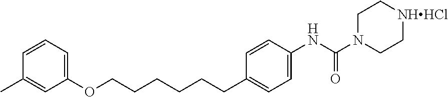

Synthesized according to procedure 4, purified via trituration with diethyl ether, white solid, 151 mg, 87% yield. H NMR (500 MHz, MeOD) δ 7.28-7.24 (m, 2H), 7.13-7.09 (m, 3H), 6.73-6.69 (m, 2H), 6.68-6.65 (m, 1H), 3.92 (t, J=6.4 Hz, 2H), 3.80-3.75 (m, 4H), 3.29-3.26 (m, 4H), 2.59 (t, J=7.6 Hz, 2H), 2.29 (s, 3H), 1.77-1.70 (m, 2H), 1.67-1.60 (m, 2H), 1.53-1.46 (m, 2H), 1.43-1.36 (m, 2H). ¹³C NMR (126 MHz, MeOD) δ 160.6, 157.6, 140.4, 139.4, 137.9, 130.1, 129.7, 122.4, 122.3, 116.2, 112.4, 68.7, 44.5, 42.4, 36.2, 32.7, 30.4, 30.0, 27.0, 21.6. HRMS: (ESI) [M+H]⁺ calc. for C₂₄H₃₄N₃O₂, 396.2646, observed, 396.2645.

### N-(4-(5-(m-tolyloxy)pentyl)phenyl)piperazine-1-carboxamide hydrochloride (3f)

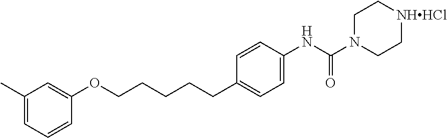

Synthesized according to procedure 4, purified via trituration with diethyl ether, white solid, 149 mg, 90% yield. ¹H NMR (500 MHz, MeOD) δ 7.28-7.24 (m, 2H), 7.15-7.08 (m, 3H), 6.73-6.70 (m, 1H), 6.70 (d, J=2.2 Hz, 1H), 6.67-6.65 (m, 1H), 3.92 (t, J=6.4 Hz, 2H), 3.79-3.75 (m, 4H), 3.29-3.25 (m, 4H), 2.61 (t, J=7.6 Hz, 2H), 2.29 (s, 3H), 1.80-1.74 (m, 2H), 1.71-1.63 (m, 2H), 1.53-1.45 (m, 2H). ¹³C NMR (126 MHz, MeOD) δ 160.6, 157.6, 140.4, 139.2, 138.0, 130.1, 129.7, 122.4, 122.3, 116.2, 112.4, 68.7, 44.5, 42.4, 36.2, 32.5, 30.3, 26.8, 21.6. HRMS: (ESI) [M+H]⁺ calc. for C₂₃H₃₂N₃O₂, 382.2489, observed, 382.2497.

### N-(4-(5-(3,5-dimethylphenoxy)pentyl)phenyl)piperazine-1-carboxamide hydrochloride (3g)

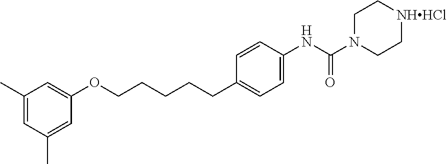

Synthesized according to procedure 4, white solid, 79 mg, 91% yield. ¹H NMR (500 MHz, MeOD) δ 7.28-7.25 (m, 2H), 7.14-7.10 (m, 2H), 6.56-6.54 (m, 1H), 6.50-6.48 (m, 2H), 3.90 (t, J=6.4 Hz, 2H), 3.81-3.75 (m, 4H), 3.30-3.25 (m, 4H), 2.60 (t, J=7.6 Hz, 2H), 2.24 (s, 6H), 1.80-1.71 (m, 2H), 1.71-1.62 (m, 2H), 1.53-1.43 (m, 2H). ¹³C NMR (126 MHz, MeOD) δ 160.6, 157.6, 140.1, 139.3, 138.0, 129.7, 123.2, 122.4, 113.2, 68.7, 44.5, 42.4, 36.2, 32.5, 30.3, 26.8, 21.5. HRMS: (ESI) [M+H]⁺ calc. for C₂₄H₃₄N₃O₂, 396.2646, observed, 396.2657.

### N-(4-(5-(o-tolyloxy)pentyl)phenyl)piperazine-1-carboxamide hydrochloride (3h)

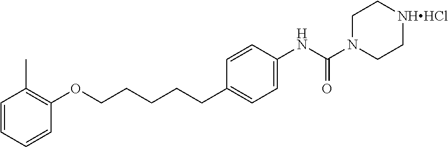

Synthesized according to procedure 4, white solid, 99 mg, 91% yield. ¹H NMR (500 MHz, MeOD) δ 7.28-7.24 (m, 2H), 7.14-7.10 (m, 2H), 7.10-7.06 (m, 2H), 6.84-6.82 (m, 1H), 6.80-6.76 (m, 1H), 3.95 (t, J=6.3 Hz, 2H), 3.79-3.75 (m, 4H), 3.29-3.25 (m, 4H), 2.62 (t, J=7.5 Hz, 2H), 2.15 (s, 3H), 1.86-1.78 (m, 2H), 1.72-1.65 (m, 2H), 1.57-1.49 (m, 2H). ¹³C NMR (126 MHz, MeOD) δ 158.5, 157.6, 139.3, 138.0, 131.4, 129.7, 127.9, 127.6, 122.4, 121.2, 112.0, 68.8, 44.5, 42.4, 36.2, 32.5, 30.4, 26.9, 16.4. HRMS: (ESI) [M+H]⁺ calc. for C₂₃H₃₂N₃O₂, 382.2489, observed, 382.2495.

### N-(4-(3-(4-ethylphenoxy)propyl)phenyl)piperazine-1-carboxamide hydrochloride (3i)

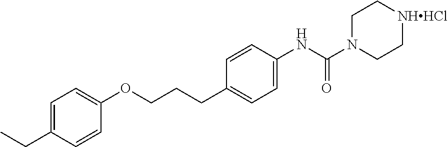

Synthesized according to procedure 4, white solid, 119 mg, 92% yield. ¹H NMR (500 MHz, MeOD) δ 7.31-7.22 (m, 2H), 7.19-7.11 (m, 2H), 7.11-7.04 (m, 2H), 6.87-6.70 (m, 2H), 3.91 (t, J=6.3 Hz, 2H), 3.83-3.71 (m, 4H), 3.30-3.22 (m, 4H), 2.85-2.66 (m, 2H), 2.56 (q, J=7.6 Hz, 2H), 2.09-1.95 (m, 2H), 1.19 (t, J=7.6 Hz, 3H). ¹³C NMR (126 MHz, MeOD) δ 158.5, 157.6, 138.3, 138.2, 137.5, 129.8, 129.7, 122.5, 115.4, 67.9, 44.5, 42.4, 32.5, 32.3, 29.0, 16.5. HRMS: (ESI) [M+H]⁺ calc. for C₂₂H₃₀N₃O₂, 368.2333, observed, 368.2325.

### N-(4-(3-(3-ethylphenoxy)butyl)phenyl)piperazine-1-carboxamide hydrochloride (3j)

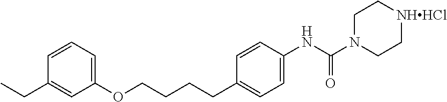

Synthesized according to procedure 4, purified via trituration with diethyl ether, white solid, 55 mg, 89% yield. ¹H NMR (500 MHz, MeOD) δ 7.29-7.25 (m, 2H), 7.16-7.11 (m, 3H), 6.76-6.73 (m, 1H), 6.73-6.71 (m, 1H), 6.70-6.67 (m, 1H), 3.98-3.93 (m, 2H), 3.80-3.75 (m, 4H), 3.30-3.25 (m, 4H), 2.67-2.63 (m, 2H), 2.59 (q, J=7.6 Hz, 2H), 1.79-1.74 (m, 4H), 1.21 (t, J=7.6 Hz, 3H). ¹³C NMR (126 MHz, MeOD) δ 160.6, 157.6, 147.0, 139.0, 138.1, 130.2, 129.7, 122.4, 121.1, 115.1, 112.6, 68.6, 44.5, 42.4, 35.9, 30.0, 29.9, 29.2, 16.2. HRMS: (ESI) [M+H]⁺ calc. for C₂₃H₃₂N₃O₂, 382.2489, observed, 382.2491.

### N-(4-(3-(3-propylphenoxy)propyl)phenyl)piperazine-1-carboxamide hydrochloride (3k)

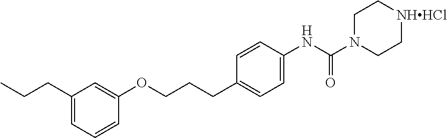

Synthesized according to procedure 4, purified via trituration with diethyl ether, white solid, 22 mg, 85% yield. ¹H NMR (500 MHz, MeOD) δ 7.29-7.26 (m, 2H), 7.17-7.14 (m, 2H), 7.14-7.11 (m, 1H), 6.75-6.72 (m, 1H), 6.71-6.67 (m, 2H), 3.93 (t, J=6.2 Hz, 2H), 3.79-3.75 (m, 4H), 3.29-3.26 (m, 4H), 2.76 (t, J=7.5 Hz, 2H), 2.56-2.52 (m, 2H), 2.07-2.01 (m, 2H), 1.62 (h, J=7.4 Hz, 2H), 0.93 (t, J=7.3 Hz, 3H). ¹³C NMR (126 MHz, MeOD) δ 160.5, 157.6, 145.4, 138.4, 138.2, 130.2, 129.8, 122.5, 121.9, 115.8, 112.7, 67.7, 44.5, 42.4, 39.1, 32.5, 32.3, 25.7, 14.1. HRMS: (ESI) [M+H]⁺ calc. for C₂₃H₃₂N₃O₂, 382.2489, observed, 382.2493.

### N-(4-(4-(3-isopropylphenoxy)butyl)phenyl)piperazine-1-carboxamide hydrochloride (3l)

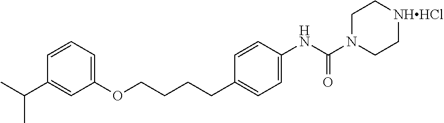

Synthesized according to procedure 4, white solid, 122 mg, 93% yield. ¹H NMR (500 MHz, MeOD) δ 7.30-7.25 (m, 2H), 7.17-7.12 (m, 3H), 6.79-6.76 (m, 1H), 6.75-6.73 (m, 1H), 6.70-6.67 (m, 1H), 3.98-3.93 (m, 2H), 3.81-3.75 (m, 4H), 3.30-3.25 (m, 4H), 2.84 (hept, J=6.9 Hz, 1H), 2.68-2.61 (m, 2H), 1.81-1.74 (m, 4H), 1.22 (d, J=6.9 Hz, 6H). ¹³C NMR (126 MHz, MeOD) δ 160.6, 157.6, 151.7, 139.0, 138.1, 130.3, 129.7, 122.4, 119.7, 113.8, 112.6, 68.7, 44.5, 42.4, 35.9, 35.5, 30.0, 29.2, 24.4. HRMS: (ESI) [M+H]⁺ calc. for C₂₄H₃₄N₃O₂, 396.2646, observed, 396.2649.

### N-(4-(4-(3-cyclopropylphenoxy)butyl)phenyl)piperazine-1-carboxamide hydrochloride (3m)

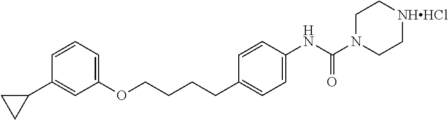

Synthesized according to procedure 4, white solid, 79 mg, 91% yield. ¹H NMR (500 MHz, MeOD) δ 7.30-7.25 (m, 2H), 7.16-7.12 (m, 2H), 7.10 (t, J=7.9 Hz, 1H), 6.66-6.63 (m, 1H), 6.63-6.60 (m, 1H), 6.59-6.58 (m, 1H), 3.96-3.92 (m, 2H), 3.80-3.75 (m, 4H), 3.29-3.25 (m, 4H), 2.67-2.62 (m, 2H), 1.85 (tt, J=8.4, 5.0 Hz, 1H), 1.79-1.74 (m, 4H), 0.95-0.89 (m, 2H), 0.66-0.61 (m, 2H). ¹³C NMR (126 MHz, MeOD) δ 160.6, 157.6, 151.7, 139.0, 138.1, 130.3, 129.7, 122.4, 119.7, 113.8, 112.6, 68.7, 44.5, 42.4, 35.9, 35.5, 30.0, 29.2, 24.4. HRMS: (ESI) [M+H]⁺ calc. for C₂₄H₃₂N₃O₂, 394.2489, observed, 394.2495.

### N-(4-(4-(3-(tert-butyl)phenoxy)butyl)phenyl)piperazine-1-carboxamide hydrochloride (3n)

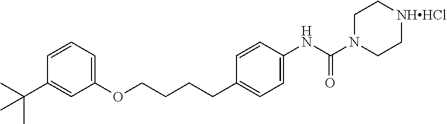

Synthesized according to procedure 4, white solid, 144 mg, 88% yield. ¹H NMR (500 MHz, MeOD) δ 7.29-7.26 (m, 2H), 7.18-7.14 (m, 3H), 6.96-6.94 (m, 1H), 6.90-6.89 (m, 1H), 6.70-6.68 (m, 1H), 3.98-3.95 (m, 2H), 3.79-3.76 (m, 4H), 3.29-3.27 (m, 4H), 2.67-2.64 (m, 2H), 1.80-1.77 (m, 4H), 1.29 (s, 9H). ¹³C NMR (126 MHz, MeOD) δ 160.4, 157.6, 153.9, 139.1, 138.1, 130.0, 129.7, 122.4, 118.7, 113.3, 112.0, 68.7, 44.5, 42.4, 36.0, 35.5, 31.8, 30.0, 29.2. HRMS: (ESI) [M+H]⁺ calc. for C₂₅H₃₆N₃O₂, 410.2802, observed, 410.2799.

### N-(4-(4-(4-(trifluoromethoxy)phenoxy)pentyl)phenyl)piperazine-1-carboxamide hydrochloride (3o)

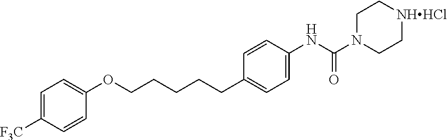

Synthesized according to procedure 4, white solid, 88 mg, 89% yield. ¹H NMR (500 MHz, MeOD) δ 7.57-7.53 (m, 2H), 7.28-7.24 (m, 2H), 7.14-7.11 (m, 2H), 7.04-7.00 (m, 2H), 4.03 (t, J=6.4 Hz, 2H), 3.79-3.76 (m, 4H), 3.29-3.26 (m, 4H), 2.62 (t, J=7.5 Hz, 2H), 1.85-1.78 (m, 2H), 1.72-1.65 (m, 2H), 1.55-1.47 (m, 2H). ¹⁹F NMR (376 MHz, MeOD) δ −62.96. ¹³C NMR (126 MHz, MeOD) δ 163.3, 157.6, 139.1, 138.0, 129.7, 127.9 (q, J=3.8 Hz), 126.1 (q, J=270.0 Hz), 124.5, 123.5 (q, J=32.5 Hz), 122.4, 122.1, 115.7, 69.3, 44.5, 42.4, 36.1, 32.4, 30.1, 26.6. HRMS: (ESI) [M+H]⁺ calc. for C₂₃H₂₉F₃N₃O₃, 436.2206, observed, 436.2211.

### N-(4-(4-(3-(trifluoromethoxy)phenoxy)pentyl)phenyl)piperazine-1-carboxamide hydrochloride (3p)

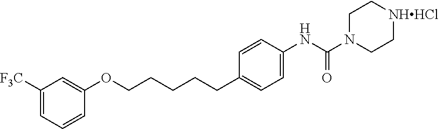

Synthesized according to procedure 4, white solid, 190 mg, 95% yield. ¹H NMR (500 MHz, MeOD) δ 7.47-7.41 (m, 1H), 7.28-7.25 (m, 2H), 7.21-7.16 (m, 1H), 7.15-7.10 (m, 4H), 4.01 (t, J=6.4 Hz, 2H), 3.80-3.75 (m, 4H), 3.30-3.25 (m, 4H), 2.61 (t, J=7.5 Hz, 2H), 1.85-1.78 (m, 2H), 1.72-1.64 (m, 2H), 1.55-1.46 (m, 2H). ¹⁹F NMR (376 MHz, MeOD) δ −64.21. ¹³C NMR (126 MHz, MeOD) δ 160.9, 157.6, 139.2, 138.0, 132.8 (q, J=31.9 Hz), 131.4, 129.7, 126.6 (q, J=271.8 Hz), 122.4, 119.2 (q, J=1.4 Hz), 118.0 (q, J=4.0 Hz), 112.2 (q, J=3.9 Hz), 69.3, 44.5, 42.4, 36.1, 32.5, 30.1, 26.6. HRMS: (ESI) [M+H]⁺ calc. for C₂₃H₂₉F₃N₃O₃, 436.2206, observed, 436.2207.

### N-(4-(4-(3-(methoxy)phenoxy)butyl)phenyl)piperazine-1-carboxamide hydrochloride (3q)

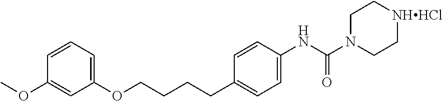

Synthesized according to procedure 4, white solid, 81 mg, 93% yield. ¹H NMR (500 MHz, MeOD) δ 7.29-7.25 (m, 2H), 7.16-7.13 (m, 3H), 7.12 (m, 1H), 6.49-6.47 (m, 1H), 6.47-6.46 (m, 1H), 6.45-6.43 (m, 1H), 3.97-3.92 (m, 2H), 3.79-3.75 (m, 4H), 3.75 (s, 3H), 3.29-3.25 (m, 4H), 2.67-2.62 (m, 2H), 1.80-1.74 (m, 4H). ¹³C NMR (126 MHz, MeOD) δ 162.4, 161.8, 157.6, 139.0, 138.1, 130.8, 129.7, 122.4, 107.7, 107.1, 101.9, 68.8, 55.6, 44.5, 42.4, 35.9, 29.9, 29.2. HRMS: (ESI) [M+H]⁺ calc. for C₂₂H₃₀N₃O₃, 384.2282, observed, 384.2278.

### N-(4-(4-(3-(trifluoromethoxy)phenoxy)butyl)phenyl)piperazine-1-carboxamide hydrochloride (3r)

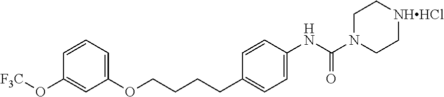

Synthesized according to procedure 4, white solid, 55 mg, 83% yield. ¹H NMR (600 MHz, MeOD) δ 7.32 (t, J=8.3 Hz, 1H), 7.29-7.26 (m, 2H), 7.16-7.13 (m, 2H), 6.92-6.87 (m, 1H), 6.84-6.79 (m, 1H), 6.80-6.75 (m, 1H), 4.01-3.97 (m, 2H), 3.80-3.76 (m, 4H), 3.29-3.26 (m, 4H), 2.67-2.64 (m, 2H), 1.81-1.76 (m, 4H). ¹⁹F NMR (565 MHz, MeOD) δ −59.4. ¹³C NMR (126 MHz, MeOD) δ 161.8, 157.6, 151.4, 151.4, 140.5, 138.9, 138.1, 131.5, 129.7, 124.5, 122.9 (q, J=255.3 Hz), 122.4, 122.1, 114.3, 113.6, 108.7, 69.3, 44.5, 42.4, 42.4, 35.9, 29.7, 29.1. HRMS: (ESI) [M+H]⁺ calc. for C₂₂H₂₇F₃N₃O₃, 438.1999, observed, 438.2003.

### N-(4-(4-(3-cyanophenoxy)butyl)phenyl)piperazine-1-carboxamide hydrochloride (3s)

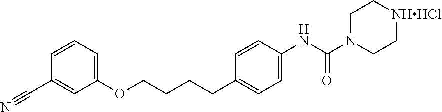

Synthesized according to procedure 4, white solid, 112 mg, 86% yield. ¹H NMR (500 MHz, MeOD) δ 7.45-7.40 (m, 1H), 7.30-7.24 (m, 3H), 7.22-7.19 (m, 2H), 7.16-7.13 (m, 2H), 4.03-3.99 (m, 2H), 3.80-3.76 (m, 4H), 3.31-3.25 (m, 4H), 2.68-2.63 (m, 2H), 1.83-1.75 (m, 4H). ¹³C NMR (126 MHz, MeOD) δ 160.8, 157.6, 138.9, 138.2, 131.7, 129.7, 125.3, 122.4, 121.1, 119.7, 118.4, 114.1, 69.3, 44.5, 42.4, 35.8, 29.6, 29.0. HRMS: (ESI) [M+H]⁺ calc. for C₂₂H₂₇N₄O₂, 379.2129, observed, 379.2130.

### N-(4-(4-(naphthalen-2-yloxy)butyl)phenyl)piperazine-1-carboxamide hydrochloride (3t)

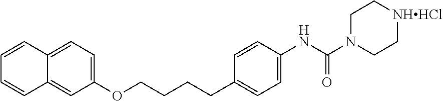

Synthesized according to procedure 4, purified via trituration with diethyl ether, white solid, 125 mg, 95% yield. ¹H NMR (500 MHz, MeOD) δ 7.75-7.71 (m, 3H), 7.42-7.37 (m, 1H), 7.31-7.26 (m, 3H), 7.19-7.15 (m, 3H), 7.10 (dd, J=9.0, 2.5 Hz, 1H), 4.09 (t, J=5.9 Hz, 2H), 3.79-3.76 (m, 4H), 3.29-3.26 (m, 4H), 2.68 (t, J=6.9 Hz, 2H), 1.88-1.79 (m, 4H). ¹³C NMR (126 MHz, MeOD) δ 158.4, 157.6, 139.0, 138.1, 136.2, 130.4, 130.3, 129.7, 128.5, 127.8, 127.2, 124.5, 122.4, 119.9, 107.5, 68.8, 44.5, 42.4, 36.0, 29.9, 29.3. HRMS: (ESI) [M+H]⁺ calc. for C₂₅H₃₀N₃O₂, 404.2333, observed, 404.2335.

### N-(4-(4-(3-methoxy-5-methylphenoxy)butyl)phenyl)piperazine-1-carboxamide hydrochloride (3u)

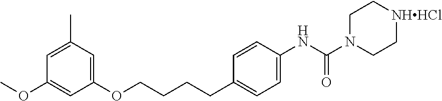

Synthesized according to procedure 4, white solid, 79 mg, 91% yield. ¹H NMR (500 MHz, MeOD) δ 7.29-7.25 (m, 2H), 7.16-7.13 (m, 2H), 6.33-6.29 (m, 2H), 6.25-6.23 (m, 1H), 3.94-3.90 (m, 2H), 3.80-3.75 (m, 4H), 3.73 (s, 3H), 3.29-3.26 (m, 5H), 2.67-2.62 (m, 2H), 2.25 (s, 3H), 1.79-1.74 (m, 4H). ¹³C NMR (126 MHz, MeOD) δ 162.2, 161.6, 157.6, 141.2, 139.0, 138.1, 129.7, 122.4, 108.6, 107.9, 99.0, 68.7, 55.6, 44.5, 42.4, 35.9, 29.9, 29.2, 21.8. HRMS: (ESI) [M+H]⁺ calc. for C₂₃H₃₂N₃O₃, 398.2438, observed, 398.2445.

### N-(4-(6-(4-fluorophenoxy)hexyl)phenyl)piperazine-1-carboxamide hydrochloride (3v)

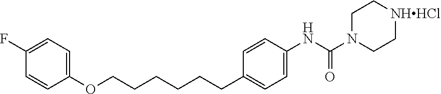

Synthesized according to procedure 4, white solid, 138 mg, 90% yield. ¹H NMR (500 MHz, MeOD) δ 7.27-7.23 (m, 2H), 7.13-7.09 (m, 2H), 7.00-6.94 (m, 2H), 6.89-6.83 (m, 2H), 3.91 (t, J=6.4 Hz, 2H), 3.79-3.75 (m, 4H), 3.29-3.26 (m, 4H), 2.59 (t, J=7.6 Hz, 2H), 1.77-1.71 (m, 2H), 1.66-1.59 (m, 2H), 1.53-1.46 (m, 2H), 1.42-1.35 (m, 2H). ¹⁹F NMR (376 MHz, MeOD) δ −126.75-−126.83 (m). ¹³C NMR (126 MHz, MeOD) δ 158.5 (d, J=234.3 Hz), 156.8 (d, J=2.0 Hz), 139.3, 138.0, 129.7, 122.4, 116.6 (d, J=4.5 Hz), 116.5 (d, J=10.9 Hz), 69.5, 44.5, 42.4, 36.1, 32.7, 30.3, 29.9, 27.0. HRMS: (ESI) [M+H]⁺ calc. for C₂₃H₃₁FN₃O₂, 400.2395, observed, 400.2394.

### N-(4-(6-(3-fluorophenoxy)hexyl)phenyl)piperazine-1-carboxamide hydrochloride (3w)

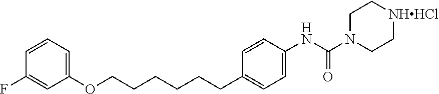

Synthesized according to procedure 4, white solid, 169 mg, 97% yield. ¹H NMR (500 MHz, MeOD) δ 7.28-7.20 (m, 3H), 7.13-7.09 (m, 2H), 6.72-6.68 (m, 1H), 6.65-6.60 (m, 2H), 3.94 (t, J=6.4 Hz, 2H), 3.81-3.74 (m, 4H), 3.30-3.25 (m, 4H), 2.58 (t, J=7.6 Hz, 2H), 1.79-1.71 (m, 2H), 1.67-1.59 (m, 2H), 1.53-1.46 (m, 2H), 1.43-1.35 (m, 2H). ¹⁹F NMR (376 MHz, MeOD) δ −114.20-−114.28 (m). ¹³C NMR (126 MHz, MeOD) δ 165.1 (d, J=243.1 Hz), 162.1 (d, J=10.9 Hz), 157.6, 139.3, 138.0, 131.4 (d, J=10.3 Hz), 129.6, 122.4, 111.5 (d, J=2.8 Hz), 107.9 (d, J=21.6 Hz), 102.9 (d, J=25.0 Hz), 69.2, 44.5, 42.4, 36.1, 32.7, 30.2, 29.9, 26.9. HRMS: (ESI) [M+H]⁺ calc. for C₂₃H₃₁FN₃O₂, 400.2395, observed, 400.2402.

### N-(4-(6-(2-fluorophenoxy)hexyl)phenyl)piperazine-1-carboxamide hydrochloride (3x)

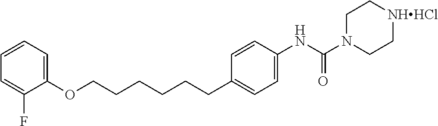

Synthesized according to procedure 4, white solid, 133 mg, 87% yield. ¹H NMR (500 MHz, MeOD) δ 7.27-7.23 (m, 2H), 7.13-7.10 (m, 2H), 7.08-7.02 (m, 3H), 6.91-6.86 (m, 1H), 4.02 (t, J=6.4 Hz, 2H), 3.79-3.74 (m, 4H), 3.29-3.25 (m, 4H), 2.59 (t, J=7.6 Hz, 2H), 1.82-1.74 (m, 2H), 1.67-1.60 (m, 2H), 1.55-1.48 (m, 2H), 1.44-1.36 (m, 2H). ¹⁹F NMR (376 MHz, MeOD) δ −137.10-−137.20 (m). ¹³C NMR (126 MHz, CDCl₃) δ 157.6, 154.1 (d, J=244.1 Hz), 148.5 (d, J=10.6 Hz), 139.3, 138.0, 129.7, 129.6, 125.5 (d, J=3.9 Hz), 122.4, 122.0 (d, J=6.9 Hz), 116.9 (d, J=18.2 Hz), 116.2 (d, J=1.7 Hz), 70.3, 44.5, 42.4, 36.1, 32.7, 30.3, 29.9, 26.9. HRMS: (ESI) [M+H]⁺ calc. for C₂₃H₃₁FN₃O₂, 400.2395, observed, 400.2402.

### N-(4-(6-(3,5-difluorophenoxy)hexyl)phenyl)piperazine-1-carboxamide hydrochloride (3y)

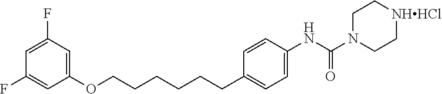

Synthesized according to procedure 4, white solid, 197 mg, 90% yield. ¹H NMR (600 MHz, MeOD) δ 7.28-7.25 (m, 2H), 7.13-7.09 (m, 2H), 6.53-6.49 (m, 2H), 6.49-6.44 (m, 1H), 3.93 (t, J=6.4 Hz, 2H), 3.80-3.76 (m, 4H), 3.29-3.24 (m, 4H), 2.58 (t, J=7.6 Hz, 2H), 1.77-1.71 (m, 2H), 1.65-1.59 (m, 2H), 1.51-1.45 (m, 2H), 1.41-1.34 (m, 2H). ¹⁹F NMR (565 MHz, MeOD) δ −111.56-−111.62 (m). ¹³C NMR (126 MHz, MeOD) δ 165.2 (dd, J=244.5, 16.0 Hz), 162.9 (t, J=13.8 Hz), 157.6, 139.3, 138.0, 129.6, 122.4, 99.4-99.0 (m), 96.6 (t, J=26.4 Hz), 69.7, 44.5, 42.4, 36.1, 32.6, 30.0, 29.9, 26.9. HRMS: (ESI) [M+H]⁺ calc. for C₂₃H₃₀F₂N₃O₂, 418.2301, observed, 418.2314.

### N-(4-(6-(2,3-difluorophenoxy)hexyl)phenyl)piperazine-1-carboxamide hydrochloride (3z)

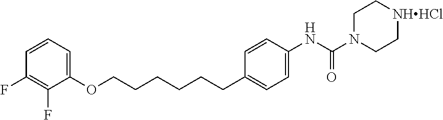

Synthesized according to procedure 4, white solid, 201 mg, 92% yield. ¹H NMR (600 MHz, MeOD) δ 7.28-7.24 (m, 2H), 7.13-7.09 (m, 2H), 7.06-7.01 (m, 1H), 6.89-6.85 (m, 1H), 6.82-6.77 (m, 1H), 4.04 (t, J=6.4 Hz, 2H), 3.80-3.76 (m, 4H), 3.29-3.25 (m, 4H), 2.58 (t, J=7.6 Hz, 2H), 1.82-1.75 (m, 2H), 1.67-1.60 (m, 2H), 1.54-1.48 (m, 2H), 1.43-1.36 (m, 2H). ¹⁹F NMR (565 MHz, MeOD) δ −140.80-−140.88 (m), -162.95-−163.04 (m). ¹³C NMR (126 MHz, MeOD) δ 157.6, 152.6 (dd, J=244.6, 10.3 Hz), 150.2 (dd, J=7.8, 3.1 Hz), 142.5 (dd, J=245.6, 14.2 Hz), 139.3, 138.0, 129.6, 124.7 (dd, J=8.8, 5.2 Hz), 122.4, 111.2 (d, J=2.9 Hz), 109.7 (d, J=17.9 Hz), 70.7, 44.5, 42.4, 36.1, 32.7, 30.2, 29.9, 26.8. HRMS: (ESI) [M+H]⁺ calc. for C₂₃H₃₀F₂N₃O₂, 418.2301, observed, 418.2299.

### N-(4-(6-(2,6-difluorophenoxy)hexyl)phenyl)piperazine-1-carboxamide hydrochloride (3aa)

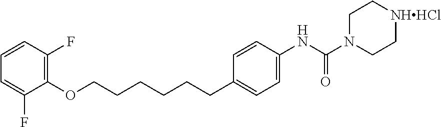

Synthesized according to procedure 4, purified via trituration with diethyl ether, white solid, 98 mg, 89% yield. ¹H NMR (500 MHz, MeOD) δ 7.28-7.24 (m, 2H), 7.13-7.09 (m, 2H), 7.06-6.98 (m, 1H), 6.98-6.92 (m, 2H), 4.09 (t, J=6.4 Hz, 2H), 3.79-3.76 (m, 4H), 3.29-3.26 (m, 4H), 2.58 (t, J=7.6 Hz, 2H), 1.75-1.69 (m, 2H), 1.62 (p, J=7.6 Hz, 2H), 1.55-1.48 (m, 2H), 1.38 (p, J=7.5 Hz, 2H). ¹⁹F NMR (376 MHz, MeOD) δ −130.45-−130.54 (m). ¹³C NMR (126 MHz, MeOD) δ 157.7 (dd, J=246.9, 5.6 Hz), 157.6, 139.3, 138.0, 137.0 (t, J=14.3 Hz), 129.6, 124.3 (t, J=9.4 Hz), 122.4, 113.2 (dd, J=17.4, 5.6 Hz), 75.8 (t, J=2.9 Hz), 44.5, 42.4, 36.1, 32.7, 31.0, 29.9, 26.6. HRMS: (ESI) [M+H]⁺ calc. for C₂₃H₃₀F₂N₃O₂, 418.2301, observed, 418.2301.

### N-(4-(6-(2,4,6-trifluorophenoxy)hexyl)phenyl)piperazine-1-carboxamide hydrochloride (3ab)

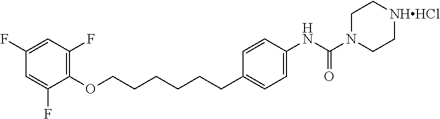

Synthesized according to procedure 4, white solid, 80 mg, 91% yield. ¹H NMR (600 MHz, MeOD) δ 7.28-7.23 (m, 2H), 7.14-7.09 (m, 2H), 6.92-6.83 (m, 2H), 4.04 (t, J=6.4 Hz, 2H), 3.83-3.72 (m, 4H), 3.30-3.25 (m, 4H), 2.58 (t, J=7.6 Hz, 2H), 1.71 (dt, J=8.4, 6.6 Hz, 2H), 1.65-1.58 (m, 2H), 1.51 (dtd, J=9.5, 7.4, 5.6 Hz, 2H), 1.42-1.34 (m, 2H). ¹⁹F NMR (565 MHz, MeOD) δ −116.92-−116.97 (m), −127.16-−127.22 (m). ¹³C NMR (126 MHz, MeOD) δ 159.6 (t, J=14.6 Hz), 158.6 (dd, J=15.2, 7.8 Hz), 157.6 (t, J=15.0, 14.5 Hz), 157.6, 156.7 (dd, J=15.2, 7.9 Hz), 134.0 (td, J=15.0, 14.5, 5.3 Hz), 129.6, 122.4, 101.9-101.5 (m), 76.1, 44.5, 42.4, 36.1, 32.7, 30.9, 29.8, 26.6. HRMS: (ESI) [M+H]⁺ calc. for C₂₈H₃₂N₅O₅, 436.2206, observed, 436.2206.

### N-(4-(6-(pyridin-3-yloxy)hexyl)phenyl)piperazine-1-carboxamide bis-trifluoroacetic acid (3ac)

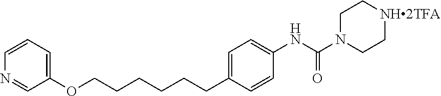

Synthesized according to procedure 4, yellow oil, 26 mg, 60% yield. ¹H NMR (600 MHz, MeOD) δ 8.54 (s, 1H), 8.43 (s, 1H), 8.18 (dd, J=8.8, 2.1 Hz, 1H), 8.00-7.95 (m, 1H), 7.26-7.22 (m, 2H), 7.12-7.08 (m, 2H), 4.20 (t, J=6.4 Hz, 2H), 3.79-3.76 (m, 4H), 3.27-3.24 (m, 4H), 2.58 (t, J=7.6 Hz, 2H), 1.88-1.82 (m, 2H), 1.63 (p, J=7.6 Hz, 2H), 1.53 (p, J=7.5 Hz, 2H), 1.41 (p, J=8.2 Hz, 2H). ¹³C NMR (151 MHz, MeOD) δ 161.8, 161.5, 161.3, 161.0, 159.4, 157.6, 139.2, 138.0, 134.9, 133.1, 130.5, 129.6, 129.3, 122.5, 71.4, 44.4, 42.3, 36.1, 32.6, 29.8, 29.7, 26.7. HRMS: (ESI) [M+H]⁺ calc. for C₂₂H₃₁N₄O₂, 383.2442, observed, 383.2450.

### N-(4-(3-(4-cyclopropylphenoxy)propyl)phenyl)piperazine-1-carboxamide hydrochloride (3ad)

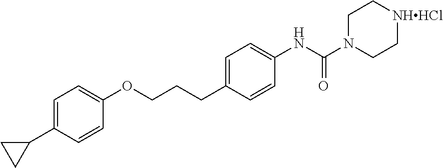

Synthesized according to procedure 4, white solid, 172 mg, 90% yield. ¹H NMR (500 MHz, MeOD) δ 7.27 (d, J=8.3 Hz, 2H), 7.14 (d, J=8.3 Hz, 2H), 6.97 (d, J=8.6 Hz, 2H), 6.77 (d, J=8.5 Hz, 2H), 3.90 (t, J=6.2 Hz, 2H), 3.80-3.76 (m, 4H), 3.29-3.25 (m, 4H), 2.75 (t, J=7.5 Hz, 2H), 2.02 (p, J=6.5 Hz, 2H), 1.83 (tt, J=8.4, 5.1 Hz, 1H), 0.90-0.84 (m, 2H), 0.57 (q, J=5.1, 4.6 Hz, 2H). ¹³C NMR (126 MHz, MeOD) δ 158.4, 157.6, 138.3, 138.2, 137.1, 129.8, 127.7, 122.5, 115.4, 67.9, 44.5, 42.4, 32.5, 32.3, 15.4, 8.9. HRMS: (ESI) [M+H]⁺ calc. for C₂₃H₃₀N₃O₂, 380.2333, observed, 380.2335.

### N-(4-(6-(2-ethylphenoxy)hexyl)phenyl)piperazine-1-carboxamide hydrochloride (3ae)

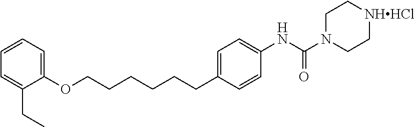

Synthesized according to procedure 4, white solid, 112 mg, 85% yield. ¹H NMR (600 MHz, MeOD) δ 7.26 (d, J=8.4 Hz, 2H), 7.13-7.07 (m, 4H), 6.86-6.79 (m, 2H), 3.95 (t, J=6.2 Hz, 2H), 3.79-3.75 (m, 4H), 3.29-3.25 (m, 4H), 2.64-2.56 (m, 4H), 1.82-1.75 (m, 2H), 1.63 (p, J=7.6 Hz, 2H), 1.54 (p, J=7.5 Hz, 2H), 1.41 (p, J=7.6 Hz, 2H), 1.16 (t, J=7.5 Hz, 3H). ¹³C NMR (151 MHz, MeOD) δ 158.1, 157.6, 139.3, 138.0, 133.6, 130.0, 129.6, 127.9, 122.4, 121.3, 112.1, 68.7, 44.5, 42.4, 36.1, 32.7, 30.5, 29.9, 27.2, 24.5, 15.0. HRMS: (ESI) [M+H]⁺ calc. for C₂₅H₃₆N₃O₂, 410.2802, observed, 410.2819.

### N-(4-(6-(2-trifluoromethylphenoxy)hexyl)phenyl)piperazine-1-carboxamide hydrochloride (3af)

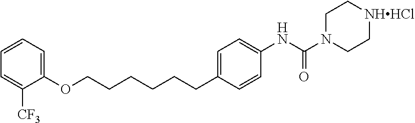

Synthesized according to procedure 4, white solid, 112 mg, 85% yield. ¹H NMR (600 MHz, MeOD) δ 7.56-7.51 (m, 2H), 7.27-7.24 (m, 2H), 7.14-7.09 (m, 3H), 7.02 (t, J=7.6 Hz, 1H), 4.06 (t, J=6.2 Hz, 2H), 3.80-3.75 (m, 4H), 3.30-3.25 (m, 4H), 2.58 (t, J=7.6 Hz, 2H), 1.81-1.76 (m, 2H), 1.63 (p, J=7.6 Hz, 2H), 1.54 (p, J=7.5 Hz, 2H), 1.39 (p, J=7.6 Hz, 2H). ¹⁹F NMR (565 MHz, MeOD) δ −63.68. ¹³C NMR (151 MHz, MeOD) δ 158.4, 157.6, 139.4, 137.9, 134.8, 129.6, 127.8 (q, J=5.3 Hz), 125.3 (q, J=271.5 Hz), 122.4, 120.9, 119.6 (q, J=30.5 Hz), 114.1, 69.5, 44.5, 42.4, 36.1, 32.7, 30.1, 29.7, 26.8. HRMS: (ESI) [M+H]⁺ calc. for C₂₄H₃₁F₃N₃O₂, 450.2363, observed, 450.2367.

### N-(4-(5-(2-ethylphenoxy)pentyl)phenyl)piperazine-1-carboxamide hydrochloride (3ag)

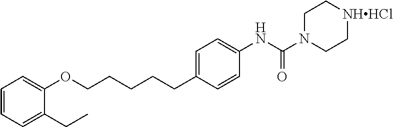

Synthesized according to procedure 4, white solid, 115 mg, 88% yield. ¹H NMR (600 MHz, MeOD) δ 7.28-7.25 (m, 2H), 7.13-7.11 (m, 2H), 7.10-7.07 (m, 2H), 6.85-6.80 (m, 2H), 3.94 (t, J=6.2 Hz, 2H), 3.79-3.76 (m, 4H), 3.28-3.25 (m, 4H), 2.59 (dt, J=15.1, 7.5 Hz, 5H), 1.84-1.78 (m, 2H), 1.68 (p, J=7.6 Hz, 2H), 1.56-1.50 (m, 2H), 1.14 (t, J=7.5 Hz, 3H). ¹³C NMR (151 MHz, MeOD) δ 158.1, 157.6, 139.2, 138.0, 133.7, 129.9, 129.7, 127.9, 122.4, 121.3, 112.1, 68.7, 44.5, 42.4, 36.2, 32.5, 30.4, 26.9, 24.5, 15.0. HRMS: (ESI) [M+H]⁺ calc. for C₂₄H₃₄N₃O₂, 396.2646, observed, 396.2653.

### N-(4-(4-(3-cyclopropyl-2-fluorophenoxy)butyl)phenyl)piperazine-1-carboxamide hydrochloride (3ah)

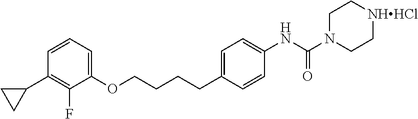

Synthesized according to procedure 4, white solid, 121 mg, 86% yield. ¹H NMR (600 MHz, MeOD) δ 7.30-7.26 (m, 2H), 7.14 (d, J=8.5 Hz, 2H), 6.92 (td, J=8.0, 1.4 Hz, 1H), 6.81 (td, J=8.1, 1.4 Hz, 1H), 6.48-6.43 (m, 1H), 4.02-3.98 (m, 2H), 3.80-3.76 (m, 4H), 3.28-3.25 (m, 4H), 2.65 (t, J=6.9 Hz, 2H), 2.09-2.04 (m, 1H), 1.81-1.76 (m, 4H), 0.97-0.93 (m, 2H), 0.69-0.66 (m, 2H). ¹⁹F NMR (565 MHz, MeOD) δ −144.90 (t, J=7.1 Hz). ¹³C NMR (151 MHz, MeOD) δ 157.6, 153.1 (d, J=242.7 Hz), 148.2 (d, J=11.2 Hz), 139.0, 138.1, 132.7 (d, J=11.9 Hz), 129.7, 124.8 (d, J=4.7 Hz), 122.5, 118.0 (d, J=3.0 Hz), 112.9, 70.1, 44.5, 42.4, 35.9, 29.8, 29.1, 9.3, 9.3, 8.3. HRMS: (ESI) [M+H]⁺ calc. for C₂₄H₃₁FN₃O₂, 412.2395, observed, 412.2403.

### N-(4-(4-(3-cyclopropyl-5-fluorophenoxy)butyl)phenyl)piperazine-1-carboxamide hydrochloride (3ai)

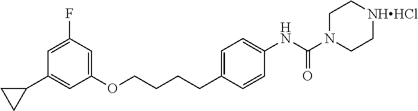

Synthesized according to procedure 4, white solid, 155 mg, 96% yield. ¹H NMR (600 MHz, MeOD) δ 7.30-7.27 (m, 2H), 7.15-7.12 (m, 2H), 6.44-6.43 (m, 1H), 6.40 (dt, J=10.8, 2.3 Hz, 1H), 6.33 (dt, J=10.0, 1.8 Hz, 1H), 3.93-3.91 (m, 2H), 3.81-3.76 (m, 4H), 3.28-3.24 (m, 4H), 2.63 (t, J=6.8 Hz, 2H), 1.88-1.83 (m, 1H), 1.78-1.72 (m, 4H), 0.97-0.92 (m, 2H), 0.67-0.63 (m, 2H). ¹⁹F NMR (565 MHz, MeOD) δ −114.92 (t, J=10.4 Hz). ¹³C NMR (151 MHz, MeOD) δ 165.1 (d, J=242.1 Hz), 161.8 (d, J=12.0 Hz), 157.6, 149.1 (d, J=9.9 Hz), 138.9, 138.1, 129.7, 122.4, 108.9 (d, J=2.4 Hz), 105.0 (d, J=22.4 Hz), 99.7 (d, J=25.4 Hz), 69.0, 44.5, 42.4, 35.9, 29.8, 29.1, 16.3, 16.3, 10.0. HRMS: (ESI) [M+H]⁺ calc. for C₂₄H₃₁FN₃O₂, 412.2395, observed, 412.2404.

### N-(4-(4-(3-cyclobutylphenoxy)propyl)-2-fluorophenyl)piperazine-1-carboxamide hydrochloride (3aj)

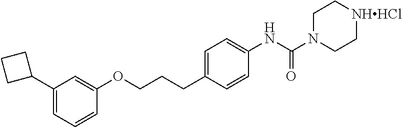

Synthesized according to procedure 4, white solid, 51 mg, 84% yield. ¹H NMR (500 MHz, MeOD) δ 7.30-7.26 (m, 2H), 7.17-7.13 (m, 3H), 6.79-6.76 (m, 1H), 6.73 (s, 1H), 6.68 (dd, J=8.0, 2.3 Hz, 1H), 3.93 (t, J=6.3 Hz, 2H), 3.80-3.75 (m, 4H), 3.51 (p, J=8.6 Hz, 1H), 3.29-3.25 (m, 4H), 2.79-2.72 (m, 2H), 2.36-2.28 (m, 2H), 2.17-2.07 (m, 2H), 2.07-1.97 (m, 3H), 1.89-1.81 (m, 1H). ¹³C NMR (126 MHz, MeOD) δ 160.5, 157.6, 149.0, 138.3, 138.2, 130.2, 129.8, 122.5, 119.6, 113.6, 112.7, 67.7, 44.5, 42.4, 41.8, 32.5, 32.3, 30.7, 19.0. HRMS: (ESI) [M+H]⁺ calc. for C₂₄H₃₂N₃O₂, 394.2489, observed, 394.2496.

### N-(4-(5-(3-chlorophenoxy)pentyl)phenyl)piperazine-1-carboxamide hydrochloride (3aq)

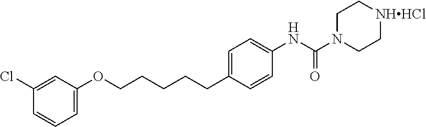

Synthesized according to procedure 4, white solid, 32 mg, 81% yield. ¹H NMR (600 MHz, MeOD) δ 7.27-7.25 (m, 2H), 7.23-7.20 (m, 1H), 7.13 (d, J=8.5 Hz, 2H), 6.91-6.89 (m, 2H), 6.83-6.81 (m, 1H), 3.95 (t, J=6.4 Hz, 2H), 3.79-3.75 (m, 4H), 3.29-3.26 (m, 4H), 2.61 (t, J=7.6 Hz, 2H), 1.82-1.75 (m, 2H), 1.67 (p, J=7.6 Hz, 2H), 1.53-1.46 (m, 2H). ¹³C NMR (151 MHz, MeOD) δ 161.5, 157.6, 139.2, 138.0, 135.8, 131.5, 129.7, 122.4, 121.5, 115.8, 114.1, 69.2, 44.5, 42.4, 36.1, 32.5, 30.1, 26.6. HRMS: (ESI) [M+H]⁺ calc. for C₂₂H₂₉ClN₃O₂, 402.1943, observed, 402.1951.

### N-(4-(4-(naphthalen-1-yloxy)butyl)phenyl)piperazine-1-carboxamide hydrochloride (3au)

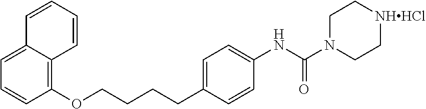

Synthesized according to procedure 4, white solid, 86 mg, 89% yield. ¹H NMR (500 MHz, DMSO) δ 9.31 (brs, 2H), 8.74 (s, 1H), 8.16-8.12 (m, 1H), 7.87-7.83 (m, 1H), 7.53-7.46 (m, 2H), 7.46-7.43 (m, 1H), 7.42-7.36 (m, 3H), 7.11 (d, J=8.5 Hz, 2H), 6.94 (d, J=7.2 Hz, 1H), 4.16 (t, J=6.0 Hz, 2H), 3.70-3.65 (m, 4H), 3.13-3.06 (m, 4H), 2.63 (t, J=7.3 Hz, 2H), 1.89-1.77 (m, 4H). ¹³C NMR (126 MHz, DMSO) δ 154.8, 154.1, 137.9, 135.6, 134.0, 128.2, 127.5, 126.4, 126.3, 125.2, 125.0, 121.5, 119.9, 119.7, 105.1, 67.6, 42.5, 40.9, 34.1, 28.3, 27.7. HRMS: (ESI) [M+H]⁺ calc. for C₂₅H₃₀N₃O₂, 404.2333, observed, 404.2339.

### N-(4-(6-(cyclopentyloxy)hexyl)phenyl)piperazine-1-carboxamide hydrochloride (3av)

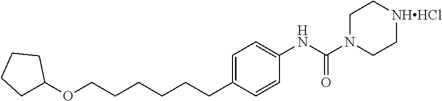

Synthesized according to procedure 4, white solid, 49 mg, 82% yield. ¹H NMR (500 MHz, MeOD) δ 7.28-7.24 (m, 2H), 7.12-7.09 (m, 2H), 3.92-3.86 (m, 1H), 3.80-3.75 (m, 4H), 3.37 (t, J=6.6 Hz, 2H), 3.29-3.25 (m, 4H), 2.57 (t, J=7.6 Hz, 2H), 1.76-1.65 (m, 4H), 1.64-1.56 (m, 4H), 1.55-1.48 (m, 4H), 1.40-1.31 (m, 4H). ¹³C NMR (126 MHz, MeOD) δ 157.6, 139.4, 138.0, 129.6, 122.4, 82.7, 69.8, 44.5, 42.4, 36.2, 33.2, 32.7, 30.9, 30.0, 27.2, 24.4. HRMS: (ESI) [M+H]⁺ calc. for C₂₂H₃₆N₃O₂, 374.2802, observed, 374.2805.

### N-(4-(5-(benzyloxy)pentyl)phenyl)piperazine-1-carboxamide hydrochloride (3aw)

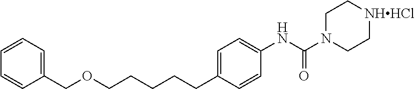

Synthesized according to procedure 4, white solid, 61 mg, 86% yield. ¹H NMR (500 MHz, MeOD) δ 7.35-7.29 (m, 4H), 7.28-7.24 (m, 3H), 7.12-7.08 (m, 2H), 4.47 (s, 2H), 3.79-3.75 (m, 4H), 3.47 (t, J=6.5 Hz, 2H), 3.29-3.25 (m, 4H), 2.57 (t, J=7.6 Hz, 2H), 1.66-1.57 (m, 4H), 1.44-1.36 (m, 2H). ¹³C NMR (126 MHz, MeOD) δ 157.6, 139.8, 139.3, 138.0, 129.7, 129.4, 128.9, 128.6, 122.4, 73.8, 71.3, 44.5, 42.4, 36.2, 32.5, 30.6, 26.8. HRMS: (ESI) [M+H]⁺ calc. for C₂₃H₃₂N₃O₂, 382.2489, observed, 382.2497.

### N-(4-(3-(3-phenylpropoxy)propyl)phenyl)piperazine-1-carboxamide hydrochloride (3ax)

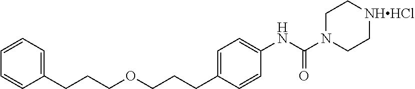

Synthesized according to procedure 4, white solid, 66 mg, 87% yield. ¹H NMR (500 MHz, MeOD) δ 7.30-7.23 (m, 4H), 7.20-7.17 (m, 2H), 7.17-7.11 (m, 3H), 3.80-3.76 (m, 4H), 3.41 (t, J=6.4 Hz, 4H), 3.29-3.25 (m, 4H), 2.70-2.63 (m, 4H), 1.90-1.81 (m, 4H). ¹³C NMR (126 MHz, MeOD) δ 157.6, 143.2, 138.6, 138.2, 129.7, 129.5, 129.3, 126.8, 122.4, 71.0, 70.9, 44.5, 42.4, 33.3, 32.7, 32.6, 32.6. HRMS: (ESI) [M+H]⁺ calc. for C₂₃H₃₂N₃O₂, 382.2489, observed, 382.2510.

### N-(4-(6-(cyclobutyloxy)hexyl)phenyl)piperazine-1-carboxamide hydrochloride (3ay)

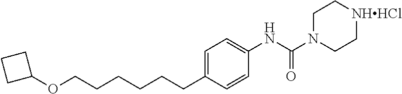

Synthesized according to procedure 4, white solid, 66 mg, 86% yield. ¹H NMR (500 MHz, MeOD) δ 7.26 (d, J=8.3 Hz, 2H), 7.11 (d, J=8.3 Hz, 2H), 3.91 (p, J=7.4 Hz, 1H), 3.80-3.75 (m, 4H), 3.34-3.32 (m, 2H), 3.29-3.26 (m, 4H), 2.57 (t, J=7.6 Hz, 2H), 2.22-2.16 (m, 2H), 1.92-1.83 (m, 2H), 1.68 (q, J=10.0 Hz, 1H), 1.60 (p, J=7.7 Hz, 2H), 1.55-1.47 (m, 3H), 1.40-1.30 (m, 4H). ¹³C NMR (126 MHz, MeOD) δ 157.6, 139.3, 138.0, 129.6, 122.4, 74.4, 68.8, 44.5, 42.4, 36.2, 32.7, 31.4, 30.8, 30.0, 27.1, 13.3. HRMS: (ESI) [M+H]⁺ calc. for C₂₁H₃₄N₃O₂, 360.2646, observed, 360.2648.

### 4-((4-(6-(cyclohexyloxy)hexyl)phenyl)carbamoyl)piperazin-1-ium chloride, (3az)

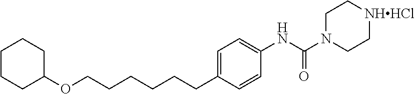

Synthesized according to procedure 4, off-white solid, 82 mg, 71%. ¹H NMR (500 MHz, d₄-MeOD) δ 7.26 (d, J=8.2 Hz, 2H), 7.11 (d, J=8.4 Hz, 2H), 3.82-3.75 (m, 4H), 3.45 (t, J=6.5 Hz, 2H), 3.29-3.21 (m, 5H), 2.57 (t, J=7.6 Hz, 2H), 1.93-1.85 (m, 2H), 1.78-1.68 (m, 2H), 1.64-1.50 (m, 5H), 1.43-1.19 (m, 9H). ¹³C NMR (126 MHz, d₄-MeOD) δ 157.6, 139.3, 138.0, 129.6, 122.4, 78.9, 69.0, 44.5, 42.4, 36.2, 33.4, 32.7, 31.1, 30.0, 27.1, 26.9, 25.2. HRMS (ESI⁺) found 432.2100 (C₂₃H₃₈N₃O₂), required 388.2964 [M+H]⁺.

### 4-((4-(4-((5,6,7,8-tetrahydronaphthalen-1-yl)oxy)butyl)phenyl)carbamoyl)piperazin-1-ium chloride, 3ba

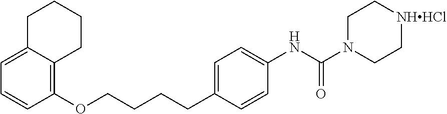

Synthesized according to procedure 4, off-white solid, 58 mg, 77%. ¹H NMR (600 MHz, d₄-MeOD) δ 7.28 (d, J=8.4 Hz, 2H), 7.14 (d, J=8.4 Hz, 2H), 6.97 (t, J=7.9 Hz, 1H), 6.62 (t, J=8.6 Hz, 2H), 3.96-3.92 (m, 2H), 3.80-3.76 (m, 4H), 3.29-3.26 (m, 5H), 2.71 (t, J=6.1 Hz, 2H), 2.67-2.64 (m, 1H), 2.61 (t, J=6.3 Hz, 2H), 1.84-1.70 (m, 8H). ¹³C NMR (126 MHz, d₄-MeOD) δ 158.0, 157.6, 139.2, 139.1, 138.1, 129.7, 126.8, 126.7, 122.5, 122.2, 108.9, 68.6, 44.5, 42.4, 35.9, 30.6, 30.0, 29.3, 24.2, 24.1, 24.1. HRMS (ESI⁺) found 408.2650 (C₂₅H₃₄N₃O₂), required 408.2651 [M+H]⁺.

### 4-((4-(4-((5,6,7,8-tetrahydronaphthalen-1-yl)oxy)butyl)phenyl)carbamoyl)piperazin-1-ium chloride, 3bb

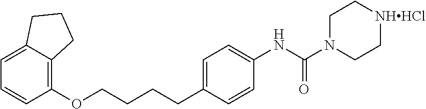

Synthesized according to procedure 4, off-white solid, 73 mg, 84%. ¹H NMR (500 MHz, d₄-MeOD) δ 7.27 (d, J=7.8 Hz, 2H), 7.14 (d, J=8.4 Hz, 2H), 7.04 (t, J=7.8 Hz, 1H), 6.78 (d, J=7.4 Hz, 1H), 6.65 (d, J=8.1 Hz, 1H), 3.98 (td, J=5.5, 2.8 Hz, 2H), 3.81-3.75 (m, 4H), 3.29-3.25 (m, 4H), 2.87 (t, J=7.3 Hz, 2H), 2.81 (t, J=7.4 Hz, 2H), 2.65 (q, J=5.2 Hz, 2H), 2.03 (p, J=7.4 Hz, 2H), 1.81-1.76 (m, 4H). ¹³C NMR (126 MHz, d₄-MeOD) δ 157.6, 156.8, 147.0, 139.1, 138.1, 132.7, 129.7, 128.6, 122.5, 117.7, 109.9, 68.7, 44.5, 42.4, 35.9, 34.1, 30.3, 30.0, 29.2, 26.0. HRMS (ESI⁺) found 394.2492 (C₂₄H₃₂N₃O₂), required 394.2489 [M+H]⁺.

### 4-((4-(7-methoxyheptyl)phenyl)carbamoyl)piperazin-1-ium chloride, 3bc

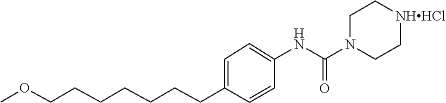

Synthesized according to procedure 4, off-white solid, 59 mg, 82%. ¹H NMR (500 MHz, d₄-MeOD) δ 7.25 (d, J=8.5 Hz, 2H), 7.10 (d, J=8.5 Hz, 2H), 3.80-3.75 (m, 4H), 3.37 (t, J=6.6 Hz, 2H), 3.31 (s, 3H), 3.29-3.25 (m, 4H), 2.56 (t, J=7.6 Hz, 2H), 1.63-1.50 (m, 4H), 1.38-1.28 (m, 6H). ¹³C NMR (126 MHz, d₄-MeOD) δ 157.6, 139.4, 137.9, 129.6, 122.4, 73.9, 58.7, 44.5, 42.4, 36.2, 32.7, 30.6, 30.4, 30.2, 27.1. HRMS (ESI⁺) found 334.2489 (C₁₉H₃₂N₃O₂), required 334.2489 [M+H]⁺.

### 4-((4-((4-(phenoxymethyl)cyclohexyl)methyl)phenyl)carbamoyl)piperazin-1-ium chloride, 3bd

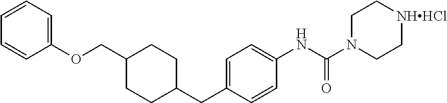

Synthesized according to procedure 4, off-white solid, 5 mg, 30%, d.r. 88:12. ¹H NMR (600 MHz, d₄-MeOD) δ 7.30-7.27 (m, 2H), 7.27-7.19 (m, 2H), 7.14-7.09 (m, 2H), 6.94-6.87 (m, 3H), 3.91 (d, J=6.9 Hz, 0.4H), 3.80 (t, J=5.3 Hz, 4H), 3.76 (d, J=6.3 Hz, 1.6H), 3.31-3.28 (m, 4H), 2.60 (d, J=7.6 Hz, 0.4H), 2.50 (d, J=7.1 Hz, 1.6H), 2.03-1.38 (m, 7H), 1.12-1.01 (m, 3H). ¹³C NMR (151 MHz, d₄-MeOD) 159.3, 156.2, 136.6, 136.4, 129.0, 120.8, 120.0, 114.0, 72.9, 43.1, 42.9, 41.0, 40.0, 37.8, 32.1, 29.4, 28.0. HRMS (ESI⁺) found 408.2644 (C₂₅H₃₄N₃O₂), required 408.2646 [M+H]⁺.

### 4-((4-(8-methoxyoctyl)phenyl)carbamoyl)piperazin-1-ium chloride, 3be

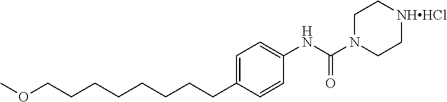

Synthesized according to procedure 4, off-white solid, 50 mg, 63%. ¹H NMR (600 MHz, d₄-MeOD) δ 7.28 (d, J=8.4 Hz, 2H), 7.12 (d, J=8.5 Hz, 2H), 3.82-3.78 (m, 4H), 3.39 (t, J=6.5 Hz, 2H), 3.31-3.27 (m, 4H), 2.58 (t, J=7.6 Hz, 2H), 1.66-1.52 (m, 4H), 1.39-1.27 (m, 8H). ¹³C NMR (151 MHz, d₄-MeOD) δ 157.6, 139.4, 137.9, 129.6, 122.4, 73.9, 58.7, 44.5, 42.4, 36.3, 32.8, 30.6, 30.6, 30.5, 30.2, 27.2. HRMS (ESI⁺) found 348.2654 (C₂₀H₃₄N₃O₂), required 348.2646 [M+H]⁺.

### 4-((4-((3-(phenoxymethyl)cyclohexyl)methyl)phenyl)carbamoyl)piperazin-1-ium chloride, 3bf

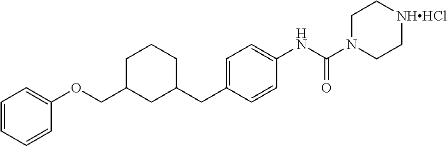

Synthesized according to procedure 4, off-white solid, 9 mg, 78%, dr. 70:30. ¹H NMR (600 MHz, d₄-MeOD) δ 7.28-7.25 (m, 2H), 7.25-7.20 (m, 2H), 7.11-7.06 (m, 2H), 6.91-6.83 (m, 3H), 3.78 (t, J=5.3 Hz, 4H), 3.75-3.70 (m, 2H), 3.27 (t, J=5.3 Hz, 4H), 2.53-2.45 (m, 2H), 1.89-1.84 (m, 2H), 1.83-1.70 (m, 2H), 1.66-1.38 (m, 3H), 1.35-1.26 (m, 1H), 1.01 (qd, J=12.8, 3.7 Hz, 1H), 0.94 (qd, J=12.8, 3.6 Hz, 1H). Observed signals of minor diastereomer 2.61-2.54 (m, 2H), 2.20-2.13 (m, 1H), 1.95-1.90 (m, 1H), 0.78 (q, J=12.1 Hz, 2H). ¹³C NMR (126 MHz, d₄-MeOD) δ 160.7, 157.6, 138.0, 137.6, 130.4, 130.4, 122.3, 121.4, 115.5, 74.4, 44.5, 42.4, 40.9, 39.2, 37.4, 34.2, 30.9, 26.8, 21.9. Observed signals of minor diastereomer 160.7, 157.6, 138.0, 130.4, 130.3, 122.3, 121.5, 115.6, 72.6, 44.5, 41.2, 36.2, 34.4, 33.8, 32.2, 29.8. HRMS (ESI⁺) found 408.2635 (C₂₅H₃₄N₃O₂), required 408.2651 [M+H]⁺.

### 4-((4-((3-(phenoxymethyl)cyclopentyl)methyl)phenyl)carbamoyl)piperazin-1-ium chloride, 3bg

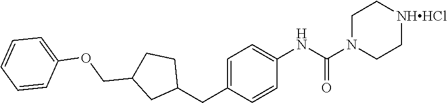

Synthesized according to procedure 4, off-white solid, 11 mg, 97%, dr. 60:40. ¹H NMR (600 MHz, d₆-DMSO) δ 9.23 (s, 2H), 8.72 (s, 1H), 7.36-7.31 (m, 2H), 7.29-7.21 (m, 2H), 7.08-7.03 (m, 2H), 6.92-6.86 (m, 3H), 3.82 (dd, J=6.9, 2.1 Hz, 1.2H, major diastereomer), 3.77 (d, J=7.1 Hz, 0.8H, minor diastereomer), 3.66 (t, J=4.6 Hz, 4H), 3.10 (t, J=4.9 Hz, 4H), 2.58-2.52 (m, 2H), 2.40 (hept, J=7.3 Hz, 0.4H, minor diastereomer), 2.29 (hept, J=8.0 Hz, 0.6H, major diastereomer), 2.20-2.05 (m, 1H), 1.91-1.82 (m, 1H), 1.76-1.63 (m, 2H), 1.52-1.40 (m, 1H), 1.32-1.17 (m, 1H), 0.98-0.91 (m, 1H). ¹³C NMR (151 MHz, d₆-DMSO) δ 158.8, 154.9, 137.9, 135.4, 129.5, 128.5, 120.4, 119.8, 114.4, 71.8, 42.6, 41.8, 40.9, 40.5, 38.5, 36.2, 32.0, 28.0. Observed signals for minor diastereomer: 128.6, 119.7, 71.7, 42.7, 40.9, 40.6, 37.3, 34.9, 31.1, 28.6. HRMS (ESI⁺) found 394.2488 (C₂₄H₃₂N₃O₂), required 394.2489 [M+H]⁺.

### 4-((4-(4-(phenoxymethyl)benzyl)phenyl)carbamoyl)piperazin-1-ium chloride, 3bh

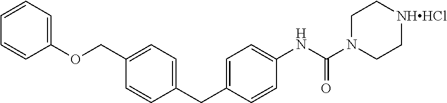

Synthesized according to procedure 4, off-white solid, 11 mg, 97%. ¹H NMR (600 MHz, d₄-MeOD) δ 7.34 (d, J=7.9 Hz, 2H), 7.28 (d, J=8.5 Hz, 2H), 7.25 (dd, J=8.7, 7.3 Hz, 2H), 7.20 (d, J=7.9 Hz, 2H), 7.13 (d, J=8.4 Hz, 2H), 6.98-6.95 (m, 2H), 6.93-6.89 (m, 1H), 5.02 (s, 2H), 3.93 (s, 2H), 3.77 (t, J=5.1 Hz, 4H), 3.27 (t, J=5.2 Hz, 4H). ¹³C NMR (151 MHz, d₄-MeOD) δ 160.2, 157.5, 142.6, 138.4, 138.0, 136.5, 130.4, 130.1, 129.9, 128.8, 122.4, 121.8, 115.9, 70.7, 44.5, 42.4, 41.9. HRMS (ESI⁺) found 402.2193 (C₂₅H₂₈N₃O₂), required 402.2176 [M+H]⁺.

### N-(4-(4-(3-(trifluoromethyl)phenoxy)butyl)phenyl)piperazine-1-carboxamide hydrochloride, 3bi

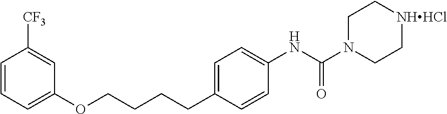

Synthesized according to procedure 4, white solid, 47 mg, 38% yield. ¹H NMR (500 MHz, MeOD) δ 7.44 (t, 1H), 7.30-7.25 (m, 2H), 7.20 (d, 1H), 7.17-7.11 (m, 4H), 4.06-4.00 (m, 2H), 3.83-3.75 (m, 4H), 3.31-3.25 (m, 4H), 2.66 (t, 2H), 1.85-1.74 (m, 4H). ¹³C NMR (126 MHz, MeOD) δ 159.4, 156.2, 137.5, 136.7, 131.5 (q, ²J_{CF3}=31.3 Hz), 130.0, 128.3, 125.2, 123.1, 121.0, 120.7, 117.8, 116.6 (q, ³J_{CF3}=4.2 Hz), 110.8 (q, ³J_{CF3}=4.2 Hz), 67.8, 43.1, 41.0, 34.5, 28.3, 27.7. ¹⁹F NMR (376 MHz, MeOD) δ −64.10. HRMS: (ESI) [M+H]⁺ calc. for C₂₂H₂₇F₃N₃O₂⁺, 422.2050, observed, 422.2034.

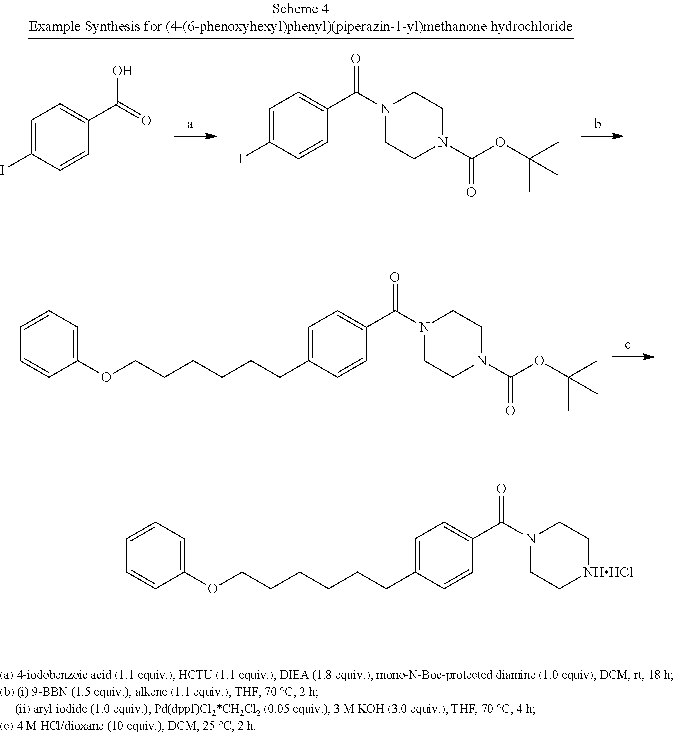

### tert-butyl 4-(4-iodobenzoyl)piperazine-1-carboxylate

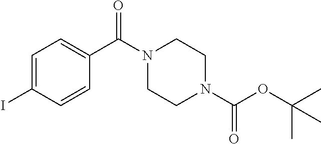 [SMILES: CC(C)(C)OC(=O)N1CCN(C(=O)c2ccc(I)cc2)CC1]

Synthesized according to procedure 6, white solid, 780 mg, 93%. ¹H NMR (400 MHz, MeOD) δ 7.84 (d, J=8.4 Hz, 2H), 7.21 (d, J=8.4 Hz, 2H), 3.79-3.35 (m, 8H), 1.47 (s, 9H). ¹³C NMR (101 MHz, MeOD) δ 171.7, 156.2, 139.0, 136.0, 130.0, 97.1, 81.7, 44.7, 43.3, 28.6. HRMS: (ESI) [M+H]+ calc. for C₁₆H₂₂₁N₂O₃, 417.0670, observed, 417.0672.

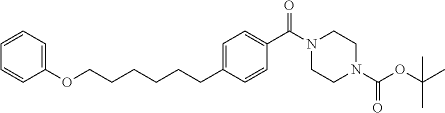 [SMILES: CC(C)(C)OC(=O)N1CCN(C(=O)c2ccc(CCCCCCOc3ccccc3)cc2)CC1]

### tert-butyl 4-(4-(6-phenoxyhexyl)benzoyl)piperazine-1-carboxylate (4a)

Synthesized according to procedure 3, white solid, 182 mg, 91%. ¹H NMR (500 MHz, CDCl₃) δ 7.32-7.30 (m, 2H), 7.29-7.25 (m, 2H), 7.23-7.19 (m, 2H), 6.95-6.91 (m, 1H), 6.91-6.86 (m, 2H), 3.95 (t, J=6.4 Hz, 2H), 3.77-3.35 (m, 8H), 2.64 (t, J=7.7 Hz, 2H), 1.82-1.73 (m, 2H), 1.70-1.60 (m, 2H), 1.55-1.43 (m, 11H), 1.45-1.36 (m, 2H). ¹³C NMR (126 MHz, CDCl₃) δ 171.0, 159.2, 154.7, 145.1, 132.8, 129.6, 128.7, 127.3, 120.6, 114.6, 80.5, 71.0, 67.8, 44.0, 35.8, 32.2, 31.3, 29.3, 29.1, 28.5, 26.3, 26.0, 22.1. HRMS: (ESI) [M+H]⁺ calc. for C₂₈H₃₉N₂O₄, 467.2904, observed, 467.2903.

### tert-butyl 4-(4-(3-(3-propylphenoxy)propyl)benzoyl)piperazine-1-carboxylate (4b)

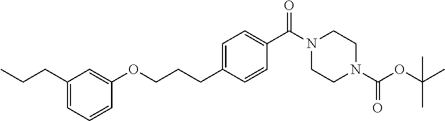 [SMILES: CCCc1cccc(OCCCc2ccc(C(=O)N3CCN(C(=O)OC(C)(C)C)CC3)cc2)c1]

Synthesized according to procedure 3, white solid, 214 mg, 96%. ¹H NMR (500 MHz, CDCl₃) δ 7.36-7.32 (m, 2H), 7.28-7.25 (m, 2H), 7.19 (t, J=7.8 Hz, 1H), 6.80-6.76 (m, 1H), 6.74-6.73 (m, 1H), 6.73-6.70 (m, 1H), 3.96 (t, J=6.2 Hz, 2H), 3.79-3.35 (m, 8H), 2.88-2.82 (m, 2H), 2.59-2.54 (m, 2H), 2.14-2.07 (m, 2H), 1.70-1.59 (m, 2H), 1.48 (s, 10H), 0.95 (t, J=7.3 Hz, 3H). ¹³C NMR (126 MHz, CDCl₃) δ 170.8, 159.0, 154.7, 144.5, 143.9, 133.2, 129.3, 128.9, 127.4, 121.1, 115.0, 111.6, 80.5, 66.6, 38.2, 32.2, 30.8, 28.5, 24.6, 14.0. HRMS: (ESI) [M+H]⁺ calc. for C₂₈H₃₉N₂O₄, 467.2904, observed, 467.2908.

### tert-butyl 4-(4-(4-(3-(tert-butyl)phenoxy)butyl)benzoyl)piperazine-1-carboxylate (4c)

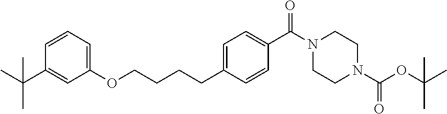 [SMILES: CC(C)(C)OC(=O)N1CCN(C(=O)c2ccc(CCCCOc3cccc(C(C)(C)C)c3)cc2)CC1]

Synthesized according to procedure 3, white solid, 74 mg, 62%. ¹H NMR (500 MHz, CDCl₃) δ 7.35-7.31 (m, 2H), 7.26-7.19 (m, 3H), 6.99-6.96 (m, 1H), 6.93 (t, J=2.2 Hz, 1H), 6.71-6.68 (m, 1H), 4.00-3.94 (m, 2H), 3.81-3.33 (m, 8H), 2.76-2.67 (m, 2H), 1.84-1.80 (m, 4H), 1.47 (s, 9H), 1.30 (s, 9H). ¹³C NMR (126 MHz, CDCl₃) δ 170.9, 159.0, 154.7, 153.1, 144.6, 133.0, 129.1, 128.8, 127.4, 118.0, 112.7, 110.6, 80.5, 67.6, 44.0, 35.6, 34.9, 31.5, 29.1, 28.5, 27.9. HRMS: (ESI) [M+H]⁺ calc. for C₃₀H₄₃N₂O₄, 495.3217, observed, 495.3226.

### (4-(6-phenoxyhexyl)phenyl)(piperazin-1-yl)methanone hydrochloride (5a)

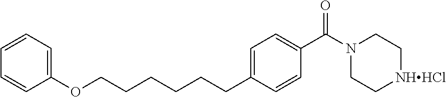

Synthesized according to procedure 4, white solid, 145 mg, 92% yield. ¹H NMR (500 MHz, MeOD) δ 7.43-7.37 (m, 2H), 7.36-7.30 (m, 2H), 7.28-7.20 (m, 2H), 6.90 (m, 1H), 6.89-6.86 (m, 2H), 3.95 (t, J=6.4 Hz, 2H), 3.85 (m, 4H), 3.28 (m, 4H), 2.70 (t, J=7.6 Hz, 2H), 1.81-1.72 (m, 2H), 1.72-1.64 (m, 2H), 1.52 (m, 2H), 1.46-1.38 (m, 2H). ¹³C NMR (126 MHz, MeOD) δ 172.9, 160.6, 147.3, 132.7, 130.4, 129.9, 128.5, 121.5, 115.5, 68.7, 44.4, 36.6, 32.4, 30.3, 29.9, 27.0. HRMS: (ESI) [M+H]⁺ calc. for C₂₃H₃₁N₃O₂, 367.2380, observed, 367.2382.

### piperazin-1-yl(4-(3-(3-propylphenoxy)propyl)phenyl)methanone hydrochloride (5b)

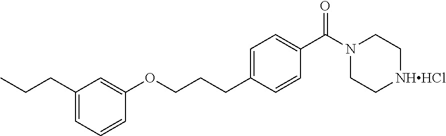

Synthesized according to procedure 4, white solid, 170 mg, 92% yield. ¹H NMR (500 MHz, MeOD) δ 7.44-7.41 (m, 2H), 7.37 (d, J=8.2 Hz, 2H), 7.14 (t, J=7.8 Hz, 1H), 6.76-6.73 (m, 1H), 6.72-6.71 (m, 1H), 6.71-6.68 (m, 1H), 3.95 (t, J=6.2 Hz, 2H), 3.92-3.76 (m, 4H), 3.31-3.24 (m, 4H), 2.89-2.85 (m, 2H), 2.56-2.52 (m, 2H), 2.14-2.04 (m, 2H), 1.69-1.57 (m, 2H), 0.93 (t, J=7.3 Hz, 3H). ¹³C NMR (126 MHz, MeOD) δ 172.8, 160.4, 146.3, 145.4, 133.0, 130.2, 130.1, 128.6, 121.9, 115.8, 112.6, 67.6, 44.4, 39.1, 33.0, 32.0, 25.7, 14.1. HRMS: (ESI) [M+H]⁺ calc. for C₂₃H₃₀N₂O₂, 367.2380, observed, 367.2382.

### (4-(4-(3-(tert-butyl)phenoxy)butyl)phenyl)(piperazin-1-yl)methanone hydrochloride (5c)

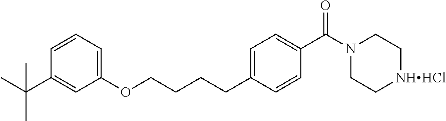

Synthesized according to procedure 4, white solid, 28 mg, 92% yield. ¹H NMR (500 MHz, MeOD) δ 7.44-7.41 (m, 2H), 7.36 (d, J=8.2 Hz, 2H), 7.17 (t, J=8.0 Hz, 1H), 6.97-6.94 (m, 1H), 6.90 (t, J=2.2 Hz, 1H), 6.70-6.68 (m, 1H), 3.98 (t, J=5.9 Hz, 2H), 3.90-3.74 (m, 4H), 3.30-3.27 (m, 4H), 2.76 (t, J=7.1 Hz, 2H), 1.86-1.79 (m, 4H), 1.29 (s, 9H). ¹³C NMR (126 MHz, MeOD) δ 172.9, 160.4, 153.9, 147.0, 132.9, 130.0, 130.0, 128.5, 118.7, 113.3, 111.9, 68.6, 44.4, 36.4, 35.5, 31.8, 30.0, 29.0. HRMS: (ESI) [M+H]⁺ calc. for C₂₅H₃₄N₂O₂, 395.2693, observed, 395.2682.

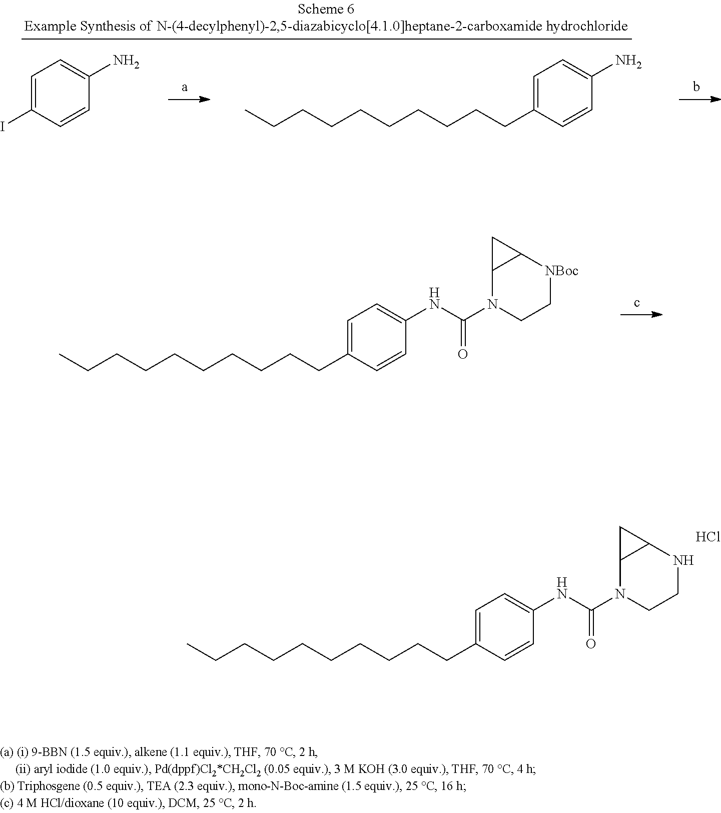 [SMILES: Nc1ccc(I)cc1 ; CCCCCCCCCCc1ccc(N)cc1 ; CCCCCCCCCCc1ccc(NC(=O)N2CCN(C(=O)OC(C)(C)C)C3CC32)cc1 ; CCCCCCCCCCc1ccc(NC(=O)N2CCNC3CC32)cc1 ; Cl]

### tert-butyl 5-((4-decylphenyl)carbamoyl)-2,5-diazabicyclo[4.1.0]heptane-2-carboxylate (6a)

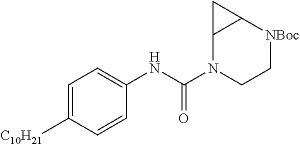

Synthesized by General Procedures herein. 112 mg, 52%, clear oil. ¹H NMR (500 MHz, CDCl₃) δ 7.36-7.32 (m, 2H), 7.15-7.10 (m, 2H), 6.94-6.87 (m, 1H), 4.18-4.05 (m, 1H), 3.51-3.42 (m, 1H), 3.37-3.12 (m, 2H), 3.10-2.86 (m, 2H), 2.60-2.53 (m, 2H), 1.62-1.55 (m, 2H), 1.51 (s, 10H), 1.36-1.22 (m, 16H), 0.89 (t, J=6.8 Hz, 3H), 0.69 (dt, J=12.5, 5.6 Hz, 1H). ¹³C NMR (126 MHz, CDCl₃) δ 156.0, 155.1, 138.0, 136.2, 128.8, 119.9, 80.6*, 80.3*, 42.9, 41.7, 39.8*, 39.5*, 35.3, 31.9, 31.6, 29.6, 29.6, 29.5, 29.3, 29.3, 28.4, 27.4, 22.7, 14.8, 14.6*, 14.1*. HRMS: (ESI) [M+H]⁺ calc. for C₂₇H₄₄N₃O₃+458.3377, observed, 458.3364.

### tert-butyl 7-((4-decylphenyl)carbamoyl)-4,7-diazaspiro[2.5]octane-4-carboxylate (6b)

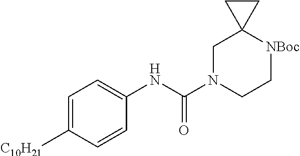

Synthesized by General Procedures herein. 48 mg, 24%, clear oil. ¹H NMR (500 MHz, CDCl₃) δ 7.23-7.19 (m, 2H), 7.09-7.06 (m, 2H), 6.42 (s, 1H), 3.59-3.56 (m, 2H), 3.47-3.43 (m, 2H), 3.30 (s, 2H), 2.53 (t, J=7.7 Hz, 2H), 1.61-1.53 (m, 2H), 1.47 (s, 9H), 1.30-1.23 (m, 14H), 1.02-0.97 (m, 2H), 0.89-0.82 (m, 5H). ¹³C NMR (126 MHz, CDCl₃) δ 155.6, 155.5, 138.0, 136.4, 128.8, 120.3, 80.4, 50.3, 45.7, 43.4, 37.9, 35.3, 31.9, 31.6, 29.6, 29.6, 29.5, 29.3, 29.3, 28.4, 22.7, 14.1, 13.9. ¹³C NMR (126 MHz, CDCl₃) δ 155.6, 155.5, 138.0, 136.4, 128.8, 120.3, 80.4, 50.3, 45.7, 43.4, 37.9, 35.3, 31.9, 31.6, 29.6, 29.6, 29.5, 29.3, 29.3, 28.4, 22.7, 14.1, 13.9.

### tert-butyl 6-((4-decylphenyl)carbamoyl)-2,6-diazaspiro[3.3]heptane-2-carboxylate (6c)

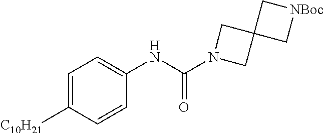

Synthesized by General Procedures herein. 59 mg, 27%, clear oil. ¹H NMR (500 MHz, CDCl₃) δ 7.29-7.23 (m, 2H), 7.09-7.05 (m, 2H), 6.38 (s, 1H), 4.04 (d, J=33.4 Hz, 8H), 2.53 (t, J=7.7 Hz, 2H), 1.60-1.52 (m, 2H), 1.44 (s, 9H), 1.33-1.22 (m, 14H), 0.88 (t, J=6.8 Hz, 3H). ¹³C NMR (126 MHz, CDCl₃) δ 156.6, 155.9, 138.1, 136.0, 128.8, 119.9, 79.9, 59.5, 35.3, 32.0, 31.9, 31.6, 29.6, 29.5, 29.3, 29.3, 28.4, 22.7, 14.1. HRMS: (ESI) [M+H]⁺ calc. for C₂₇H₄₄N₃O₃+458.3377, observed, 458.3373.

### tert-butyl 1-((4-decylphenyl)carbamoyl)-1,6-diazaspiro[3.3]heptane-6-carboxylate (6d)

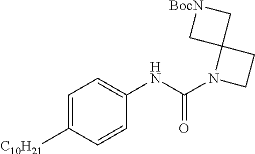

Synthesized by General Procedures herein. 53 mg, 22%, clear oil. ¹H NMR (500 MHz, CDCl₃) δ 7.33-7.28 (m, 2H), 7.11-7.07 (m, 2H), 4.56 (d, J=9.5 Hz, 2H), 3.99 (d, J=9.6 Hz, 2H), 3.90 (t, J=7.2 Hz, 2H), 2.56-2.52 (m, 2H), 2.42 (t, J=7.2 Hz, 2H), 1.60-1.53 (m, 2H), 1.32-1.23 (m, 15H), 0.88 (t, J=6.9 Hz, 3H). ¹³C NMR (126 MHz, CDCl₃) δ 156.4, 154.1, 137.9, 135.9, 128.8, 119.6, 80.0, 63.1, 60.4, 45.2, 35.3, 31.9, 31.6, 29.6, 29.6, 29.5, 29.3, 29.3, 28.3, 27.9, 22.7, 14.1. HRMS: (ESI) [M+H]⁺ calc. for C₂₇H₄₄N₃O₃+458.3377 observed, 458.3383.

### tert-butyl 3-((4-decylphenyl)carbamoyl)-3,6-diazabicyclo[3.1.1]heptane-6-carboxylate (6e)

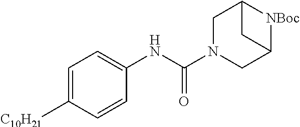

Synthesized by General Procedures herein. 125 mg, 34%, clear oil. ¹H NMR (500 MHz, CDCl₃) δ 7.33-7.29 (m, 2H), 7.10-7.06 (m, 2H), 6.63 (s, 1H), 4.20-3.92 (m, 4H), 3.43 (s, 2H), 2.60-2.49 (m, 3H), 1.61-1.54 (m, 2H), 1.43 (s, 8H), 1.38 (d, J=8.8 Hz, 1H), 1.34-1.22 (m, 15H), 0.91-0.86 (m, 3H). ¹³C NMR (126 MHz, CDCl₃) δ 156.5, 155.8, 137.8, 136.5, 128.7, 120.3, 80.7, 58.5*, 57.4*, 45.0*, 44.3*, 35.3, 31.9, 31.6, 29.6, 29.6, 29.4, 29.3, 28.5, 28.3, 22.7, 14.2. HRMS: (ESI) [M+H]⁺ calc. for C₂₇H₄₄N₃O₃+458.3377, observed, 458.3367.

### N-(4-decylphenyl)-2,5-diazabicyclo[4.1.0]heptane-2-carboxamide hydrochloride (7a)

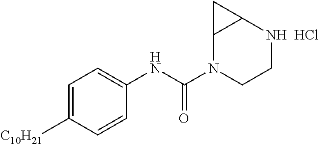

Synthesized by General Procedures herein, white solid, 20 mg, 77% yield. ¹H NMR (600 MHz, MeOD) δ 7.38-7.32 (m, 2H), 7.16-7.12 (m, 2H), 4.19-4.13 (m, 1H), 3.42-3.38 (m, 1H), 3.28-3.24 (m, 2H), 3.13-3.05 (m, 2H), 2.59 (t, J=7.6 Hz, 2H), 1.61 (h, J=6.9 Hz, 2H), 1.47 (q, J=7.5 Hz, 1H), 1.36-1.25 (m, 16H), 0.95-0.89 (m, 3H). ¹³C NMR (151 MHz, MeOD) δ 156.5, 138.4, 136.2, 128.2, 121.8, 40.8, 36.0, 34.9, 31.7, 31.4, 29.4, 29.3, 29.2, 29.1, 28.9, 28.6, 25.9, 22.3, 13.1, 10.5. HRMS: (ESI) [M+H]⁺ calc. for C₂₂H₃₆N₃O⁺ 358.2853, observed, 358.2855.

### N-(4-decylphenyl)-4,7-diazaspiro[2.5]octane-7-carboxamide hydrochloride (7b)

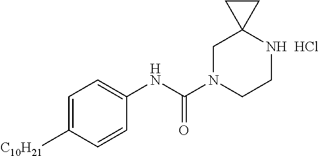

Synthesized by General Procedures herein, white solid, 33 mg, 83% yield. ¹H NMR (500 MHz, MeOD) δ 7.28-7.23 (m, 2H), 7.12-7.08 (m, 2H), 3.87 (t, J=5.2 Hz, 2H), 3.70 (s, 2H), 3.39 (t, J=5.3 Hz, 2H), 2.56 (t, J=7.6 Hz, 2H), 1.63-1.54 (m, 2H), 1.34-1.26 (m, 14H), 1.15-1.10 (m, 2H), 1.10-1.05 (m, 2H), 0.92-0.87 (m, 3H). ¹³C NMR (126 MHz, MeOD) δ 157.7, 139.5, 137.9, 129.6, 122.5, 68.1, 44.8, 42.3, 39.2, 36.3, 33.1, 32.8, 30.7, 30.7, 30.6, 30.5, 30.3, 23.7, 14.4, 10.2. HRMS: (ESI) [M+H]⁺ calc. for C₂₃H₃₈N₃O⁺ 372.3009, observed, 372.3013.

### N-(4-decylphenyl)-2,6-diazaspiro[3.3]heptane-2-carboxamide 2,2,2-trifluoroacetate (7c)

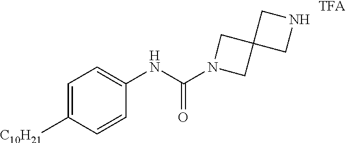

Synthesized by General Procedures herein. 25 mg, 97%, white solid. ¹H NMR (500 MHz, MeOD) δ 7.27 (d, J=8.1 Hz, 2H), 7.05 (d, J=8.2 Hz, 2H), 4.23 (d, J=17.3 Hz, 8H), 2.53 (t, J=7.6 Hz, 2H), 1.60-1.52 (m, 2H), 1.28 (d, J=13.6 Hz, 15H), 0.88 (t, J=6.8 Hz, 3H). ¹³C NMR (126 MHz, MeOD) δ 159.3, 139.0, 137.8, 129.6, 121.6, 60.1, 56.6, 36.6, 36.3, 33.1, 32.8, 30.7, 30.7, 30.6, 30.5, 30.3, 23.7, 14.4. HRMS: (ESI) [M+H]⁺ calc. for C₂₂H₃₆N₃O⁺ 358.2853, observed, 358.2864.

### N-(4-decylphenyl)-1,6-diazaspiro[3.3]heptane-1-carboxamide 2,2,2-trifluoroacetate (7d)

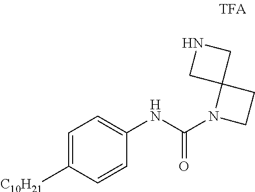

Synthesized by General Procedures herein. 25 mg, 97%, yellow oil. ¹H NMR (500 MHz, MeOD) δ 7.35-7.30 (m, 2H), 7.11-7.05 (m, 2H), 4.72-4.66 (m, 2H), 4.26-4.19 (m, 2H), 4.00-3.92 (m, 2H), 3.31-3.26 (m, 1H), 2.57-2.47 (m, 4H), 1.63-1.50 (m, 2H), 1.32-1.23 (m, 14H), 0.92-0.85 (m, 3H). ¹³C NMR (126 MHz, MeOD) δ 154.4, 136.4, 134.5, 126.7, 119.1, 63.5, 55.2, 44.5, 33.3, 30.1, 29.9, 27.8, 27.8, 27.7, 27.5, 27.3, 25.1, 20.8, 11.5. HRMS: (ESI) [M+H]⁺ calc. for C₂₂H₃₆N₃O⁺ 358.2853, observed, 358.2861.

### N-(4-decylphenyl)-3,6-diazabicyclo[3.1.1]heptane-3-carboxamide hydrochloride (7e)

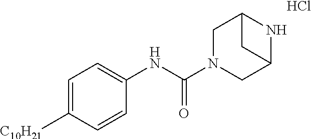

Synthesized by General Procedure herein. 55 mg, 50%, brown solid. ¹H NMR (500 MHz, MeOD) δ 7.35-7.31 (m, 2H), 7.14-7.09 (m, 2H), 4.48 (d, J=6.5 Hz, 2H), 4.05-4.00 (m, 3H), 3.07-3.00 (m, 1H), 2.59-2.55 (m, 2H), 1.93 (d, J=10.6 Hz, 1H), 1.64-1.54 (m, 2H), 1.38-1.24 (m, 16H), 0.90 (t, J=6.9 Hz, 3H). ¹³C NMR (126 MHz, MeOD) δ 156.8, 138.3, 136.4, 128.2, 121.7, 58.3, 45.8, 34.9, 31.7, 31.4, 29.4, 29.3, 29.2, 29.1, 28.9, 28.3, 22.4, 13.1. HRMS: (ESI) [M+H]⁺ calc. for C₂₂H₃₆N₃O⁺ 358.2853, observed, 358.2850.

### tert-butyl 3-(4-iodobenzoyl)-3,8-diazabicyclo[3.2.1]octane-8-carboxylate

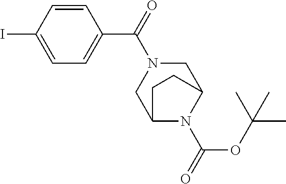 [SMILES: CC(C)(C)OC(=O)N1C2CCC1CN(C(=O)c1ccc(I)cc1)C2]

Purified via column chromatography (35% ethyl acetate/hexanes). White solid (90%, 482 mg). ¹H NMR (400 MHz, cdcl₃) δ 7.76 (d, J=8.4 Hz, 2H), 7.21 (d, J=8.4 Hz, 2H), 4.90-4.67 (m, 1H), 4.14-3.68 (m, 3H), 3.30-2.85 (m, 2H), 2.02-1.71 (m, 4H), 1.45 (s, 9H). ¹³C NMR (101 MHz, cdcl₃) δ 167.6, 155.8, 137.8, 135.2, 129.1, 96.8, 80.4, 56.6, 51.4*, 50.0, 48.9*, 28.5, 27.7, 26.2*. Material isolated as an approximately 1:1 ratio of rotamers. HRMS: (ESI) [M+H]⁺ calc. for C₁₈H₂₄IN₂O₃, 443.0826, observed, 443.0832.

### tert-butyl 3-(4-decylbenzoyl)-3,8-diazabicyclo[3.2.1]octane-8-carboxylate (8a)

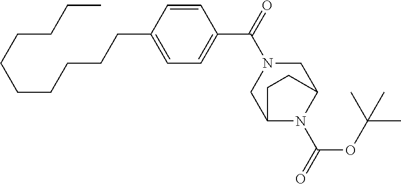 [SMILES: CCCCCCCCCCc1ccc(C(=O)N2CC3CCC(C2)N3C(=O)OC(C)(C)C)cc1]

Purified via column chromatography (30-40% ethyl acetate/hexanes). Clear oil (74%, 367 mg). ¹H NMR (400 MHz, cdcl₃) δ 7.38 (d, J=8.1 Hz, 2H), 7.19 (d, J=8.0 Hz, 2H), 4.91-4.65 (m, 1H), 4.20-3.66 (m, 3H), 3.29-2.93 (m, 2H), 2.60 (t, J=7.7 Hz, 2H), 1.99-1.68 (m, 4H), 1.59 (p, J=7.3 Hz, 2H), 1.44 (s, 9H), 1.34-1.18 (m, 14H), 0.86 (t, J=6.8 Hz, 3H). ¹³C NMR (101 MHz, cdcl₃) δ 168.7, 155.9, 145.6, 133.0, 128.6, 127.5, 80.2, 56.5, 51.2*, 50.1, 49.0*, 35.9, 32.0, 31.3, 29.7, 29.6, 29.5, 29.4, 29.3, 28.4, 27.7, 26.1*, 22.7, 14.2. Material isolated as an approximately 1:1 ratio of rotamers. HRMS: (ESI) [2M+H]⁺ calc. for C₅₆H₈₉N₄O₆, 913.6777, observed, 913.6774.

### (2,5-diazabicyclo[2.2.1]heptan-2-yl)(4-decylphenyl)methanone hydrochloride (9a)

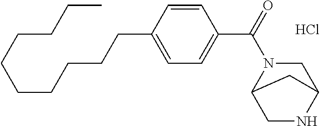 [SMILES: CCCCCCCCCCc1ccc(C(=O)N2CC3CC2CN3)cc1 ; Cl]

Purified via trituration with ethyl acetate and diethyl ether. White solid (65%, 56 mg). ¹H NMR (400 MHz, cd₃od) δ 7.57-7.42 (m, 2H), 7.36-7.23 (m, 2H), 5.01-4.42 (m, 2H), 3.86-3.79 (m, 1H), 3.74-3.52 (m, 2H), 3.46-3.38 (m, 1H), 2.67 (t, J=7.8 Hz, 2H), 2.25 (dd, J=45.0, 11.8 Hz, 1H), 2.05 (dd, J=34.3, 11.4 Hz, 1H)*, 1.63 (p, J=6.7 Hz, 2H), 1.40-1.21 (m, 14H), 0.90 (t, J=6.9 Hz, 3H). ¹³C NMR (101 MHz, cd₃od) δ 172.6, 171.3*, 147.9*, 147.6, 133.5, 133.3*, 129.9, 129.7*, 128.8*, 128.5, 59.9, 59.7*, 58.9, 56.3*, 54.1, 53.8*, 52.9, 50.4*, 37.4, 36.8, 35.8*, 33.0, 32.5, 30.7, 30.7, 30.6, 30.4, 30.3, 23.7, 14.5. Material isolated as an approximately 1:1 ratio of rotamers. HRMS: (ESI) [M+H]⁺ calc. for C₂₂H₃₅N₂O, 343.2744, observed, 343.2738.

### (3,8-diazabicyclo[3.2.1]octan-3-yl)(4-decylphenyl)methanone hydrochloride (9b)

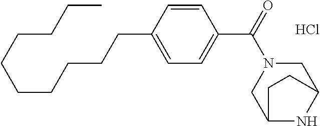 [SMILES: CCCCCCCCCCc1ccc(C(=O)N2CC3CCC(C2)N3)cc1 ; Cl]

Purified via trituration with ethyl acetate and diethyl ether. White solid (93%, 80 mg). ¹H NMR (400 MHz, cd₃od) δ 7.50 (d, J=8.1 Hz, 2H), 7.33 (d, J=8.1 Hz, 2H), 4.80-4.19 (m, 2H), 3.43-3.26 (m, 4H), 2.67 (t, J=7.7 Hz, 2H), 2.27-2.18 (m, 2H), 2.11-1.99 (m, 2H), 1.64 (p, J=7.3 Hz, 2H), 1.42-1.22 (m, 14H), 0.90 (t, J=6.9 Hz, 3H). ¹³C NMR (126 MHz, MeOD) δ 171.0, 147.9, 132.9, 129.9, 128.7, 56.5, 51.4*, 49.9, 36.8, 33.1, 32.5, 30.7, 30.7, 30.6, 30.5, 30.3, 27.6, 26.3*, 23.7, 14.4. Material isolated as an approximately 1:1 ratio of rotamers. HRMS: (ESI) [M+H]⁺ calc. for C₂₃H₃₇N₂O, 357.2900, observed, 357.2911.

### tert-butyl 3-((4-iodophenyl)carbamoyl)-3,8-diazabicyclo[3.2.1]octane-8-carboxylate

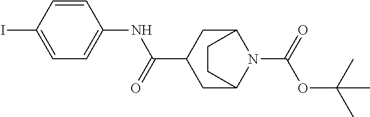 [SMILES: CC(C)(C)OC(=O)N1C2CCC1CC(C(=O)Nc1ccc(I)cc1)C2]

Purified via column chromatography (30-40% ethyl acetate/hexanes). White solid (99%, 745 mg). ¹H NMR (400 MHz, cdcl₃) δ 7.53 (d, J=8.5 Hz, 2H), 7.16 (d, J=8.5 Hz, 2H), 6.80 (s, 1H), 4.31-4.23 (m, 2H), 3.78 (dd, J=55.8, 13.2 Hz, 2H), 3.07 (dd, J=45.4, 13.2 Hz, 2H), 2.00-1.66 (m, 4H), 1.45 (s, 9H). ¹³C NMR (101 MHz, cdcl₃) δ 156.0, 153.3, 138.9, 137.8, 121.8, 86.1, 80.3, 54.2*, 53.5, 49.8, 48.6*, 28.5, 27.3*, 27.0. HRMS: (ESI) [M+H]⁺ calc. for C₁₈H₂₅IN₃O₃, 458.0935, observed, 458.0945.

### tert-butyl 3-((4-decylphenyl)carbamoyl)-3,8-diazabicyclo[3.2.1]octane-8-carboxylate (6f)

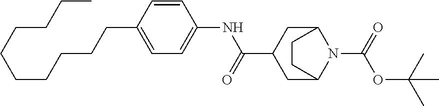 [SMILES: CCCCCCCCCCc1ccc(NC(=O)C2CC3CCC(C2)N3C(=O)OC(C)(C)C)cc1]

Purified via column chromatography (30-40% ethyl acetate/hexanes). White solid (60%, 483 mg). ¹H NMR (400 MHz, cdcl₃) δ 7.28 (d, J=8.4 Hz, 2H), 7.22 (s, 1H), 7.04 (d, J=8.4 Hz, 2H), 4.33-4.25 (m, 2H), 3.72 (dd, J=57.3, 13.2 Hz, 2H), 3.06 (dd, J=43.5, 12.7 Hz, 2H), 2.52 (t, J=7.7 Hz, 2H), 1.89-1.80 (m, 2H), 1.71-1.51 (m, 4H), 1.45 (s, 9H), 1.34-1.23 (m, 14H), 0.88 (t, J=6.8 Hz, 3H). ¹³C NMR (101 MHz, cdcl₃) δ 155.9, 154.1, 137.7, 136.6, 128.6, 120.3, 80.0, 53.8*, 53.2, 49.7*, 48.5, 35.3, 31.9, 31.6, 29.6, 29.6, 29.5, 29.3, 29.3, 28.4, 27.2, 26.9*, 22.7, 14.1. HRMS: (ESI) [M+H]⁺ calc. for C₂₈H₄₆N₃O₃, 472.3534, observed, 472.3530.

### N-(4-decylphenyl)-3,8-diazabicyclo[3.2.1]octane-3-carboxamide hydrochloride (7f)

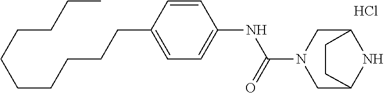 [SMILES: CCCCCCCCCCc1ccc(NC(=O)N2CC3CCC(C2)N3)cc1 ; Cl]

Purified via trituration with ethyl acetate and diethyl ether. White solid (87%, 75 mg). ¹H NMR (400 MHz, cd₃od) δ 7.34 (d, J=8.5 Hz, 2H), 7.12 (d, J=8.5 Hz, 2H), 4.67-4.59 (m, 2H), 3.38-3.22 (m, 4H), 2.57 (t, J=7.6 Hz, 2H), 2.26-2.20 (m, 2H), 2.12-2.03 (m, 2H), 1.60 (p, J=7.1 Hz, 2H), 1.39-1.22 (m, 14H), 0.91 (t, J=6.7 Hz, 3H). ¹³C NMR (101 MHz, cd₃od) δ 156.18, 139.37, 137.80, 129.60, 122.12, 53.37, 36.26, 33.05, 32.79, 30.73, 30.71, 30.60, 30.45, 30.26, 27.12, 23.72, 14.45. HRMS: (ESI) [M+H]⁺ calc. for C₂₃H₃₈N₃O, 372.3009, observed, 372.3010.

### N-(hex-5-en-1-yl)aniline

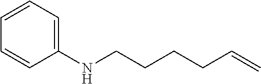

Synthesized according to procedure 7, yellow oil, 180 mg, 64% yield. ¹H NMR (500 MHz, CDCl₃) δ 7.22-7.16 (m, 2H), 6.71 (t, J=7.3 Hz, 1H), 6.64-6.60 (m, 2H), 5.84 (ddt, J=16.9, 10.2, 6.7 Hz, 1H), 5.05 (dq, J=17.1, 1.7 Hz, 1H), 4.99 (ddt, J=10.1, 2.0, 1.1 Hz, 1H), 3.13 (t, J=7.1 Hz, 2H), 2.13 (q, J=7.2 Hz, 2H), 1.69-1.61 (m, 2H), 1.56-1.49 (m, 2H). ¹³C NMR (126 MHz, CDCl₃) δ 148.5, 138.7, 129.4, 117.3, 114.9, 112.8, 43.9, 33.6, 29.1, 26.5. HRMS: (ESI) [M+H]⁺ calc. for C₁₂H₁₈N, 176.1434, observed, 176.1438.

### tert-butyl 4-((4-(6-(phenylamino)hexyl)phenyl)carbamoyl)piperazine-1-carboxylate (2ak)

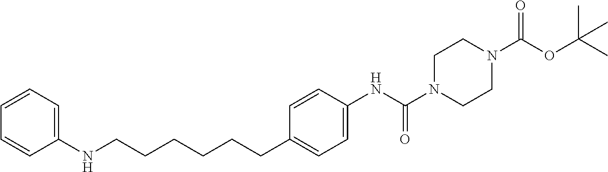 [SMILES: CC(C)(C)OC(=O)N1CCN(C(=O)Nc2ccc(CCCCCCNc3ccccc3)cc2)CC1]

Synthesized according to procedure 3, yellow solid, 140 mg, 63% yield. HRMS: (ESI) [M+H]⁺ calc. for C₁₂H₁₈N, 176.1434, observed, 176.1438. ¹H NMR (500 MHz, CDCl₃) δ 7.25-7.22 (m, 2H), 7.19-7.14 (m, 2H), 7.08 (d, J=8.4 Hz, 2H), 6.67 (tt, J=7.4, 1.0 Hz, 1H), 6.60-6.57 (m, 2H), 6.44-6.40 (m, 1H), 3.50-3.43 (m, 8H), 3.08 (t, J=7.1 Hz, 2H), 2.59-2.52 (m, 2H), 1.60 (p, J=7.3 Hz, 4H), 1.48 (s, 9H), 1.44-1.33 (m, 6H). ¹³C NMR (126 MHz, CDCl₃) δ 155.3, 154.7, 148.6, 137.9, 136.5, 129.3, 128.9, 120.5, 117.2, 112.8, 80.4, 44.0, 35.3, 31.5, 29.6, 29.1, 28.5, 27.1. HRMS: (ESI) [M+H]⁺ calc. for C₂₈H₄₁N₄O₃, 481.3173, observed, 481.3166.

### N-(4-(6-(phenylamino)hexyl)phenyl)piperazine-1-carboxamide hydrogen chloride (3ak)

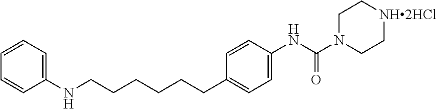

Synthesized according to procedure 4, yellow solid, 55 mg, 91% yield. ¹H NMR (500 MHz, MeOD) δ 7.60-7.55 (m, 2H), 7.54-7.51 (m, 1H), 7.49-7.45 (m, 2H), 7.29-7.24 (m, 2H), 7.13-7.08 (m, 2H), 3.80-3.77 (m, 4H), 3.66 (s, 1H), 3.39-3.33 (m, 2H), 3.29-3.26 (m, 4H), 2.58 (t, J=7.6 Hz, 2H), 1.78-1.67 (m, 2H), 1.67-1.57 (m, 2H), 1.49-1.42 (m, 2H), 1.42-1.35 (m, 2H). ¹³C NMR (126 MHz, MeOD) δ 157.6, 139.1, 138.1, 137.2, 131.5, 130.6, 129.6, 123.4, 122.4, 68.1, 53.1, 44.5, 42.4, 36.0, 32.4, 29.6, 27.2, 27.1. HRMS: (ESI) [M+H]⁺ calc. for C₂₃H₃₃N₄O, 381.2649, observed, 381.2655.

### N-phenylhex-5-enamide

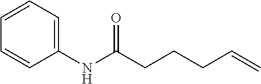

Synthesized according to procedure 8, yellow solid, 320 mg, 97% yield. ¹H NMR (400 MHz, CDCl₃) δ 7.51 (d, J=7.9 Hz, 2H), 7.31 (t, J=7.9 Hz, 2H), 7.24 (br, 1H), 7.10 (t, J=7.4 Hz, 1H), 5.81 (ddt, J=16.9, 10.1, 6.7 Hz, 1H), 5.09-4.98 (m, 2H), 2.36 (t, J=7.5 Hz, 2H), 2.15 (q, J=7.1 Hz, 2H), 1.84 (p, J=7.4 Hz, 2H). ¹³C NMR (101 MHz, CDCl₃) δ 171.3, 138.0, 129.5, 129.1, 124.4, 119.9, 115.6, 37.0, 33.2, 24.7. HRMS: (ESI) [M+H]⁺ calc. for C₁₂H₁₆NO, 190.1226, observed, 190.1228.

### tert-butyl 4-((4-(6-oxo-6-(phenylamino)hexyl)phenyl)carbamoyl)piperazine-1-carboxylate (2al)

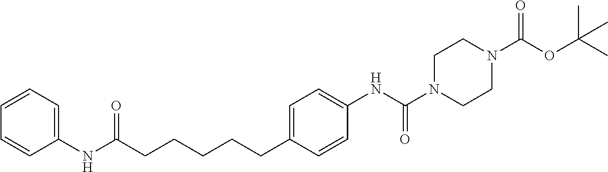 [SMILES: CC(C)(C)OC(=O)N1CCN(C(=O)Nc2ccc(CCCCCC(=O)Nc3ccccc3)cc2)CC1]

Synthesized according to procedure 3, white solid, 130 mg, 38% yield. ¹H NMR (600 MHz, CDCl₃) δ 7.67 (br, 1H), 7.52 (d, J=7.9 Hz, 2H), 7.29 (t, J=7.9 Hz, 2H), 7.18 (d, J=8.3 Hz, 2H), 7.10-7.04 (m, 3H), 6.56 (br, 1H), 3.46 (s, 8H), 2.55 (t, J=7.4 Hz, 2H), 2.25 (t, J=7.6 Hz, 2H), 1.67 (p, J=7.7 Hz, 2H), 1.58 (p, J=7.6 Hz, 2H), 1.48 (s, 9H), 1.32 (p, J=7.8 Hz, 2H). ¹³C NMR (151 MHz, CDCl₃) δ 171.8, 155.5, 154.8, 138.2, 138.0, 136.4, 129.0, 129.0, 124.2, 121.0, 119.9, 80.5, 44.0, 37.7, 35.1, 31.2, 28.6, 28.5, 25.6. HRMS: (ESI) [M+H]⁺ calc. for C₂₈H₃₉N₄O₄, 495.2966, observed, 495.2961.

### N-(4-(6-oxo-6-(phenylamino)hexyl)phenyl)piperazine-1-carboxamide hydrogen chloride (3al)

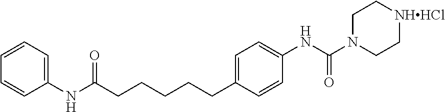

Synthesized according to procedure 4, white solid, 44 mg, 84% yield. ¹H NMR (600 MHz, MeOD) δ 9.77 (s, 1H), 7.54-7.51 (m, 2H), 7.31-7.27 (m, 2H), 7.26-7.23 (m, 2H), 7.11 (d, J=8.4 Hz, 2H), 7.08 (t, J=7.4 Hz, 1H), 3.80-3.75 (m, 4H), 3.28-3.25 (m, 4H), 2.59 (t, J=7.6 Hz, 2H), 2.35 (t, J=7.5 Hz, 2H), 1.72 (p, J=7.6 Hz, 2H), 1.65 (p, J=7.6 Hz, 2H), 1.41 (p, J=7.7 Hz, 2H). ¹³C NMR (151 MHz, MeOD) δ 174.7, 174.7, 157.6, 140.0, 139.9, 139.2, 138.0, 129.8, 129.7, 125.1, 122.4, 121.4, 121.3, 44.5, 42.4, 38.0, 37.9, 36.1, 32.5, 29.8, 26.8. HRMS: (ESI) [M+H]⁺ calc. for C₂₃H₃₁N₄O₂, 395.2442, observed, 395.2442.

### tert-butyl 4-((6-iodopyridin-3-yl)carbamoyl)piperazine-1-carboxylate

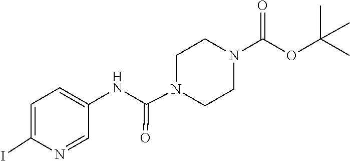 [SMILES: CC(C)(C)OC(=O)N1CCN(C(=O)Nc2ccc(I)nc2)CC1]

Synthesized according to procedure 11, yellow solid, 244 mg, 41% yield. ¹H NMR (600 MHz, CDCl₃) δ 8.20 (d, J=2.6 Hz, 1H), 7.65 (dd, J=8.6, 2.8 Hz, 1H), 7.58 (d, J=8.6 Hz, 1H), 7.14 (s, 1H), 3.49-3.44 (m, 8H), 1.46 (s, 9H). ¹³C NMR (151 MHz, CDCl₃) δ 154.7, 154.5, 142.1, 136.4, 134.7, 129.6, 108.9, 80.7, 43.9, 28.5. HRMS: (ESI) [M+H]⁺ calc. for C₁₅H₂₂IN₄O₃, 433.0731, observed, 433.0743.

### tert-butyl 4-((2-bromopyrimidin-5-yl)carbamoyl)piperazine-1-carboxylate

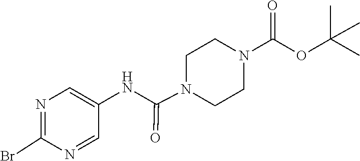 [SMILES: CC(C)(C)OC(=O)N1CCN(C(=O)Nc2cnc(Br)nc2)CC1]

Synthesized according to procedure 11 (In THE instead of DCM), yellow solid, 310 mg, 56% yield. ¹H NMR (500 MHz, CDCl₃) δ 8.72 (d, J=21.8 Hz, 2H), 6.92 (s, 1H), 3.53-3.48 (m, 7H), 1.47 (s, 9H). ¹³C NMR (126 MHz, CDCl₃) δ 154.7, 153.8, 150.4, 145.2, 134.2, 80.9, 44.0, 28.5. HRMS: (ESI) [M+H]⁺ calc. for C₁₄H₂₁BrN₅O₃, 386.0822, observed, 386.0828.

### tert-butyl 4-((2-fluoro-4-iodophenyl)carbamoyl)piperazine-1-carboxylate

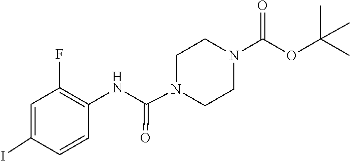 [SMILES: CC(C)(C)OC(=O)N1CCN(C(=O)Nc2ccc(I)cc2F)CC1]

Synthesized according to procedure 11, white solid, 277 mg, 49% yield. ¹H NMR (500 MHz, CDCl₃) δ 7.86 (t, J=8.5 Hz, 1H), 7.43-7.37 (m, 2H), 6.56-6.53 (m, 1H), 3.53-3.48 (m, 8H), 1.47 (s, 9H). ¹⁹F NMR (376 MHz, cdcl₃) δ −130.23-−130.31 (m). ¹³C NMR (126 MHz, CDCl₃) δ 154.7, 153.9, 152.1 (d, J=246.4 Hz), 133.8 (d, J=3.6 Hz), 127.5 (d, J=9.5 Hz), 123.8 (d, J=21.8 Hz), 122.9 (d, J=1.2 Hz), 83.9 (d, J=8.0 Hz), 80.6, 43.9, 28.5. HRMS: (ESI) [M+H]⁺ calc. for C₁₆H₂₂FIN₃O₃, 450.0684, observed, 450.0689.

### tert-butyl 4-((6-bromonaphthalen-2-yl)carbamoyl)piperazine-1-carboxylate

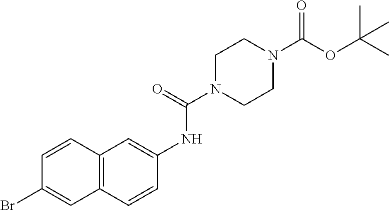 [SMILES: CC(C)(C)OC(=O)N1CCN(C(=O)Nc2ccc3cc(Br)ccc3c2)CC1]

Synthesized according to procedure 11, yellow solid, 610 mg, 78% yield. ¹H NMR (500 MHz, DMSO) δ 8.87 (s, 1H), 8.05 (dd, J=7.7, 1.7 Hz, 2H), 7.78 (d, J=9.0 Hz, 1H), 7.72 (d, J=8.8 Hz, 1H), 7.65 (dd, J=8.9, 2.1 Hz, 1H), 7.52 (dd, J=8.8, 2.0 Hz, 1H), 3.49-3.45 (m, 4H), 3.40-3.36 (m, 4H), 1.42 (s, 9H). ¹³C NMR (126 MHz, DMSO) δ 154.9, 153.9, 138.8, 132.1, 130.2, 129.2, 129.2, 129.0, 127.1, 122.0, 116.7, 114.7, 79.1, 43.6, 28.1. HRMS: (ESI) [M+H]⁺ calc. for C₂₀H₂₅BrN₃O₃, 434.1074, observed, 434.1081.

### tert-butyl 4-((7-bromonaphthalen-2-yl)carbamoyl)piperazine-1-carboxylate

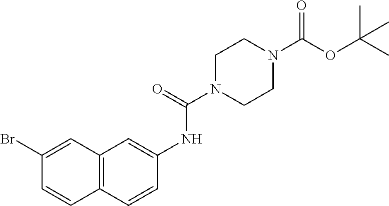 [SMILES: CC(C)(C)OC(=O)N1CCN(C(=O)Nc2ccc3ccc(Br)cc3c2)CC1]

Synthesized according to procedure 11, yellow solid, 427 mg, 96% yield. ¹H NMR (500 MHz, CDCl₃) δ 7.88 (d, J=1.7 Hz, 1H), 7.82 (d, J=1.6 Hz, 1H), 7.72 (d, J=8.8 Hz, 1H), 7.62 (d, J=8.7 Hz, 1H), 7.43 (dd, J=8.7, 1.9 Hz, 2H), 6.59 (brs, 1H), 3.53 (s, 8H), 1.49 (s, 9H). ¹³C NMR (126 MHz, CDCl₃) δ 154.9, 154.7, 137.4, 135.3, 129.4, 129.3, 128.8, 128.6, 128.1, 120.9, 120.7, 115.0, 80.6, 44.1, 28.5. HRMS: (ESI) [M+H]⁺ calc. for C₂₀H₂₅BrN₃O₃, 434.1074, observed, 434.1079.

### tert-butyl 4-((6-(3-(4-cyclopropylphenoxy)propyl)pyridin-3-yl)carbamoyl)piperazine-1-carboxylate (2am)

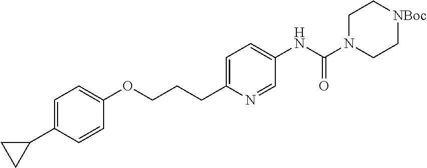 [SMILES: CC(C)(C)OC(=O)N1CCN(C(=O)Nc2ccc(CCCOc3ccc(C4CC4)cc3)nc2)CC1]

Synthesized according to procedure 3, white solid, 60 mg, 39% yield ¹H NMR (500 MHz, CDCl₃) δ 8.42 (s, 1H), 7.94 (d, J=7.5 Hz, 1H), 7.15 (d, J=8.2 Hz, 1H), 6.98 (d, J=8.6 Hz, 2H), 6.78 (d, J=8.6 Hz, 2H), 6.65 (s, 1H), 3.95 (t, J=6.2 Hz, 2H), 3.51 (s, 8H), 2.95 (t, J=7.5 Hz, 2H), 2.18 (p, J=6.4 Hz, 2H), 1.87-1.80 (m, 1H), 1.48 (s, 9H), 0.91-0.84 (m, 2H), 0.62-0.58 (m, 2H). ¹³C NMR (126 MHz, CDCl₃) δ 157.1, 154.9, 154.7, 135.9, 126.9, 114.5, 80.6, 67.1, 43.9, 33.7, 29.5, 28.5, 14.7, 8.7. HRMS: (ESI) [M+H]⁺ calc. for C₂₇H₃₇N₄O₄, 481.2809, observed, 481.2816.

### Tert-butyl 4-((6-(4-(3-cyclopropylphenoxy)butyl)132yridine-3-yl)carbamoyl)piperazine-1-carboxylate (2an)

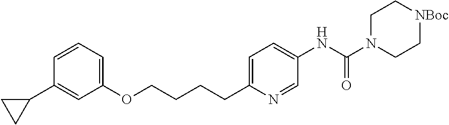 [SMILES: CC(C)(C)OC(=O)N1CCN(C(=O)Nc2ccc(CCCCOc3cccc(C4CC4)c3)nc2)CC1]

Synthesized according to procedure 3, white solid, 30 mg, 22% yield. ¹H NMR (600 MHz, CDCl₃) δ 8.33 (s, 1H), 7.84 (dd, J=8.5, 2.6 Hz, 1H), 7.12 (t, J=7.9 Hz, 1H), 7.09 (d, J=8.5 Hz, 1H), 7.04 (s, 1H), 6.65-6.62 (m, 2H), 6.58-6.57 (m, 1H), 3.93 (t, J=6.2 Hz, 2H), 3.49-3.44 (m, 8H), 2.78 (t, J=7.5 Hz, 2H), 1.88-1.76 (m, 5H), 1.46 (s, 9H), 0.93-0.90 (m, 2H), 0.68-0.65 (m, 2H). ¹³C NMR (151 MHz, CDCl₃) δ 159.2, 156.5, 155.1, 154.7, 145.8, 140.9, 133.7, 129.3, 128.8, 122.7, 118.1, 112.1, 111.2, 80.5, 67.5, 44.0, 37.2, 29.0, 28.5, 26.5, 15.5, 9.3. HRMS: (ESI) [M+H]⁺ calc. for C₂₈H₃₉N₄O₄, 495.2966, observed, 495.2974.

### tert-butyl 4-((4-(4-(3-cyclopropylphenoxy)butyl)-2-fluorophenyl)carbamoyl)piperazine-1-carboxylate (2ao)

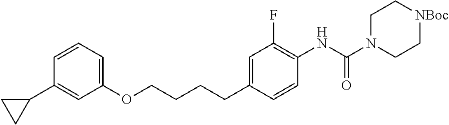 [SMILES: CC(C)(C)OC(=O)N1CCN(C(=O)Nc2ccc(CCCCOc3cccc(C4CC4)c3)cc2F)CC1]

Synthesized according to procedure 3, white solid, 64 mg, 40% yield. ¹H NMR (600 MHz, CDCl₃) δ 7.92 (t, J=8.4 Hz, 1H), 7.14 (t, J=7.9 Hz, 1H), 6.95-6.93 (m, 1H), 6.92-6.89 (m, 1H), 6.68-6.64 (m, 2H), 6.60-6.58 (m, 1H), 6.48 (d, J=3.3 Hz, 1H), 3.94 (t, J=5.9 Hz, 2H), 3.54-3.48 (m, 8H), 2.63 (t, J=7.1 Hz, 2H), 1.89-1.82 (m, 1H), 1.81-1.74 (m, 4H), 1.48 (s, 9H), 0.96-0.91 (m, 2H), 0.70-0.66 (m, 2H). ¹⁹F NMR (565 MHz, CDCl₃) δ −132.64-−132.69 (m). ¹³C NMR (151 MHz, CDCl₃) δ 159.3, 154.6 (d, J=33.2 Hz), 152.8 (d, J=241.1 Hz), 145.9, 138.1 (d, J=6.9 Hz), 129.3, 124.8 (d, J=10.0 Hz), 124.5 (d, J=3.1 Hz), 121.7, 118.2, 114.7 (d, J=19.0 Hz), 112.2, 111.2, 80.5, 67.6, 44.0, 34.9, 28.9, 28.5, 27.8, 15.6, 9.4. HRMS: (ESI) [M+H]⁺ calc. for C₂₉H₃₉FN₃O₄, 512.2919, observed, 512.2934.

### tert-butyl 4-((2-decylpyrimidin-5-yl)carbamoyl)piperazine-1-carboxylate (2ap)

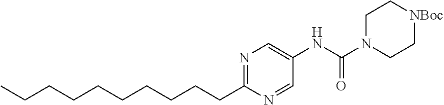 [SMILES: CCCCCCCCCCc1ncc(NC(=O)N2CCN(C(=O)OC(C)(C)C)CC2)cn1]

Synthesized according to procedure 3, white solid, 90 mg, 52% yield. ¹H NMR (500 MHz, CDCl₃) δ 8.88 (s, 2H), 3.58-3.49 (m, 8H), 2.96-2.91 (m, 2H), 1.79 (p, J=7.7 Hz, 2H), 1.48 (s, 9H), 1.38-1.21 (m, 14H), 0.87 (t, J=7.0 Hz, 3H). ¹³C NMR (126 MHz, CDCl₃) δ 165.2, 154.7, 154.3, 148.3, 132.3, 80.7, 44.1, 38.4, 32.0, 29.7, 29.7, 29.6, 29.5, 28.9, 28.5, 22.8, 14.3. HRMS: (ESI) [M+H]⁺ calc. for C₂₄H₄₂N₅O₃, 448.3282, observed, 448.3290.

### tert-butyl 4-((6-octylnaphthalen-2-yl)carbamoyl)piperazine-1-carboxylate (2ar)

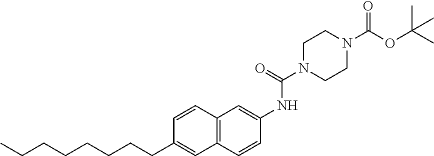 [SMILES: CCCCCCCCc1ccc2cc(NC(=O)N3CCN(C(=O)OC(C)(C)C)CC3)ccc2c1]

Synthesized according to procedure 3, white solid, 155 mg, 72% yield. ¹H NMR (600 MHz, CDCl₃) δ 7.86 (d, J=1.7 Hz, 1H), 7.68 (d, J=8.8 Hz, 1H), 7.64 (d, J=8.4 Hz, 1H), 7.52 (s, 1H), 7.37 (dd, J=8.8, 2.1 Hz, 1H), 7.28 (dd, J=8.4, 1.5 Hz, 1H), 6.72 (s, 1H), 3.48 (s, 8H), 2.75-2.69 (m, 2H), 1.67 (p, J=7.7 Hz, 2H), 1.48 (s, 9H), 1.38-1.22 (m, 10H), 0.87 (t, J=7.0 Hz, 3H). ¹³C NMR (151 MHz, CDCl₃) δ 155.2, 154.7, 139.3, 135.5, 132.3, 130.4, 128.1, 128.0, 127.2, 126.0, 120.7, 116.3, 80.4, 43.9, 36.0, 31.9, 31.4, 29.5, 29.4, 29.3, 28.4, 22.7, 14.2. HRMS: (ESI) [M+H]⁺ calc. for C₂₈H₄₂N₃O₃, 468.3221, observed, 468.3224.

### tert-butyl 4-((7-heptylnaphthalen-2-yl)carbamoyl)piperazine-1-carboxylate (2as)

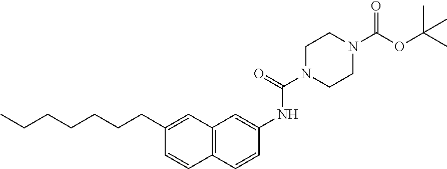 [SMILES: CCCCCCCc1ccc2ccc(NC(=O)N3CCN(C(=O)OC(C)(C)C)CC3)cc2c1]

Synthesized according to procedure 3, white solid, 121 mg, 58% yield. ¹H NMR (600 MHz, CDCl₃) δ 7.83 (s, 1H), 7.70 (d, J=8.7 Hz, 1H), 7.67 (d, J=8.3 Hz, 1H), 7.48 (s, 1H), 7.33 (d, J=8.3 Hz, 1H), 7.22 (dd, J=8.3, 1.2 Hz, 1H), 6.70 (brs, 1H), 3.49 (s, 8H), 2.74-2.69 (m, 2H), 1.67 (p, J=7.5 Hz, 2H), 1.48 (s, 9H), 1.37-1.24 (m, 8H), 0.88 (t, J=7.0 Hz, 3H). ¹³C NMR (151 MHz, CDCl₃) δ 155.1, 154.7, 141.1, 136.3, 134.2, 128.7, 128.3, 127.4, 126.3, 125.8, 119.7, 115.9, 80.4, 43.9, 36.1, 31.9, 31.3, 29.3, 29.2, 28.4, 22.7, 14.2. HRMS: (ESI) [M+H]⁺ calc. for C₂₇H₄₀N₃O₃, 454.3064, observed, 454.3069.

### tert-butyl 4-((7-(3-phenoxypropyl)naphthalen-2-yl)carbamoyl)piperazine-1-carboxylate (2at)

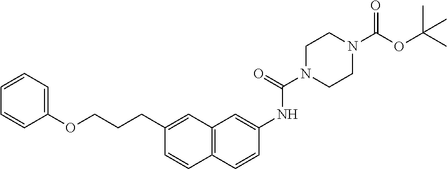 [SMILES: CC(C)(C)OC(=O)N1CCN(C(=O)Nc2ccc3ccc(CCCOc4ccccc4)cc3c2)CC1]

Synthesized according to procedure 3, white solid, 146 mg, 52% yield. ¹H NMR (600 MHz, CDCl₃) δ 7.85 (s, 1H), 7.75 (d, J=8.8 Hz, 1H), 7.72 (d, J=8.3 Hz, 1H), 7.57 (s, 1H), 7.39 (dd, J=8.7, 1.9 Hz, 1H), 7.32-7.27 (m, 4H), 6.96 (t, J=7.3 Hz, 1H), 6.94-6.91 (m, 2H), 6.60 (brs, 1H), 4.01 (t, J=6.3 Hz, 2H), 3.55 (s, 8H), 2.97 (t, J=7.6 Hz, 2H), 2.22-2.16 (m, 2H), 1.51 (s, 9H). ¹³C NMR (151 MHz, CDCl₃) δ 159.1, 155.1, 154.7, 139.8, 136.5, 134.3, 129.6, 128.9, 128.5, 127.8, 126.3, 126.2, 120.7, 120.0, 115.9, 114.6, 80.5, 66.9, 44.0, 32.5, 30.8, 28.5. HRMS: (ESI) [M+Na]⁺ calc. for C₂₉H₃₅N₃O₄Na, 524.2287, observed, 524.2277.

### N-(6-(3-(4-cyclopropylphenoxy)propyl)pyridin-3-yl)piperazine-1-carboxamide bis-hydrochloride (3am)

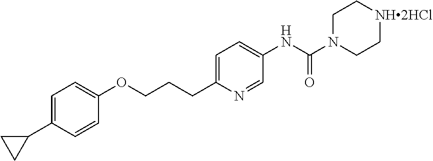

Synthesized according to procedure 4, yellow solid, 20 mg, 72% yield. ¹H NMR (500 MHz, MeOD) δ 9.04 (d, J=2.4 Hz, 1H), 8.44 (dd, J=8.9, 2.5 Hz, 1H), 7.88 (d, J=8.9 Hz, 1H), 6.96 (d, J=8.6 Hz, 2H), 6.71-6.67 (m, 2H), 4.04 (t, J=5.6 Hz, 2H), 3.89-3.86 (m, 4H), 3.34-3.32 (m, 4H), 3.20 (t, J=7.4 Hz, 2H), 2.27 (p, J=6.1 Hz, 2H), 1.85-1.79 (m, 1H), 0.90-0.85 (m, 2H), 0.58-0.53 (m, 2H). ¹³C NMR (126 MHz, MeOD) δ 157.8, 155.7, 151.6, 139.9, 137.6, 137.2, 132.0, 128.5, 127.8, 115.2, 67.7, 44.4, 42.4, 31.2, 29.7, 15.3, 9.0. HRMS: (ESI) [M+H]⁺ calc. for C₂₂H₂₉N₄O₂, 381.2285, observed, 381.2288.

### N-(6-(4-(3-cyclopropylphenoxy)butyl)pyridin-3-yl)piperazine-1-carboxamide bis-hydrochloride (3an)

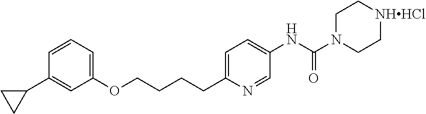

Synthesized according to procedure 4, white solid, 20 mg, 71% yield. ¹H NMR (500 MHz, MeOD) δ 9.04 (d, J=2.4 Hz, 1H), 8.44 (dd, J=8.9, 2.5 Hz, 1H), 7.89 (d, J=8.9 Hz, 1H), 7.11 (t, J=7.9 Hz, 1H), 6.67-6.61 (m, 2H), 6.59 (d, J=2.0 Hz, 1H), 4.00 (t, J=6.0 Hz, 2H), 3.88-3.84 (m, 4H), 3.34-3.32 (m, 4H), 3.10-3.04 (m, 2H), 2.03-1.95 (m, 2H), 1.91-1.81 (m, 3H), 0.96-0.90 (m, 2H), 0.66-0.61 (m, 2H). ¹³C NMR (126 MHz, MeOD) δ 160.4, 155.6, 151.6, 147.1, 139.9, 137.2, 132.2, 130.2, 128.3, 119.0, 112.9, 112.3, 68.0, 44.4, 42.4, 33.4, 29.7, 27.0, 16.2, 9.6. HRMS: (ESI) [M+H]⁺ calc. for C₂₄H₃₁N₄O₂, 395.2442, observed, 395.2448.

### N-(4-(4-(3-cyclopropylphenoxy)butyl)-2-fluorophenyl)piperazine-1-carboxamide hydrochloride (3ao)

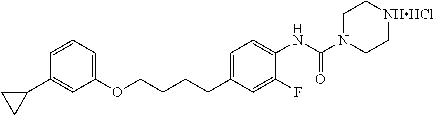

Synthesized according to procedure 4, white solid, 52 mg, 93% yield. ¹H NMR (500 MHz, MeOD) δ 7.32 (t, J=8.3 Hz, 1H), 7.10 (t, J=7.9 Hz, 1H), 7.04-6.97 (m, 2H), 6.68-6.62 (m, 2H), 6.62 (d, J=7.7 Hz, 1H), 6.62-6.57 (m, 1H), 3.95 (t, J=5.8 Hz, 2H), 3.82-3.76 (m, 4H), 3.31-3.26 (m, 4H), 2.70-2.64 (m, 2H), 1.88-1.82 (m, 1H), 1.81-1.75 (m, 4H), 0.95-0.89 (m, 2H), 0.66-0.62 (m, 2H). ¹⁹F NMR (376 MHz, MeOD) δ −126.26-−126.32 (m). ¹³C NMR (126 MHz, MeOD) δ 160.6, 157.7, 157.4 (d, J=245.7 Hz), 147.0, 142.9 (d, J=6.9 Hz), 130.2, 127.6 (d, J=1.6 Hz), 125.2 (d, J=3.2 Hz), 125.1 (d, J=12.2 Hz), 118.8, 116.5 (d, J=20.0 Hz), 113.0, 112.3, 68.5, 44.4, 42.5, 35.9, 29.9, 28.9, 16.2, 9.6. HRMS: (ESI) [M+H]⁺ calc. for C₂₄H₃₁FN₃O₂, 412.2395, observed, 412.2398.

### N-(2-decylpyrimidin-5-yl)piperazine-1-carboxamide bis-hydrochloride (3ap)

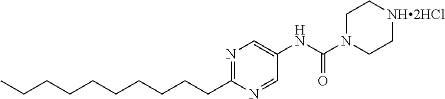

Synthesized according to procedure 4, yellow solid, 32 mg, 76% yield. ¹H NMR (600 MHz, MeOD) δ 9.31 (s, 2H), 3.91-3.86 (m, 4H), 3.34-3.32 (m, 4H), 3.09-3.03 (m, 2H), 1.88 (p, J=7.7 Hz, 2H), 1.45-1.35 (m, 4H), 1.35-1.26 (m, 10H), 0.90 (t, J=7.0 Hz, 3H). ¹³C NMR (151 MHz, MeOD) δ 160.7, 155.4, 147.9, 136.6, 44.3, 42.4, 36.0, 33.1, 30.7, 30.6, 30.5, 30.4, 30.1, 28.8, 23.8, 14.5. HRMS: (ESI) [M+H]⁺ calc. for C₁₉H₃₄N₅O, 348.2758, observed, 348.2754.

### N-(6-octylnaphthalen-2-yl)piperazine-1-carboxamide hydrochloride (3ar)

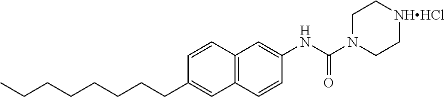

Synthesized according to procedure 4, white solid, 111 mg, 86% yield. ¹H NMR (600 MHz, MeOD) δ 7.83 (s, 1H), 7.72 (d, J=7.2 Hz, 2H), 7.67 (d, J=6.9 Hz, 2H), 7.56 (s, 1H), 7.47 (d, J=7.1 Hz, 1H), 7.31 (d, J=6.8 Hz, 1H), 3.83 (s, 4H), 3.37-3.32 (m, 4H) 2.74 (s, 2H), 1.69 (s, 2H), 1.40-1.24 (m, 10H), 0.89 (s, 3H). ¹³C NMR (151 MHz, MeOD) δ 157.6, 140.5, 137.3, 133.7, 132.0, 128.9, 128.2, 127.1, 122.5, 118.2, 44.5, 42.5, 37.0, 33.0, 32.6, 30.6, 30.4, 23.7, 14.4. HRMS: (ESI) [M+H]⁺ calc. for C₂₃H₃₄N₃O, 368.2696, observed, 368.2690.

### N-(7-heptylnaphthalen-2-yl)piperazine-1-carboxamide hydrochloride (3as)

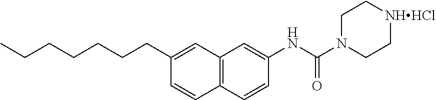

Synthesized according to procedure 4, white solid, 87 mg, 84% yield. ¹H NMR (600 MHz, MeOD) δ 7.80 (s, 1H), 7.75-7.68 (m, 2H), 7.51 (s, 1H), 7.44 (d, J=7.9 Hz, 1H), 7.24 (d, J=7.2 Hz, 1H), 3.83 (s, 4H), 3.40-3.31 (m, 4H), 2.75 (s, 2H), 1.70 (s, 2H), 1.40-1.25 (m, 10H), 0.90 (s, 3H). ¹³C NMR (151 MHz, MeOD) δ 157.5, 142.1, 138.0, 135.5, 130.3, 129.0, 128.5, 127.3, 126.8, 121.6, 117.9, 44.5, 42.5, 37.1, 33.0, 32.6, 30.3, 23.7, 14.4. HRMS: (ESI) [M+H]⁺ calc. for C₂₂H₃₂N₃O, 354.2540, observed, 354.2545.

### N-(7-(3-phenoxypropyl)naphthalen-2-yl)piperazine-1-carboxamide hydrochloride (3at)

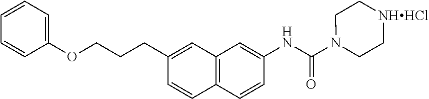

Synthesized according to procedure 4, white solid, 66 mg, 84% yield. ¹H NMR (600 MHz, MeOD) δ 7.80 (d, J=2.0 Hz, 1H), 7.74 (d, J=8.9 Hz, 1H), 7.72 (d, J=8.4 Hz, 1H), 7.56 (s, 1H), 7.46 (dd, J=8.8, 2.1 Hz, 1H), 7.29 (dd, J=8.3, 1.6 Hz, 1H), 7.26-7.23 (m, 2H), 6.91-6.88 (m, 3H), 3.98 (t, J=6.2 Hz, 2H), 3.84-3.81 (m, 4H), 3.31-3.28 (m, 4H), 2.97-2.92 (m, 2H), 2.18-2.12 (m, 2H). ¹³C NMR (151 MHz, MeOD) δ 160.5, 157.5, 141.1, 138.2, 135.5, 130.4, 130.4, 129.1, 128.7, 127.3, 127.1, 121.7, 121.6, 117.9, 115.5, 67.8, 44.5, 42.5, 33.3, 32.1. HRMS: (ESI) [M+H]⁺ calc. for C₂₄H₂₈N₃O₂, 390.2176, observed, 390.2170.

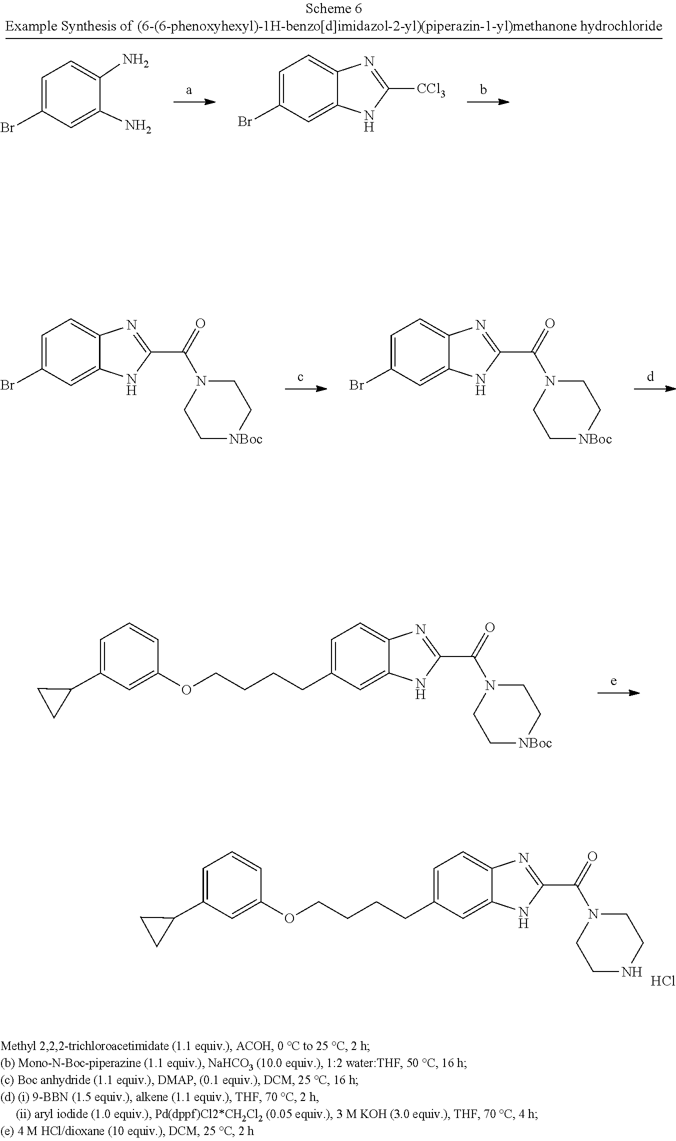

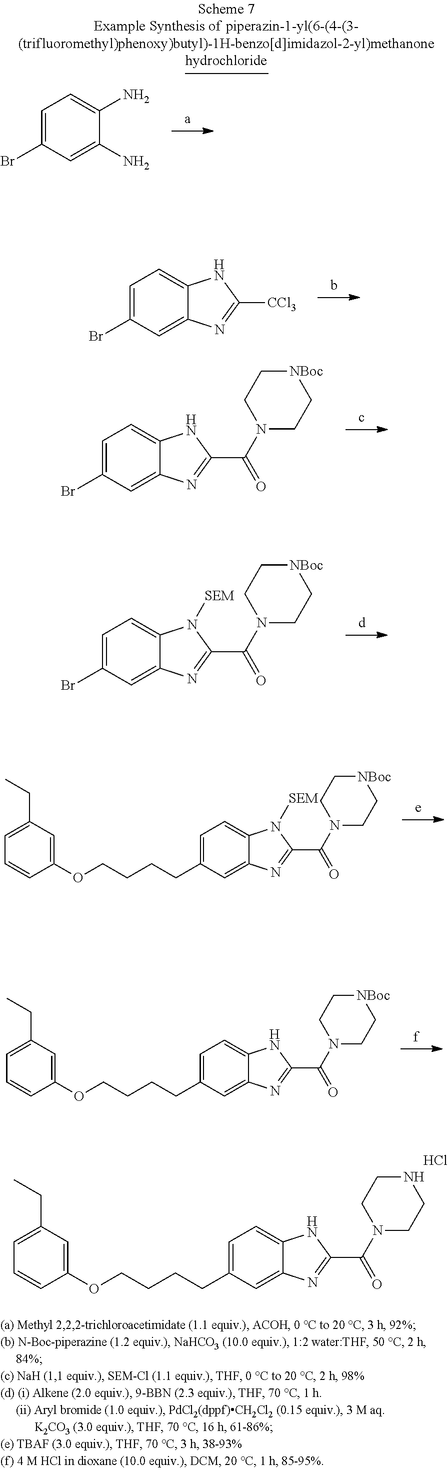

### tert-butyl 4-(6-bromo-1H-benzo[d]imidazole-2-carbonyl)piperazine-1-carboxylate (15a)

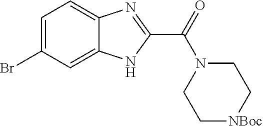 [SMILES: CC(C)(C)OC(=O)N1CCN(C(=O)c2nc3ccc(Br)cc3[nH]2)CC1]

Synthesized according to General Procedure 9. Purified via column chromatography (30-50% ethyl acetate/hexanes). White solid (79%, 410 mg). ¹H NMR (500 MHz, CDCl₃) δ 11.77 (s, 1H), 7.72-7.67 (m, 1H), 7.51-7.37 (m, 2H), 4.80-4.72 (m, 2H), 3.91 (t, J=5.2 Hz, 2H), 3.68-3.59 (m, 5H), 1.52 (s, 9H). ¹³C NMR (126 MHz, CDCl₃) δ 158.6*, 158.5*, 154.7, 145.9*, 145.6*, 144.5*, 142.2*, 134.0*, 131.9*, 128.4*, 126.9*, 124.0*, 122.5*, 118.7*, 116.2*, 115.0*, 113.2*, 80.6, 46.8, 43.5, 28.50. Material isolated as an approximately 1:1 ratio of isomers. HRMS: (ESI) [M+H]⁺ calc. for C₁₇H₂₂BrN₄O₃⁺ 409.0870, observed, 409.0884.

### tert-butyl 6-bromo-2-(4-(tert-butoxycarbonyl)piperazine-1-carbonyl)-1H-benzo[d]imidazole-1-carboxylate

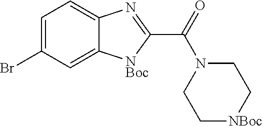 [SMILES: CC(C)(C)OC(=O)N1CCN(C(=O)c2nc3ccc(Br)cc3n2C(=O)OC(C)(C)C)CC1]

Synthesized by General Procedure 10. 452 mg, 89%, white solid. ¹H NMR (600 MHz, CDCl₃) δ 8.18 (d, J=1.9 Hz, 1H), 7.91 (d, J=1.8 Hz, 1H), 7.86 (d, J=8.7 Hz, 1H), 7.62 (d, J=8.5 Hz, 1H), 7.52 (ddd, J=17.9, 8.6, 1.9 Hz, 2H), 3.78 (t, J=5.3 Hz, 5H), 3.59 (t, J=5.3 Hz, 5H), 3.53-3.48 (m, 4H), 3.35 (q, J=5.5 Hz, 4H), 1.65 (d, J=2.4 Hz, 18H), 1.48 (s, 18H). ¹³C NMR (151 MHz, CDCl₃) δ 160.5, 154.6, 147.3, 147.1, 147.1, 146.7, 143.6, 141.3, 132.9, 131.0, 129.2, 128.4, 123.8, 122.1, 119.6, 118.1, 118.0, 116.1, 87.4, 87.3, 80.7, 46.6, 41.9, 28.5, 28.0, 28.0. HRMS: (ESI) [M+H]⁺ calc. for C₂₂H₃₀BrN₄O₅⁺ 509.1394 observed, 509.1416.

### tert-butyl 4-(6-(6-phenoxyhexyl)-1H-benzo[d]imidazole-2-carbonyl)piperazine-1-carboxylate (17a)

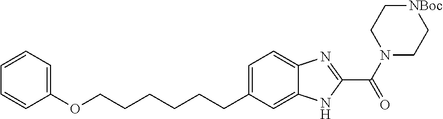 [SMILES: CC(C)(C)OC(=O)N1CCN(C(=O)c2nc3ccc(CCCCCCOc4ccccc4)cc3[nH]2)CC1]

Synthesized according to General Procedure 3. Purified via column chromatography (30-40% ethyl acetate/hexanes). Clear oil (60%, 60 mg). ¹H NMR (500 MHz, MeOD) δ 7.76 (d, J=8.6 Hz, 1H), 7.67 (d, J=1.3 Hz, 1H), 7.52 (dd, J=8.6, 1.5 Hz, 1H), 7.26-7.20 (m, 2H), 6.90-6.83 (m, 3H), 4.12 (t, J=5.3 Hz, 4H), 3.93 (t, J=6.3 Hz, 2H), 3.44 (t, J=5.3 Hz, 4H), 2.85 (t, J=7.6 Hz, 2H), 1.74 (dq, J=8.5, 6.4 Hz, 4H), 1.55-1.49 (m, 2H), 1.48-1.41 (m, 2H). ¹³C NMR (126 MHz, MeOD) δ 160.5*, 156.7*, 144.5*, 142.1*, 130.4*, 129.8*, 121.5*, 115.9*, 115.5*, 114.8*, 68.7, 44.2, 41.7, 36.9, 32.7, 30.3, 29.9, 27.0. HRMS: (ESI) [M+H]⁺ calc. for C₃₄H₄₇N₄O₆⁺ 607.3490 observed, 607.3486.

### tert-butyl 4-(6-(4-(3-cyclopropylphenoxy)butyl)-1H-benzo[d]imidazole-2-carbonyl)piperazine-1-carboxylate (17b)

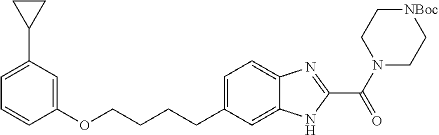 [SMILES: CC(C)(C)OC(=O)N1CCN(C(=O)c2nc3ccc(CCCCOc4cccc(C5CC5)c4)cc3[nH]2)CC1]

Synthesized according to General Procedure 3. Purified via column chromatography (20-40% ethyl acetate/hexanes). White solid (64%, 52 mg). ¹H NMR (400 MHz, cdcl₃) δ 10.61-10.38 (m, 1H), 7.72 (d, J=8.4 Hz, OH), 7.63 (s, OH), 7.45-7.41 (m, OH), 7.32 (dd, J=1.6, 0.8 Hz, OH), 7.23 (dd, J=8.3, 1.6 Hz, OH), 7.18 (dd, J=8.4, 1.6 Hz, OH), 7.14 (t, J=7.9 Hz, 1H), 6.70-6.64 (m, 2H), 6.59 (t, J=2.2 Hz, 1H), 4.76 (dt, J=7.4, 4.2 Hz, 2H), 3.96 (td, J=6.0, 2.8 Hz, 2H), 3.83 (s, 2H), 3.59 (dt, J=10.6, 5.5 Hz, 4H), 2.82 (t, J=7.0 Hz, 2H), 1.88-1.81 (m, 4H), 1.50 (d, J=0.6 Hz, 9H), 0.97-0.90 (m, 2H), 0.71-0.64 (m, 2H). ¹³C NMR (126 MHz, CDCl₃) δ 159.3*, 159.2*, 154.7, 145.8*, 145.0*, 144.7*, 143.6*, 141.7*, 140.0*, 137.6*, 129.3*, 126.5*, 124.6*, 120.7*, 120.1*, 118.1*, 112.2*, 111.2*, 80.5, 71.0, 67.6, 46.8, 43.3, 36.1, 35.8, 29.0, 28.9, 28.5, 28.4, 28.3, 26.3, 15.5, 9.3. Material isolated as an approximately 1:1 ratio of rotamers. HRMS: (ESI) [M+H]⁺ calc. for C₃₀H₃₉N₄O₄⁺ 519.2966, observed, 519.2987.

### (6-(6-phenoxyhexyl)-1H-benzo[d]imidazol-2-yl)(piperazin-1-yl)methanone hydrochloride (18a)

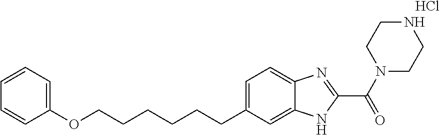 [SMILES: Cl ; O=C(c1nc2ccc(CCCCCCOc3ccccc3)cc2[nH]1)N1CCNCC1]

Synthesized by General Procedure 4. 39 mg, 89%, off-white solid. ¹H NMR (500 MHz, MeOD) δ 7.76 (d, J=8.6 Hz, 1H), 7.67 (d, J=1.3 Hz, 1H), 7.52 (dd, J=8.6, 1.5 Hz, 1H), 7.26-7.20 (m, 2H), 6.90-6.83 (m, 3H), 4.12 (t, J=5.3 Hz, 4H), 3.93 (t, J=6.3 Hz, 2H), 3.44 (t, J=5.3 Hz, 5H), 2.85 (t, J=7.6 Hz, 2H), 1.75 (dtd, J=15.1, 7.5, 5.3 Hz, 4H), 1.55-1.49 (m, 2H), 1.48-1.41 (m, 2H). ¹³C NMR (126 MHz, MeOD) δ 160.5, 156.7, 144.5, 142.1, 130.4, 129.8, 121.5, 115.9, 115.5, 114.8, 68.7, 44.2, 41.7, 36.9, 32.7, 30.3, 29.9, 27.0. HRMS: (ESI) [M+H]⁺ calc. for C₂₄H₃₁N₄O₂⁺ 407.2442 observed, 407.2452.

### (6-(4-(3-cyclopropylphenoxy)butyl)-1H-benzo[d]142midazole-2-yl)(piperazin-1-yl)methanone hydrochloride (18b)

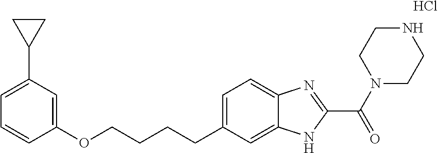 [SMILES: Cl ; O=C(c1nc2ccc(CCCCOc3cccc(C4CC4)c3)cc2[nH]1)N1CCNCC1]

Synthesized according to General Procedure 4. Purified via trituration with diethyl ether. White solid (73%, 51 mg). ¹H NMR (500 MHz, CD₃OD) δ 7.78 (d, J=8.5 Hz, 1H), 7.69 (s, 1H), 7.54 (d, J=8.5 Hz, 1H), 7.08 (t, J=8.0 Hz, 1H), 6.66-6.55 (m, 3H), 4.13 (t, J=5.1 Hz, 4H), 3.96 (t, J=6.1 Hz, 2H), 3.44 (t, J=5.2 Hz, 4H), 2.91 (t, J=7.5 Hz, 2H), 1.93-1.76 (m, 5H), 0.96-0.87 (m, 2H), 0.67-0.59 (m, 2H). ¹³C NMR (126 MHz, CD₃OD) δ 160.5, 156.7, 147.0, 144.2, 142.1, 133.5, 131.8, 130.2, 129.8, 118.8, 115.9, 114.9, 112.9, 112.3, 68.4, 44.2, 36.6, 29.9, 29.3, 16.2, 9.6. HRMS: (ESI) [M+H]⁺ calc. for C₂₅H₃₁N₄O₂⁺ 419.2442 observed, 419.2448.

### 6-bromo-2-(trichloromethyl)-1H-benzo[d]imidazole

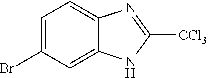

Synthesized according to General Procedure 18. Purified via precipitation. White solid (92%, 15.49 g). ¹H NMR (400 MHz, cdcl₃) δ 9.95 (s, 1H), 8.01 (d, J=1.6 Hz, 1H), 7.73 (d, J=8.7 Hz, 1H), 7.68 (d, J=1.8 Hz, 1H), 7.53-7.45 (m, 1H), 7.40 (d, J=8.7 Hz, 1H). ¹³C NMR (126 MHz, DMSO) δ 151.7, 139.6, 137.1, 127.0, 119.2, 118.2, 116.0 88.4. HRMS: (ESI) [M+H]⁺ calc. for C₈H₅BrCl₃N₂⁺ 312.8696, observed, 312.8667.

### tert-butyl 4-(6-bromo-1H-benzo[d]imidazole-2-carbonyl)piperazine-1-carboxylate

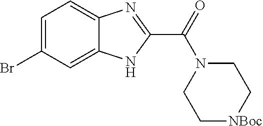 [SMILES: CC(C)(C)OC(=O)N1CCN(C(=O)c2nc3ccc(Br)cc3[nH]2)CC1]

Synthesized according to General Procedure 9. Purified via column chromatography (30-50% ethyl acetate/hexanes). White solid (79%, 410 mg). ¹H NMR (500 MHz, CDCl₃) δ 11.77 (s, 1H), 7.72-7.67 (m, 1H), 7.51-7.37 (m, 2H), 4.80-4.72 (m, 2H), 3.91 (t, J=5.2 Hz, 2H), 3.68-3.59 (m, 5H), 1.52 (s, 9H). ¹³C NMR (126 MHz, CDCl₃) δ 158.6*, 158.5*, 154.7, 145.9*, 145.6*, 144.5*, 142.2*, 134.0*, 131.9*, 128.4*, 126.9*, 124.0*, 122.5*, 118.7*, 116.2*, 115.0*, 113.2*, 80.6, 46.8, 43.5, 28.50. Material isolated as an approximately 1:1 ratio of isomers. HRMS: (ESI) [M+H]⁺ calc. for C₁₇H₂₂BrN₄O₃⁺ 409.0870, observed, 409.0884.

### tert-butyl 4-(5-bromo-1-((2-(trimethylsilyl)ethoxy)methyl)-1H-benzo[d]imidazole-2-carbonyl)piperazine-1-carboxylate

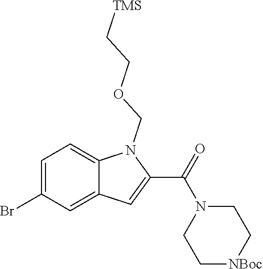 [SMILES: CC(C)(C)OC(=O)N1CCN(C(=O)c2cc3cc(Br)ccc3n2COCC[Si](C)(C)C)CC1]

Synthesized according to General Procedure 19. Purified via extraction. Yellow oil (98%, 1280 mg). ¹H NMR (400 MHz, CDCl₃) δ 7.94 (d, J=1.8 Hz, 0.5H), 7.77-7.71 (m, 0.5H), 7.65 (dd, J=8.7, 1.3 Hz, 1H), 7.51-7.43 (m, 2H), 5.75 (dd, J=6.6, 1.3 Hz, 2H), 3.90-3.77 (m, 4H), 3.62-3.51 (m, 6H), 1.48 (d, J=1.4 Hz, 9H), 0.91-0.83 (m, 2H), −0.06 (d, J=1.3 Hz, 9H). HRMS: (ESI) [M+H]⁺ calc. for C₂₃H₃₆BrN₄O₄Si⁺ 539.1684, observed, 539.1686.

### tert-butyl 4-(6-(4-(2-ethylphenoxy)butyl)-1-((2-(trimethylsilyl)ethoxy)methyl)-1H-benzo[d]imidazole-2-carbonyl)piperazine-1-carboxylate

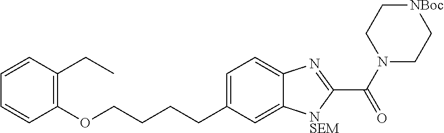 [SMILES: CCc1ccccc1OCCCCc1ccc2nc(C(=O)N3CCN(C(=O)OC(C)(C)C)CC3)n(COCC[Si](C)(C)C)c2c1]

Synthesized according to General Procedure 20. Purified via column chromatography (40-50% ethyl acetate/hexanes). Yellow oil (61%, 145 mg). ¹H NMR (500 MHz, CDCl₃) δ 7.74 (d, J=8.3 Hz, 0.5H), 7.65 (s, 0.5H), 7.51 (d, J=8.3 Hz, 0.5H), 7.41 (d, J=1.6 Hz, 0.5H), 7.28 (dd, J=8.4, 1.6 Hz, 0.5H), 7.24 (dd, J=8.4, 1.6 Hz, 0.5H), 7.20-7.12 (m, 3H), 6.94-6.86 (m, 1H), 6.84 (d, J=8.1 Hz, 1H), 5.80 (s, 2H), 4.05-3.97 (m, 3H), 3.96-3.88 (m, 2H), 3.83 (t, J=5.1 Hz, 2H), 3.63-3.53 (m, 6H), 2.88 (q, J=7.5 Hz, 2H), 2.71-2.63 (m, 3H), 1.97-1.86 (m, 5H), 1.51 (s, 9H), 1.25-1.18 (m, 4H), 0.95-0.85 (m, 2H), −0.03 (d, J=1.5 Hz, 9H). ¹³C NMR (126 MHz, CDCl₃) δ 160.3, 156.8, 156.8, 156.7, 154.7, 144.6, 144.2, 141.9, 140.0, 139.7, 138.0, 135.2, 133.3, 132.8, 132.8, 132.8, 129.0, 129.0, 128.9, 126.8, 126.8, 125.9, 124.7 (d, J=271 Hz), 120.5, 120.5, 120.4, 120.4, 120.0, 111.1, 111.1, 111.0, 111.0, 110.8, 110.3, 80.5, 73.7, 73.6, 67.6, 67.5, 66.6, 66.6, 47.3, 42.4, 36.1, 35.7, 29.1, 29.0, 28.5, 28.5, 23.4, 17.9, 14.3, 14.3, −1.3. Material isolated as an approximately 1:1 ratio of regioisomers. HRMS: (ESI) [M+H]⁺ calc. for C₃₅H₅₃N₄O₅Si⁺ 637.3780, observed,

### tert-butyl 4-(6-(4-(3-isopropylphenoxy)butyl)-1-((2-(trimethylsilyl)ethoxy)methyl)-1H-benzo[d]imidazole-2-carbonyl)piperazine-1-carboxylate (17h)

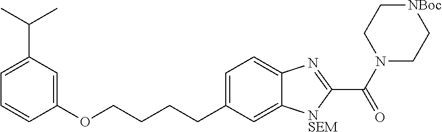 [SMILES: CC(C)c1cccc(OCCCCc2ccc3nc(C(=O)N4CCN(C(=O)OC(C)(C)C)CC4)n(COCC[Si](C)(C)C)c3c2)c1]

Synthesized according to General Procedure 20. Purified via column chromatography (20-30% ethyl acetate/hexanes). Yellow oil (82%, 198 mg). ¹H NMR (500 MHz, CDCl₃) δ 7.72 (d, J=8.2 Hz, OH), 7.64 (s, OH), 7.50 (d, J=8.4 Hz, OH), 7.41 (d, J=1.5 Hz, OH), 7.27-7.19 (m, 2H), 6.86-6.82 (m, 1H), 6.79 (p, J=1.3 Hz, 1H), 6.73 (dd, J=7.9, 2.4 Hz, 1H), 5.79 (d, J=1.8 Hz, 2H), 4.03-3.97 (m, 2H), 3.94-3.89 (m, 2H), 3.86-3.79 (m, 2H), 3.60-3.53 (m, 6H), 2.87 (p, J=7.0 Hz, 3H), 1.91-1.83 (m, 4H), 1.50 (s, 9H), 1.26 (d, J=6.9 Hz, 6H), 0.94-0.88 (m, 2H), −0.04 (d, J=1.5 Hz, 9H). ¹³C NMR (126 MHz, CDCl₃) δ 160.3, 159.2, 159.2, 154.7, 150.8, 144.6, 144.3, 141.9, 140.0, 139.7, 138.0, 135.2, 133.3, 129.3, 126.0, 124.8, 120.6, 120.0, 118.9, 113.1, 111.3, 110.8, 110.4, 80.5, 73.7, 73.6, 67.7, 67.6, 66.6, 47.4, 42.5, 36.2, 35.8, 34.3, 32.2, 29.1, 28.5, 26.3, 24.1, 18.0, −1.3. Material isolated as an approximately 1:1 mixture of regioisomers. Material isolated as an approximately 1:1 ratio of regioisomers. HRMS: (ESI) [M+H]⁺ calc. for C₃₆H₅₅N₄O₅Si⁺ 651.3936, observed, 651.3916.

### tert-butyl 4-(6-(4-(3-(dimethylamino)phenoxy)butyl)-1-((2-(trimethylsilyl)ethoxy)methyl)-1H-benzo[d]imidazole-2-carbonyl)piperazine-1-carboxylate (17i)

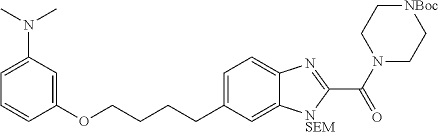 [SMILES: CN(C)c1cccc(OCCCCc2ccc3nc(C(=O)N4CCN(C(=O)OC(C)(C)C)CC4)n(COCC[Si](C)(C)C)c3c2)c1]

Synthesized according to General Procedure 20. Purified via column chromatography (20-40% ethyl acetate/hexanes). Yellow oil (86%, 187 mg). ¹H NMR (500 MHz, CDCl₃) δ 7.72 (d, J=8.3 Hz, 0.5H), 7.63 (s, 0.5H), 7.50 (d, J=8.3 Hz, 0.5H), 7.40 (d, J=1.5 Hz, 0.5H), 7.29-7.21 (m, 0.5H), 7.15 (dd, J=9.4, 7.1 Hz, 1H), 6.40-6.35 (m, 1H), 6.32-6.28 (m, 2H), 5.82-5.77 (m, 2H), 4.04-3.97 (m, 2H), 3.94-3.89 (m, 2H), 3.86-3.80 (m, 2H), 3.63-3.53 (m, 6H), 2.94 (s, 6H), 2.86 (q, J=7.4 Hz, 2H), 1.93-1.81 (m, 4H), 1.50 (s, 9H), 1.28 (t, J=7.1 Hz, 2H), 0.90 (ddd, J=9.6, 7.5, 2.3 Hz, 2H), −0.04 (d, J=1.1 Hz, 9H). ¹³C NMR (126 MHz, CDCl₃) δ 160.2, 160.2, 154.7, 152.1, 144.6, 144.2, 141.9, 140.0, 139.8, 138.0, 135.2, 133.3, 129.8, 126.0, 124.8, 120.5, 120.0, 110.8, 110.3, 105.8, 105.8, 102.1, 99.8, 99.8, 80.5, 73.7, 73.6, 67.6, 67.6, 66.6, 47.4, 42.4, 40.7, 36.2, 35.8, 29.1, 29.0, 28.5, 17.9, −1.3. Material isolated as an approximately 1:1 ratio of regioisomers. Material isolated as an approximately 1:1 ratio of rotamers. HRMS: (ESI) [M+H]⁺ calc. for C35H54N5O5Si+ 652.3889, observed, 652.3871.

### tert-butyl 4-(6-(4-(3-methoxyphenoxy)butyl)-1-((2-(trimethylsilyl)ethoxy)methyl)-1H-benzo[d]imidazole-2-carbonyl)piperazine-1-carboxylate (18j)

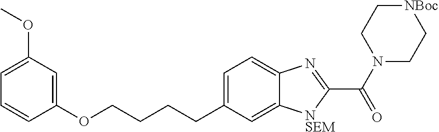 [SMILES: COc1cccc(OCCCCc2ccc3nc(C(=O)N4CCN(C(=O)OC(C)(C)C)CC4)n(COCC[Si](C)(C)C)c3c2)c1]

Synthesized according to General Procedure 20. Purified via column chromatography (20-30% ethyl acetate/hexanes). Yellow oil (86%, 213 mg). ¹H NMR (500 MHz, CDCl₃) δ 7.74 (d, J=8.3 Hz, 1H), 7.65 (s, 1H), 7.52 (d, J=8.3 Hz, 1H), 7.41 (d, J=1.4 Hz, 1H), 7.29-7.17 (m, 2H), 6.55-6.51 (m, 2H), 6.49 (d, J=2.4 Hz, 1H), 5.86-5.78 (m, 2H), 4.02-3.98 (m, 2H), 3.95-3.91 (m, 2H), 3.86-3.83 (m, 2H), 3.82 (s, 3H), 3.63-3.53 (m, 6H), 2.87 (q, J=7.3 Hz, 2H), 1.93-1.85 (m, 5H), 1.52 (s, 9H), 0.91 (ddd, J=9.5, 7.5, 2.3 Hz, 2H), −0.02 (d, J=1.5 Hz, 9H). ¹³C NMR (126 MHz, CDCl₃) δ 171.2, 160.8, 160.3, 160.3, 154.6, 144.5, 144.2, 141.8, 139.9, 139.5, 137.8, 135.1, 133.2, 129.8, 125.9, 124.6, 120.5, 119.9, 110.7, 110.2, 106.7, 106.6, 106.2, 106.1, 100.9, 100.9, 80.4, 73.6, 73.5, 67.7, 67.7, 66.5, 60.4, 55.3, 47.2, 42.3, 36.1, 35.6, 32.1, 28.9, 28.8, 28.4, 28.3, 26.2, 22.0, 21.1, 17.8, 14.2, −1.4. Material isolated as an approximately 1:1 ratio of regioisomers. HRMS: (ESI) [M+H]⁺ calc. for C₃₄H₅₁N₄O₆Si⁺ 639.3572, observed, 639.3560.

### tert-butyl 4-(6-(4-(3-(trifluoromethyl)phenoxy)butyl)-1-((2-(trimethylsilyl)ethoxy)methyl)-1H-benzo[d]imidazole-2-carbonyl)piperazine-1-carboxylate (18k)

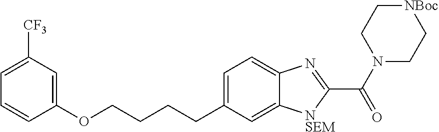 [SMILES: CC(C)(C)OC(=O)N1CCN(C(=O)c2nc3ccc(CCCCOc4cccc(C(F)(F)F)c4)cc3n2COCC[Si](C)(C)C)CC1]

Synthesized according to General Procedure 20. Purified via column chromatography (10-30% ethyl acetate/hexanes). Yellow oil (70%, 158 mg). ¹H NMR (500 MHz, CDCl₃) δ 7.70 (d, J=8.3 Hz, 1H), 7.61 (s, OH), 7.48 (d, J=8.4 Hz, OH), 7.40-7.33 (m, 2H), 7.26-7.15 (m, 2H), 7.10 (q, J=2.4 Hz, 1H), 7.04 (dd, J=8.3, 2.5 Hz, 1H), 5.77 (d, J=3.6 Hz, 2H), 4.01 (dt, J=5.9, 2.9 Hz, 2H), 3.89 (dt, J=7.7, 3.0 Hz, 2H), 3.80 (t, J=5.1 Hz, 2H), 3.62-3.50 (m, 6H), 2.85 (q, J=7.3 Hz, 2H), 1.86 (dt, J=11.9, 6.0 Hz, 5H), 1.48 (s, 10H), 0.87 (ddd, J=9.1, 7.3, 1.8 Hz, 2H), −0.07 (d, J=2.8 Hz, 9H). ¹³C NMR (126 MHz, CDCl₃) δ 160.3, 159.3, 159.3, 154.7, 144.7, 144.3, 141.9, 140.1, 139.5, 137.8, 135.2, 133.4, 130.0, 126.0, 124.7, 120.6, 120.0, 118.1, 117.4, 117.4, 117.3, 80.5, 73.7, 73.7, 71.0, 68.1, 68.1, 66.7, 47.4, 42.5, 36.1, 35.7, 32.2, 28.9, 28.7, 28.5, 28.4, 28.3, 26.3, 22.1, 18.0, −1.3. Material isolated as an approximately 1:1 ratio of regioisomers. HRMS: (ESI) [M+H]⁺ calc. for C₃₄H₄₈F₃N₄O₅Si⁺ 677.3341, observed, 677.3320.

### tert-butyl 4-(6-(6-phenoxyhexyl)-1H-benzo[d]imidazole-2-carbonyl)piperazine-1-carboxylate (17a)

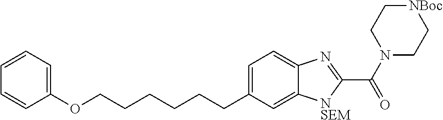 [SMILES: CC(C)(C)OC(=O)N1CCN(C(=O)c2nc3ccc(CCCCCCOc4ccccc4)cc3n2COCC[Si](C)(C)C)CC1]

Synthesized according to General Procedure 20. Purified via column chromatography (30-40% ethyl acetate/hexanes). Clear oil (60%, 60 mg). ¹H NMR (500 MHz, MeOD) δ 7.76 (d, J=8.6 Hz, 1H), 7.67 (d, J=1.3 Hz, 1H), 7.52 (dd, J=8.6, 1.5 Hz, 1H), 7.26-7.20 (m, 2H), 6.90-6.83 (m, 3H), 4.12 (t, J=5.3 Hz, 4H), 3.93 (t, J=6.3 Hz, 2H), 3.44 (t, J=5.3 Hz, 4H), 2.85 (t, J=7.6 Hz, 2H), 1.74 (dq, J=8.5, 6.4 Hz, 4H), 1.55-1.49 (m, 2H), 1.48-1.41 (m, 2H). ¹³C NMR (126 MHz, MeOD) δ 160.5*, 156.7*, 144.5*, 142.1*, 130.4*, 129.8*, 121.5*, 115.9*, 115.5*, 114.8*, 68.7, 44.2, 41.7, 36.9, 32.7, 30.3, 29.9, 27.0. HRMS: (ESI) [M+H]⁺ calc. for C₃₄H₄₇N₄O₆⁺ 607.3490 observed, 607.3486.

### tert-butyl 4-(6-(4-(m-tolyloxy)butyl)-1H-benzo[d]imidazole-2-carbonyl)piperazine-1-carboxylate (17e)

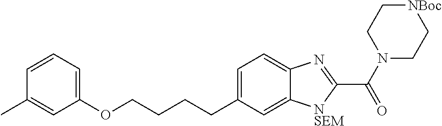 [SMILES: Cc1cccc(OCCCCc2ccc3nc(C(=O)N4CCN(C(=O)OC(C)(C)C)CC4)n(COCC[Si](C)(C)C)c3c2)c1]

Synthesized according to General Procedure 20. Purified via column chromatography (35-50% ethyl acetate/hexanes). Clear oil (93%, 287 mg). ¹H NMR (400 MHz, cdcl₃) δ 11.21-10.83 (m, 1H), 7.71 (d, J=8.4 Hz, 0.5H), 7.30 (s, 0.5H), 7.17-7.12 (m, 0.5H), 6.76-6.68 (m, 0.5H), 3.94-3.91 (m, 2H), 3.86 (h, J=3.6 Hz, 2H), 3.59 (q, J=5.9 Hz, 4H), 2.78 (t, J=7.6 Hz, 2H), 2.31 (s, 4H), 1.86-1.70 (m, 5H), 1.50 (s, 9H). ¹³C NMR (151 MHz, CDCl₃) δ 159.2*, 159.1*, 154.7, 141.7*, 139.6*, 129.3*, 126.6*, 124.7*, 121.5*, 121.5*, 121.4*, 120.6*, 115.4*, 111.4*, 80.5, 67.8, 67.7, 46.7, 43.2, 36.4, 36.1, 31.8, 31.7, 29.3, 29.3, 29.2, 28.5, 25.9, 25.8, 21.7. Material isolated as an approximately 1:1 ratio of rotamers. HRMS: (ESI) [M+H]⁺ calc. for C₂₈H₃₇N₄O₄⁺ 493.2809, observed, 493.2824.

### tert-butyl 4-(6-(4-(2-ethylphenoxy)butyl)-1H-benzo[d]imidazole-2-carbonyl)piperazine-1-carboxylate (17f)

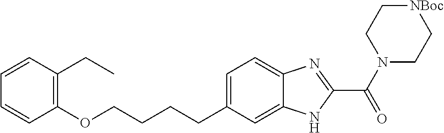 [SMILES: CCc1ccccc1OCCCCc1ccc2nc(C(=O)N3CCN(C(=O)OC(C)(C)C)CC3)[nH]c2c1]

Synthesized according to General Procedure 20. Purified via column chromatography (40-50% ethyl acetate/hexanes). White solid (72%, 83 mg). ¹H NMR (500 MHz, CDCl₃) δ 12.01-11.62 (m, 1H), 7.74 (d, J=8.4 Hz, 0.5H), 7.66 (s, 0.5H), 7.45 (d, J=8.3 Hz, 0.5H), 7.32 (s, 0.5H), 7.27-7.22 (m, 0.5H), 7.19 (dd, J=8.4, 1.6 Hz, 0.5H), 7.14 (t, J=7.2 Hz, 2H), 6.88 (t, J=7.4 Hz, 1H), 6.81 (d, J=8.1 Hz, 1H), 4.83-4.73 (m, 2H), 3.99 (t, J=5.7 Hz, 2H), 3.91 (s, 2H), 3.63 (s, 4H), 2.84 (q, J=6.6 Hz, 2H), 2.66 (qd, J=7.5, 3.7 Hz, 2H), 1.96-1.81 (m, 4H), 1.52 (s, 9H), 1.23-1.16 (m, 3H). ¹³C NMR (126 MHz, CDCl₃) δ 159.1*, 156.8*, 154.7, 145.0*, 144.7*, 143.5*, 141.7*, 139.9*, 137.5*, 133.3*, 132.7*, 131.5*, 129.0*, 129.0*, 126.8*, 126.4*, 124.5*, 120.7*, 120.4*, 120.0*, 111.7*, 111.0*, 80.4, 67.5, 46.8, 43.3, 36.0, 35.7, 29.0, 28.5, 28.3, 23.4, 14.3. Material isolated as an approximately 1:1 ratio of rotamers. HRMS: (ESI) [M+H]⁺ calc. for C₂₉H₃₉N₄O₄⁺ 507.2966, observed.

### tert-butyl 4-(6-(4-(3-ethylphenoxy)butyl)-1H-benzo[d]imidazole-2-carbonyl)piperazine-1-carboxylate (18g)

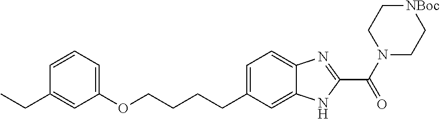 [SMILES: CCc1cccc(OCCCCc2ccc3nc(C(=O)N4CCN(C(=O)OC(C)(C)C)CC4)[nH]c3c2)c1]

Synthesized according to General Procedure 20. Purified via column chromatography (40-50% ethyl acetate/hexanes). Clear oil (93%, 258 mg). ¹H NMR (600 MHz, CDCl₃) δ 10.74-10.51 (m, 1H), 7.71 (d, J=8.4 Hz, 1H), 7.62 (s, 1H), 7.43 (d, J=8.3 Hz, OH), 7.31 (s, 1H), 7.23-7.15 (m, 2H), 6.78 (d, J=7.5 Hz, 1H), 6.76-6.73 (m, 1H), 6.70 (dt, J=8.1, 4.2 Hz, 1H), 4.76 (q, J=6.0 Hz, 2H), 3.97-3.92 (m, 2H), 3.83 (q, J=4.5 Hz, 2H), 3.63-3.55 (m, 4H), 2.78 (t, J=7.7 Hz, 2H), 2.63-2.57 (m, 2H), 1.85-1.79 (m, 2H), 1.75 (q, J=7.7 Hz, 2H), 1.51 (s, OH), 1.58-1.47 (m, 9H), 1.22 (td, J=7.6, 1.3 Hz, 3H). ¹³C NMR (151 MHz, CDCl₃) δ 159.2*, 146.0*, 143.7*, 141.7*, 140.5*, 132.8*, 129.3*, 126.7*, 124.7*, 120.6*, 120.3*, 120.3*, 114.4*, 111.5*, 110.9*, 80.5, 67.8, 67.7, 46.7, 43.2, 36.4, 36.1, 31.8, 31.7, 29.4, 29.3, 29.1, 28.5, 25.9, 25.8, 22.1, 15.7. Material isolated as an approximately 1:1 ratio of rotamers. HRMS: (ESI) [M+H]⁺ calc. for C₂₉H₃₉N₄O₄⁺ 507.2966, observed, 507.2960.

### tert-butyl 4-(6-(4-(3-isopropylphenoxy)butyl)-1H-benzo[d]imidazole-2-carbonyl)piperazine-1-carboxylate (17h)

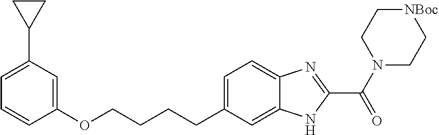 [SMILES: CC(C)(C)OC(=O)N1CCN(C(=O)c2nc3ccc(CCCCOc4cccc(C5CC5)c4)cc3[nH]2)CC1]

Synthesized according to General Procedure 20. Purified via column chromatography (40-50% ethyl acetate/hexanes). White solid (98%, 155 mg). ¹H NMR (600 MHz, MeOD) δ 7.66-7.35 (m, 2H), 7.18 (d, J=7.5 Hz, 1H), 7.13 (td, J=7.9, 1.5 Hz, 1H), 6.76 (d, J=7.6 Hz, 1H), 6.72 (t, J=2.1 Hz, 1H), 6.66 (dd, J=8.1, 2.6 Hz, 1H), 4.44-4.31 (m, 2H), 3.95 (t, J=5.9 Hz, 2H), 3.77 (t, J=5.3 Hz, 2H), 3.55 (t, J=5.4 Hz, 4H), 2.81 (h, J=7.0 Hz, 3H), 1.89-1.76 (m, 4H), 1.46 (dt, J=6.1, 3.4 Hz, 9H), 1.19 (dd, J=6.8, 2.8 Hz, 6H). ¹³C NMR (151 MHz, MeOD) δ 160.5*, 159.8*, 155.7, 151.4*, 145.3*, 129.9*, 119.5*, 113.6*, 112.1*, 81.6, 68.4, 47.5, 43.6, 36.6, 36.5, 34.9, 29.6, 29.1, 28.8, 24.4. Material isolated as an approximately 1:1 ratio of rotamers. HRMS: (ESI) [M+H]⁺ calc. for C₃₀H₄₁N₄O₄⁺ 521.3122, observed, 521.3111.

### tert-butyl 4-(6-(4-(3-cyclopropylphenoxy)butyl)-1H-benzo[d]imidazole-2-carbonyl)piperazine-1-carboxylate (17b)

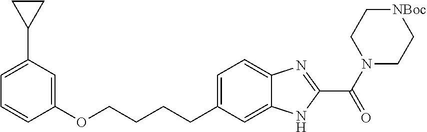 [SMILES: CC(C)(C)OC(=O)N1CCN(C(=O)c2nc3ccc(CCCCOc4cccc(C5CC5)c4)cc3[nH]2)CC1]

Synthesized according to General Procedure 20. Purified via column chromatography (20-40% ethyl acetate/hexanes). White solid (64%, 52 mg). ¹H NMR (400 MHz, cdcl₃) δ 10.61-10.38 (m, 1H), 7.72 (d, J=8.4 Hz, OH), 7.63 (s, OH), 7.45-7.41 (m, OH), 7.32 (dd, J=1.6, 0.8 Hz, OH), 7.23 (dd, J=8.3, 1.6 Hz, OH), 7.18 (dd, J=8.4, 1.6 Hz, OH), 7.14 (t, J=7.9 Hz, 1H), 6.70-6.64 (m, 2H), 6.59 (t, J=2.2 Hz, 1H), 4.76 (dt, J=7.4, 4.2 Hz, 2H), 3.96 (td, J=6.0, 2.8 Hz, 2H), 3.83 (s, 2H), 3.59 (dt, J=10.6, 5.5 Hz, 4H), 2.82 (t, J=7.0 Hz, 2H), 1.88-1.81 (m, 4H), 1.50 (d, J=0.6 Hz, 9H), 0.97-0.90 (m, 2H), 0.71-0.64 (m, 2H). ¹³C NMR (126 MHz, CDCl₃) δ 159.3*, 159.2*, 154.7, 145.8*, 145.0*, 144.7*, 143.6*, 141.7*, 140.0*, 137.6*, 129.3*, 126.5*, 124.6*, 120.7*, 120.1*, 118.1*, 112.2*, 111.2*, 80.5, 71.0, 67.6, 46.8, 43.3, 36.1, 35.8, 29.0, 28.9, 28.5, 28.4, 28.3, 26.3, 15.5, 9.3. Material isolated as an approximately 1:1 ratio of rotamers. HRMS: (ESI) [M+H]⁺ calc. for C₃₀H₃₉N₄O₄⁺ 519.2966, observed, 519.2987.

### tert-butyl 4-(6-(3-(3-cyclopropylphenoxy)propyl)-1H-benzo[d]imidazole-2-carbonyl)piperazine-1-carboxylate (171)

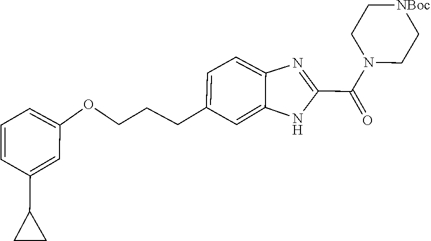 [SMILES: CC(C)(C)OC(=O)N1CCN(C(=O)c2nc3ccc(CCCOc4cccc(C5CC5)c4)cc3[nH]2)CC1]

Synthesized according to General Procedure 20. Purified via column chromatography (30-50% ethyl acetate/hexanes). Orange oil (53%, 224 mg). 1H NMR (500 MHz, CDCl₃) δ 11.86-11.56 (m, 1H), 7.72 (d, J=8.4 Hz, 0.5H), 7.65 (s, 0.5H), 7.43 (d, J=8.2 Hz, 0.5H), 7.32 (d, J=1.5 Hz, 0.5H), 7.24-7.11 (m, 2H), 6.70-6.59 (m, 3H), 4.76 (q, J=5.2 Hz, 2H), 3.95 (td, J=6.2, 3.7 Hz, 2H), 3.88 (q, J=5.3 Hz, 2H), 3.61 (dq, J=10.8, 3.5 Hz, 4H), 2.94 (td, J=7.6, 3.1 Hz, 2H), 2.19-2.09 (m, 2H), 1.85 (ttd, J=8.0, 5.0, 2.3 Hz, 1H), 1.51 (s, 9H), 1.26 (d, J=7.1 Hz, 1H), 0.93 (tdd, J=6.2, 4.4, 1.4 Hz, 2H), 0.68 (ddd, J=6.9, 3.1, 1.5 Hz, 2H). ¹³C NMR (126 MHz, CDCl₃) δ 159.2*, 159.1*, 154.7, 145.8*, 145.1*, 144.8*, 143.6*, 141.7*, 139.1*, 136.8*, 133.3*, 131.5*, 129.3*, 126.5*, 124.5*, 120.8*, 120.1*, 118.1*, 118.1*, 112.2*, 112.2*, 111.3*, 111.2*, 80.4, 66.6, 46.8, 43.2, 32.6, 32.4, 31.2, 28.5, 15.5, 9.3. Material isolated as an approximately 1:1 ratio of rotamers. HRMS: (ESI) [M+H]⁺ calc. for C₂₉H₃₇N₄O₄⁺ 505.2809, observed, 505.2819.

### tert-butyl 4-(6-(4-(3-(dimethylamino)phenoxy)butyl)-1H-benzo[d]imidazole-2-carbonyl)piperazine-1-carboxylate (18i)

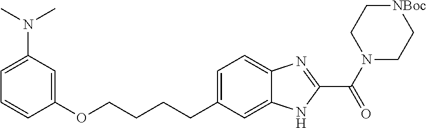 [SMILES: CN(C)c1cccc(OCCCCc2ccc3nc(C(=O)N4CCN(C(=O)OC(C)(C)C)CC4)[nH]c3c2)c1]

Synthesized according to General Procedure 20. Purified via column chromatography (40-60% ethyl acetate/hexanes). White solid (89%, 113 mg). ¹H NMR (500 MHz, CDCl₃) δ 11.88-11.28 (m, 1H), 7.76-7.59 (m, 1H), 7.46-7.28 (m, 0.5H), 7.25-7.15 (m, 0.5H), 7.12 (t, J=8.1 Hz, 1H), 6.38-6.32 (m, 1H), 6.30-6.23 (m, 2H), 4.77 (q, J=5.2 Hz, 2H), 4.04-3.96 (m, 2H), 3.93-3.85 (m, 2H), 3.67-3.56 (m, 4H), 2.91 (s, 6H), 2.85-2.77 (m, 2H), 1.90-1.78 (m, 4H), 1.50 (s, 9H). ¹³C NMR (126 MHz, CDCl₃) δ 160.2*, 159.0*, 154.7, 152.1*, 144.7*, 143.5*, 141.7*, 139.9*, 137.5*, 133.2*, 131.4*, 129.8*, 129.8*, 126.5*, 124.5*, 120.7*, 120.0*, 111.6*, 111.1*, 105.8*, 102.1*, 99.8*, 80.4, 80.4, 67.6, 67.6, 46.7, 43.3, 40.7, 29.0, 28.5, 28.3. Material isolated as an approximately 1:1 ratio of rotamers. HRMS: (ESI) [M+H]⁺ calc. for C₂₉H₄₀N₅O₄⁺ 522.3075, observed, 522.3098.

### tert-butyl 4-(6-(4-(3-methoxyphenoxy)butyl)-1H-benzo[d]imidazole-2-carbonyl)piperazine-1-carboxylate (17j)

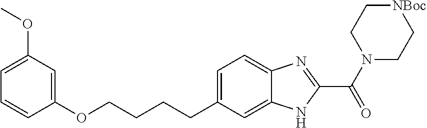 [SMILES: COc1cccc(OCCCCc2ccc3nc(C(=O)N4CCN(C(=O)OC(C)(C)C)CC4)[nH]c3c2)c1]

Synthesized according to General Procedure 20. Purified via column chromatography (30-50% ethyl acetate/hexanes). White solid (93%, 132 mg). ¹H NMR (500 MHz, CDCl₃) δ 11.95-11.51 (m, 1H), 7.71 (d, J=8.4 Hz, 0.5H), 7.63 (s, 0.5H), 7.42 (d, J=8.3 Hz, 0.5H), 7.30 (s, 0.5H), 7.21 (d, J=8.3 Hz, 0.5H), 7.19-7.13 (m, 2H), 6.50-6.43 (m, 3H), 4.81-4.72 (m, 2H), 3.97-3.91 (m, 2H), 3.92-3.87 (m, 2H), 3.77 (s, 3H), 3.66-3.55 (m, 4H), 2.85-2.78 (m, 2H), 1.88-1.78 (m, 4H), 1.50 (s, 9H). ¹³C NMR (126 MHz, CDCl₃) δ 160.9*, 160.4*, 159.0*, 154.7, 145.0*, 144.7*, 143.5*, 141.7*, 139.8*, 137.5*, 133.3*, 131.4*, 129.9*, 129.9*, 126.4*, 124.5*, 120.7*, 120.0*, 111.6*, 111.1*, 106.7*, 106.2*, 101.0*, 80.4, 67.8, 55.3, 46.8, 43.3, 36.0, 35.8, 28.9, 28.8, 28.5, 28.3, 28.3. Material isolated as an approximately 1:1 ratio of rotamers. HRMS: (ESI) [M+H]⁺ calc. for C₂₈H₃₇N₄O₅⁺ 509.2758, observed, 509.2784.

### tert-butyl 4-(6-(4-(3-(trifluoromethyl)phenoxy)butyl)-1H-benzo[d]imidazole-2-carbonyl)piperazine-1-carboxylate (17k)

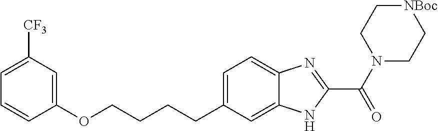 [SMILES: CC(C)(C)OC(=O)N1CCN(C(=O)c2nc3ccc(CCCCOc4cccc(C(F)(F)F)c4)cc3[nH]2)CC1]

Synthesized according to General Procedure 20. Purified via column chromatography (30-50% ethyl acetate/hexanes). White solid (70%, 158 mg). ¹H NMR (500 MHz, CDCl₃) δ 12.02-11.65 (m, 1H), 7.72 (d, J=8.4 Hz, 0.5H), 7.63 (s, 0.5H), 7.43 (d, J=8.3 Hz, 0.5H), 7.34 (dd, J=16.8, 8.8 Hz, 1.5H), 7.24-7.19 (m, 0.5H), 7.17 (d, J=7.9 Hz, 1.5H), 7.09 (t, J=2.1 Hz, 1H), 7.02 (dd, J=8.4, 2.5 Hz, 1H), 4.79-4.73 (m, 2H), 4.01-3.96 (m, 2H), 3.94-3.87 (m, 2H), 3.64-3.57 (m, 4H), 2.85-2.78 (m, 2H), 1.90-1.79 (m, 4H), 1.50 (s, 9H). ¹³C NMR (126 MHz, CDCl₃) δ 159.2*, 159.1*, 154.7, 145.1*, 144.7*, 143.6*, 141.7*, 139.7*, 137.3*, 133.3*, 132.2*, 131.8 (q, J=32 Hz), 130.0*, 126.4*, 124.4*, 124.1 (d, J=271 Hz), 120.8*, 120.0*, 118.0*, 117.2 (q, J=3.7 Hz), 111.7*, 111.2 (q, J=3.7 Hz), 111.1*, 80.5, 68.1, 46.8, 43.3, 36.0, 35.7, 28.5, 28.2. HRMS: (ESI) [M+H]⁺ calc. for C₂₈H₃₄F₃N₄O₄⁺ 547.2527, observed, 547.2521.

### (6-(6-phenoxyhexyl)-1H-benzo[d]imidazol-2-yl)(piperazin-1-yl)methanone hydrochloride (18a)

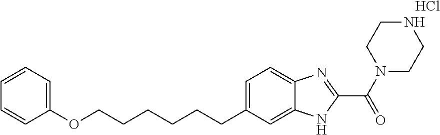 [SMILES: Cl ; O=C(c1nc2ccc(CCCCCCOc3ccccc3)cc2[nH]1)N1CCNCC1]

Synthesized according to General Procedure 4. Purified via trituration with diethyl ether. White solid (89%, 39 mg). ¹H NMR (500 MHz, CD₃OD) δ 7.76 (d, J=8.6 Hz, 1H), 7.67 (d, J=1.3 Hz, 1H), 7.52 (dd, J=8.6, 1.5 Hz, 1H), 7.26-7.20 (m, 2H), 6.90-6.83 (m, 3H), 4.12 (t, J=5.3 Hz, 4H), 3.93 (t, J=6.3 Hz, 2H), 3.44 (t, J=5.3 Hz, 5H), 2.85 (t, J=7.6 Hz, 2H), 1.75 (dtd, J=15.1, 7.5, 5.3 Hz, 4H), 1.55-1.49 (m, 2H), 1.48-1.41 (m, 2H). ¹³C NMR (126 MHz, CD₃OD) δ 160.5, 156.7, 144.5, 142.1, 130.4, 129.8, 121.5, 115.9, 115.5, 114.8, 68.7, 44.2, 41.7, 36.9, 32.7, 30.3, 29.9, 27.0. HRMS: (ESI) [M+H]⁺ calc. for C₂₄H₃₁N₄O₂⁺ 407.2442 observed, 407.2452.

### piperazin-1-yl(6-(4-(m-tolyloxy)butyl)-1H-benzo[d]imidazol-2-yl)methanone hydrochloride (18m)

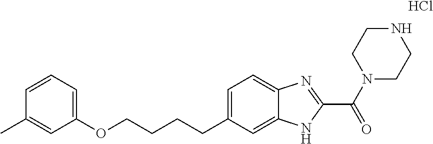 [SMILES: Cl ; Cc1cccc(OCCCCc2ccc3nc(C(=O)N4CCNCC4)[nH]c3c2)c1]

Synthesized according to General Procedure 4. Purified via trituration with diethyl ether. White solid (89%, 222 mg). ¹H NMR (600 MHz, CD₃OD) δ 7.80 (d, J=8.5 Hz, 1H), 7.70 (s, 1H), 7.55 (dd, J=8.6, 1.4 Hz, 1H), 7.09 (t, J=7.8 Hz, 1H), 6.71-6.61 (m, 3H), 4.10 (t, J=5.2 Hz, 4H), 3.91 (t, J=6.3 Hz, 2H), 3.46 (t, J=5.3 Hz, 4H), 2.86 (t, J=7.6 Hz, 2H), 2.27 (s, 3H), 1.83-1.73 (m, 4H), 1.57-1.49 (m, 2H). ¹³C NMR (151 MHz, CD₃OD) δ 160.4, 156.2, 144.7, 141.6, 140.4, 132.7, 130.8, 130.1, 122.3, 116.1, 115.6, 114.7, 112.3, 68.5, 44.1, 41.6, 36.9, 32.5, 30.2, 26.7, 21.6. HRMS: (ESI) [M+H]⁺ calc. for C₂₃H₂₉N₄O₂⁺ 393.2285, observed, 393.2153.

### (6-(4-(2-ethylphenoxy)butyl)-1H-benzo[d]imidazol-2-yl)(piperazin-1-yl)methanone hydrochloride (18f)

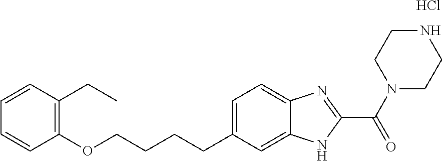 [SMILES: Cl ; CCc1ccccc1OCCCCc1ccc2nc(C(=O)N3CCNCC3)[nH]c2c1]

Synthesized according to General Procedure 4. Purified via trituration with diethyl ether. White solid (72%, 52 mg). ¹H NMR (500 MHz, CD₃OD) δ 7.79 (d, J=8.6 Hz, 1H), 7.70 (s, 1H), 7.58 (dd, J=8.6, 1.5 Hz, 1H), 7.12-7.07 (m, 2H), 6.87-6.79 (m, 2H), 4.11 (t, J=5.4 Hz, 4H), 4.01 (t, J=6.0 Hz, 2H), 3.46-3.41 (m, 4H), 2.95 (t, J=7.5 Hz, 2H), 2.60 (q, J=7.5 Hz, 2H), 1.99-1.84 (m, 4H), 1.14 (t, J=7.5 Hz, 3H). ¹³C NMR (126 MHz, CD₃OD) δ 156.6, 155.2, 143.0, 140.7, 132.2, 132.0, 130.3, 128.6, 128.4, 126.5, 120.0, 114.5, 113.4, 110.8, 67.0, 42.8, 35.3, 28.6, 28.0, 23.1, 13.6. HRMS: (ESI) [M+H]⁺ calc. for C₂₄H₃₁N₄O₂⁺ 407.2442, observed, 407.2458.

### (6-(4-(3-ethylphenoxy)butyl)-1H-benzo[d]imidazol-2-yl)(piperazin-1-yl)methanone hydrochloride (18g)

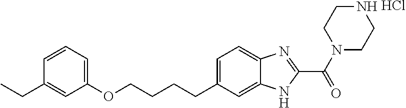 [SMILES: Cl ; CCc1cccc(OCCCCc2ccc3nc(C(=O)N4CCNCC4)[nH]c3c2)c1]

Synthesized according to General Procedure 4. Purified via trituration with diethyl ether. White solid (85%, 36 mg). ¹H NMR (600 MHz, CD₃OD) δ 7.76 (d, J=8.5 Hz, 1H), 7.66 (d, J=1.5 Hz, 1H), 7.53 (dd, J=8.5, 1.5 Hz, 1H), 7.13 (t, J=7.8 Hz, 1H), 6.76-6.73 (m, 1H), 6.70 (t, J=2.1 Hz, 1H), 6.69-6.65 (m, 1H), 3.94 (t, J=6.3 Hz, 2H), 3.45-3.40 (m, 4H), 2.89 (t, J=7.6 Hz, 2H), 2.58 (q, J=7.6 Hz, 2H), 1.85-1.76 (m, 4H), 1.58-1.51 (m, 2H), 1.20 (t, J=7.6 Hz, 3H). HRMS: (ESI) [M+H]⁺ calc. for C₂₄H₃₁N₄O₂⁺ 407.2442, observed, 407.2458.

### (6-(4-(3-isopropylphenoxy)butyl)-1H-benzo[d]imidazol-2-yl)(piperazin-1-yl)methanone hydrochloride (18h)

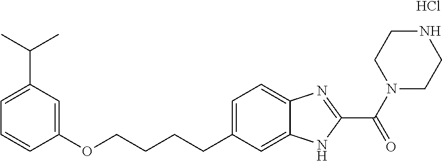 [SMILES: Cl ; CC(C)c1cccc(OCCCCc2ccc3nc(C(=O)N4CCNCC4)[nH]c3c2)c1]

Synthesized according to General Procedure 4. Purified via trituration with diethyl ether. White solid (79%, 115 mg). ¹H NMR (500 MHz, CD₃OD) δ 7.80 (d, J=8.6 Hz, 1H), 7.72 (s, 1H), 7.59 (dd, J=8.6, 1.5 Hz, 1H), 7.14 (t, J=7.9 Hz, 1H), 6.77 (dt, J=7.7, 1.3 Hz, 1H), 6.75-6.72 (m, 1H), 6.68 (ddd, J=8.2, 2.6, 0.9 Hz, 1H), 4.10 (t, J=5.4 Hz, 4H), 3.99 (t, J=6.1 Hz, 2H), 3.44 (t, J=5.3 Hz, 4H), 2.93 (t, J=7.5 Hz, 2H), 2.83 (p, J=6.9 Hz, 1H), 1.94-1.79 (m, 4H), 1.21 (d, J=6.9 Hz, 6H). ¹³C NMR (126 MHz, CD₃OD) δ 160.5, 156.4, 151.7, 144.5, 141.9, 133.1, 131.3, 130.3, 130.0, 119.7, 115.8, 114.8, 113.8, 112.6, 68.5, 44.2, 36.7, 35.4, 29.9, 29.3, 24.4. HRMS: (ESI) [M+H]⁺ calc. for C₂₅H₃₃N₄O₂⁺ 421.2598, observed, 421.2591.

### (6-(4-(3-cyclopropylphenoxy)butyl)-1H-benzo[d]imidazol-2-yl)(piperazin-1-yl)methanone hydrochloride (18b)

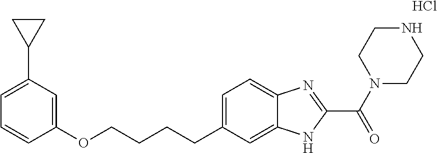 [SMILES: Cl ; O=C(c1nc2ccc(CCCCOc3cccc(C4CC4)c3)cc2[nH]1)N1CCNCC1]

Synthesized according to General Procedure 4. Purified via trituration with diethyl ether. White solid (73%, 51 mg). ¹H NMR (500 MHz, CD₃OD) δ 7.78 (d, J=8.5 Hz, 1H), 7.69 (s, 1H), 7.54 (d, J=8.5 Hz, 1H), 7.08 (t, J=8.0 Hz, 1H), 6.66-6.55 (m, 3H), 4.13 (t, J=5.1 Hz, 4H), 3.96 (t, J=6.1 Hz, 2H), 3.44 (t, J=5.2 Hz, 4H), 2.91 (t, J=7.5 Hz, 2H), 1.93-1.76 (m, 5H), 0.96-0.87 (m, 2H), 0.67-0.59 (m, 2H). ¹³C NMR (126 MHz, CD₃OD) δ 160.5, 156.7, 147.0, 144.2, 142.1, 133.5, 131.8, 130.2, 129.8, 118.8, 115.9, 114.9, 112.9, 112.3, 68.4, 44.2, 36.6, 29.9, 29.3, 16.2, 9.6. HRMS: (ESI) [M+H]⁺ calc. for C₂₅H₃₁N₄O₂⁺ 419.2442 observed, 419.2448.

### (6-(3-(3-cyclopropylphenoxy)propyl)-1H-benzo[d]imidazol-2-yl)(piperazin-1-yl)methanone (181)

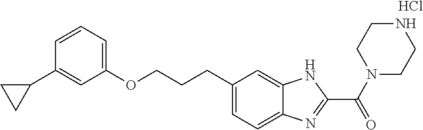 [SMILES: Cl ; O=C(c1nc2ccc(CCCOc3cccc(C4CC4)c3)cc2[nH]1)N1CCNCC1]

Synthesized according to General Procedure 4. Purified via trituration with diethyl ether. White solid (88%, 100 mg). ¹H NMR (400 MHz, CD₃OD) δ 7.79 (dd, J=8.5, 0.7 Hz, 1H), 7.70 (s, 1H), 7.57 (dd, J=8.6, 1.5 Hz, 1H), 7.10 (t, J=7.9 Hz, 1H), 6.68-6.57 (m, 3H), 4.11 (t, J=5.4 Hz, 4H), 3.95 (t, J=6.1 Hz, 2H), 3.46-3.42 (m, 4H), 3.04 (dd, J=8.3, 6.7 Hz, 2H), 2.21-2.11 (m, 2H), 1.84 (tt, J=8.4, 5.1 Hz, 1H), 0.96-0.87 (m, 2H), 0.65-0.60 (m, 2H). ¹³C NMR (151 MHz, CD₃OD) δ 160.4, 157.3, 147.0, 142.9, 142.7, 134.3, 132.8, 130.2, 129.3, 118.9, 116.2, 115.2, 112.9, 112.2, 67.4, 44.3, 41.6, 33.3, 32.2, 16.2, 9.7.

### (6-(4-(3-(dimethylamino)phenoxy)butyl)-1H-benzo[d]imidazol-2-yl)(piperazin-1-yl)methanone hydrochloride (18i)

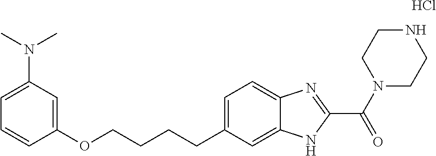 [SMILES: Cl ; CN(C)c1cccc(OCCCCc2ccc3nc(C(=O)N4CCNCC4)[nH]c3c2)c1]

Synthesized according to General Procedure 4. Purified via trituration with diethyl ether.). ¹H NMR (400 MHz, CD₃OD) δ 7.81 (dd, J=8.5, 2.6 Hz, 1H), 7.73 (d, J=5.3 Hz, 1H), 7.62-7.56 (m, 1H), 7.47 (td, J=8.2, 3.0 Hz, 1H), 7.27 (q, J=2.5 Hz, 1H), 7.21 (dt, J=8.2, 2.5 Hz, 1H), 7.08 (dt, J=8.5, 2.5 Hz, 1H), 4.10-4.03 (m, 6H), 3.63-3.58 (m, 2H), 3.45-3.41 (m, 4H), 3.29-3.27 (m, 4H), 2.93 (dt, J=7.5, 3.7 Hz, 2H), 1.89 (d, J=14.5 Hz, 4H). ¹³C NMR (151 MHz, CD₃OD) δ 161.8, 156.2, 145.0, 144.5, 141.7, 132.8, 132.4, 130.9, 130.2, 128.8, 116.0, 115.7, 114.8, 69.4, 68.1, 47.2, 44.1, 36.5, 29.6, 29.1, 15.5. HRMS: (ESI) [M+H]⁺ calc. for C₂₄H₃₂N₅O₂⁺ 422.2551, observed, 422.2573.

### (6-(4-(3-methoxyphenoxy)butyl)-1H-benzo[d]imidazol-2-yl)(piperazin-1-yl)methanone hydrochloride (18j)

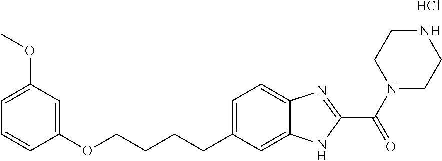 [SMILES: Cl ; COc1cccc(OCCCCc2ccc3nc(C(=O)N4CCNCC4)[nH]c3c2)c1]

Synthesized according to General Procedure 4. Purified via trituration with diethyl ether. White solid (98%, 113 mg). ¹H NMR (400 MHz, CD₃OD) δ 7.77 (dd, J=8.6, 0.8 Hz, 1H), 7.69 (s, 1H), 7.55 (dd, J=8.6, 1.5 Hz, 1H), 7.09 (t, J=8.2 Hz, 1H), 6.46-6.41 (m, 2H), 6.39 (t, J=2.4 Hz, 1H), 4.10-4.05 (m, 5H), 3.94 (t, J=6.1 Hz, 2H), 3.71 (s, 3H), 3.47-3.38 (m, 5H), 2.90 (t, J=7.4 Hz, 2H), 1.91-1.74 (m, 4H). ¹³C NMR (151 MHz, CD₃OD) δ 162.3, 161.6, 156.4, 144.4, 141.9, 133.1, 131.3, 130.7, 130.6, 130.6, 130.0, 115.8, 114.8, 101.9, 68.5, 55.6, 44.2, 41.6, 36.6, 29.8, 29.2. HRMS: (ESI) [M+H]⁺ calc. for C₂₃H₂₉N₄O₃⁺ 409.2234, observed, 409.2222.

### piperazin-1-yl(6-(4-(3-(trifluoromethyl)phenoxy)butyl)-1H-benzo[d]imidazol-2-yl)methanone hydrochloride (18k)

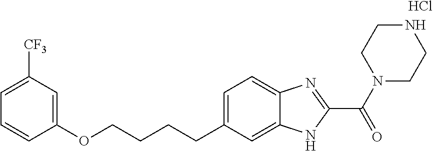 [SMILES: Cl ; O=C(c1nc2ccc(CCCCOc3cccc(C(F)(F)F)c3)cc2[nH]1)N1CCNCC1]

Synthesized according to General Procedure 4. Purified via trituration with diethyl ether. White solid (98%, 136 mg). ¹H NMR (600 MHz, MeOD) δ 7.81 (d, J=8.6 Hz, 1H), 7.73 (s, 1H), 7.59 (dd, J=8.6, 1.5 Hz, 1H), 7.44 (t, z 8.0 Hz, 1H), 7.21-7.10 (m, 3H), 4.10 (t, J=5.4 Hz, 4H), 4.06 (t, J=6.0 Hz, 2H), 3.45 (t, J=5.4 Hz, 4H), 2.94 (t, J=7.4 Hz, 2H), 1.94-1.83 (m, 4H). ¹³C NMR (151 MHz, MeOD) δ 160.7, 156.3, 144.4, 141.8, 132.9, 132.7 (q, J=32 Hz) 131.4, 131.1, 130.0, 125.5 (d, J=271 Hz), 119.2, 118.0 (q, J=3.7 Hz), 115.8, 114.8, 112.1 (q, J=3.7 Hz), 69.0, 68.1, 44.1, 36.6, 29.7, 29.1. HRMS: (ESI) [M+H]⁺ calc. for C₂₃H₂₆F₃N₄O₂⁺ 447.2002, observed, 447.1979.

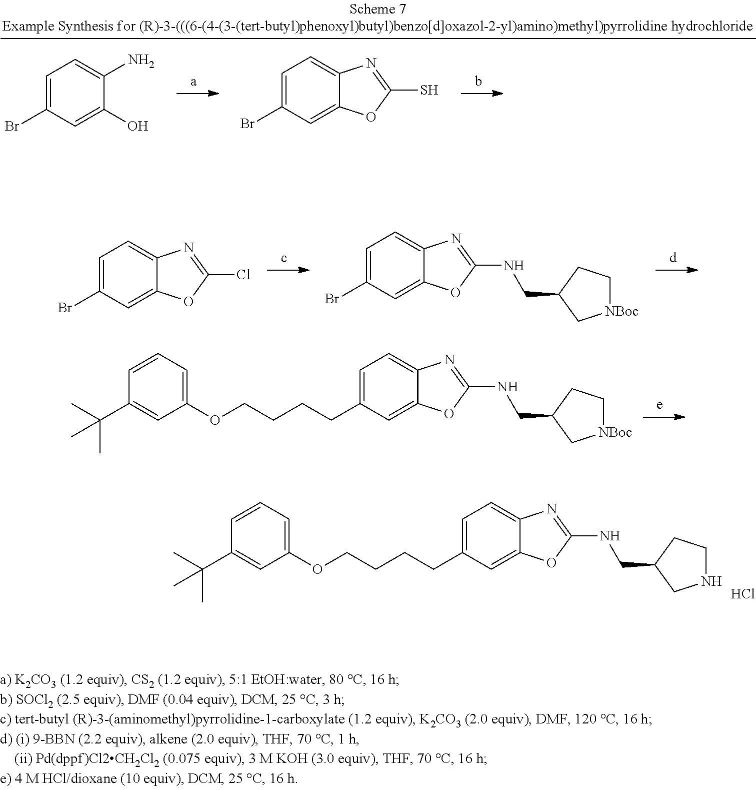

### 6-bromobenzo[d]oxazole-2-thiol, 9

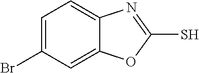 [SMILES: Sc1nc2ccc(Br)cc2o1]

To a stirring solution of potassium carbonate (2.7 g, 1.2 eq., 19 mmol) and 2-amino-5-bromophenol (3.0 g, 1.0 eq., 16 mmol) in ethanol (44 mL) and water (8.9 mL) was added carbon disulfide (1.5 g, 1.2 mL, 1.2 eq., 19 mmol). The reaction was heated under reflux for 16 hours, monitoring by TLC. The reaction mixture was diluted with water, and acetic acid was added dropwise to precipitate a white solid. The reaction was filtered, and the precipitate was redissolved in ethyl acetate, then concentrated in vacuo, to give 6-bromobenzo[d]oxazole-2-thiol (3.31 g, 14.4 mmol, 90%) as an amorphous off-white solid. ¹H NMR (500 MHz, d₆-DMSO) δ 14.04 (s, 1H), 7.86 (d, J=1.7 Hz, 1H), 7.48 (dd, J=8.3, 1.8 Hz, 1H), 7.19 (d, J=8.4 Hz, 1H). ¹³C NMR (126 MHz, d₆-DMSO) δ 180.7, 149.2, 131.4, 128.4, 116.0, 113.7, 112.3.

### 6-bromo-2-chlorobenzo[d]oxazole, 10

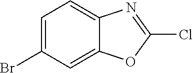 [SMILES: Clc1nc2ccc(Br)cc2o1]

To a stirring solution of 6-bromobenzo[d]oxazole-2-thiol 9 (0.45 g, 1 Eq, 2.0 mmol) in dichloromethane (6 mL) was added sequentially sulfurous dichloride (0.58 g, 0.36 mL, 2.5 Eq, 4.9 mmol) and N,N-dimethylformamide (5.7 mg, 6.1 μL, 0.04 Eq, 78 mol) under a nitrogen atmosphere. The reaction was stirred at room temperature for 3 hours, monitoring by TLC. The reaction mixture was quenched with water and extracted with ethyl acetate (3×5 mL). The organic extracts were combined, washed with brine (3×10 mL), dried with sodium sulfate, filtered, and concentrated in vacuo. The residue was purified by flash column chromatography (Teledyne ISCO, silica 10 g, Ø=2.5 cm, hexane/ethyl acetate 0-30%) to give 6-bromo-2-chlorobenzo[d]oxazole (0.682 g, 2.93 mmol, 75%) as a yellow amorphous solid. ¹H NMR (600 MHz, CDCl₃) δ 7.69 (dd, J=1.7, 0.5 Hz, 1H), 7.54 (dd, J=8.5, 0.5 Hz, 1H), 7.50 (dd, J=8.5, 1.8 Hz, 1H). ¹³C NMR (151 MHz, CDCl₃) δ 151.8, 151.4, 140.2, 128.6, 120.7, 118.6, 113.9.

### tert-butyl (R)-3-(((6-bromobenzo[d]oxazol-2-yl)amino)methyl)pyrrolidine-1-carboxylate

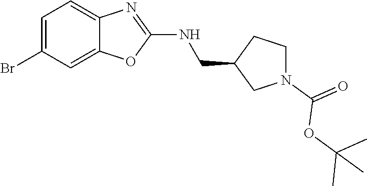 [SMILES: CC(C)(C)OC(=O)N1CC[C@H](CNc2nc3ccc(Br)cc3o2)C1]

To a stirred solution of 6-bromo-2-chlorobenzo[d]oxazole 10 (0.66 g, 1.0 Eq, 2.8 mmol) in N,N-dimethylformamide (9.5 mL) was added potassium carbonate (0.78 g, 2.0 Eq, 5.7 mmol) and tert-butyl (R)-3-(aminomethyl)pyrrolidine-1-carboxylate (0.68 g, 1.2 Eq, 3.4 mmol). The reaction was heated to 120° C. for 16 hours, monitoring by TLC. The reaction mixture was diluted with ethyl acetate (20 mL), washed with saturated lithium bromide solution (3×20 mL), dried with sodium sulfate, filtered, and concentrated in vacuo. The brown residue was purified by flash column chromatography (Teledyne ISCO, silica 25 g, Ø=2.5 cm, hexane/ethyl acetate 0→100%) to give tert-butyl (R)-3-(((6-bromobenzo[d]oxazol-2-yl)amino)methyl)pyrrolidine-1-carboxylate (0.68 g, 1.7 mmol, 60%) as a pale yellow amorphous solid. ¹H NMR (600 MHz, CDCl₃) δ 7.39 (d, J=6.8 Hz, 1H), 7.30-7.27 (m, 1H), 7.20 (dd, J=8.4, 3.1 Hz, 1H), 5.50 (d, J=76.2 Hz, 1H), 3.51 (dddd, J=54.4, 26.0, 11.7, 6.9 Hz, 4H), 3.34 (ddt, J=26.1, 10.7, 7.6 Hz, 1H), 3.13 (ddd, J=53.7, 10.8, 6.8 Hz, 1H), 2.68-2.55 (m, 1H), 2.09-2.05 (m, 1H), 1.74-1.63 (m, 1H), 1.45 (s, 9H). ¹³C NMR (126 MHz, CDCl₃) δ 162.2, 154.5, 148.9, 142.1, 127.0, 117.1, 112.9, 112.3, 79.4, 49.4, 49.0, 45.5, 45.4, 45.2, 44.9, 38.8, 38.0, 29.2, 28.5, 28.3.

### (S)-3-(((tert-butoxycarbonyl)(6-hydroxybenzo[d]oxazol-2-yl)amino)methyl)pyrrolidine-1-carboxylate

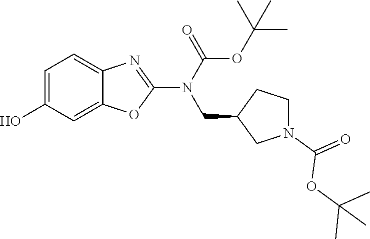 [SMILES: CC(C)(C)OC(=O)N1CC[C@H](CN(C(=O)OC(C)(C)C)c2nc3ccc(O)cc3o2)C1]

To a stirring solution of tert-butyl (S)-3-(((tert-butoxycarbonyl)(6-(4,4,5,5-tetramethyl-1,3,2-dioxaborolan-2-yl)benzo[d]oxazol-2-yl)amino)methyl)pyrrolidine-1-carboxylate (0.31 g, 1.0 eq., 0.57 mmol) in THE (0.50 M) was added sodium perborate monohydrate (0.17 g, 3.0 eq., 1.7 mmol) and water (0.57 mL). The reaction was stirred at room temperature, monitoring by TLC, then diluted with water (10 mL) and extracted with ethyl acetate (3×10 mL). The organic extracts were combined, washed with brine (2×15 mL), dried with sodium sulfate, filtered, and concentrated in vacuo. The residue was purified by flash column chromatography (Teledyne ISCO, silica 25 g, Ø=2.5 cm, hexane/ethyl acetate 0→100%) to give tert-butyl (S)-3-(((tert-butoxycarbonyl)(6-hydroxybenzo[d]oxazol-2-yl)amino)methyl)pyrrolidine-1-carboxylate (0.212 g, 0.489 mmol, 86%) as an orange oil. ¹H NMR (500 MHz, CDCl₃) δ 7.75 (s, 1H), 7.36 (dd, J=10.7, 8.5 Hz, 1H), 6.95 (dd, J=13.1, 2.3 Hz, 1H), 6.84-6.78 (m, 1H), 3.98-3.86 (m, 2H), 3.54-3.38 (m, 2H), 3.34-3.23 (m, 1H), 3.15-3.03 (m, 1H), 2.62 (dp, J=14.3, 7.2 Hz, 1H), 2.00-1.90 (m, 1H), 1.73-1.60 (m, 1H), 1.51 (d, J=8.0 Hz, 9H), 1.43 (d, J=10.8 Hz, 9H). ¹³C NMR (126 MHz, CDCl₃) δ 156.3, 154.7, 154.4, 152.1, 152.0, 149.9, 133.4, 118.9, 118.9, 112.9, 97.6, 83.4, 83.3, 79.6, 60.5, 50.5, 50.4, 49.3, 48.9, 45.3, 44.9, 38.5, 37.6, 29.0, 28.5, 28.4, 28.0. HRMS (ESI⁺) found 456.2102 (C₂₂H₃₁N₃O₆), required 456.2105 [M+Na]⁺.

### tert-Butyl (R)-3-(((6-(4-(3-(tert-butyl)phenoxy)butyl)benzo[d]oxazol-2-yl)amino)methyl)pyrrolidine-1-carboxylate (20a)

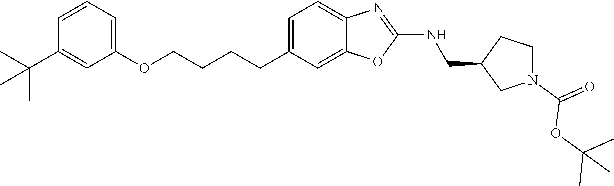 [SMILES: CC(C)(C)OC(=O)N1CC[C@H](CNc2nc3ccc(CCCCOc4cccc(C(C)(C)C)c4)cc3o2)C1]

Synthesized according to General Procedure 12. 0.100 g, 64%, yellow oil. ¹H NMR (500 MHz, CDCl₃) δ 7.28-7.24 (m, 1H), 7.20 (td, J=7.9, 1.0 Hz, 1H), 7.10 (s, 1H), 7.01 (d, J=8.1 Hz, 1H), 6.99-6.95 (m, 1H), 6.92 (t, J=2.2 Hz, 1H), 6.73-6.67 (m, 1H), 5.20 (s, 1H), 3.96 (t, J=5.9 Hz, 2H), 3.48 (d, J=29.2 Hz, 4H), 3.34 (q, J=9.7 Hz, 1H), 3.21-3.05 (m, 1H), 2.73 (t, J=6.7 Hz, 2H), 2.67-2.57 (m, 1H), 2.10-2.05 (m, 1H), 1.85-1.78 (m, 4H), 1.77-1.64 (m, 1H), 1.45 (s, 9H), 1.30 (s, 9H). ¹³C NMR (126 MHz, CDCl₃) δ 161.6, 158.8, 154.5, 152.9, 148.7, 140.7, 135.7, 128.9, 124.2, 117.7, 115.9, 112.6, 110.4, 108.7, 79.3, 67.6, 49.4, 48.9, 45.6, 45.5, 45.2, 44.9, 38.9, 38.0, 35.6, 34.7, 31.6, 31.3, 29.2, 28.8, 28.5, 28.4, 22.6, 14.1. HRMS (ESI⁺) found 522.3334 (C₃₁H₄₄N₃O₄), required 522.3332 [M+H]⁺.

### tert-Butyl (R)-3-(((6-(6-(2,6-difluorophenoxy)hexyl)benzo[d]oxazol-2-yl)amino)methyl)pyrrolidine-1-carboxylate (20b)

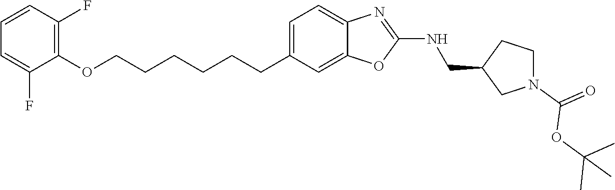 [SMILES: CC(C)(C)OC(=O)N1CC[C@H](CNc2nc3ccc(CCCCCCOc4c(F)cccc4F)cc3o2)C1]

Synthesized according to General Procedure 12. 0.118 g, 80%, yellow oil. ¹H NMR (500 MHz, CDCl₃) δ 7.24 (s, 1H), 7.08 (s, 1H), 6.99 (d, J=8.0 Hz, 1H), 6.96-6.83 (m, 3H), 5.18 (s, 1H), 4.10 (t, J=6.5 Hz, 2H), 3.60-3.40 (m, 4H), 3.41-3.28 (m, 1H), 3.21-3.05 (m, 1H), 2.66 (t, J=7.7 Hz, 2H), 2.63-2.56 (m, 1H), 2.10-2.03 (m, 1H), 1.79-1.60 (m, 5H), 1.56-1.48 (m, 2H), 1.46 (s, 9H), 1.44-1.35 (m, 2H). ¹³C NMR (126 MHz, CDCl₃) δ 161.5, 156.3 (dd, J=248.1, 5.8 Hz), 154.5, 148.6, 140.2, 136.3, 135.8 (t, J=14.1 Hz), 124.2, 122.5 (t, J=9.3 Hz), 115.8, δ 112.1 (dd, J=17.1, 5.6 Hz), 108.7, 79.3, 74.7, 74.7, 74.7, 60.4, 49.4, 48.9, 45.6, 45.5, 45.2, 44.9, 35.8, 31.8, 29.9, 28.8, 28.5, 25.5, 14.2. ¹⁹F NMR (376 MHz, CDCl₃) δ −128.2. HRMS (ESI⁺) found 530.2825 (C₂₉H₃₈F₂N₃O₄), required 530.2830 [M+H]⁺.

### tert-Butyl (R)-3-(((6-(6-(3,5-difluorophenoxy)hexyl)benzo[d]oxazol-2-yl)amino)methyl)pyrrolidine-1-carboxylate (20c)

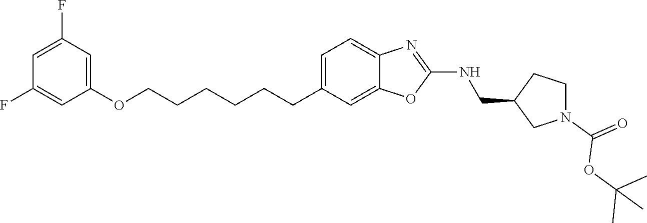 [SMILES: CC(C)(C)OC(=O)N1CC[C@H](CNc2nc3ccc(CCCCCCOc4cc(F)cc(F)c4)cc3o2)C1]

Synthesized according to General Procedure 12. 0.12 g, 82%, yellow oil. ¹H NMR (500 MHz, CDCl₃) δ 7.24 (s, 1H), 7.08 (s, 1H), 6.99 (d, J=7.8 Hz, 1H), 6.43-6.35 (m, 3H), 5.27 (s, 1H), 3.89 (t, J=6.5 Hz, 2H), 3.61-3.41 (m, 4H), 3.40-3.28 (m, 1H), 3.21-3.05 (m, 1H), 2.66 (t, J=7.6 Hz, 2H), 2.64-2.57 (m, 1H), 2.10-2.05 (m, 1H), 1.80-1.60 (m, 5H), 1.50-1.43 (m, 11H), 1.42-1.35 (m, 2H). ¹³C NMR (126 MHz, CDCl₃) δ 163.6 (dd, J=245.7, 15.9 Hz), 161.0 (t, J=13.8 Hz), 154.5, 148.6, 140.3, 136.2, 124.2, 115.8, 108.7, 98.2 (dd, J=21.3, 6.7 Hz), 96.0 (t, J=26.0 Hz), 79.3, 68.5, 49.4, 48.9, 45.5, 45.2, 44.9, 38.9, 38.0, 35.8, 31.8, 29.2, 28.9, 28.7, 28.5, 25.8. ¹⁹F NMR (376 MHz, CDCl₃) δ −109.5. HRMS (ESI⁺) found 530.2831 (C₂₉H₃₈F₂N₃O₄), required 530.2830 [M+H]⁺.

### tert-Butyl (R)-3-(((6-(6-phenoxyhexyl)benzo[d]oxazol-2-yl)amino)methyl)pyrrolidine-1-carboxylate, 20e

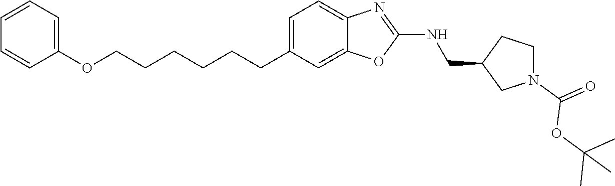 [SMILES: CC(C)(C)OC(=O)N1CC[C@H](CNc2nc3ccc(CCCCCCOc4ccccc4)cc3o2)C1]

Synthesized according to General Procedure 12. 0.060 g, 41%, yellow oil. ¹H NMR (500 MHz, CDCl₃) δ 7.29-7.23 (m, 3H), 7.07 (s, 1H), 6.98 (d, J=8.0 Hz, 1H), 6.92 (td, J=7.3, 1.2 Hz, 1H), 6.88 (d, J=7.7 Hz, 2H), 5.09 (s, 1H), 3.94 (t, J=6.5 Hz, 2H), 3.61-3.27 (m, 5H), 3.20-3.04 (m, 1H), 2.69-2.55 (m, 3H), 2.10-2.01 (m, 1H), 1.81-1.74 (m, 2H), 1.69-1.61 (m, 3H), 1.56-1.32 (m, 13H). ¹³C NMR (126 MHz, CDCl₃) δ 161.6, 159.0, 154.6, 148.7, 140.6, 136.2, 129.4, 124.2, 120.4, 115.9, 114.4, 108.6, 79.3, 67.7, 49.4, 45.5, 38.9, 35.8, 31.9, 28.9, 28.5, 25.9.

### tert-Butyl (R)-3-(((6-(6-(2-oxobenzo[d]oxazol-3(2H)-yl)hexyl)benzo[d]oxazol-2-yl)amino)methyl)pyrrolidine-1-carboxylate, 20f

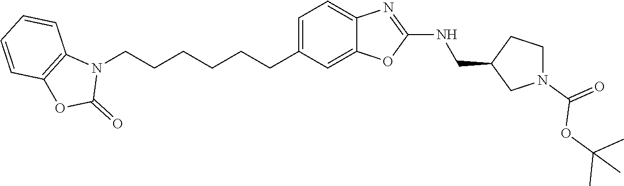 [SMILES: CC(C)(C)OC(=O)N1CC[C@H](CNc2nc3ccc(CCCCCCn4c(=O)oc5ccccc54)cc3o2)C1]

Synthesized according to General Procedure 12. 0.103 g, 80%, yellow oil. ¹H NMR (600 MHz, CDCl₃) δ 7.22 (d, J=7.7 Hz, 1H), 7.19 (d, J=7.6 Hz, 1H), 7.16 (td, J=7.8, 1.2 Hz, 1H), 7.09 (td, J=7.8, 1.3 Hz, 1H), 7.03 (d, J=4.2 Hz, 1H), 6.97-6.91 (m, 2H), 5.36 (s, 1H), 3.80 (t, J=7.3 Hz, 2H), 3.60-3.40 (m, 4H), 3.39-3.28 (m, 1H), 3.11 (ddd, J=47.7, 10.9, 6.9 Hz, 1H), 2.66-2.57 (m, 3H), 2.09-2.01 (m, 1H), 1.75 (p, J=7.3 Hz, 2H), 1.72-1.64 (m, 1H), 1.60 (p, J=7.0 Hz, 2H), 1.44 (d, J=4.3 Hz, 9H), 1.42-1.33 (m, 4H). ¹³C NMR (151 MHz, CDCl₃) δ 161.6, 154.6, 154.5, 148.6, 142.6, 140.6, 135.8, 131.1, 124.1, 124.0, 123.7, 122.2, 115.8, 110.0, 108.6, 108.2, 79.3, 79.3, 49.4, 48.9, 45.5, 45.4, 45.1, 44.9, 42.2, 38.8, 37.9, 35.7, 31.7, 30.9, 28.6, 28.5, 27.7, 26.5. HRMS (ESI⁺) found 535.2932 (C₃₀H₃₉N₄O₅), required 535.2915 [M+H]⁺.

### tert-Butyl (R)-3-(((6-(5-benzamidopentyl)benzo[d]oxazol-2-yl)amino)methyl)pyrrolidine-1-carboxylate, 20g

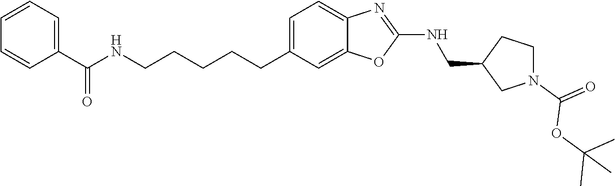 [SMILES: CC(C)(C)OC(=O)N1CC[C@H](CNc2nc3ccc(CCCCCNC(=O)c4ccccc4)cc3o2)C1]

Synthesized according to General Procedure 12. 0.052 g, 49%, yellow oil. ¹H NMR (600 MHz, CDCl₃) δ 7.74-7.71 (m, 2H), 7.50-7.46 (m, 1H), 7.42 (td, J=7.3, 1.3 Hz, 2H), 7.24 (d, J=8.5 Hz, 1H), 7.06 (s, 1H), 7.00-6.95 (m, 1H), 6.12 (s, 1H), 5.17 (s, 1H), 3.59-3.40 (m, 6H), 3.38-3.28 (m, 1H), 3.12 (ddd, J=47.8, 10.9, 6.9 Hz, 1H), 2.66 (t, J=7.6 Hz, 2H), 2.61 (h, J=7.4 Hz, 1H), 2.08-2.02 (m, 1H), 1.79-1.59 (m, 6H), 1.48-1.38 (m, 10H). ¹³C NMR (151 MHz, CDCl₃) δ 167.5, 161.6, 154.5, 148.7, 140.7, 135.8, 134.8, 131.3, 128.5, 126.8, 124.1, 115.9, 108.6, 79.3, 49.4, 48.9, 45.6, 45.5, 45.2, 44.9, 39.9, 38.8, 38.0, 35.7, 31.6, 31.5, 29.5, 29.2, 28.5, 26.3, 22.6. HRMS (ESI⁺) found 529.2788 (C₂₉H₃₈N₄O₄Na), required 529.2785 [M+Na]⁺.

### tert-Butyl (R)-3-(((6-(6-(2-fluorophenoxy)hexyl)benzo[d]oxazol-2-yl)amino)methyl)pyrrolidine-1-carboxylate, 20h

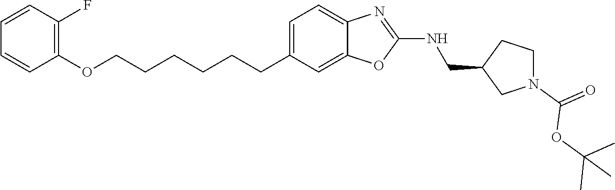 [SMILES: CC(C)(C)OC(=O)N1CC[C@H](CNc2nc3ccc(CCCCCCOc4ccccc4F)cc3o2)C1]

Synthesized according to General Procedure 12. 0.065 g, 46%, yellow oil. ¹H NMR (600 MHz, CDCl₃) δ 7.25-7.21 (m, 1H), 7.09-7.00 (m, 3H), 7.00-6.96 (m, 1H), 6.93 (td, J=8.3, 1.6 Hz, 1H), 6.86 (tdd, J=7.8, 4.6, 1.6 Hz, 1H), 5.85 (s, 1H), 4.00 (t, J=6.6 Hz, 2H), 3.60-3.41 (m, 4H), 3.39-3.28 (m, 1H), 3.12 (ddd, J=47.5, 10.9, 6.6 Hz, 1H), 2.68-2.57 (m, 3H), 2.08-2.02 (m, 1H), 1.85-1.77 (m, 2H), 1.74-1.60 (m, 3H), 1.54-1.36 (m, 13H). ¹³C NMR (151 MHz, CDCl₃) δ 161.7, 154.5 (d, J=7.0 Hz), 152.7 (d, J=245.4 Hz), 148.5, 147.1 (d, J=10.5 Hz), 140.2, 136.1, 124.2 (d, J=3.9 Hz), 120.7 (d, J=6.9 Hz), 116.0 (d, J=18.2 Hz), 115.6, 114.7 (d, J=2.1 Hz), 108.7, 106.2, 79.3, 69.2, 49.4, 48.9, 45.4, 45.3, 45.2, 44.9, 38.9, 38.0, 35.8, 31.8, 29.1, 28.8, 28.5, 25.7. ¹⁹F NMR (376 MHz, CDCl₃) δ −134.67. HRMS (ESI⁺) found 512.2926 (C₂₉H₃₉FN₃O₄), required 512.2919 [M+H]⁺.

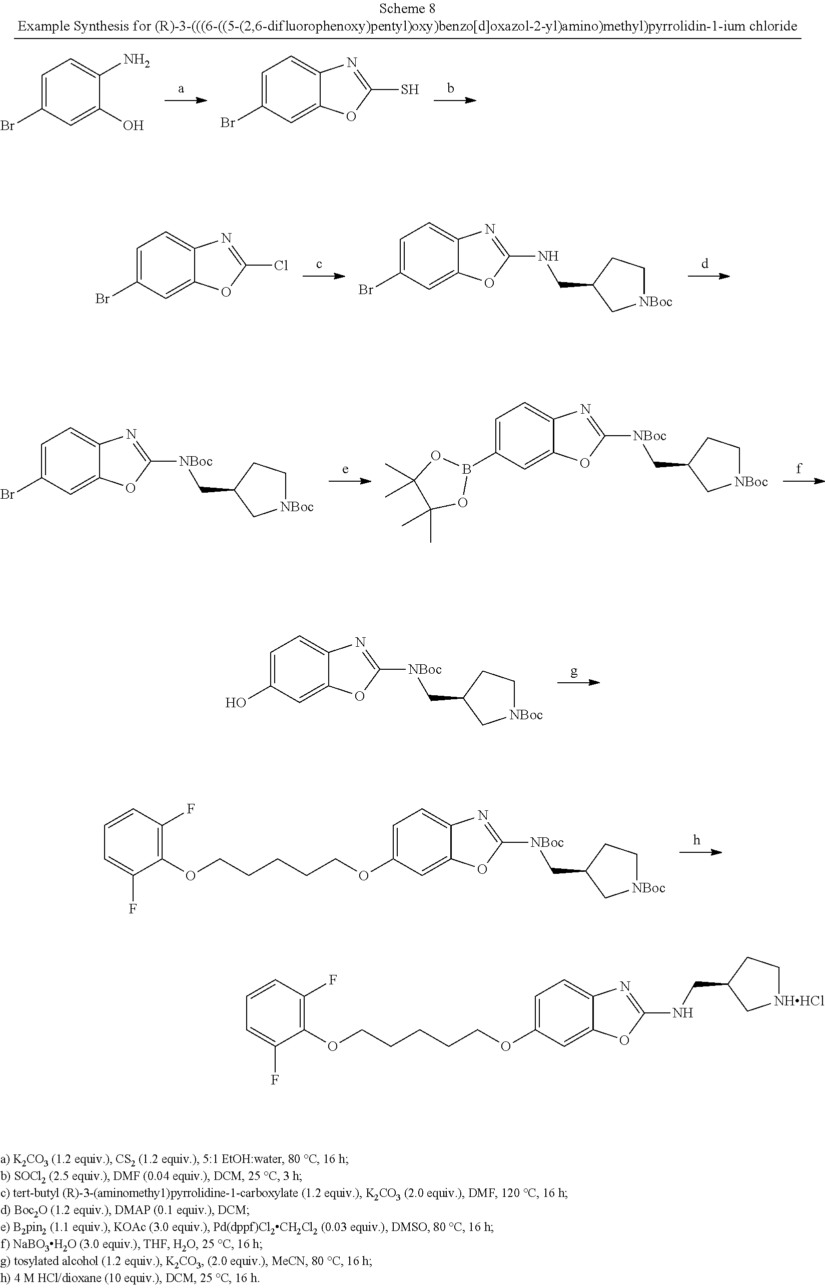

### tert-Butyl (S)-3-(((6-bromobenzo[d]oxazol-2-yl)(tert-butoxycarbonyl)amino)methyl)pyrrolidine-1-carboxylate

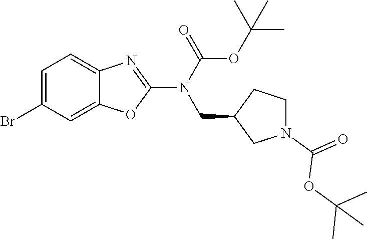 [SMILES: CC(C)(C)OC(=O)N1CC[C@H](CN(C(=O)OC(C)(C)C)c2nc3ccc(Br)cc3o2)C1]

To a stirring solution of tert-butyl (R)-3-(((6-bromobenzo[d]oxazol-2-yl)amino)methyl)pyrrolidine-1-carboxylate 5 (1.19 g, 1.0 eq., 3.00 mmol) in dichloromethane (7.51 mL) was added Boc₂O (786 mg, 0.828 mL, 1.2 eq., 3.60 mmol) and DMAP (36.7 mg, 0.1 eq., 300 mol) as a solution in dichloromethane (7.51 mL). The reaction was stirred overnight at 25° C., then quenched with water, extracted with ethyl acetate (3×20 mL), washed with brine (3×30 mL), dried with sodium sulfate, filtered, and concentrated in vacuo. The residue was purified by flash column chromatography (TeledyneISCO, 40 g SiO₂, Ø=5 cm, hexane/ethyl acetate 0→50%) to give tert-butyl (S)-3-(((6-bromobenzo[d]oxazol-2-yl)(tert-butoxycarbonyl)amino)methyl)pyrrolidine-1-carboxylate (1.255 g, 2.528 mmol, 84%) as an orange oil. ¹H NMR (500 MHz, CDCl₃) δ 7.61 (d, J=1.7 Hz, 1H), 7.47-7.38 (m, 2H), 4.04-3.94 (m, 2H), 3.57-3.39 (m, 2H), 3.36-3.24 (m, 1H), 3.16-3.06 (m, 1H), 2.67 (dp, J=14.3, 7.2 Hz, 1H), 1.96 (dq, J=12.3, 6.3 Hz, 1H), 1.73-1.61 (m, 1H), 1.56 (d, J=3.5 Hz, 9H), 1.43 (d, J=9.8 Hz, 9H). ¹³C NMR (126 MHz, CDCl₃) δ 158.1, 154.4, 151.4, 149.5, 139.9, 127.8, 119.8, 116.3, 113.5, 83.9, 79.2, 50.5, 50.4, 49.2, 48.9, 45.2, 44.8, 38.4, 37.5, 29.1, 28.5, 28.0. HRMS (ESI⁺) found 518.1283 (C₂₂H₃₀BrN₃O₅Na), required 518.1261 [M+Na]⁺.

### tert-butyl (S)-3-(((tert-butoxycarbonyl)(6-(4,4,5,5-tetramethyl-1,3,2-dioxaborolan-2-yl)benzo[d]oxazol-2-yl)amino)methyl)pyrrolidine-1-carboxylate

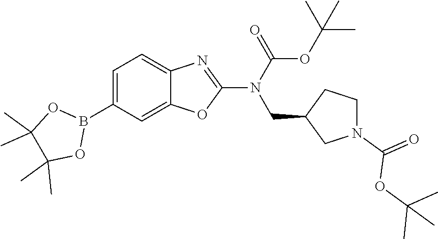 [SMILES: CC(C)(C)OC(=O)N1CC[C@H](CN(C(=O)OC(C)(C)C)c2nc3ccc(B4OC(C)(C)C(C)(C)O4)cc3o2)C1]

Synthesized according to General Procedure 14. 0.107 g, 65%, off-white amorphous solid. ¹H NMR (600 MHz, CDCl₃) δ 7.87 (s, 1H), 7.75 (d, J=7.9 Hz, OH), 7.57 (d, J=7.9 Hz, 1H), 4.06-3.97 (m, 2H), 3.56-3.39 (m, 2H), 3.36-3.24 (m, 1H), 3.18-3.06 (m, 1H), 2.75-2.64 (m, 1H), 2.00-1.92 (m, 1H), 1.73-1.64 (m, 2H), 1.56 (s, 9H), 1.43 (s, 9H), 1.36 (s, 12H). ¹¹B NMR (128 MHz, CDCl₃) δ 30.0.

### tert-Butyl (S)-3-(((tert-butoxycarbonyl)(6-((5-(2,6-difluorophenoxy)pentyl)oxy)benzo[d]oxazol-2-yl)amino)methyl)pyrrolidine-1-carboxylate, 20i

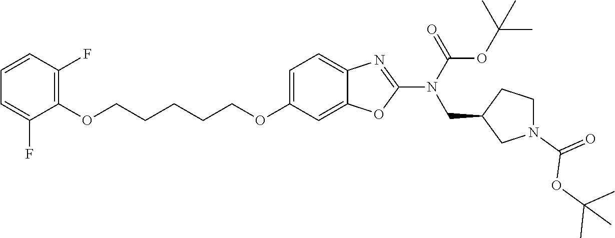 [SMILES: CC(C)(C)OC(=O)N1CC[C@H](CN(C(=O)OC(C)(C)C)c2nc3ccc(OCCCCCOc4c(F)cccc4F)cc3o2)C1]

Synthesized according to General Procedure 17. 18 mg, 49%. ¹H NMR (500 MHz, CDCl₃) δ 7.46 (d, J=8.7 Hz, 1H), 7.00 (s, 1H), 6.98-6.84 (m, 4H), 4.16 (t, J=6.4 Hz, 2H), 4.00 (t, J=6.4 Hz, 2H), 3.97-3.89 (m, 2H), 3.56-3.38 (m, 1H), 3.34-3.23 (m, 1H), 3.13-3.04 (m, 1H), 2.64 (dp, J=14.8, 7.4 Hz, 1H), 1.95 (dq, J=12.1, 6.2 Hz, 1H), 1.86 (tt, J=14.8, 6.6 Hz, 4H), 1.73-1.62 (m, 4H), 1.53 (d, J=3.8 Hz, 9H), 1.43 (d, J=11.0 Hz, 9H). ¹³C NMR (126 MHz, CDCl₃) δ 156.8, 156.7, 156.3 (dd, J=248.2, 5.6 Hz), 154.5, 152.0, 149.9, 135.7 (t, J=14.2 Hz), 134.1, 122.6 (t, J=9.3 Hz), 119.0, 112.7, 112.4-111.9 (m), 96.2, 83.2, 79.1, 74.4 (t, J=2.7 Hz), 68.6, 50.5, 50.5, 49.3, 48.9, 45.2, 44.9, 38.5, 37.6, 29.6, 29.1, 28.9, 28.1, 22.3. HRMS (ESI⁺) found 632.3159 (C₃₃H₄₃F₂N₃O₇), required 632.3142 [M+H]⁺.

### tert-Butyl (S)-3-(((tert-butoxycarbonyl)(6-(3-(3-cyclopropylphenoxy)propoxy)benzo[d]oxazol-2-yl)amino)methyl)pyrrolidine-1-carboxylate, 20j

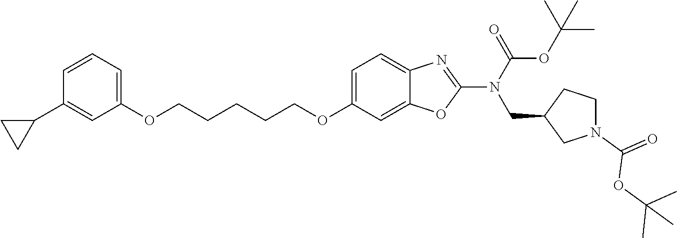 [SMILES: CC(C)(C)OC(=O)N1CC[C@H](CN(C(=O)OC(C)(C)C)c2nc3ccc(OCCCCCOc4cccc(C5CC5)c4)cc3o2)C1]

Synthesized according to General Procedure 17. 30 mg, 53%, colorless oil. ¹H NMR (500 MHz, CDCl₃) δ 7.46 (dd, J=8.4, 2.1 Hz, 1H), 7.15 (t, J=7.9 Hz, 1H), 7.03 (d, J=2.3 Hz, 1H), 6.90 (dd, J=8.7, 2.2 Hz, 1H), 6.72-6.66 (m, 2H), 6.65-6.61 (m, 1H), 4.17 (dt, J=8.2, 6.1 Hz, 4H), 4.01-3.89 (m, 2H), 3.58-3.39 (m, 2H), 3.35-3.24 (m, 1H), 3.14-3.05 (m, 1H), 2.64 (dp, J=14.7, 7.4 Hz, 1H), 2.27 (p, J=6.1 Hz, 2H), 1.96 (dq, J=12.2, 6.3 Hz, 1H), 1.86 (tt, J=8.4, 5.1 Hz, 1H), 1.73-1.61 (m, 1H), 1.54 (d, J=3.9 Hz, 9H), 1.43 (d, J=12.6 Hz, 9H), 0.98-0.90 (m, 2H), 0.68 (dt, J=6.6, 4.7 Hz, 2H). ¹³C NMR (126 MHz, CDCl₃) δ 158.9, 156.8, 156.6, 154.5, 151.9, 149.9, 145.8, 134.2, 129.2, 119.0, 118.4, 118.2, 112.7, 112.1, 112.0, 111.1, 111.1, 96.3, 83.3, 79.1, 65.8, 65.3, 64.1, 50.6, 50.5, 49.3, 48.9, 45.2, 44.9, 38.5, 37.6, 29.3, 29.1, 28.5, 28.1, 15.4, 9.2.

### tert-Butyl (S)-3-(((tert-butoxycarbonyl)(6-((4-((2,6-difluorophenoxy)methyl)cyclohexyl)oxy)benzo[d]oxazol-2-yl)amino)methyl)pyrrolidine-1-carboxylate, 20k

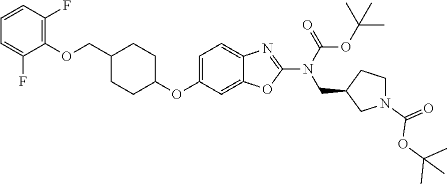 [SMILES: CC(C)(C)OC(=O)N1CC[C@H](CN(C(=O)OC(C)(C)C)c2nc3ccc(OC4CCC(COc5c(F)cccc5F)CC4)cc3o2)C1]

Synthesized according to General Procedure 17. 22 mg, 36%, colorless oil. ¹H NMR (600 MHz, CDCl₃) δ 7.45 (dd, J=8.8, 4.6 Hz, 1H), 7.05-7.01 (m, 1H), 6.96-6.84 (m, 4H), 4.56-4.51 (m, 1H), 3.99 (d, J=6.6 Hz, 2H), 3.97-3.89 (m, 2H), 3.55-3.39 (m, 2H), 3.34-3.23 (m, 1H), 3.08 (ddd, J=12.9, 10.8, 7.1 Hz, 1H), 2.69-2.57 (m, 1H), 2.08 (d, J=13.8 Hz, 1H), 1.99-1.88 (m, 2H), 1.77-1.71 (m, 3H), 1.69-1.57 (m, 4H), 1.53 (d, J=6.4 Hz, 9H), 1.42 (d, J=16.0 Hz, 9H). ¹³C NMR (151 MHz, CDCl₃) δ 156.7, 156.2 (dd, J=248.1, 5.9 Hz), 155.1, 154.4, 151.9 (d, J=11.6 Hz), 149.9 (d, J=4.6 Hz), 136.0 (t, J=14.3 Hz), 134.1, 122.5 (t, J=9.1 Hz), 118.9 (d, J=4.8 Hz), 114.3, 112.4-111.6 (m), 98.1, 83.3 (d, J=4.1 Hz), 79.5-78.9 (m), 73.0, 50.5, 50.4, 49.2, 48.8, 45.2, 44.8, 38.5, 37.5, 37.3, 31.6, 31.2, 29.1, 28.7, 28.5, 28.4, 28.0, 27.3, 23.5. ¹⁹F NMR (376 MHz, CDCl₃) δ −128.22, −128.30.

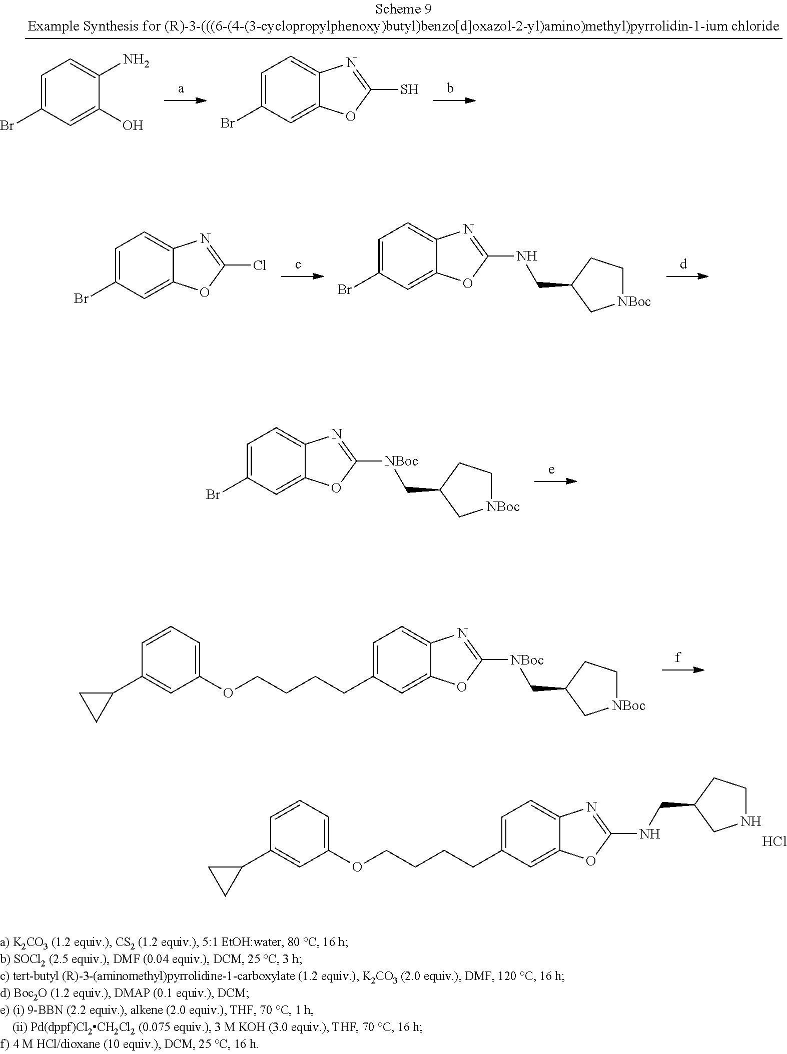

### tert-Butyl (S)-3-(((tert-butoxycarbonyl)(6-(4-(3-cyclopropylphenoxy)butyl)benzo[d]oxazol-2-yl)amino)methyl)pyrrolidine-1-carboxylate, 20l

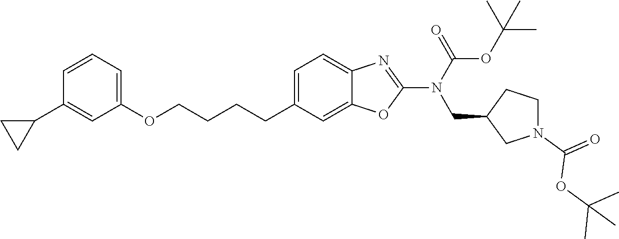 [SMILES: CC(C)(C)OC(=O)N1CC[C@H](CN(C(=O)OC(C)(C)C)c2nc3ccc(CCCCOc4cccc(C5CC5)c4)cc3o2)C1]

Synthesized according to General Procedure 12. 75 mg, 47%, yellow oil. ¹H NMR (600 MHz, CDCl₃) δ 7.49 (d, J=8.1 Hz, 1H), 7.30 (s, 1H), 7.19-7.11 (m, 2H), 6.70-6.64 (m, 2H), 6.62-6.58 (m, 1H), 4.03-3.92 (m, 4H), 3.56-3.40 (m, 2H), 3.35-3.25 (m, 1H), 3.15-3.07 (m, 1H), 2.78 (t, J=6.9 Hz, 2H), 2.71-2.61 (m, 1H), 1.96 (m, 1H), 1.90-1.72 (m, 5H), 1.71-1.59 (m, 1H), 1.55 (s, 9H), 1.43 (d, J=11.2 Hz, 9H), 0.96-0.90 (m, 2H), 0.72-0.65 (m, 2H). ¹³C NMR (151 MHz, CDCl₃) δ 159.1, 157.3, 154.5, 149.4, 145.7, 138.7, 138.6, 129.2, 129.2, 124.9, 118.4, 118.0, 112.0, 111.9, 111.0, 111.0, 109.7, 83.4, 79.1, 67.4, 60.4, 50.4, 49.2, 48.9, 45.2, 44.9, 38.5, 37.5, 35.7, 29.6, 29.1, 28.8, 28.5, 28.3, 28.1, 25.8, 15.4, 9.3.

### (R)-3-(((6-(4-(3-(tert-butyl)phenoxy)butyl)benzo[d]oxazol-2-yl)amino)methyl)pyrrolidine hydrochloride (21a)

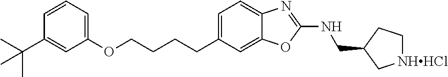

Synthesized according to General Procedure 4. 70 mg, 89%, off-white amorphous solid. ¹H NMR (500 MHz, d₄-MeOD) δ 7.48 (d, J=1.4 Hz, 1H), 7.38 (d, J=8.1 Hz, 1H), 7.32 (dd, J=8.1, 1.4 Hz, 1H), 7.16 (t, J=8.0 Hz, 1H), 6.95 (ddd, J=7.9, 1.9, 0.9 Hz, 1H), 6.89 (t, J=2.2 Hz, 1H), 6.69 (ddd, J=8.2, 2.5, 0.9 Hz, 1H), 3.98 (t, J=5.9 Hz, 2H), 3.68 (dd, J=7.4, 2.6 Hz, 2H), 3.57 (dd, J=11.9, 7.9 Hz, 1H), 3.52-3.44 (m, 1H), 3.37-3.32 (m, 1H), 3.12 (dd, J=11.9, 8.1 Hz, 1H), 2.83 (p, J=8.1 Hz, 3H), 2.33 (dtd, J=12.6, 7.5, 4.8 Hz, 1H), 1.92-1.77 (m, 5H), 1.29 (s, 9H). ¹³C NMR (126 MHz, d₄-MeOD) δ 160.3, 160.2, 153.9, 147.2, 141.7, 130.0, 129.0, 127.8, 118.7, 113.2, 113.2, 111.9, 111.8, 68.5, 46.4, 46.0, 38.6, 36.4, 35.5, 31.8, 29.9, 29.4, 29.1. HRMS (ESI⁺) found 422.2799 (C₂₆H₃₆N₃O₂), required 422.2808 [M+H]⁺.

### (R)-3-(((6-(6-(2,6-difluorophenoxy)hexyl)benzo[d]oxazol-2-yl)amino)methyl)pyrrolidin-1-ium chloride (21b)

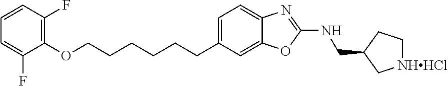

Synthesized according to General Procedure 4. 70 mg, 89%, off-white amorphous solid. ¹H NMR (600 MHz, d₄-MeOD) δ 7.46 (d, J=1.4 Hz, 1H), 7.37 (d, J=8.1 Hz, 1H), 7.30 (dd, J=8.1, 1.4 Hz, 1H), 7.04 (ddt, J=9.2, 7.5, 5.9 Hz, 1H), 6.99-6.93 (m, 2H), 4.09 (t, J=6.3 Hz, 2H), 3.70-3.63 (m, 2H), 3.57 (dd, J=11.9, 7.9 Hz, 1H), 3.47 (ddd, J=11.7, 8.4, 4.8 Hz, 1H), 3.37-3.32 (m, 1H), 3.11 (dd, J=11.9, 8.2 Hz, 1H), 2.83 (p, J=7.8 Hz, 1H), 2.77 (t, J=7.6 Hz, 2H), 2.33 (dtd, J=12.5, 7.5, 4.7 Hz, 1H), 1.87 (dq, J=13.2, 8.5 Hz, 1H), 1.77-1.66 (m, 4H), 1.54 (dtd, J=9.4, 7.4, 5.6 Hz, 2H), 1.46-1.37 (m, 2H). ¹³C NMR (126 MHz, d₄-MeOD) δ 160.1, 157.7 (dd, J=246.8, 5.6 Hz), 147.2, 142.0, 136.9 (t, J=14.4 Hz), 128.6, 127.8, 124.3 (t, J=9.4 Hz), 113.2 (dd, J=17.3, 5.5 Hz), 113.1, 111.8, 75.7 (t, J=2.9 Hz), 46.4, 46.0, 38.6, 36.6, 32.7, 30.9, 29.7, 29.1, 26.6. ¹⁹F NMR (376 MHz, d₄-MeOD) δ −130.45. HRMS (ESI⁺) found 430.2307 (C₂₄H₃₀F₂N₃O₂), required 430.2306 [M+H]⁺.

### (R)-3-(((6-(6-(3,5-difluorophenoxy)hexyl)benzo[d]oxazol-2-yl)amino)methyl)pyrrolidin-1-ium chloride (21c)

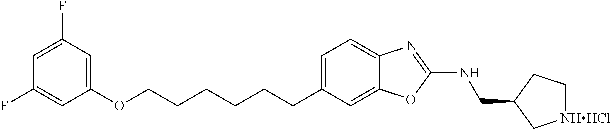

Synthesized according to General Procedure 4. 59 mg, 61%, off-white amorphous solid. ¹H NMR (600 MHz, d₄-MeOD) δ 7.45 (s, 1H), 7.37 (d, J=8.1 Hz, 1H), 7.29 (d, J=8.1 Hz, 1H), 6.53-6.45 (m, 3H), 3.95 (t, J=6.3 Hz, 2H), 3.72-3.64 (m, 2H), 3.58 (dd, J=11.9, 7.9 Hz, 1H), 3.51-3.45 (m, 1H), 3.37-3.33 (m, 1H), 3.13 (dd, J=11.9, 8.0 Hz, 1H), 2.84 (p, J=7.7 Hz, 1H), 2.77 (t, J=7.6 Hz, 2H), 2.38-2.30 (m, 1H), 1.88 (dq, J=13.2, 8.4 Hz, 1H), 1.79-1.73 (m, 2H), 1.70 (p, J=7.6 Hz, 2H), 1.51 (p, J=7.2 Hz, 2H), 1.41 (p, J=7.6 Hz, 2H). ¹³C NMR (151 MHz, d₄-MeOD) δ 165.1 (dd, J=244.5, 16.0 Hz), 162.8 (t, J=13.8 Hz), 160.2, 147.2, 141.9, 128.9, 127.7, 113.1, 111.8, 99.2 (dd, J=23.2, 6.1 Hz), 96.6 (t, J=26.5 Hz), 69.7, 46.4, 46.0, 38.6, 36.6, 32.7, 29.9, 29.7, 29.1, 26.8. ¹⁹F NMR (376 MHz, d₄-MeOD) δ −111.5. HRMS (ESI⁺) found 430.2303 (C₂₄H₃₀F₂N₃O₂), required 430.2306 [M+H]⁺.

### (R)-3-(((6-bromobenzo[d]oxazol-2-yl)amino)methyl)pyrrolidin-1-ium chloride, 1u

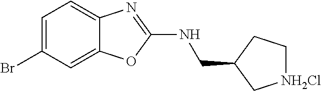

Synthesized according to General Procedure 4. 40 mg, 0.12 mmol, 95%, off-white amorphous solid. ¹H NMR (500 MHz, d₄-MeOD) δ 7.82 (d, J=1.8 Hz, 1H), 7.59 (dd, J=8.4, 1.7 Hz, 1H), 7.39 (d, J=8.4 Hz, 1H), 3.69-3.65 (m, 2H), 3.59-3.53 (m, 1H), 3.48 (td, J=7.6, 3.9 Hz, 1H), 3.37-3.33 (m, 1H), 3.12 (dd, J=11.9, 8.0 Hz, 1H), 2.84 (h, J=7.7 Hz, 1H), 2.38-2.26 (m, 1H), 1.93-1.82 (m, 1H). ¹³C NMR (126 MHz, d₄-MeOD) δ 160.8, 147.7, 132.0, 130.4, 117.4, 115.4, 115.1, 72.4, 46.4, 46.0, 38.6, 29.1. HRMS (ESI⁺) found 296.0399 (C₁₂H₁₅BrN₃O), required 296.0399 [M+H]⁺.

### (R)-3-(((6-(6-phenoxyhexyl)benzo[d]oxazol-2-yl)amino)methyl)pyrrolidin-1-ium chloride, 21e

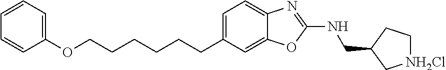

Synthesized according to General Procedure 4. 9 mg, 20%, yellow amorphous solid. ¹H NMR (600 MHz, d₄-MeOD) δ 7.23 (dd, J=8.8, 7.3 Hz, 2H), 7.17-7.13 (m, 2H), 7.01 (dd, J=8.0, 1.6 Hz, 1H), 6.90-6.85 (m, 3H), 3.93 (t, J=6.4 Hz, 2H), 3.51-3.39 (m, 4H), 3.30-3.26 (m, 1H), 3.06 (dd, J=11.9, 8.1 Hz, 1H), 2.76 (p, J=7.6 Hz, 1H), 2.68 (t, J=7.6 Hz, 2H), 2.24 (dtd, J=12.8, 7.6, 5.0 Hz, 1H), 1.83 (dq, J=13.3, 8.3 Hz, 1H), 1.78-1.73 (m, 2H), 1.66 (p, J=7.5 Hz, 2H), 1.55-1.48 (m, 2H), 1.44-1.37 (m, 2H). ¹³C NMR (151 MHz, d₄-MeOD) δ 163.9, 160.5, 149.7, 140.8, 137.9, 130.4, 125.5, 125.2, 121.5, 115.9, 115.4, 109.8, 68.7, 46.4, 45.3, 39.4, 36.7, 33.1, 30.3, 29.9, 29.0, 27.0. HRMS (ESI⁺) found 394.2491 (C₂₄H₃₂ClN₃O₂), required 394.2495 [M+H]⁺.

### (R)-3-(((6-(6-(2-oxobenzo[d]oxazol-3(2H)-yl)hexyl)benzo[d]oxazol-2-yl)amino)methyl)pyrrolidin-1-ium chloride, 21f

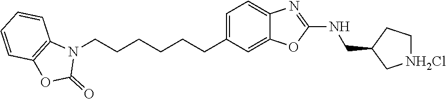

Synthesized according to General Procedure 4. 59 mg, 61%, off-white amorphous solid. ¹H NMR (600 MHz, d₄-MeOD) δ 7.28 (d, J=1.4 Hz, 1H), 7.27-7.21 (m, 3H), 7.19-7.11 (m, 3H), 3.85 (t, J=7.0 Hz, 2H), 3.58 (d, J=7.2 Hz, 2H), 3.53 (dd, J=11.9, 7.9 Hz, 1H), 3.45 (ddd, J=11.7, 8.4, 4.9 Hz, 1H), 3.33 (d, J=8.2 Hz, 1H), 3.09 (dd, J=11.9, 8.1 Hz, 1H), 2.80 (hept, J=7.7 Hz, 1H), 2.68 (t, J=7.6 Hz, 2H), 2.29 (dtd, J=12.7, 7.6, 4.9 Hz, 1H), 1.86 (dq, J=13.3, 8.4 Hz, 1H), 1.80-1.73 (m, 2H), 1.67-1.60 (m, 2H), 1.39 (p, J=3.8 Hz, 4H). ¹³C NMR (151 MHz, d₄-MeOD) δ 160.5, 155.0, 147.0, 142.6, 138.6, 131.1, 125.3, 123.8, 122.2, 113.0, 109.5, 109.4, 108.7, 45.0, 44.3, 41.6, 37.6, 35.2, 31.3, 28.2, 27.7, 27.2, 26.0. HRMS (ESI⁺) found 435.2402 (C₂₅H₃₁ClN₄O₃), required 435.2396 [M+H]⁺.

### (R)-3-(((6-(5-benzamidopentyl)benzo[d]oxazol-2-yl)amino)methyl)pyrrolidin-1-ium chloride, 21g

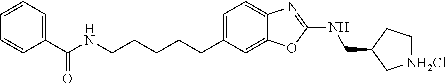

Synthesized according to General Procedure 4. 38 mg, 84%, off-white amorphous solid. ¹H NMR (600 MHz, d₄-MeOD) δ 7.79-7.74 (m, 2H), 7.57-7.49 (m, 1H), 7.45 (t, J=7.7 Hz, 2H), 7.39-7.36 (m, 1H), 7.29 (d, J=8.0 Hz, 1H), 7.22 (dd, J=8.1, 1.3 Hz, 1H), 3.61 (d, J=7.0 Hz, 2H), 3.56-3.51 (m, 1H), 3.46 (ddd, J=12.4, 8.1, 4.6 Hz, 1H), 3.36 (t, J=7.1 Hz, 2H), 3.36-3.32 (m, 1H), 3.10 (dd, J=11.8, 7.9 Hz, 1H), 2.81 (p, J=7.6 Hz, 1H), 2.74 (t, J=7.5 Hz, 2H), 2.31 (dtd, J=12.4, 7.4, 4.7 Hz, 1H), 1.86 (dq, J=13.0, 8.3 Hz, 1H), 1.72 (q, J=7.6 Hz, 2H), 1.66 (p, J=7.4 Hz, 2H), 1.46-1.38 (m, 2H). ¹³C NMR (151 MHz, d₄-MeOD) δ 170.2, 160.2, 147.2, 141.7, 135.8, 132.6, 129.5, 129.1, 128.2, 128.2, 127.7, 113.1, 111.8, 46.4, 46.0, 40.7, 38.6, 36.5, 32.4, 30.2, 29.2, 27.2. HRMS (ESI⁺) found 407.2449 (C₂₄H₃₁ClN₄O₂), required 407.2447 [M+H]⁺.

### (R)-3-(((6-(6-(2-fluorophenoxy)hexyl)benzo[d]oxazol-2-yl)amino)methyl)pyrrolidin-1-ium chloride, 21h

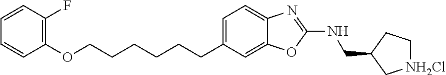

Synthesized according to General Procedure 4. 12 mg, 21%, off-white amorphous solid. ¹H NMR (600 MHz, d₄-MeOD) δ 7.32 (s, 1H), 7.28 (d, J=8.0 Hz, 1H), 7.19-7.16 (m, 1H), 7.10-7.03 (m, 3H), 6.90 (dddd, J=8.9, 7.4, 4.5, 2.3 Hz, 1H), 4.03 (t, J=6.3 Hz, 2H), 3.59 (d, J=7.0 Hz, 2H), 3.58-3.43 (m, 3H), 3.11 (dd, J=11.9, 8.1 Hz, 1H), 2.81 (p, J=7.7 Hz, 1H), 2.74 (t, J=7.6 Hz, 2H), 2.30 (tdd, J=9.4, 7.5, 4.8 Hz, 1H), 1.87 (dq, J=13.2, 8.4 Hz, 1H), 1.80 (dt, J=14.6, 6.6 Hz, 2H), 1.70 (p, J=7.6 Hz, 2H), 1.60-1.52 (m, 2H), 1.47-1.40 (m, 2H). ¹³C NMR (126 MHz, d₄-MeOD δ 154.1 (d, J=244.0 Hz), 148.5 (d, J=10.3 Hz), 140.0, 126.6, 126.0, 125.5 (d, J=4.0 Hz), 122.0 (d, J=6.9 Hz), 116.9 (d, J=18.3 Hz), 116.1, 114.5, 110.8, 110.6, 70.2, 46.4, 45.6, 39.2, 39.1, 36.6, 32.9, 30.2, 29.8, 29.1, 26.9. ¹⁹F NMR (376 MHz, d₄-MeOD) δ −137.0. HRMS (ESI⁺) found 412.2390 (C₂₄H₃₁FN₃O₂), required 412.2400 [M+H]⁺.

### (R)-3-(((6-((5-(2,6-difluorophenoxy)pentyl)oxy)benzo[d]oxazol-2-yl)amino)methyl)pyrrolidin-1-ium chloride, 21i

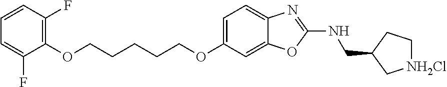

Synthesized according to General Procedure 4. 7 mg, 50%, off-white amorphous solid. ¹H NMR (500 MHz, d₄-MeOD) δ 7.35 (d, J=8.7 Hz, 1H), 7.23 (d, J=2.3 Hz, 1H), 7.09-6.91 (m, 4H), 4.14 (t, J=6.2 Hz, 2H), 4.06 (t, J=6.3 Hz, 2H), 3.71-3.60 (m, 2H), 3.57 (dd, J=11.9, 7.9 Hz, 1H), 3.53-3.43 (m, 1H), 3.38-3.31 (m, 1H), 3.12 (dd, J=12.0, 8.2 Hz, 1H), 2.83 (hept, J=7.7 Hz, 1H), 2.38-2.28 (m, 1H), 1.92-1.79 (m, 5H), 1.76-1.66 (m, 2H). ¹³C NMR (126 MHz, d₄-MeOD) δ 159.0, 157.2, 156.3 (dd, J=246.9, 5.6 Hz), 135.5 (t, J=14.3 Hz), 123.4, 123.0 (t, J=9.4 Hz), 113.0, 112.4, 112.1-111.6 (m), 97.6, 74.3 (t, J=2.9 Hz), 68.7, 45.0, 44.5, 37.3, 29.4, 28.5, 27.7, 22.1. ¹⁹F NMR (376 MHz, d₄-MeOD) δ −130.4. HRMS (ESI⁺) found 432.2100 (C₂₃H₂₈F₂N₃O₃), required 432.2099 [M+H]⁺.

### (R)-3-(((6-(3-(3-cyclopropylphenoxy)propoxy)benzo[d]oxazol-2-yl)amino)methyl)pyrrolidin-1-ium chloride, 21j

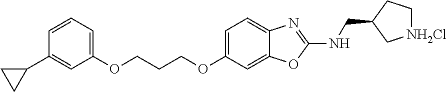

Synthesized according to General Procedure 4. 50 mg, 63%, off-white amorphous solid. ¹H NMR (600 MHz, d₄-MeOD) δ 7.34 (d, J=8.7 Hz, 1H), 7.26 (d, J=2.3 Hz, 1H), 7.11 (t, J=7.8 Hz, 1H), 7.04 (dd, J=8.8, 2.3 Hz, 1H), 6.69 (ddd, J=8.2, 2.5, 1.0 Hz, 1H), 6.65-6.60 (m, 2H), 4.22 (t, J=6.1 Hz, 2H), 4.14 (t, J=6.1 Hz, 2H), 3.67-3.60 (m, 2H), 3.56 (dd, J=11.9, 7.9 Hz, 1H), 3.46 (ddd, J=11.7, 8.4, 4.8 Hz, 1H), 3.37-3.32 (m, 1H), 3.09 (dd, J=11.9, 8.2 Hz, 1H), 2.81 (hept, J=7.8 Hz, 1H), 2.32 (dtd, J=12.5, 7.6, 4.8 Hz, 1H), 2.25 (p, J=6.1 Hz, 2H), 1.90-1.81 (m, 2H), 0.96-0.90 (m, 2H), 0.67-0.62 (m, 2H). ¹³C NMR (126 MHz, d₄-MeOD) δ 157.5, 155.5, 145.0, 144.1, 127.3, 122.1, 116.1, 111.4, 110.9, 110.0, 109.4, 96.2, 63.9, 62.2, 43.4, 43.0, 35.7, 27.4, 26.2, 13.2, 6.7. HRMS (ESI⁺) found 408.2291 (C₂₄H₃N₃O₃), required 408.2282 [M+H]⁺.

### (R)-3-(((6-((4-((2,6-difluorophenoxy)methyl)cyclohexyl)oxy)benzo[d]oxazol-2-yl)amino)methyl)pyrrolidin-1-ium chloride, 21k

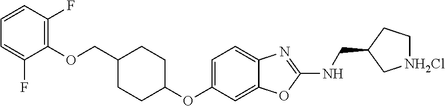

Synthesized according to General Procedure 4. 15 mg, 91%, d.r. 86:14, off-white amorphous solid. ¹H NMR (600 MHz, d₄-MeOD) δ 7.36 (d, J=8.6 Hz, 0.89H), 7.34 (app. s, 0.11H), 7.28 (d, J=2.3 Hz, 0.83H), 7.26 (d, J=2.2 Hz, 0.17H), 7.08-6.94 (m, 4H), 4.66 (p, J=3.0 Hz, 1H), 4.01-3.94 (m, 2H), 3.69-3.61 (m, 2H), 3.57 (dd, J=11.9, 7.9 Hz, 1H), 3.52-3.42 (m, 1H), 3.37-3.32 (m, 1H), 3.11 (dd, J=11.9, 8.2 Hz, 1H), 2.82 (hept, J=7.7 Hz, 1H), 2.33 (dtd, J=12.5, 7.5, 4.8 Hz, 1H), 2.09-2.00 (m, 2H), 1.94-1.80 (m, 2H), 1.76-1.65 (m, 3H), 1.63-1.54 (m, 2H), 1.52-1.27 (m, 1H). ¹³C NMR (151 MHz, d₄-MeOD) δ 160.3, 157.6 (dd, J=246.7, 5.6 Hz), 156.9, 147.9, 137.2 (t, J=14.3 Hz), 124.3 (t, J=9.4 Hz), 116.0, 113.8, 113.3 (dd, J=18.1, 5.0 Hz), 100.7, 80.7 (t, J=2.7 Hz), 74.4, 46.4, 45.9, 38.7, 38.6, 32.3, 29.7, 29.1, 28.2, 24.5. Minor diastereomer signals observed: δ 157.1, 147.9, 124.6, 115.8, 113.8, 100.5, 80.4 (t, J=2.8 Hz), 78.5, 39.0, 28.5. ¹⁹F NMR (376 MHz, d₄-MeOD) δ −130.4. HRMS (ESI⁺) found 458.2255 (C₂₅H₃₀F₂N₃O₃), required 458.2250 [M+H]⁺.

### (R)-3-(((6-(4-(3-cyclopropylphenoxy)butyl)benzo[d]oxazol-2-yl)amino)methyl)pyrrolidin-1-ium chloride, 21l

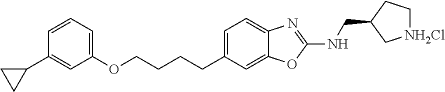

Synthesized according to General Procedure 4. 40 mg, 0.090 mmol, 78%, off-white amorphous solid. ¹H NMR (600 MHz, d₄-MeOD) δ 7.49 (d, J=1.4 Hz, 1H), 7.38 (d, J=8.1 Hz, 1H), 7.32 (dd, J=8.1, 1.4 Hz, 1H), 7.10 (t, J=7.9 Hz, 1H), 6.64 (ddd, J=8.2, 2.5, 0.9 Hz, 1H), 6.61 (d, J=7.7 Hz, 1H), 6.57 (t, J=2.1 Hz, 1H), 3.95 (t, J=6.0 Hz, 2H), 3.72-3.63 (m, 2H), 3.57 (dd, J=11.9, 7.9 Hz, 1H), 3.48 (ddd, J=12.6, 8.3, 4.7 Hz, 1H), 3.37-3.32 (m, 1H), 3.12 (dd, J=11.9, 8.1 Hz, 1H), 2.88-2.77 (m, 3H), 2.37-2.31 (m, 1H), 1.92-1.76 (m, 6H), 0.95-0.90 (m, 2H), 0.66-0.61 (m, 2H). ¹³C NMR (151 MHz, d₄-MeOD) δ 159.1, 158.7, 145.8, 145.6, 140.3, 128.8, 127.4, 126.4, 117.4, 111.7, 111.4, 110.8, 110.5, 67.0, 45.0, 44.6, 37.2, 35.0, 28.4, 28.0, 27.8, 14.8, 8.3. HRMS (ESI⁺) found 406.2487 (C₂₅H₃₂N₃O₂), required 406.2489 [M+H]⁺.

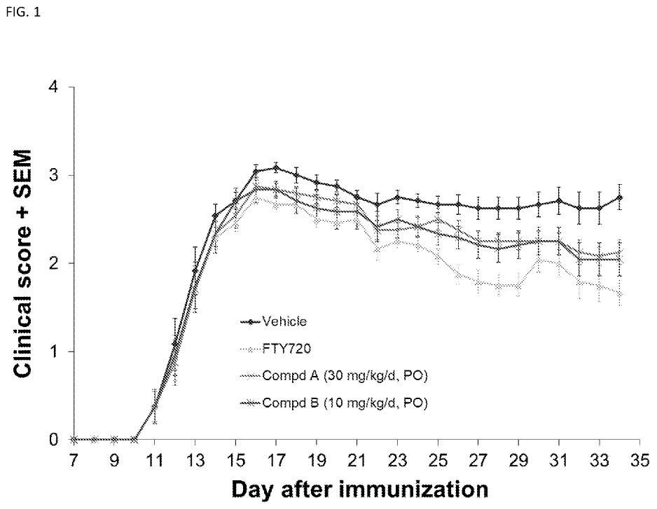

Treatment of Autoimmune Disorders: Sphingosine 1-phosphate (S1P) is important for the correct positioning of immune cells. Further, drugs that interfere with S1P signaling modulate the immune response at the level of lymphocyte trafficking. Such drugs are approved for treatment of autoimmune diseases such as multiple sclerosis and ulcerative colitis. The new chemical entities claimed herein modulate lymphocyte trafficking by blocking a different node in the S1P signaling pathway. Therefore, these molecules are expected to be efficacious in treating patients suffering from a condition requiring immunosuppression such as autoimmune disorders and organ transplantation. Among the numerous autoimmune disorders are multiple sclerosis, inflammatory bowel diseases (for example, ulcerative colitis and Crohn's disease), systemic lupus erythematosus, rheumatoid arthritis, type 1 diabetes, scleroderma, Sjogren's syndrome, atopic dermatitis, uveitis, vasculitis, and so forth. Transplanted organs include kidney, heart, liver, lung, intestine, stomach, pancreatic beta cells, and stem cells. Included are complications such as graft vs host disease that can arise from organ or stem cell transplants. FIG. 1. shows a representative data set for Spns2-dependent S1P transport blockers (STBs) in the EAE (Experimental Autoimmune Encephalomyelitis) model of multiple sclerosis. Mice (female, C57BL/6 strain, 11 weeks old) are vaccinated with MOG33-35 peptide and treated with pertussis toxin on day 0. As animals become symptomatic, they are assigned randomly to treatment groups (N=12) and the clinical scores are determined until day 35. Treatments are administered once daily by oral gavage. Positive control (FTY720 a/k/a fingolimod) dose is 3 mg/kg/d. STBs Compd A and Compd B, both in accordance with embodiments disclosed herein, are administered at 30 and 10 mg/kg/d respectively.

1. A compound according to Formula I: 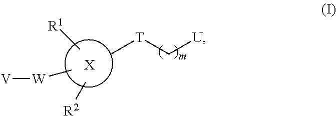
wherein
X is a C₆-C₁₀-aryl or 5- to 10-membered heteroaryl (wherein 1 to 4 heteroaryl ring members are independently selected from N, O, and S);
R¹ and R² are independently selected from the group consisting of H, C₁-C₆-alkyl, C₁-C₆-alkoxy, C₁-C₆-haloalkoxy, C₃-C₈-cycloalkyl, C₁-C₆-haloalkyl, CN, and halo;
W is a bond, O, NH, —NHC(O)—, or —O—(N═)C(R)— (wherein R is H or C₁-C₆-alkyl);
U is either:
(A) an optionally fused, bridged, or spiro-fused 6- to 8-membered heterocycloalkyl wherein 2 ring atoms are N, wherein
V is selected from the group consisting of H, C₁-C₁₄-alkyl, C₂-C₁₂-alkenyl, (C₆-C₁₀)aryl, (C₆-C₁₀)heteroaryl, —C₁-C₁₀-alkyl-(C₆-C₁₀)aryl, —C₂-C₁₂-alkenyl-(C₆-C₁₀)aryl, —C₁-C₁₀-alkyl-(C₃-C₈)cycloalkyl, -(3- to 14-membered heterocycloalkyl) (wherein 1-4 heterocycloalkyl members are independently selected from N, O, and S), —(C₁-C₁₀)alkyl-(3- to 14-membered heterocycloalkyl) (wherein 1-4 heterocycloalkyl members are independently selected from N, O, and S);
OR
(B) a monocyclic 4- to 7-membered heterocycloalkyl (wherein 1-4 heterocycloalkyl members are independently selected from N, O, and S), wherein
V is selected from the group consisting of H, —C₁-C₁₄-alkyl, —C₁-C₁₀-alkyl-O—(C₃-C₁₄)cycloalkyl, —C₁-C₁₀-alkyl-O—(C₆-C₁₀)aryl, —C₁-C₁₀-alkyl-NR^{x}—(C₆-C₁₀)aryl, —C₁-C₁₀-alkyl-NR^{x}C(O)(C₆-C₁₀)aryl, —C₁-C₁₀-alkyl-C(O)NR^{x}(C₆-C₁₀)aryl, —C₂-C₁₂-alkenyl-O—(C₆-C₁₀)aryl, —C₁-C₁₀-alkyl-O—(C₆-C₁₀)heteroaryl (wherein 1 to 4 heteroaryl ring members are independently selected from N, O, and S), and —C₂-C₁₂-alkenyl-O—(C₆-C₁₀)heteroaryl (wherein 1 to 4 heteroaryl ring members are independently selected from N, O, and S);
T is —C(O)— or —NR^{x}C(O)—;
R is H or C₁-C₆-alkyl;
m is an integer selected from 0, 1, 2, 3, 4, 5, and 6;
wherein each alkyl, alkoxy, alkenyl, aryl, cycloalkyl, heterocycloalkyl, and heteroaryl is optionally substituted with 1-5 substituents independently selected from the group consisting of hydroxy, halo, C₁-C₆-alkyl, C₁-C₆-haloalkoxy, C₁-C₆-haloalkyl, —NR′₂, —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkoxy, C₃-C₁₄-cycloalkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl); and
wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S;
or a pharmaceutically acceptable salt thereof.

2. The compound or pharmaceutically acceptable salt thereof according to claim 1, wherein:
U is (A) an optionally substituted and optionally fused, bridged, or spiro-fused 6- to 8-membered heterocycloalkyl wherein 2 ring atoms are N, and
V is selected from the group consisting of H, C₁-C₁₄-alkyl, C₂-C₁₂-alkenyl, (C₆-C₁₀)aryl, (C₆-C₁₀)heteroaryl, —C₁-C₁₀-alkyl-(C₆-C₁₀)aryl, —C₂-C₁₂-alkenyl-(C₆-C₁₀)aryl, —C₁-C₁₀-alkyl-(C₃—C₈)cycloalkyl, -(3- to 14-membered heterocycloalkyl) (wherein 1-4 heterocycloalkyl members are independently selected from N, O, and S), —(C₁-C₁₀)alkyl-(3- to 14-membered heterocycloalkyl) (wherein 1-4 heterocycloalkyl members are independently selected from N, O, and S).

3. The compound or pharmaceutically acceptable salt thereof according to claim 2, wherein V is C₁-C₁₄-alkyl.

4. The compound or pharmaceutically acceptable salt thereof according to claim 3, wherein V is C₈-C₁₂-alkyl.

5. The compound or pharmaceutically acceptable salt thereof according to claim 2, wherein U is selected from the group consisting of optionally substituted:

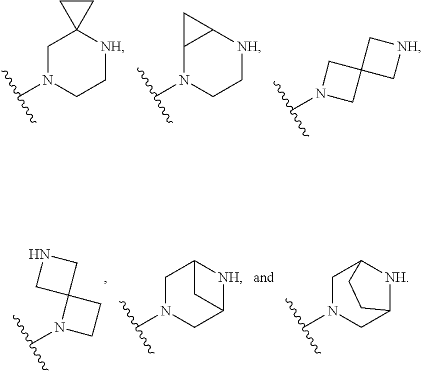

6. The compound or pharmaceutically acceptable salt thereof according to claim 1, wherein:
U is (B) an optionally substituted monocyclic 4- to 7-membered heterocycloalkyl (wherein 1-4 heterocycloalkyl members are independently selected from N, O, and S), and
V is selected from the group consisting of —C₁-C₁₀-alkyl-O—(C₆-C₁₀)aryl, —C₁-C₁₀-alkyl-NR^{x}—(C₆-C₁₀)aryl, —C₁-C₁₀-alkyl-NR^{x}C(O)(C₆-C₁₀)aryl, —C₁-C₁₀-alkyl-C(O)NR^{x}(C₆-C₁₀)aryl, —C₂-C₁₂-alkenyl-O—(C₆-C₁₀)aryl, —C₁-C₁₀-alkyl-O—(C₆-C₁₀)heteroaryl (wherein 1 to 4 heteroaryl ring members are independently selected from N, O, and S), and —C₂-C₁₂-alkenyl-O—(C₆-C₁₀)heteroaryl (wherein 1 to 4 heteroaryl ring members are independently selected from N, O, and S).

7. The compound or pharmaceutically acceptable salt thereof according to claim 6, wherein U is a monocyclic 5- to 7-membered heterocycloalkyl (wherein 1 or 2 heterocycloalkyl members are independently selected from N and O).

8. The compound or pharmaceutically acceptable salt thereof according to claim 7, wherein U is selected from optionally substituted pyrrolidinyl, oxazolidinyl, isoxazolidinyl, imidazolinyl, pyrazolidinyl, piperidinyl, piperazinyl, and morpholinyl.

9. The compound or pharmaceutically acceptable salt thereof according to claim 8, wherein U is piperazinyl.

10. The compound or pharmaceutically acceptable salt thereof according to claim 6, wherein V is —C₁-C₁₀-alkyl-O—(C₆-C₁₀)aryl.

11. The compound or pharmaceutically acceptable salt thereof according to claim 10, wherein V is —C₃-C₈-alkyl-O-phenyl.

12. The compound or pharmaceutically acceptable salt thereof according to claim 11, wherein X is C₆-C₁₀-aryl.

13. The compound or pharmaceutically acceptable salt thereof according to claim 12, wherein X is phenyl.

14. The compound or pharmaceutically acceptable salt thereof according to claim 1, wherein W is a bond or O.

15. The compound or pharmaceutically acceptable salt thereof according to claim 14, wherein W is a bond.

16. The compound or pharmaceutically acceptable salt thereof according to claim 1, wherein T is —NR^{x}C(O)—.

17. The compound or pharmaceutically acceptable salt thereof according to claim 1, wherein T is —C(O)—.

18. The compound or pharmaceutically acceptable salt thereof according to claim 1, wherein m is 0.

19. The compound or pharmaceutically acceptable salt thereof according to claim 1, wherein:
U is (A) an optionally substituted and optionally fused, bridged, or spiro-fused 6- to 8-membered heterocycloalkyl wherein 2 ring atoms are N;
X is C₆-C₁₀-aryl;
T is —NR^{x}C(O)— wherein R^{x} is H;
V is C₈-C₁₂-alkyl; and
W is a bond; and
m is 0.

20. The compound or pharmaceutically acceptable salt thereof according to claim 1, wherein:
U is (B) an optionally substituted 5- to 7-membered heterocycloalkyl (wherein 1 or 2 heterocycloalkyl members are independently selected from N and O);
X is C₆-C₁₀-aryl;
T is —NR^{x}C(O)— wherein R^{x} is H;
V is —C₁-C₁₀-alkyl-O—(C₆-C₁₀)aryl; and
W is a bond; and
m is 0.

21. The compound or pharmaceutically acceptable salt thereof according to claim 1, wherein the compound is selected from the following table:

| | |
|---|---|
| 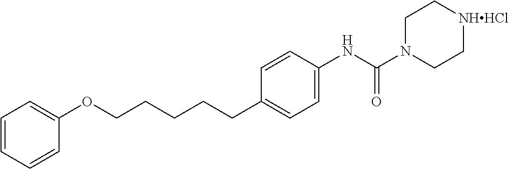 | 3a |
| 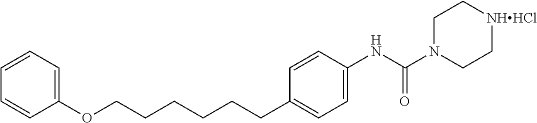 | 3b |
| 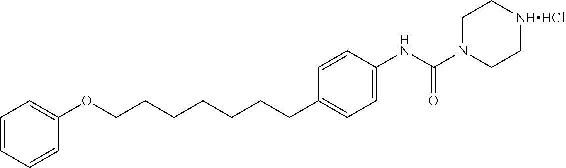 | 3c |
| 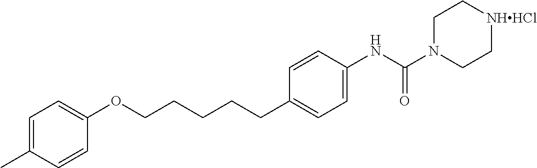 | 3d |
| 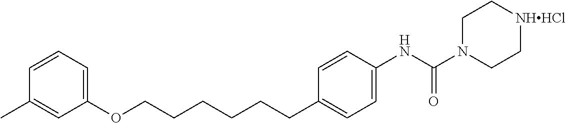 | 3e |
| 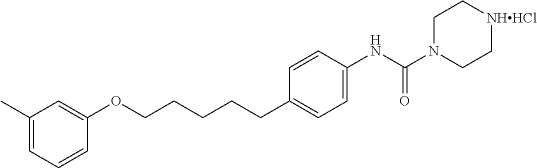 | 3f |
| 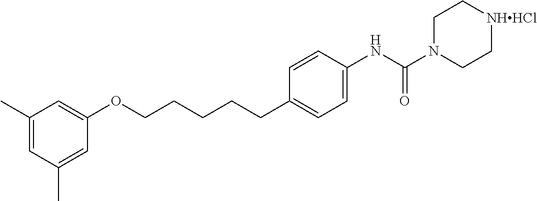 | 3g |
| 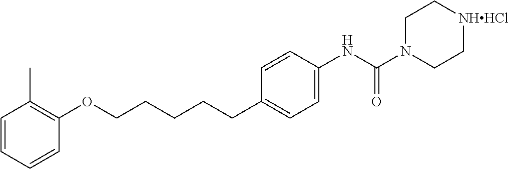 | 3h |
| 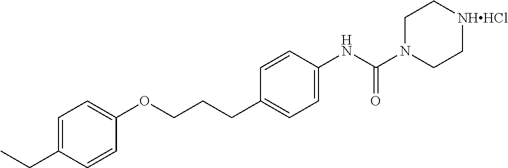 | 3i |
| 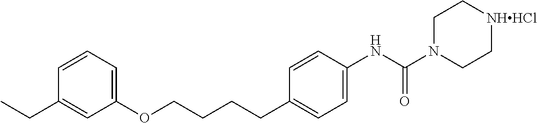 | 3j |
| 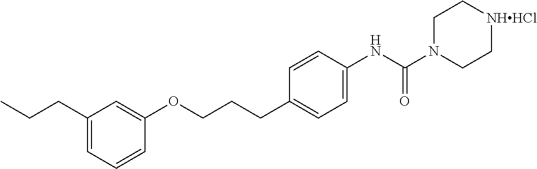 | 3k |
| 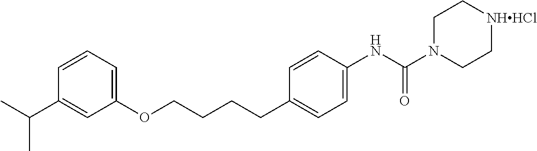 | 3l |
| 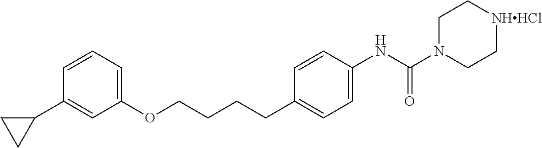 | 3m |
| 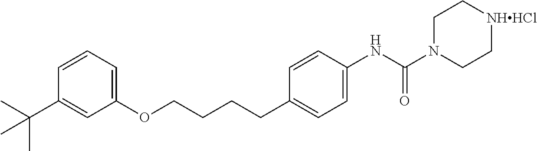 | 3n |
| 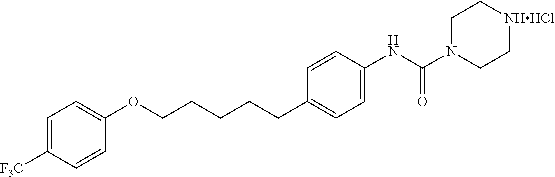 | 3o |
| 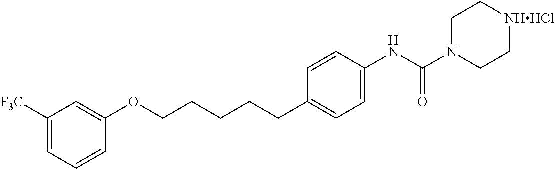 | 3p |
| 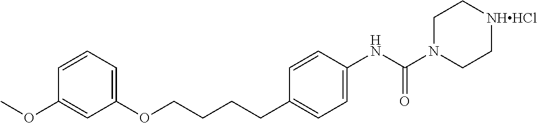 | 3q |
| 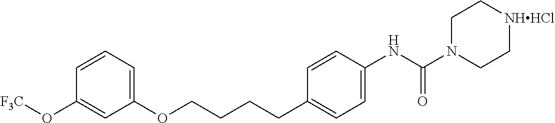 | 3r |
| 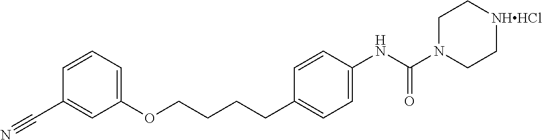 | 3s |
| 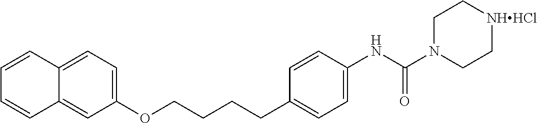 | 3t |
| 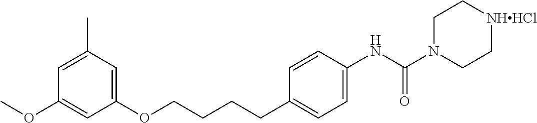 | 3u |
| 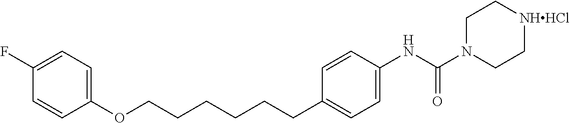 | 3v |
| 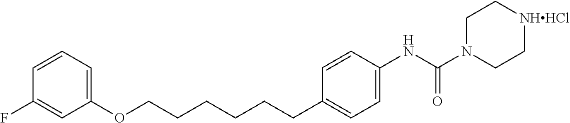 | 3w |
| 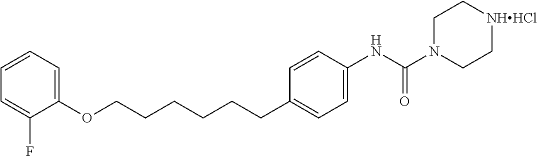 | 3x |
| 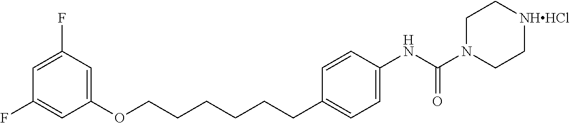 | 3y |
| 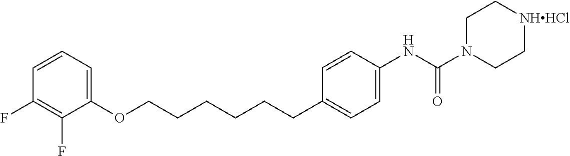 | 3z |
| 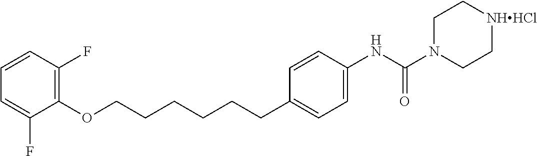 | 3aa |
| 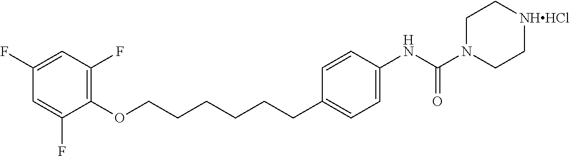 | 3ab |
| 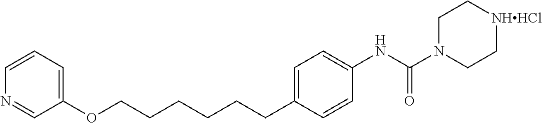 | 3ac |
| 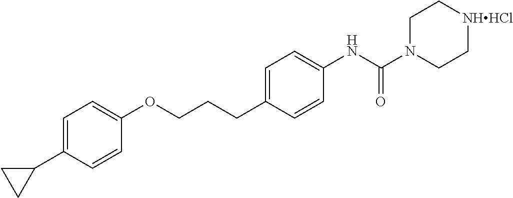 | 3ad |
| 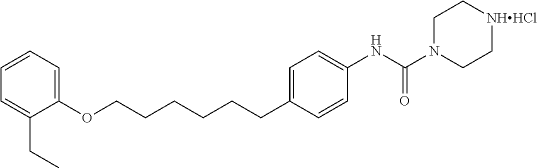 | 3ae |
| 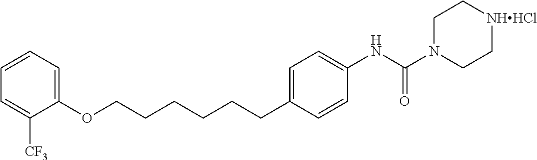 | 3af |
| 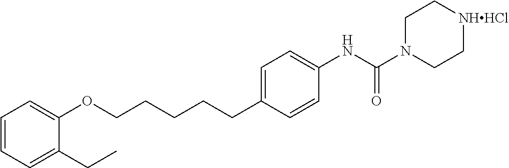 | 3ag |
| 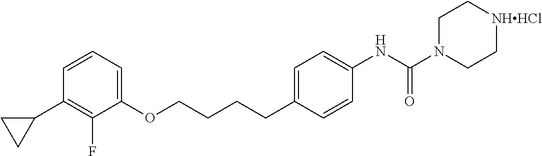 | 3ah |
| 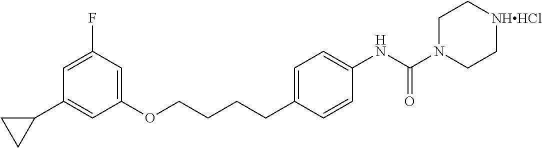 | 3ai |
| 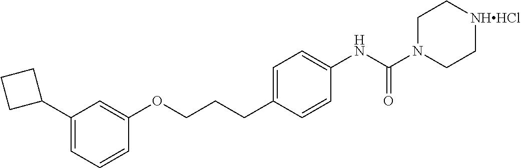 | 3aj |
| 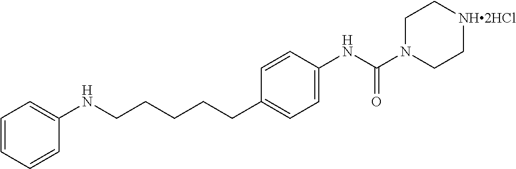 | 3ak |
| 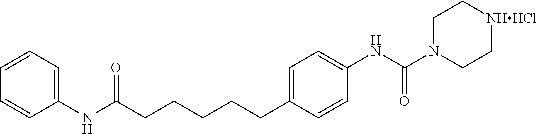 | 3al |
| 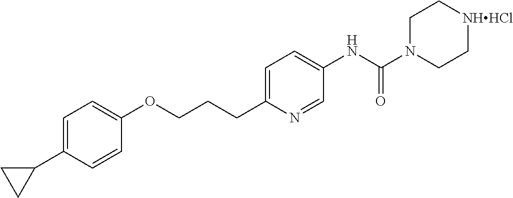 | 3am |
| 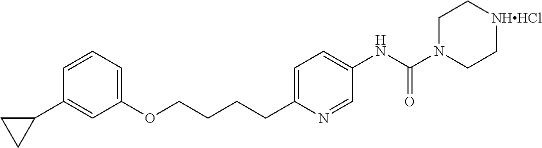 | 3an |
| 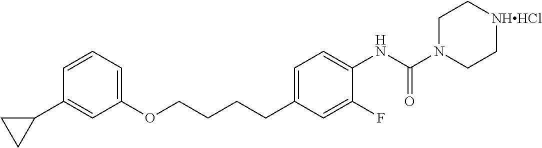 | 3ao |
| 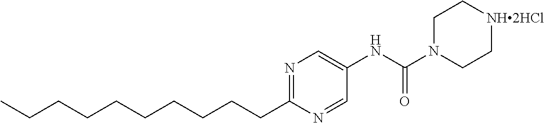 | 3ap |
| 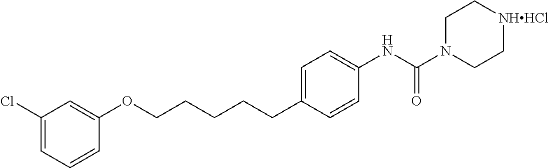 | 3aq |
| 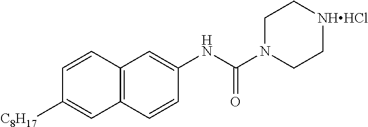 | 3ar |
| 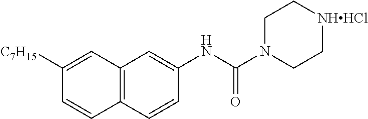 | 3as |
| 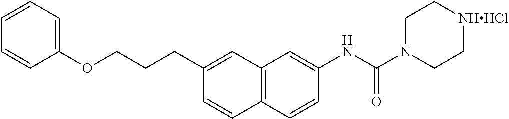 | 3at |
| 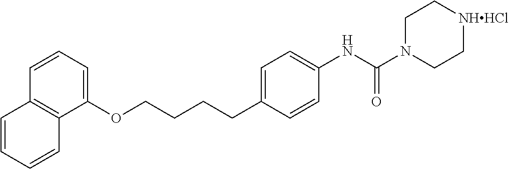 | 3au |
| 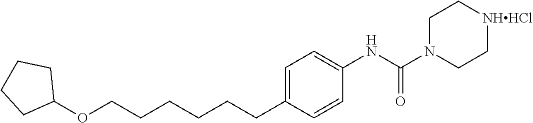 | 3av |
| 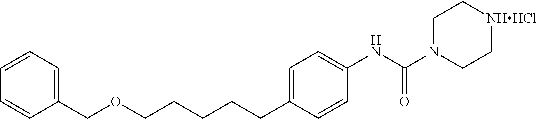 | 3aw |
| 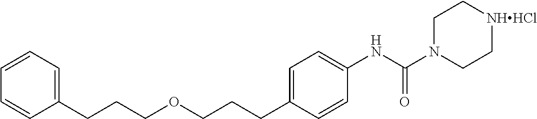 | 3ax |
| 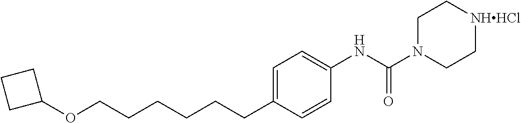 | 3ay |
| 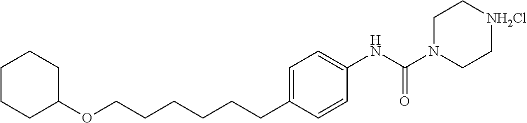 | 3az |
| 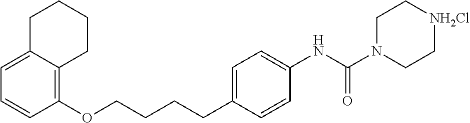 | 3ba |
| 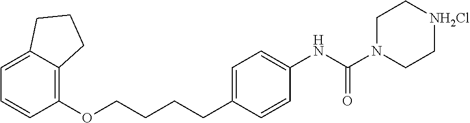 | 3bb |
| 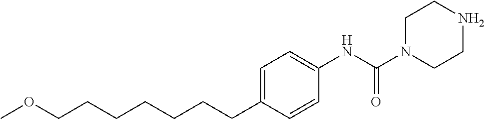 [SMILES: COCCCCCCCc1ccc(NC(=O)N2CCNCC2)cc1] | 3bc |
| 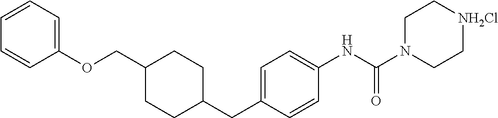 | 3bd |
| 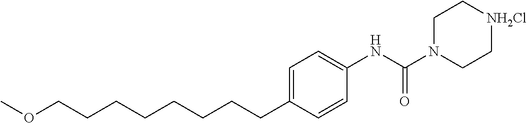 | 3be |
| 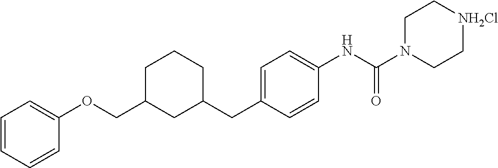 | 3bf |
| 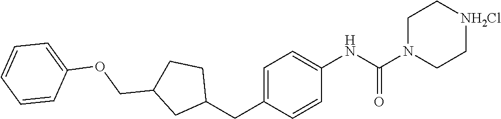 | 3bg |
| 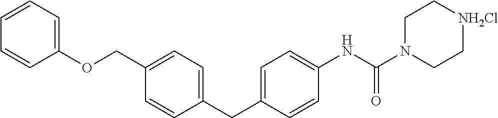 | 3bh |
| 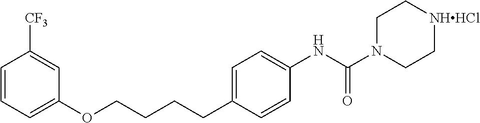 | 3bi |
| 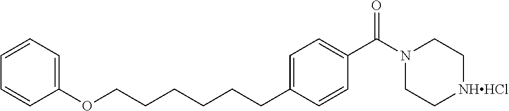 | 5a |
| 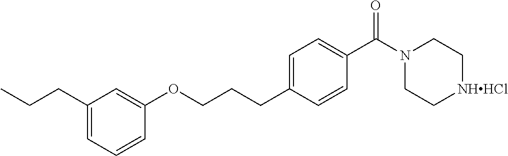 | 5b |
| 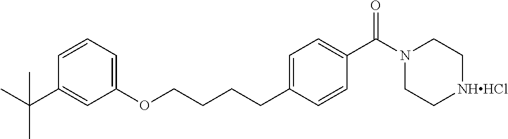 | 5c |
| 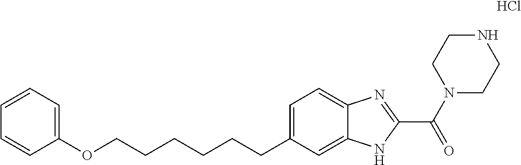 [SMILES: Cl ; O=C(c1nc2ccc(CCCCCCOc3ccccc3)cc2[nH]1)N1CCNCC1] | 18a |
| 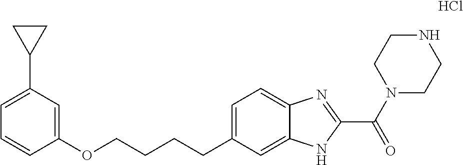 [SMILES: Cl ; O=C(c1nc2ccc(CCCCOc3cccc(C4CC4)c3)cc2[nH]1)N1CCNCC1] | 18b |
| 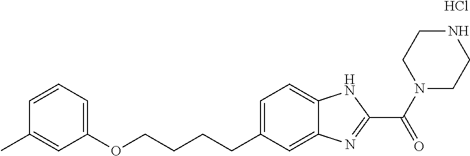 [SMILES: Cl ; Cc1cccc(OCCCCc2ccc3[nH]c(C(=O)N4CCNCC4)nc3c2)c1] | 18e |
| 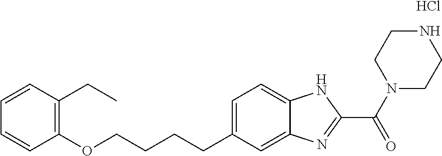 [SMILES: Cl ; CCc1ccccc1OCCCCc1ccc2[nH]c(C(=O)N3CCNCC3)nc2c1] | 18f |
| 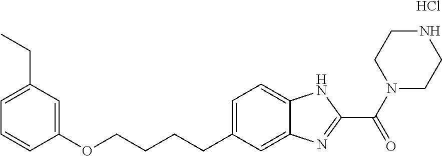 [SMILES: Cl ; CCc1cccc(OCCCCc2ccc3[nH]c(C(=O)N4CCNCC4)nc3c2)c1] | 18g |
| 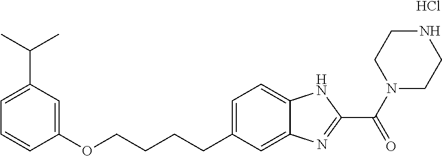 [SMILES: Cl ; CC(C)c1cccc(OCCCCc2ccc3[nH]c(C(=O)N4CCNCC4)nc3c2)c1] | 18h |
| 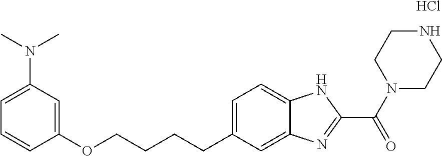 [SMILES: Cl ; CN(C)c1cccc(OCCCCc2ccc3[nH]c(C(=O)N4CCNCC4)nc3c2)c1] | 18i |
| 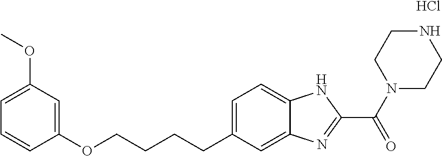 [SMILES: Cl ; COc1cccc(OCCCCc2ccc3[nH]c(C(=O)N4CCNCC4)nc3c2)c1] | 18j |
| 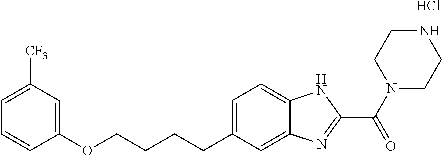 [SMILES: Cl ; O=C(c1nc2cc(CCCCOc3cccc(C(F)(F)F)c3)ccc2[nH]1)N1CCNCC1] | 18k |
| 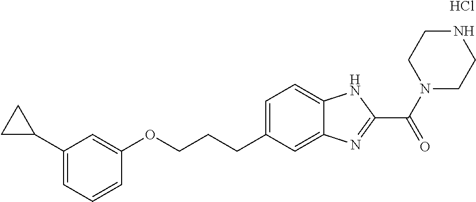 [SMILES: Cl ; O=C(c1nc2cc(CCCOc3cccc(C4CC4)c3)ccc2[nH]1)N1CCNCC1] | 18l |
| 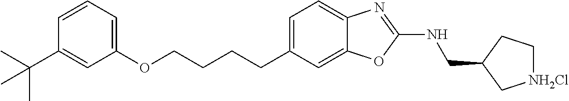 | 21a |
| 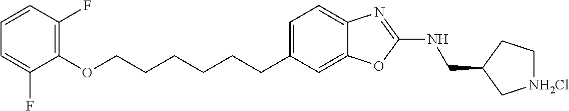 | 21b |
| 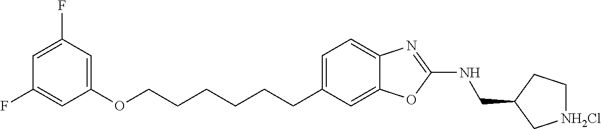 | 21c |
| 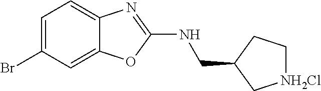 | 21d |
| 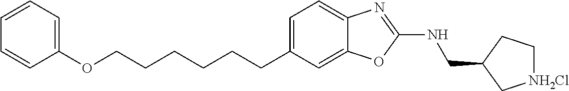 | 21e |
| 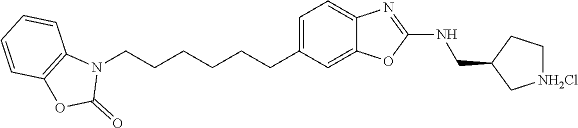 | 21f |
| 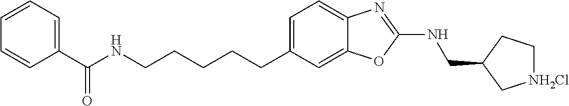 | 21g |
| 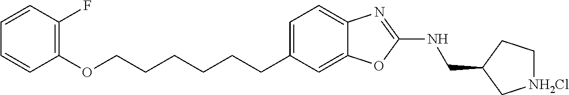 | 21h |
| 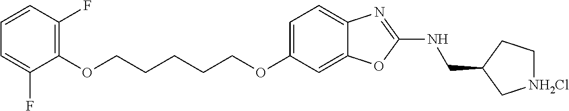 | 21i |
| 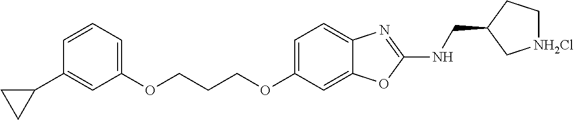 | 21j |
| 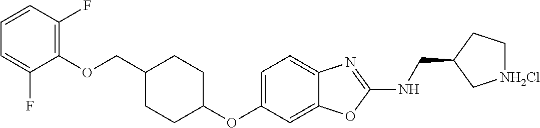 | 21k |
| 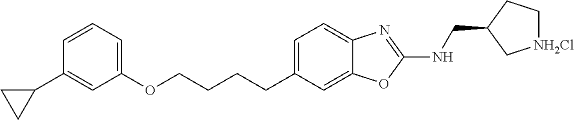 | 21l |

22. The compound or pharmaceutically acceptable salt thereof according to claim 1, wherein the compound is selected from the following table:

| Compound | Structure |
|---|---|
| 7a | 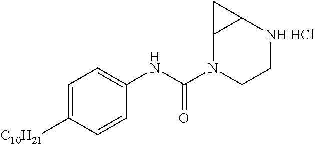 |
| 7b | 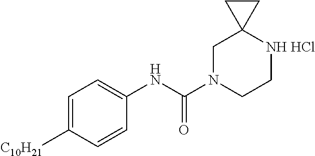 |
| 7c | 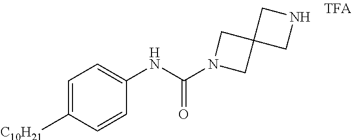 |
| 7d | 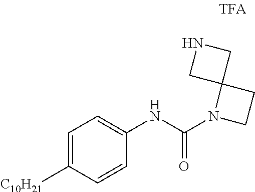 |
| 7e | 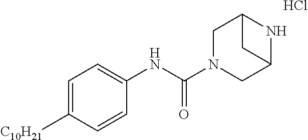 |
| 9a | 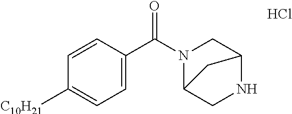 |
| 9b | 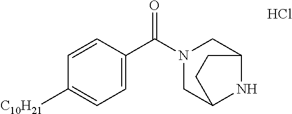 |
| 7f | 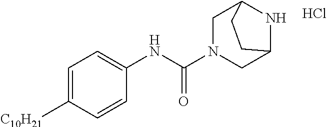 |

23. A pharmaceutical composition comprising a compound or a pharmaceutically acceptable salt thereof according to claim 1.

24. A method of inhibiting spinster homolog 2 (SPNS2), comprising contacting SPNS2 with an effective amount of a compound according to claim 1.

25. The method according to claim 24, wherein the contacting occurs in vivo.

26. A method of treating a patient afflicted by a disease, comprising administering to the patient a therapeutically effective amount of a compound according to claim 1, wherein the disease is a neoplastic disease, an allergic disease, an autoimmune disease or a fibrotic disease.

27. The method according to claim 26, wherein
the neoplastic disease is metastatic neoplasms,
the allergic disease is asthma,
the autoimmune disease is multiple sclerosis, one or more progressive forms of multiple sclerosis, type I diabetes, inflammatory bowel diseases including Crohn's disease and ulcerative colitis, Grave's disease, Addison's disease, dermatomyositis, myasthenia gravis, systemic lupus erythematosus, scleroderma, psoriasis, or atopic dermatitis, and
the fibrotic disease is chronic kidney disease.

28.-36. (canceled)
